SEQUENCE LISTING (51 sequences)

SEQ ID NO: 1 - protein, 9 aa, synthetic construct
GYSITSDYA

SEQ ID NO: 2 - protein, 7 aa, synthetic construct
ISYSGST

SEQ ID NO: 3 - protein, 18 aa, synthetic construct
AKSGGDYYGSSSYWYFDV

SEQ ID NO: 4 - protein, 37 aa, synthetic construct
DMPALEQLDLHSNVLMDIEDGAFEGLPRLTHLNLSRN

SEQ ID NO: 5 - protein, 12 aa, synthetic construct
QSLLNSRSQKNY

SEQ ID NO: 6 - skipped

SEQ ID NO: 7 - protein, 9 aa, synthetic construct
QNDHSYPFT

SEQ ID NO: 8 - protein, 41 aa, synthetic construct
HQDKVPCKMVDKKVSCQVLGLLQVPSVLPPDTETLDLSGNQ

SEQ ID NO: 9 - protein, 8 aa, synthetic construct
GFTFSNYV

SEQ ID NO: 10 - protein, 8 aa, synthetic construct
ISSGGSYT

SEQ ID NO: 11 - protein, 13 aa, synthetic construct
ARGYDNGDYVMDY

SEQ ID NO: 12 - protein, 120 aa, synthetic construct
EVKLVESGGGSVKPGGSLKLSCAASGFTFSNYVMSWVRQTPEKRLEWVATISSGGSYTYYPDSVKGRLTISRDNAKNTLYLQMSSLRSEDTAMYYCARGYDNGDYVMDYWGQGTSVTVSS

SEQ ID NO: 13 - protein, 10 aa, synthetic construct
ESVDTYGNSF

SEQ ID NO: 14 - skipped

SEQ ID NO: 15 - protein, 9 aa, synthetic construct
QQTNEHPPT

SEQ ID NO: 16 - protein, 111 aa, synthetic construct
DIVLTQSPASLAVSLGQRATISCRASESVDTYGNSFMHWYQQIPGQPPKVLIYRASNLESGIPARFSGSGSRTDFTLTINPVEAGDVATYYCQQTNEHPPTFGGGTKLEIK

SEQ ID NO: 17 - protein, 12 aa, synthetic construct
LRTLSLENSLTR

SEQ ID NO: 18 - protein, 125 aa, synthetic construct
DVQLQESGPGLVKPSQTLSLTCTVSGYSITSDYAWNWIRQFPGNKLEWMGYISYSGSTSYTPSLKSRITISRDTSKNHFFLQLSSVTAEDTATYYCAKSGGDYYGSSSYWYFDVWGQGTTVTVSS

SEQ ID NO: 19 - protein, 125 aa, synthetic construct
DVQLQESGPGLVKPSQTLSLTCTVSGYSITSDYAWNWIRQFPGNKLEWMGYISYSGSTSYTPSLKSRITISRDTSKNHFFLKLSSVTAADTATYYCAKSGGDYYGSSSYWYFDVWGQGTTVTVSS

SEQ ID NO: 20 - protein, 125 aa, synthetic construct
QVQLQESGPGLVKPSQTLSLTCTVSGYSITSDYAWNWIRQFPGNKLEWMGYISYSGSTSYTPSLKSRVTISRDTSKNHFSLKLSSVTAADTAVYYCAKSGGDYYGSSSYWYFDVWGQGTMVTVSS

SEQ ID NO: 21 - protein, 125 aa, synthetic construct
QVQLQESGPGLVKPSQTLSLTCTVSGYSITSDYAWNWIRQFPGNKLEWMGYISYSGSTSYTPSLKSRITISRDTSKNHFFLKLSSVTAADTAVYYCAKSGGDYYGSSSYWYFDVWGQGTMVTVSS

SEQ ID NO: 22 - protein, 114 aa, synthetic construct
DIVLTQSPSSLAVSAGERVTMSCKSSQSLLNSRSQKNYLAWYQQKPGQPPKLLIYGASTRGSGVPDRFSGSGSGTDFTLTISSVQAEDVAVYYCQNDHSYPFTFGSGTKLEIKR

SEQ ID NO: 23 - protein, 114 aa, synthetic construct
DIVLTQSPSSLAVSLGERATMNCKSSQSLLNSRSQKNYLAWYQQKPGQPPKLLIYGASTRGSGVPDRFSGSGSGTDFTLTISSLQAEDVAVYYCQNDHSYPFTFGQGTKLEIKR

SEQ ID NO: 24 - protein, 114 aa, synthetic construct
DIVLTQSPSSLAVSLGERVTMNCKSSQSLLNSRSQKNYLAWYQQKPGQPPKLLIYGASTRGSGVPDRFSGSGSGTDFTLTISSVQAEDVAVYYCQNDHSYPFTFGQGTKLEIKR

SEQ ID NO: 25 - DNA, 375 bp, synthetic construct
  source 1..375: mol_type = other DNA
gacgtgcagctgcaggagagcggccccggcctggtgaagcccagccagaccctgagcctgacctgcaccgtgagcggctacagcatcaccagcgactacgcctggaactggatcagacagttccccggcaacaagctggagtggatgggctacatcagctacagcggcagcaccagctacacccccagcctgaagagcagaatcaccatcagcagagacaccagcaagaaccacttcttcctgcagctgagcagcgtgaccgccgaggacaccgccacctactactgcgccaagagcggcggcgactactacggcagcagcagctactggtacttcgacgtgtggggccagggcaccaccgtgaccgtgagcagc

SEQ ID NO: 26 - DNA, 375 bp, synthetic construct
  source 1..375: mol_type = other DNA
gacgtgcagctgcaggagagcggccccggcctggtgaagcccagccagaccctgagcctgacctgcaccgtgagcggctacagcatcaccagcgactacgcctggaactggatcagacagttccccggcaacaagctggagtggatgggctacatcagctacagcggcagcaccagctacacccccagcctgaagagcagaatcaccatcagcagagacaccagcaagaaccacttcttcctgaagctgagcagcgtgaccgccgccgacaccgccacctactactgcgccaagagcggcggcgactactacggcagcagcagctactggtacttcgacgtgtggggccagggcaccaccgtgaccgtgagcagc

SEQ ID NO: 27 - DNA, 375 bp, synthetic construct
  source 1..375: mol_type = other DNA
caggtgcagctgcaggagagcggccccggcctggtgaagcccagccagaccctgagcctgacctgcaccgtgagcggctacagcatcaccagcgactacgcctggaactggatcagacagttccccggcaacaagctggagtggatgggctacatcagctacagcggcagcaccagctacacccccagcctgaagagcagaatcaccatcagcagagacaccagcaagaaccacttcttcctgaagctgagcagcgtgaccgccgccgacaccgccgtgtactactgcgccaagagcggcggcgactactacggcagcagcagctactggtacttcgacgtgtggggccagggcaccatggtgaccgtgagcagc

SEQ ID NO: 28 - DNA, 375 bp, synthetic construct
  source 1..375: mol_type = other DNA
caggtgcagctgcaggagagcggccccggcctggtgaagcccagccagaccctgagcctgacctgcaccgtgagcggctacagcatcaccagcgactacgcctggaactggatcagacagttccccggcaacaagctggagtggatgggctacatcagctacagcggcagcaccagctacacccccagcctgaagagcagagtgaccatcagcagagacaccagcaagaaccacttcagcctgaagctgagcagcgtgaccgccgccgacaccgccgtgtactactgcgccaagagcggcggcgactactacggcagcagcagctactggtacttcgacgtgtggggccagggcaccatggtgaccgtgagcagc

SEQ ID NO: 29 - DNA, 342 bp, synthetic construct
  source 1..342: mol_type = other DNA
gacatcgtgctgacccagagccccagcagcctggccgtgagcgccggcgagagagtgaccatgagctgcaagagcagccagagcctgctgaacagcagaagccagaagaactacctggcctggtaccagcagaagcccggccagccccccaagctgctgatctacggcgccagcaccagaggcagcggcgtgcccgacagattcagcggcagcggcagcggcaccgacttcaccctgaccatcagcagcgtgcaggccgaggacgtggccgtgtactactgccagaacgaccacagctaccccttcaccttcggcagcggcaccaagctggagatcaagcgt

SEQ ID NO: 30 - DNA, 342 bp, synthetic construct
  source 1..342: mol_type = other DNA
gacatcgtgctgacccagagccccagcagcctggccgtgagcctgggcgagagagccaccatgaactgcaagagcagccagagcctgctgaacagcagaagccagaagaactacctggcctggtaccagcagaagcccggccagccccccaagctgctgatctacggcgccagcaccagaggcagcggcgtgcccgacagattcagcggcagcggcagcggcaccgacttcaccctgaccatcagcagcctgcaggccgaggacgtggccgtgtactactgccagaacgaccacagctaccccttcaccttcggccagggcaccaagctggagatcaagcgt

SEQ ID NO: 31 - DNA, 342 bp, synthetic construct
  source 1..342: mol_type = other DNA
gacatcgtgctgacccagagccccagcagcctggccgtgagcctgggcgagagagtgaccatgaactgcaagagcagccagagcctgctgaacagcagaagccagaagaactacctggcctggtaccagcagaagcccggccagccccccaagctgctgatctacggcgccagcaccagaggcagcggcgtgcccgacagattcagcggcagcggcagcggcaccgacttcaccctgaccatcagcagcgtgcaggccgaggacgtggccgtgtactactgccagaacgaccacagctaccccttcaccttcggccagggcaccaagctggagatcaagcgt

SEQ ID NO: 32 - protein, 19 aa, synthetic construct
MGWSWIFLFLLSGTAGVHS

SEQ ID NO: 33 - DNA, 318 bp, synthetic construct
  source 1..318: mol_type = other DNA
acggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 34 - protein, 106 aa, synthetic construct
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - DNA, 990 bp, synthetic construct
  source 1..990: mol_type = other DNA
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 36 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - DNA, 57 bp, synthetic construct
  source 1..57: mol_type = other DNA
atgggatggagctggatctttctcttcctcctgtcaggaactgcaggtgtccactct

SEQ ID NO: 38 - DNA, 48 bp, synthetic construct
  source 1..48: mol_type = other DNA
gctctctacttgtccgggaaccaactgcggagtatcctggcctcaccc

SEQ ID NO: 39 - DNA, 48 bp, synthetic construct
  source 1..48: mol_type = other DNA
gggtgaggccaggatactccgcagttggttcccggacaagtagagagc

SEQ ID NO: 40 - DNA, 48 bp, synthetic construct
  source 1..48: mol_type = other DNA
caggccctgccctacctggagcacctcagcctggctcacaaccggctg

SEQ ID NO: 41 - DNA, 48 bp, synthetic construct
  source 1..48: mol_type = other DNA
cagccggttgtgagccaggctgaggtgctccaggtagggcagggcctg

SEQ ID NO: 42 - DNA, 48 bp, synthetic construct
  source 1..48: mol_type = other DNA
aacagcctgcatggcaatctggtggagcggctgctgggggaggcaccc

SEQ ID NO: 43 - DNA, 48 bp, synthetic construct
  source 1..48: mol_type = other DNA
gggtgcctcccccagcagccgctccaccagattgccatgcaggctgtt

SEQ ID NO: 44 - DNA, 48 bp, synthetic construct
  source 1..48: mol_type = other DNA
cgcctggcacgccacaccttctgggacatgcctgcgctggagcagctt

SEQ ID NO: 45 - DNA, 48 bp, synthetic construct
  source 1..48: mol_type = other DNA
aagctgctccagcgcaggcatgtcccagaaggtgtggcgtgccaggcg

SEQ ID NO: 46 - DNA, 48 bp, synthetic construct
  source 1..48: mol_type = other DNA
actcacctcaatctctccagaaactccctcacctgcatctccgacttc

SEQ ID NO: 47 - DNA, 48 bp, synthetic construct
  source 1..48: mol_type = other DNA
gaagtcggagatgcaggtgagggagtttctggagagattgaggtgagt

SEQ ID NO: 48 - DNA, 48 bp, synthetic construct
  source 1..48: mol_type = other DNA
ttccctgacctggccgtgttcccgagactcatctacctgaacttgtcc

SEQ ID NO: 49 - DNA, 48 bp, synthetic construct
  source 1..48: mol_type = other DNA
ggacaagttcaggtagatgagtctcgggaacacggccaggtcagggaa

SEQ ID NO: 50 - DNA, 48 bp, synthetic construct
  source 1..48: mol_type = other DNA
aatgagatcgaactggtccctgctagctttcttgagcacctgacctcc

SEQ ID NO: 51 - DNA, 48 bp, synthetic construct
  source 1..48: mol_type = other DNA
ggaggtcaggtgctcaagaaagctagcagggaccagttcgatctcatt

(19) United States
(12) Patent Application Publication
(10) Pub. No.: US 2024/0425571 A1
(43) Pub. Date: Dec. 26, 2024

(54) GLYCOPROTEIN A REPETITIONS PREDOMINANT (GARP)-BINDING ANTIBODIES AND USES THEREOF

(71) Applicants: OHIO STATE INNOVATION FOUNDATION, Columbus, OH (US); MUSC FOUNDATION FOR RESEARCH DEVELOPMENT, Charleston, SC (US)

(72) Inventor: Zihai LI, Columbus, OH (US)

(21) Appl. No.: 18/700,473

(22) PCT Filed: Oct. 11, 2022
(86) PCT No.: PCT/US2022/077920

§ 371 Date: Apr. 11, 2024

Related U.S. Application Data
(60) Provisional application No. 63/254,182, filed on Oct. 11, 2021.
(60) Provisional application No. 63/402,763, filed on Aug. 31, 2022.

(51) Int. Cl. C07K 16/18; A61P 35/00

(52) U.S. Cl. CPC C07K 16/18; A61P 35/00; C07K 2317/24; C07K 2317/76; C07K 2317/92

(57) ABSTRACT

Isolated or recombinant monoclonal antibodies that bind to GARP are provided. In some cases, antibodies of the embodiments can be used for the detection, diagnosis and/or therapeutic treatment of human diseases, such as cancer. Further provided herein are methods and compositions for treating cancer in an individual comprising administering to the individual an effective amount of an anti-platelet agent and a T cell therapy.

### CROSS-REFERENCE TO RELATED APPLICATIONS

This application claims the benefit of U.S. Provisional Application No. 63/254,182 filed on Oct. 11, 2021, and U.S. Provisional Application No. 63/402,763 filed on Aug. 31, 2022, which are incorporated herein by reference in their entireties.

### STATEMENT REGRADING FEDERAL FUNDING

The invention was made with government support under Grant Nos. R01AI070603, P01CA186866, R01CA188419, and P30CA138313 awarded by the National Institutes of Health. The government has certain rights in the invention.

### INCORPORATION OF SEQUENCE LISTING

The sequence listing that is contained in the file named “103361-134WO1.xml”, which is 48.7 KB and which was created on Oct. 11, 2022, is filed herewith by electronic submission and is incorporated by reference herein.

### BACKGROUND

### 1. Field of the Invention

The present disclosure relates generally to the fields of cancer biology, immunology and medicine. More particularly, it concerns GARP (Glycoprotein-A Repetitions Predominant Protein) targeting monoclonal antibodies for the treatment and detection of cancer, and methods of treating cancer using immunotherapy. Specifically, a method of treating cancer by combining T cell therapy with an anti-platelet agent is provided.

### 2. Description of Related Art

TGF-β is a pleiotropic cytokine widely expressed in most tissues. Aberrance in its signaling has been implicated in multiple diseases and cancer in particular (Derynck et al., 2001; Massague, 2008). In addition to growth arrest, TGF-β induces a variety of malignant cellular phenotypes including invasion, loss of cellular adhesion, epithelial-mesenchymal transition and metastasis (Bhowmick et al., 2001; Derynck et al., 2001; Oft et al., 1998). Importantly, the role of TGF-β in shaping the tumor micro-environment is a critical aspect of its function in carcinogenesis. For example, TGF-β1 is a potent inducer of angiogenesis (Roberts et al., 1986), either directly by inducing VEGF expression (Pertovaara et al., 1994) or by recruiting other cells such as monocytes which in turn secrete pro-angiogenic molecules (Sunderkotter et al., 1991). TGF-β can also manipulate the tumor micro-environment by favoring the evasion of cancer cells from immune-surveillance, via tampering the effective antitumor functions of T cells, NK cells, B cells or others (Kehrl et al., 1986; Kopp et al., 2009), through its direct effect as well as its ability to induce Foxp3+ regulatory T cells (Li and Flavell, 2008).

Biochemically, TGF-β exists in at least 4 different forms: 1) freely soluble active TGF-β; 2) soluble TGF-β associated with latency associated peptide or LAP (forming a TGF-β-LAP complex, known as latent TGF-β or LTGF-β); 3) LTGF-β associated covalently with large TGF-β-binding protein (LTBP), thus forming the TGF-β-LAP-LTBP complex; and 4) the membrane latent form of TGF-(mTGF-β) (Li and Flavell, 2008; Tran, 2012). Only LAP-free TGF-β is known to be biologically active. Therefore, a large pool of TGF-β is sequestered in the extracellular matrix in a latent form before being activated by proteases such as MMP2, MMP9 and plasmin (Lyons et al., 1990; Sato and Rifkin, 1989; Yu and Stamenkovic, 2000), which are in turn secreted by tumor cells and other cells in the tumor microenvironment. mLTGF-β is expressed by two hematopoietic cell types; platelets and regulatory T cells in association with the transmembrane protein Glycoprotein A Repetitions Predominant (GARP), also known as leucine-rich repeat containing 32 (LRRC32) (Tran et al., 2009; Wang et al., 2012). Besides its role as mLTGF-β docking receptor, GARP is critical for regulating TGFβ activation and bioavailability: GARP enhances proTGF-β maturation and cooperates with integrins in mLTGF-β activation (Wang et al., 2012). The potential role of GARP in cancer is described herein.

Passive immunization through the adoptive transfer of a large number of tumor-reactive lymphocytes, known as adoptive cell therapy (ACT) has shown promising activity experimental in the treatment for patients with metastatic melanoma, and is extensively explored for the treatment of other human cancers ACT involves the administration of large numbers of highly selective cells with high avidity for tumor antigens. These T cells can be programmed and activated ex vivo to exhibit antitumor effector functions. Furthermore, T cell infusion may be preceded by ‘conditioning’ of the patient with lymphodepleting chemotherapy or total body irradiation, which enables the diminution of immunosuppressive cell types/factors followed by the infusion of tumor-specific T cells. Although ACT appears to be promising in many aspects, extensive works needs to be done in order for the treatment to be more successful.

The encouraging clinical achievements of ACT are confronted with major obstacles which limit the clinical benefit and broader application of this approach. Whereas some of the intrinsic difficulties are attributable to the particular method employed for isolation, propagation or generation of the effector lymphocytes, others, such as the exhaustion of the proliferative and survival potential of fully differentiated T cells, seem to be a more general phenomena related to the effector phenotype. Other difficulties arise from extrinsic suppressive mechanisms exerted at the tumor site, which are mediated either by direct cell-to-cell contact with tumor cells, stromal cells and regulatory T cells (Tregs), or by inhibitory cytokines such as TGF-β. As a result, the administered T cells exhibit decreased intratumoral persistence and impaired functionality, and often fall short from executing a detectable tumoricidal effect. Thus, there is a need for methods to evade or subvert these suppressive mechanisms and augment the curative outcome of ACT.

### SUMMARY

Aspects of the present disclosure provide methods for the treatment of cancer. In one aspect, there is provided isolated monoclonal antibodies, wherein the antibodies specifically bind to GARP. In some aspects, the antibodies comprise (a) a first V_{H} CDR at least 80% 90%, 95%, 98%, 99% or 100% identical to V_{H} CDR1 of humanized PIIO-1 (SEQ ID NO: 1) or 5c5 (SEQ ID NO: 9); (b) a second V_{H} CDR at least 80% 90%, 95%, 98%, 99% or 100% identical to V_{H} CDR2 of humanized PIIO-1 (SEQ ID NO: 2) or 5c5 (SEQ ID NO: 10); (c) a third V_{H} CDR at least 80%, 90%, 95%, 98%, 99% or 100% identical to V_{H} CDR3 of humanized PIIO-1 (SEQ ID NO: 3) or 5c5 (SEQ ID NO: 11); (d) a first V_{L} CDR at least 80%, 90%, 95%, 98%, 99% or 100% identical to V_{L} CDR1 of humanized PIIO-1 (SEQ ID NO: 5) or 5c5 (SEQ ID NO: 13); (e) a second V_{L} CDR at least 80% 90%, 95%, 98%, 99% or 100% identical to V_{L} CDR2 of humanized PIIO-1 (SEQ ID NO: 6) or 5c5 (SEQ ID NO: 14), and (f) a third V_{L} CDR at least 80% 90%, 95%, 98%, 99% or 100% identical to V_{L} CDR3 of humanized PIIO-1 (SEQ ID NO: 7) or 5c5 (SEQ ID NO: 15). Thus, in one aspect disclosed herein are isolated anti-glycoprotein A repetitions predominant (GARP) monoclonal antibodies, wherein the antibodies specifically bind to GARP and comprises i) a variable heavy chain (VH) complementarity determining region 1 (CDR1), CDR2, and CDR3 as set forth in SEQ ID NO: 1, SEQ ID NO: 2, and SEQ ID NO: 3, respectively and ii) a variable light chain (VL) complementarity determining region 1 (CDR1), CDR2, and CDR3 as set forth in SEQ ID NO: 5, SEQ ID NO: 6, and SEQ ID NO: 7, respectively; or the antibody comprises i) a variable heavy chain (VH) complementarity determining region 1 (CDR1), CDR2, and CDR3 as set forth in SEQ ID NO: 9, SEQ ID NO: 10, and SEQ ID NO: 11, respectively and ii) a variable light chain (VL) complementarity determining region 1 (CDR1), CDR2, and CDR3 as set forth in SEQ ID NO: 13, SEQ ID NO: 14, and SEQ ID NO: 15, respectively.

In certain aspects, the antibody comprises a first V_{H} CDR at least 80% 90%, 95%, 98%, 99% or 100% identical to SEQ ID NO: 1, a second V_{H} CDR at least 80% 90%, 95%, 98%, 99% or 100% identical to SEQ ID NO: 2, a third V_{H} CDR at least 80% 90%, 95%, 98%, 99% or 100% identical to SEQ ID NO: 3, a first V_{L} CDR at least 80% 90%, 95%, 98%, 99% or 100% identical to SEQ ID NO: 5, a second V_{L} CDR at least 80% 90%, 95%, 98%, 99% or 100% identical to SEQ ID NO: 6, and a third V_{L} CDR at least 80% 90%, 95%, 98%, 99% or 100% identical to SEQ ID NO: 7. In a specific aspect, the antibody comprises a first V_{H} CDR is identical to SEQ ID NO: 1, a second V_{H} CDR is identical to SEQ ID NO: 2, a third V_{H} CDR is identical to SEQ ID NO: 3, a first V_{L} CDR is identical to SEQ ID NO: 5, a second V_{L} CDR is identical to SEQ ID NO: 6, and a third V_{L} CDR is identical to SEQ ID NO: 7.

In other aspects, the antibody comprises a first V_{H} CDR at least 80% 90%, 95%, 98%, 99% or 100% identical to SEQ ID NO: 9, a second V_{H} CDR at least 80% 90%, 95%, 98%, 99% or 100% identical to SEQ ID NO: 10, a third V_{H} CDR at least 80% 90%, 95%, 98%, 99% or 100% identical to SEQ ID NO: 11, a first V_{L} CDR at least 80% 90%, 95%, 98%, 99% or 100% identical to SEQ ID NO: 13, a second V_{L} CDR at least 80% 90%, 95%, 98%, 99% or 100% identical to SEQ ID NO: 14, and a third V_{L} CDR at least 80% 90%, 95%, 98%, 99% or 100% identical to SEQ ID NO: 15. In a particular aspect, the antibody comprises a first V_{H} CDR is identical to SEQ ID NO: 9, a second V_{H} CDR is identical to SEQ ID NO: 10, a third V_{H} CDR is identical to SEQ ID NO: 11, a first V_{L} CDR is identical to SEQ ID NO: 13, a second V_{L} CDR is identical to SEQ ID NO: 14, and a third V_{L} CDR is identical to SEQ ID NO: 15.

In yet other aspects, the binding site or epitope is within the extracellular domain of GARP and may comprise, consist essentially of, consist of or be located within GARP residues 171-207 for humanized PIIO-1 (DMPALEQLDLHSNVLMDIEDGAFEGLPRLTHLNLSRN; SEQ ID NO: 4) and 20-61 for 5C5 (HQDKVPCKMVDKKVSCQVLGLLQVPSVLPPDTETLDLSGNQ; SEQ ID NO: 8)

In some aspects, the antibody comprises (i) a V_{H} domain at least about 80% 90%, 95%, 98%, 99% or 100% identical to the V_{H} domain of humanized PIIO-1 (SEQ ID NO. 18, 19, 20, or 21) and a V_{L} domain at least about 80% 90%, 95%, 98%, 99% or 100% identical to the V_{L} domain of humanized PIIO-1 (SEQ ID NO: 22, 23, or 24); or (ii) a V_{H} domain at least about 80% 90%, 95%, 98%, 99% or 100% identical to the V_{H} domain of 5c5 (SEQ ID NO: 12) and a V_{L} domain at least about 80% 90%, 95%, 98%, 99% or 100% identical to the V_{L} domain of 5c5 (SEQ ID NO: 16). In a specific aspect, the antibody comprises a V_{H} domain identical to the V_{H} domain of humanized PIIO-1 (SEQ ID NO: 18, 19, 20, or 21) and a V_{L} domain identical to the V_{L} domain of humanized PIIO-1 (SEQ ID NO: 22, 23, or 24). In another particular aspect, the antibody comprises a V_{H} domain identical to the V_{H} domain of 5c5 (SEQ ID NO: 12) and a V_{L} domain identical to the V_{L} domain 5c5 (SEQ ID NO: 16). In one specific aspect, the antibody is the humanized PIIO-1 antibodies (i.e., HuPIIO-1V_{H}1/L1, HuPIIO-1V_{H}1/L2, HuPIIO-1V_{H}2/L1, HuPIIO-1V_{H}1/L3, HuPIIO-1V_{H}2/L2, HuPIIO-1V_{H}2/L3, HuPIIO-1V_{H}3/L1, HuPIIO-1V_{H}2/L3, HuPIIO-1V_{H}3/L3, HuPIIO-1V_{H}4/L1, HuPIIO-1V_{H}4/L2, and/or HuPIIO-1V_{H}4/L3) or 5c5 antibody. Accordingly, also disclosed herein are anti-GARP antibodies of any preceding aspect, wherein the antibody comprises a V_{H} domain at least about 80%, 90%, 95%, 98% or 99% identical to the V_{H} domain of the humanized PIIO-1 (huPIIO-1) antibodies as set forth in SEQ ID NO: 18, 19, 20 or 21 and/or a V_{L} domain at least about 80% 90%, 95%, 98% or 99% identical to the V_{L} domain of the huPIIO-1 antibodies as set forth in SEQ ID NO: 22, 23, or 24. In some aspects the antibody comprises a V_{H} domain as set forth in SEQ ID NO: 18, 19, 20, or 21 and/or a V_{L} domain as set forth in SEQ ID NO: 22, 23 or 24. For example, disclosed herein are anti-GARP antibodies of any preceding aspect wherein the antibody comprises a V_{H} domain as set forth in SEQ ID NO: 20 and V_{L} domain as set forth in SEQ ID NO: 23 (VH1VL1), a V_{H} domain as set forth in SEQ ID NO: 20 and V_{L} domain as set forth in SEQ ID NO: 24 (VH1VL2), a V_{H} domain as set forth in SEQ ID NO: 21 and V_{L} domain as set forth in SEQ ID NO: 23 (VH1VL1), SEQ ID NO: 20 and V_{L} domain as set forth in SEQ ID NO: 22 (VH1VL3), a V_{H} domain as set forth in SEQ ID NO: 21 and V_{L} domain as set forth in SEQ ID NO: 24 (VH2VL2), a V_{H} domain as set forth in SEQ ID NO: 21 and V_{L} domain as set forth in SEQ ID NO: 22 (VH2VL3), a V_{H} domain as set forth in SEQ ID NO: 19 and V_{L} domain as set forth in SEQ ID NO: 23 (VH3VL1), a V_{H} domain as set forth in SEQ ID NO: 19 and V_{L} domain as set forth in SEQ ID NO: 24 (VH3VL2), a V_{H} domain as set forth in SEQ ID NO: 19 and V_{L} domain as set forth in SEQ ID NO: 22 (VH3VL3), a V_{H} domain as set forth in SEQ ID NO: 18 and V_{L} domain as set forth in SEQ ID NO: 23 (VH4VL1), a V_{H} domain as set forth in SEQ ID NO: 18 and V_{L} domain as set forth in SEQ ID NO: 24 (VH4VL2), or a V_{H} domain as set forth in SEQ ID NO: 18 and V_{L} domain as set forth in SEQ ID NO: 22 (V_{H}4V_{L}3). In further aspects, the antibody is recombinant.

In additional aspects, the antibody of any preceding aspect is an IgG (such as, for example, IgG1, IgG2, IgG3, or IgG4), IgM, IgA or an antigen binding fragment thereof. In certain aspects, the antibody is a Fab′, a F(ab′)2, a F(ab′)3, a monovalent scFv, a bivalent scFv, nanobody, or a single domain antibody. In some aspects, the antibody of any preceding aspect may be a human, humanized antibody or de-immunized antibody.

Also disclosed herein are antibodies of any preceding aspect wherein the antibody is conjugated to a platelet binding agent (such as, for example, a cyclooxygenase inhibitor, adenosine diphosphate (ADP) inhibitor (including, but not limited to clopidogrel, prasugrel, or ticlopidine), phosphodiesterase inhibitor, protease-activated receptor-1 (PAR-1) antagonist, glycoprotein IIB/IIIA inhibitor, adenosine reuptake inhibitor, and thromboxane inhibitor), an imaging agent, a chemotherapeutic agent, a toxin, a radionuclide, a cytokine, or other therapeutic moieties. In certain aspects, the antibody has at least second binding specificity, such as a bispecific antibody that binds to GARP and a second target.

Humanized antibodies of the disclosure do not all perform equivalently. For example, Table G establishes that huPIIO-1VH1VL2 (and also, huPIIO-1VH2VL1) are superior to huPIIO-1VH1VL1. In addition, FIG. 16A/Table H establish that VH1VL2 has superior homogeneity versus clone huPIIO-1VH2VL1. Moreover, huPIIO-1VH1VL2 appears to have superior thermostability over the parental 4D3 chimeric antibody as described in paragraph [00243] and Table F.

Also disclosed herein are polynucleotide molecules comprising a nucleic acid sequence encoding the antibody of any preceding aspect.

A further aspect of the disclosure provides a composition comprising an antibody of any preceding aspect and aspects described herein in a pharmaceutically acceptable carrier. In some aspects, the composition can further comprise an anti-cancer agent (such as, for example, an immune checkpoint inhibitor including but not limited to antibodies that block PD-1 (such as, for example, Nivolumab (BMS-936558 or MDX1106), pembrolizumab, CT-011, AMP-224, MK-3475), PD-L1 (such as, for example, atezolizumab, avelumab, dorvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT) (such as, for example BMS-986207, OMP-313M32, MK-7684, AB-154, ASP-8374, MTIG7192A, or PVSRIPO), CD96, B- and T-lymphocyte attenuator (BTLA), V-domain Ig suppressor of T cell activation (VISTA) (such as, for example, JNJ-61610588, CA-170), TIM3 (such as, for example, TSR-022, MBG453, Sym023, INCAGN2390, LY3321367, BMS-986258, SHR-1702, RO7121661), LAG-3 (such as, for example, BMS-986016, LAG525, MK-4280, REGN3767, TSR-033, BI754111, Sym022, FS118, MGD013, and Immutep).

In still a further aspect, the disclosure provides an isolated polynucleotide molecule comprising a nucleic acid sequence encoding an antibody of any preceding aspect or other aspects described herein. For example, disclosed herein are recombinant polypeptides comprising an antibody V_{H} domain comprising CDRs 1, 2, and 3 of the V_{H} domain of the huPIIO-1 antibodies as set forth in SEQ ID NOs: 1, 2, and 3, respectively or CDRs 1, 2, and 3 of the V_{H} domain of 5c5 as set forth in SEQ ID NOs: 9, 10, and 11, respectively and/or an antibody V_{L} domain comprising CDRs 1, 2, and 3 of the V_{L} domain of the huPIIO-1 antibodies as set forth in SEQ ID NOs: 5, 6, and 7, respectively or CDRs 1, 2, and 3 of the V_{L} domain of 5c5 as set forth in SEQ ID NOs: 13, 14, and 15, respectively.

In one aspect, disclosed herein are isolated polynucleotide molecules comprising a nucleic acid sequence encoding the antibody of any or the polypeptide of any preceding aspect. For example, disclosed herein are isolated polynucleotide molecules, wherein the nucleic acid comprises SEQ ID NO: 25, SEQ ID NO: 26, SEQ ID NO: 27, SEQ ID NO: 28, SEQ ID NO: 29, SEQ ID NO: 30, and/or SEQ ID NO: 31.

In still yet a further embodiment, the disclosure provides a host cell comprising one or more polynucleotide molecule(s) encoding an antibody of any preceding aspect or a recombinant polypeptide of any preceding aspect, or the isolated nucleic acid of any preceding aspect. In some aspects, the host cell is a mammalian cell, a yeast cell, a bacterial cell, a ciliate cell or an insect cell.

Also disclosed herein are methods for treating, inhibiting, reducing, decreasing, ameliorating, and/or preventing a cancer and/or metastasis (such as, for example, breast cancer, lung cancer, head & neck cancer, prostate cancer, esophageal cancer, tracheal cancer, skin cancer, brain cancer, liver cancer, bladder cancer, stomach cancer, pancreatic cancer, ovarian cancer, uterine cancer, cervical cancer, testicular cancer, colon cancer, rectal cancer, a hematological cancer, clear cell kidney cancer, head/neck squamous cell carcinoma, lung squamous cell carcinoma, melanoma, non-small-cell lung cancer (NSCLC), renal cell cancer, small-cell lung cancer (SCLC), triple negative breast cancer, acute lymphoblastic leukemia (ALL), acute myeloid leukemia (AML), chronic lymphocytic leukemia (CLL), chronic myeloid leukemia (CML), diffuse large B-cell lymphoma (DLBCL), follicular lymphoma, Hodgkin's lymphoma (HL), mantle cell lymphoma (MCL), multiple myeloma (MM), myeloid cell leukemia-1 protein (Mcl-1), myelodysplastic syndrome (MDS), non-Hodgkin's lymphoma (NHL), or small lymphocytic lymphoma (SLL)) in a subject with a cancer comprising administering to the subject a therapeutically effective amount of an antibody of any preceding aspect or the composition of any aspect. In some aspect, the cancer is a GARP positive cancer

In one aspect disclosed herein are methods for treating, inhibiting, reducing, decreasing, ameliorating, and/or preventing a cancer and/or metastasis of any preceding aspect, wherein the antibody is in a pharmaceutically acceptable composition. In some specific aspects, the antibody is administered systemically. In other aspects, the antibody is administered intravenously, intradermally, intratumorally, intramuscularly, intraperitoneally, subcutaneously, or locally.

Also disclosed herein are methods for treating, inhibiting, reducing, decreasing, ameliorating, and/or preventing a cancer and/or metastasis of any preceding aspect, wherein the method further comprises administering to the subject at an anticancer therapy and/or an anticancer agent (such as, for example, i) a TGFβ inhibitor including, but not limited to LY2157299, trabedersen, fresolimumab, LY2382770, lucanix, or PF-03446962, and/or ii) an anti-platelet agent including, but not limited to a cyclooxygenase inhibitor, adenosine diphosphate (ADP) inhibitor (such as, for example, clopidogrel, prasugrel, or ticlopidine), phosphodiesterase inhibitor, protease-activated receptor-1 (PAR-1) antagonist, glycoprotein IIB/IIIA inhibitor, adenosine reuptake inhibitor, and thromboxane inhibitor and/or iii) an immune checkpoint inhibitor (such as, for example, antibodies that block PD-1 (such as, for example, Nivolumab (BMS-936558 or MDX1106), pembrolizumab. CT-011, AMP-224, MK-3475), PD-L1 (such as, for example, atezolizumab, avelomab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT) (such as, for example BMS-986207, OMP-313M32, MK-7684, AB-154, ASP-8374, MTIG7192A, or PVSRIPO), CD96, B- and T-lymphocyte attenuator (BTLA), V-domain Ig suppressor of T cell activation (VISTA) (such as, for example, JNJ-61610588, CA-170), TIM3 (such as, for example, TSR-022, MBG453, Sym023, INCAGN2390, LY3321367, BMS-986258, SHR-1702, RO7121661), LAG-3 (such as, for example, BMS-986016, LAG525, MK-4280, REGN3767, TSR-033, BI754111, Sym022, FS118, MGD013, and Immutep) to the subject. In some of these aspects, the second anticancer therapy is a surgical therapy, chemotherapy, radiation therapy, cryotherapy, hormonal therapy, targeted therapy, immunotherapy (such as, for example, adoptive cell transfer therapy) or cytokine therapy. In some aspects, the immunotherapy is administered before the anti-platelet agent, simultaneous with the anti-platelet agent, or after the anti-platelet agent. In some aspects, the method can further comprise lymphodepletion (such as, for example, via administration of cyclophosphamide and/or fludarabine) of the subject prior to administration of the T cell therapy. In particular aspects, the anti-platelet agent is any of the anti-GARP antibodies of any preceding aspect or fragment thereof.

In one aspect, disclosed herein are methods for treating, inhibiting, reducing, decreasing, ameliorating, and/or preventing a cancer and/or metastasis of any preceding aspect, wherein the adoptive cell transfer therapy comprises the transfer of T cells (including, but not limited to tumor infiltrating lymphocytes (TILs), chimeric antigen receptor (CAR) T cells, CD8⁺ T cells and/or CD4⁺ T cells), chimeric antigen receptor (CAR) T cells, B cells, Natural Killer (NK) cells, CAR NK cells, CAR macrophage (CARMA), and/or NK T cells. In some aspect, the T cells are tumor specific.

Also disclosed herein are methods for treating, inhibiting, reducing, decreasing, ameliorating, and/or preventing a cancer and/or metastasis of any preceding aspect wherein the tumor-specific T cells are engineered to express a T cell receptor (TCR) or chimeric antigen receptor (CAR) receptor having antigenic specificity for a tumor antigen (such as, for example, tEGFR, Her2, CD19, CD20, CD22, mesothelin, CEA, CD23, CD24, CD30, CD33, CD38, CD44, EGFR, EGP-2, EGP-4, EPHa2, ErbB2, FBP, MAGE-A1, MUC1, NY-ESO-1, and/or MART-1. In some aspects, the CAR comprises co-stimulatory molecule endodomains selected from the group consisting of CD28, CD27, 4-IBB, OX40 ICOS, and a combination thereof.

In some aspects, disclosed herein are methods for treating, inhibiting, reducing, decreasing, ameliorating, and/or preventing a cancer and/or metastasis of any preceding aspect wherein the adoptively transferred cells are autologous.

Yet still a further embodiment of the disclosure provides a method for detecting a cancer in a subject comprising obtaining a potentially cancerous tissue sample form a subject and testing the tissue sample for the presence of increased levels of GARP (including, but not limited to soluble GARP or GARP expressing cells) relative to a noncancerous control. In some aspects, the detection of GARP is obtained through the use of the anti-GARP antibodies of any preceding aspect. In some aspects, the method is further defined as an in vitro or ex vivo method.

In one aspect, disclosed herein are methods of stimulating T cells and/or B cells in a subject with a cancer comprising administering to the subject an effective amount of the anti-GARP antibody of any preceding aspect. For example, disclosed herein are methods of stimulating T cells (such as, for example Th1 CD4+ T cells, Th2 CD4+ T cells, effector CD8+ T cells (CD25+, CD45RA−+, CD45RO−, and CD127−), and/or effector memory CD8+ T cells (CD25−, CD45RA−, CD45RO+, and CD127+) and/or B cells (including, but not limited to T cells and B cells in a tumor microenvironment) in a subject with a cancer comprising administering to the subject an effective amount of an anti-GARP antibody (such as, for example, an anti-GARP antibody comprising a heavy chain CDR1, CDR2, and CDR3 as set forth in SEQ ID NO: 1, SEQ ID NO: 2, and SEQ ID NO: 3, respectively (such as, for example a heavy chain variable domain as set forth in SEQ ID NO: 18, 19, 20, or 21) and/or a light chain CDR1, CDR2, and CDR3 as set forth in SEQ ID NO: 5, SEQ ID NO: 6, and SEQ ID NO: 7, respectively (such as, for example, a light chain variable domain as set forth in SEQ ID NO: 22, 23, 24). Such antibodies can include, but are not limited to HuPIIO-1VH1/L1, HuPIIO-1VH1/L2, HuPIIO-1VH2/L1, HuPIIO-1VH1/L3, HuPIIO-1VH2/L2, HuPIIO-1VH2/L3, HuPIIO-1VH3/L1, HuPIIO-1VH2/L3, HuPIIO-1VH3/L3, HuPIIO-1VH4/L1, HuPIIO-1VH4/L2, and/or HuPIIO-1VH4/L3.

In one aspect, the T cells stimulated by any of the preceding methods are endogenous tumor infiltrating lymphocytes (TILs). Also disclosed herein are methods of stimulating T cells of any preceding aspect, wherein the CD8 T cells are TILs or chimeric antigen receptor (CAR) T cells administered to the subject as a component of an immunotherapy.

Also disclosed herein are methods of stimulating adoptively transferred donor T cells (such as, for example, Th1 CD4+ T cells, Th2 CD4+ T cells, effector CD8+ T cells (CD25+, CD45RA−+, CD45RO−, and CD127−), and/or effector memory CD8+ T cells (CD25−, CD45RA−, CD45RO+, and CD127+) in a tumor microenvironment of a subject comprising administering the T cells and an anti-GARP antibody (such as, for example, an anti-GARP antibody comprising a heavy chain CDR1, CDR2, and CDR3 as set forth in SEQ ID NO: 1, SEQ ID NO: 2, and SEQ ID NO: 3, respectively (such as, for example a heavy chain variable domain as set forth in SEQ ID NO: 18, 19, 20, or 21) and/or a light chain CDR1, CDR2, and CDR3 as set forth in SEQ ID NO: 5, SEQ ID NO: 6, and SEQ ID NO: 7, respectively (such as, for example, a light chain variable domain as set forth in SEQ ID NO: 22, 23, 24). Such antibodies can include, but are not limited to HuPIIO-1VH1/L1, HuPIIO-1VH1/L2, HuPIIO-1VH2/L1, HuPIIO-1VH1/L3, HuPIIO-1VH2/L2, HuPIIO-1VH2/L3, HuPIIO-1VH3/L1, HuPIIO-1VH2/L3, HuPIIO-1VH3/L3, HuPIIO-1VH4/L1, HuPIIO-1VH4/L2, and/or HuPIIO-1VH4/L3. In one aspect, the anti-GARP antibody can be administered prior to, concurrent with, or after the transfer of donor T cells. In one aspect, the T cells are TILs or chimeric antigen receptor (CAR) T cells administered to the subject as a component of an immunotherapy.

In one aspect, disclosed herein are methods of inducing T cell or B cell proliferation in a subject with a cancer comprising administering to the subject an effective amount of an anti-GARP antibody of any preceding aspect (such as, for example, an anti-GARP antibody comprising a heavy chain CDR1, CDR2, and CDR3 as set forth in SEQ ID NO: 1, SEQ ID NO: 2, and SEQ ID NO: 3, respectively (such as, for example a heavy chain variable domain as set forth in SEQ ID NO: 18, 19, 20, or 21) and/or a light chain CDR1, CDR2, and CDR3 as set forth in SEQ ID NO: 5, SEQ ID NO: 6, and SEQ ID NO: 7, respectively (such as, for example, a light chain variable domain as set forth in SEQ ID NO: 22, 23, 24). Such antibodies can include, but are not limited to HuPIIO-1V_{H}1/L1, HuPIIO-1VH1/L2, HuPIIO-1VH2/L1, HuPIIO-1VH1/L3, HuPIIO-1VH2/L2, HuPIIO-1VH2/L3, HuPIIO-1VH3/L1, HuPIIO-1VH2/L3, HuPIIO-1VH3/L3, HuPIIO-1VH4/L1, HuPIIO-1VH4/L2, and/or HuPIIO-1VH4/L3.

Also disclosed herein are methods of inducing T cell or B cell proliferation in a subject with a cancer comprising administering to the subject an effective amount of the anti-GARP antibody of any preceding aspect.

In one aspect, disclosed herein are methods of blocking T cell exhaustion of a CD8+ T cell comprising contacting the CD8+ T cell with an effective amount of the anti-GARP antibody of any preceding aspect. In some aspects the CD8+ T cell is contacted with the anti-GARP antibody ex vivo. In other aspects, the CD8+ T cells are located in the tumor microenvironment.

Also disclosed herein are methods of inhibiting Tregs in a tumor microenvironment in a subject comprising administering to the subject a therapeutically effective amount of the anti-GARP antibody of any preceding aspect.

In one aspect, disclosed herein are methods of blocking GARP-LTGFβ1 complex formation in a cancer comprising contact the cancer with a therapeutically effective amount of the anti-GARP antibody of any preceding aspect.

Also disclosed herein are methods of increasing the efficacy of a immune checkpoint blockade (ICB) therapy in a subject comprising administering to a subject receiving ICB therapy a therapeutically effective amount of the anti-GARP antibody of any preceding aspect.

In one aspect, disclosed herein are methods of activating T cells or B cells comprising in a subject with a cancer comprising administering to the subject an effective amount of an anti-GARP antibody of any preceding aspect. For example, disclosed herein are methods of activating T cells (such as, for example, Th1 CD4+ T cells, Th2 CD4+ T cells, effector CD8+ T cells (CD25+, CD45RA−+, CD45RO−, and CD127−), and/or effector memory CD8+ T cells (CD25−, CD45RA−, CD45RO+, and CD127+) or B cells comprising in a subject with a cancer comprising administering to the subject an effective amount of an anti-GARP antibody (such as, for example, an anti-GARP antibody comprising a heavy chain CDR1, CDR2, and CDR3 as set forth in SEQ ID NO: 1, SEQ ID NO: 2, and SEQ ID NO: 3, respectively (such as, for example a heavy chain variable domain as set forth in SEQ ID NO: 18, 19, 20, or 21) and/or a light chain CDR1, CDR2, and CDR3 as set forth in SEQ ID NO: 5, SEQ ID NO: 6, and SEQ ID NO: 7, respectively (such as, for example, a light chain variable domain as set forth in SEQ ID NO: 22, 23, 24) Such antibodies can include, but are not limited to HuPIIO-1VH1/L1, HuPIIO-1VH1/L2, HuPIIO-1VH2/L1, HuPIIO-1VH1/L3, HuPIIO-1VH2/L2, HuPIIO-1VH2/L3, HuPIIO-1VH3/L1, HuPIIO-1VH2/L3, HuPIIO-1VH3/L3, HuPIIO-1VH4/L1, HuPIIO-1VH4/L2, and/or HuPIIO-1V_{H}4/L3. In one aspect, the T cells and/or B cells are located in a tumor microenvironment.

Also disclosed herein are methods of assessing the sensitivity of a cancer to an immune checkpoint blockade (ICB) therapy comprising obtaining a cancerous tissue sample and assaying the sample for GARP expression; wherein elevated expression of GARP relative to a noncancerous control indicates the cancer is resistant to ICB therapy and low expression of GARP or equivalent expression of GARP relative to a noncancerous control indicates the cancer is sensitive to ICB therapy. In some aspects GARP expression levels can be obtained through an assay using any of the anti-GARP antibodies of any preceding aspect.

In one aspect, disclosed herein are methods of making a cancer cell sensitive to immune checkpoint blockade (ICB) therapy comprising contacting an ICB therapy resistant cancer cell with the anti-GARP of any preceding aspect.

Other objects, features and advantages of the present disclosure will become apparent from the following detailed description. It should be understood, however, that the detailed description and the specific examples, while indicating certain embodiments of the invention, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

The patent or application file contains at least one drawing executed in color. Copies of this patent or patent application publication with color drawing(s) will be provided by the Office upon request and payment of the necessary fee.

FIGS. 1A-IF show GARP upregulation in cancer correlates with poor prognostic significance. (FIG. 1A) Summary of cross-cancer alteration studies for GARP. Data were obtained from www.cbioportal.org in response to query for GARP gene LRRC32 on Nov. 16, 2015. (FIG. 1B) Specificity analysis of hGARP antibody in pre-B EV and pre-B leukemic cells expressing hGARP (FIG. 1C) Patient-matched uninvolved and primary breast cancer. Shown are representative images and the IHC GARP scores. (FIG. 1D) Representative images of GARP IHC (darkened regions) of normal tissues and cancers. Scale bar: 20 μm (FIG. TE) Expression intensity of GARP-positive cells. (FIG. 1F) Correlation between GARP expression and overall survival of colon and lung cancer (left and middle panel) as well as Gleason score of prostate cancer (right panel). The number of samples (n) are indicated. Kaplan Meier curves are shown in FIG. 1F for lung and colon cancer with p-values calculated by log-rank tests. Two sample t-tests were used to compare group differences in FIGS. 1C, 1E and the prostate cancer in FIG. 1F. HR stands for hazard ratio. *P<0.05, **P<0.01, ***P<0.001, ****P<0.0001.

FIGS. 2A-2F show shedding of membrane-bound GARP from cancer cells and its significance as a potential cancer biomarker. (FIG. 2A) GARP cleavage in the post-ER compartment occurs only in the presence of grp94. N-terminal FLAG-tagged GARP was stably expressed in WT or grp94 Pre-B KO cells. The whole cell lysate was treated with Endo H or PNGase F followed by immunoblot with FLAG antibody. (FIG. 2B) Lower fragment protein is GARP based on both immunoreactivity and mass spectrometry analysis. The peptide sequence from GARP that was identified by mass spectrometry is indicated (SEQ ID NO: 17). FIG. 2C) Soluble GARP in the serum of prostate cancer patients and control normal subjects. (FIG. 2D) Correlation analysis between GARP positivity and PSA1 level (left panel), the GARP positivity and the metastatic status of prostate cancer (right panel). (FIG. 2E) Quantification of GARP-TGF-β1 complex in the sera of prostate cancer patients and normal subjects by a sandwich ELISA. (FIG. 2F) Active TGFβ ELISA level from purified recombinant soluble GARP-Fc. The difference in distribution in FIG. 2D was calculated by Chi-squared test. Two sample t-tests were used to compare group differences in FIG. 2E. *P<0.05. ***P<0.001.

FIGS. 3A-3J show enforced GARP expression on normal mammary gland epithelial cells enhances TGF-β signaling and drives epithelial-mesenchymal cell transition (EMT) and invasion. (FIG. 3A) NMuMG cells were transfected to stably express membrane bound GARP, followed by Western blot for E-cadherin, vimentin and phosphor-SMAD-2/3. (FIG. 3B) NMuMG cells were treated with the recombinant human TGF-β1, soluble GARP, and isotype antibody control or left untreated in serum-free medium for 24 h, followed by morphological analysis. (FIG. 3C) NMuMG cells were treated for the indicated time with soluble GARP-Fc (sGARP) in serum-free medium. Vimentin upregulation was detected by Western blot analysis. (FIG. 3D) NMuMG cells were treated with increasing doses of soluble GARP, followed by immunoblot for vimentin. (FIG. 3E) Immunoblot of GARP, TGFβ and β-actin control. (FIG. 3F) ELISA quantification of soluble GARP in the condition medium of NMuMG EV, GARP, and GARP-Fc cells (FIG. 3G) In vitro scratch assay to indicate the difference in the gap closure at 24 h. (FIG. 3H) Summary of three independent scratch assays. (FIG. 3I) In vivo imaging of the luciferin-enhanced bioluminescence in mice after injection of GARP, GARP-Fc and control NMuMG cells at week 3 and 6. (FIG. 3J) Histological analysis of NMuMG-GARP tumors by H&E, and expression of vimentin and E-cadherin by IHC. Scale bar: 20 μm. Two sample f-tests were used to compare group differences in FIG. 3H. *P<0.05, **P<0.01. Two independent experiments were performed with similar findings.

FIGS. 4A-4G show GARP silencing blocks growth and metastasis of mammary carcinoma. (FIG. 4A) ShRNA knockdown of GARP mRNA in NMuMG* cells. Cells treated with scrambled shRNA (SCR) were used as control. (FIG. 4B) Flow cytometric analysis of cell surface GARP expression by GARP KD and SCR NMuMG* cells. (FIG. 4C) Immunoblot of total GARP and TGF-β level in GARP KD and SCR NMuMG cells. (FIG. 4D) MTT assay to compare the growth kinetics of NMuMG*-SCR with NMuMG*-GARP-KD cells. (FIGS. 4E-4G) NMuMG* SCR and NMuMG*-GARP KD cells were injected into NOD-RagI^{-/-} mice, followed by monitoring the tumor growth kinetics (FIG. 4G) and tumor metastasis (FIG. 4F and FIG. 4G). Tumor growth differences in FIG. 4D and FIG. 4E were calculated by 2-way ANOVA. Two sample 1-tests were used to compare group differences in FIG. 4F and FIG. 4G. **P<0.01.

FIGS. 5A-5J show GARP upregulation in murine mammary cancer cells promotes TGF-β activation, tumor growth, metastasis and immune tolerance. (FIG. 5A) Immunoblot for GARP, TGF-β and β-actin control in 4T1 cells stably engineered to express GARP, GARP-Fc or control EV. (FIG. 5B) Quantification of active TGF-β1 by ELISA in the 72 hr conditioned medium from 4T1 EV, GARP and GARP-Fc cells. (FIG. 5C) Naïve CD4⁺ T cells were stimulated with anti-CD3, and anti-CD-28 mAb in the presence of 50% 3-day condition medium from 4T1-EV, 4T1-GARP and 4T1-GARP-Fc cells. Foxp3 expression was analyzed on day 3 by flow cytometry. (FIG. 5D) Female BALB/c mice were injected in the 4^{th} mammary fat pad of indicated tumors. Tumors volume was measured every 3 days. (FIG. 5E) The weight of tumors in grams at the end point of (FIG. 5D). (FIG. 5F) Lungs were isolated and paraffin-embedded. Numbers of tumor nodules in the lungs were counted. (FIG. 5G) The 3-week tumors were isolated and embedded in OCT. Fresh frozen sections were stained for p-SMAD-2/3 mAb Scale bar: 100 μm. (FIG. 5H) Summary statistics for p-SMAD-2/3 staining intensity, defined independently by the studying pathologist. (FIGS. 5I-5J) Tumor-infiltrating lymphocytes were isolated and the numbers of CD4⁺CD25⁺Foxp3⁺ Tregs were enumerated by flow cytometry. (5I) Representative flow plots. (FIG. 5J) Summary of the percentage of Tregs in the tumor microenvironment. Tumor growth difference in FIG. 5D was calculated by 2-way ANOVA. Two sample t-tests were used to compare group differences in other Panels. *P<0.05. **P<0.01. ***P<0.001.

FIGS. 6A-6G show GARP upregulation in B16 mouse melanoma tumor diminishes the effect of the adoptive T cell immunotherapy. (FIG. 6A) Experimental scheme. (FIG. 6B) Average tumor growth kinetics of B16-GARP-Fc and B16-EV (n=6). (FIG. 6C) Difference in survival between two experimental groups as indicated. (FIG. 6D) A representative FACS plot of antigen-specific donor T cells in the peripheral blood indicated by CD8⁺CD90.1⁺ surface marker (FIG. 6E) Frequency of donor T cells in the peripheral blood of tumor-bearing mice at different time points post ACT. (FIG. 6F) A representative FACS plot of intracellular IFNγ stain of peripheral blood antigen-specific donor T cells in response to stimulation by the cognate gp 100 peptide. (FIG. 6G) Quantification of the frequency of IFNγ-producing donor T cells in the peripheral blood of mice received either B16-GARP-Fc or B16-EV. The p-value in FIG. 6C was calculated by log-rank test. Two sample t-tests were used to compare group differences in other panels. *P<0.0S. ***P<0.001.

FIGS. 7A-7F show platelet-intrinsic GARP plays critical roles in generating active TGFβ. (FIG. 7A) Depletion of platelets resulted in a complete loss of active and total TGFβ. (FIGS. 7B-7D) Expression of GARP and LAP in indicated mouse models. Platelets from Plt-Tgfβ1KO mice express similar levels of surface GARP-TGFβ1 complex when compared with WT platelets. (FIG. 7E) Measure of active TGFβ in mice. In WT mice, active TGFβ is elevated in serum compared to plasma. (FIG. 7F) Measure of total TGFβ in mice. The total latent TGFβ level in the serum is reduced in Plt-Tgfβ1KO mice but not Plt-gp96KO or Plt-GARPKO mice.

FIGS. 8A-8D show the efficacy of adoptive T cell therapy of melanoma in WT, Plt-Tgfβ1KO and Plt-GARPKO recipient mice. (FIG. 8A) Tumor growth is controlled more efficiently in Plt-GARPKO mice compared with WT mice. (FIG. 8B) Enhanced persistence and (FIG. 8C) functionality of Pmel cells in peripheral blood of Plt-GARPKO mice. (FIG. 8D) Plt-Tgfβ1KO mice, whose platelets express GARP and remain capable of activating TGFβ, do not have improved control of tumors.

FIGS. 9A-9H show platelet-derived GARP-TGFβ complex blunts anti-tumor T cell immunity. (FIGS. 9A-9C) Tumor size (9A) and overall survival of WT and Plt-GARPKO mice. The growth of MC38 is significantly diminished in Plt-GARPKO mice compared to WT mice. (FIG. 9D) MC38-bearing Plt-GARPKO mice have reduced serum levels of active TGFβ. (FIGS. 9E-9F) Immunohistochemical staining for p-Smad2/3 (p-Smad2/3) in MC38 tumor sections demonstrates a remarkable attenuation of TGFβ signaling in MC38 cells in Plt-GARPKO mice. (FIG. 9G) Reduction of both systemic myeloid-derived suppressor cells (FIG. 9H) and tumor-infiltrating regulatory T cells in Plt-GARPKO mice.

FIGS. 10A-10D show anti-platelet pharmacological agents potentiate adoptive T cell therapy of cancer. (FIG. 10A) Effect of Cy and AP on tumor growth (left). Anti-platelet agents plus adoptive T cell transfer are highly effective against B16-F1 with relapse-free survival of most mice beyond 3 months (right). (FIG. 10B) Antigen-specific T cells sustained at higher numbers in the blood, inguinal lymph nodes (ILNs) and spleens of mice receiving concurrent anti-platelet therapy and ACT. (FIG. 10C) Antiplatelet agents conferred no benefit when the transferred T cells lacked IFN-γamma (FIG. 10D) or when anti-IFN-γ neutralization antibodies were administered.

FIG. 11 shows binding affinity and thermostability assay.

FIG. 12 shows Baculovirus ELISA evaluation of non-specific antibody binding.

FIGS. 13A and 13B show reducing (FIG. 13A) and non-reducing (FIG. 13B) CE-SDS results.

FIG. 14 shows PITO-1 humanization candidate heavy chain variable region sequences. For huPIIO-1VH1, nucleic acid is SEQ ID NO: 27 and amino acid sequence is SEQ ID NO. 20. For hu PIIO-1VH2, nucleic acid is SEQ ID NO: 28 and amino acid sequence is SEQ ID NO: 21. For huPIIO-1VH3, nucleic acid is SEQ ID NO: 26 and amino acid sequence is SEQ ID NO: 19. For huPIIO-1VH4, nucleic acid is SEQ ID NO: 25 and amino acid sequence is SEQ ID NO: 18.

FIG. 15 shows PIIO-1 humanization candidate light chain variable region sequences (top three) and leader sequence for both heavy and light chains (bottom sequence; SEQ ID NO: 32 for the nucleic acid and SEQ ID NO: 37 for the amino acid.). For huPIIO-1VL1, nucleic acid is SEQ ID NO: 30 and amino acid sequence is SEQ ID NO: 23. For huPIIO-1VL2, nucleic acid is SEQ ID NO. 31 and amino acid sequence is SEQ ID NO: 24. For huPIIO-1VL3, nucleic acid is SEQ ID NO: 29 and amino acid sequence is SEQ ID NO: 22.

FIG. 16 shows human kappa constant light region sequence (top; nucleic acid is SEQ ID NO: 33; amino acid is SEQ ID NO: 34) and human IgG1 constant region heavy chain sequence (bottom; nucleic acid is SEQ ID NO: 35; amino acid is SEQ ID NO: 36).

FIGS. 17A-17E show the characterization of anti-GARP monoclonal antibodies. 17A. Surface GARP on human platelets and Tregs detected by flow cytometry and cellular specificity of a-GARP mAbs. 17B. Using 293T cells transfected with hGARP (free GARP), or hGARP andTGFβ (GARP-LAP complex), the specificity of anti-GARP Ab clones was determined by flow cytometry. 17C. 293T cells expressing mGARP/hGARP chimeras were examined for recognition by anti-GARP antibodies. 17D. pre-B-hGARP cells were incubated without or with human LTGFβ (huLTGFβ), in the presence of anti-GARP or isotype control. Cells were stained for cell surface hLTGFβ (hLAP) to determine the ability of the Ab to block binding of hLTGFβ to GARP. 17E. Jurkat-hGARP cells were treated with 2H4 anti-GARP Ab (20 μg/ml) for 24 h, followed by immunoblot for pSMAD3 level in the total cell lysate.

FIGS. 18A-18F show the generation of GARP humanized mice. A. The scheme of construct design. mLrrc32 indicates mouse allele. hLrre32^{KI} denotes human Lrrc32 knockin allele. B. PCR confirmation of genotypes of the indicated mice. HO, homozygous. C. Confirmation of GARP expression on CD41⁺ platelets by flow cytometry using species-specific GARP antibodies. D. Binding specificity of our hGARP monoclonal antibodies on platelets (left) and CD4⁺CD25⁺ Treg cells (right) from the peripheral blood (PB) of mice. E. and F. Toxicity study of huPIIO-1. hLrre32K/mice (n=5/group) were injected i.p. with 200 μg mIgG1 isotype or huPIIO-1 anti-GARP antibody twice per week (n=5/group) for 6 doses. Bodyweight or PB platelet levels measured.

FIGS. 19A-19E show humanized PIIO-1 and anti-PD1 combination therapy were effect against CMT167 lung cancer and remodeled tumor-infiltrating CD8⁺ T cell compartment. FIG. 19A shows tumor volume 18 days after s.c. injection of 1×10⁵ CMT-167 cells. Mice were treated with 4 injections of indicated antibody (day 8, 11, 14 and 17). FIG. 19B shows the frequency of tumor-infiltrating CD8⁺ T cells of Day 18 tumors (left-representative flow plots gated on CD45⁺ cells, right-data quantification). FIG. 19C shows UMAP dimension reduction of multi-color T cell exhaustion panel gated on live CD45⁺CD3⁺CD8⁺ T cells, subsampled on 5000 cells per sample. Unsupervised clustering analysis using FlowSOM algorithm with an elbow method approach for number identification. The left panel shows all cell clusters of concatenated CD8⁺ TILs. The middle and right panel show clusters 3 and 10 only in the indicated treatment groups. FIG. 19D shows Cluster 3 and 10 are highly accumulated in combination group. Analysis was done by EdgeR between anti-PD1 and the combination groups. FIG. 19E shows a heat map for expression of indicated markers of all CD8⁺ T cell subclusters. N=5-7 per group. *p<0.05, **p<0.01; A, Two way repeated measures ANOVA with multiple comparisons. B. Two-tailed independent Student's t-test. Data=mean+/−SEM.

FIGS. 20A-20F show the Impact of LRRC32 gene expression on immune landscape in human cancer and ICB responsiveness. A-C. TCGA analysis. A. Correlation of LRRC32 expression level with indicated immune pathways in multiple human cancer types. Values in each cell indicates t-statistics comparing the top ⅓ vs. the bottom ⅓ LRRC32 expression groups. B. Correlation of immune subtypes between LRRC32 expression and non-small cell lung cancer. (C1. Wound healing. C2. IFN-γ dominant. C3. Inflammatory. C4. Lymphocyte depleted. C6. TGF-β dominant) C. Box plot comparing related immune pathway enrichment in human lung cancers between high and low LRRC32 gene expression groups. Statistical significance was determined using t-tests for A and C, and Fisher's exact test for B. Significance codes: ****p<0.0001, ***p<0.001, **p<0.01, *p<0.05. D-F. Bulk RNA-seq data analysis of pre-treatment tumor samples from 167 patients with metastatic urothelial cancer (mUC) who received atezolizumab in phase 2 trial (IMvigor210). D. Box plots comparing the expression of LRRC32 gene (left) as well as LRRC32-TGFB related signatures (right, defined in Methods) in all types, immune-desert, excluded and inflamed tumors from 167 patients of IMvigor210 between responder (CR/PR, red) and non-responders (SD/PD, blue). CR, complete response; PR, partial response; SD, stable disease; PD, progressive disease. E-F. Kaplan-Meier survival plot comparing overall survival probability (y-axis) and follow-up time (months, x-axis) from IMvigor210 in all types, immune-desert, excluded, inflamed tumors. Groups were split by high (red) or low (green) expression levels of LRRC32 gene (E) and LRRC32-TGFB related signatures (F). Significance was determined by using log rank tests. *p<0.05; **p<0.01.

FIGS. 21A-21E show In vitro characterization of anti-GARP antibody PIIO-1. A. GARP expression on human regulatory T cells and platelets was evaluated by flow cytometry with PIIO-1 at 10 μg/ml. B. 293 cell line were transfected with empty vector (EV), human GARP (hGARP) only or co-transfected with hGARP and latent TGFβ1. GARP expression on indicated cell line was detected by flow cytometry with PIIO-1 at 10 μg/ml. C. Human GARP sequence was replaced by murine GARP sequentially according to the schematic diagram to generate the chimeric constructs of human and murine GARP. Transection efficiency was detected by HA tag expression on the constructs. All constructs were transfected into 293 cells. D. The crystal structure GARP (green)-LTGFβ (grey) complex (PDB DOI: 10.2210/pdb6GFF/pdb). The region of PIIO-1 recognition was highlighted by orange color E. Jurkat cell line, made to overexpress hGARP, were incubated with LTGFβ1 along with isotype control or PIIO-1 at indicated concentration for 30 min at 37° C. Human LAP expression level was detected by flow cytometry. All experiments are the representative of 2-6 independent experiments.

FIGS. 22A-22I show PIIO-1 enhanced anti-tumor efficacy of anti-PD-1 ICB in GARP+ triple negative breast cancer. A. Experimental scheme. BALB/c mice were injected with 1×10⁵ 4T1-hGARP mammary tumor cells in the mammary fat pad, followed by i.p. injection of 100 μg/mouse of PIIO-1 antibody and/or 150 μg/mouse anti-PD-1 every three days. B. Primary tumor growth curve. C. Overall survival of four group of mice. D. Summary of the incidence of tumor free mice among groups. E. Lungs were collected and sectioned at the end points of the experiment, then stained with H&E. Representative images from each group of mice are shown. Scale bar, 20 μm. The numbers of visible lung metastatic nodules are graphed and compared. F. Summary of the incidence of metastasis among groups. G. Tumors were collected at the end points, tumors were stained by IHC for pSMAD3, α-SMA. Representative images of tumor tissues from four groups of mice are shown (left). Scale bar, 50 μm. Quantification of the IHC staining (right). H. Serum were collected at the end point of each mouse. Serum total and active TGFβ were assessed by ELISA. I. Mice which had regressed tumors in combination group were monitored for 300 days, then rechallenged with 5×10⁵ wild type 4Tl mammary tumor in contralateral mammary fat pad. Naive BALB/c mice without pre-exposure to tumor were used as control. Overall survival of two groups of mice. *p<0.05; **, p<0.005; ***, p<0.001. Tumor curve analysis was performed using repeated measures 2-way analysis of variance (ANOVA). Overall survival is analyzed by Log-rank (Mantel-Cox) test. Figure E, G were analyzed by paired t-test according to the tumor collection time points. Other data was analyzed by two-tailed Student's t test with GraphPad Prism. Figure B, C were corrected for multiple testing using the Turkey procedure. All data are presented as mean±SEM.

FIGS. 23A-23G show PIIO-1 monotherapy modulates CD8+ T cells in the TME and confers protection against cancer in hLRRC32KI mice. A. 1×10⁵ MB-49 Bladder Cancer cells were injected s.c. on the right flank of hLRRC32KI mice. PIIO-1 was delivered (200 μg/mouse, i.p.) every 3 days for a total of 4 treatments starting on day 4. Representative tumor curve. B. 1×10⁵ MB-49 Bladder cancer cells were injected s.c. on the right flank of hLRRC32KI mice. PIIO-1 was delivered (200 μg/mouse, i.p.) on day 6 and 9. Tumors were collected and perform flow cytometry on day 10. Frequency of CD8+ T cells as a proportion of live CD45+ lymphocytes (left). 1×10⁵ MB-49 Bladder Cancer cells were injected s.c. on the right flank of hLRRC32KI mice. PIIO-1 was delivered (200 μg/mouse, i.p.) every three days for total 6 treatments starting on day 6. Tumors were collected and perform flow cytometry on day 22. Comparison of CD8+ T cells between ISO and PIIO-1 (right). C. Frequency of CD25+Foxp3⁺ Tregs in CD4+ tumor-infiltrating T cells (left). Frequency of CTLA4+VISTA+ Tregs in tumor (right) D. Differential expression analysis of cluster frequency of CD8+ TILs between ISO and PIIO-1. UMAP dimension reduction of tumor-infiltrating CD8+ T cells from B after staining with 33 markers and spectral flow cytometry analysis. Shown is the data gated on live CD45+CD3+CD8+ T cells, subsampled on 5000 cells per sample Unsupervised clustering analysis was done using FlowSOM algorithm with an elbow method approach for cluster number determination. E. Heatmap of D showing expression levels of indicated markers by each cluster. A-E. N=4-6 per group, data (mean+/−SEM) representative of two independent experiments. F. Differential expression analysis of cytokine production by CD8+ TILs between ISO and PIIO-1 treated tumors. 1×10⁵ MB-49 bladder cancer cells were injected s.c. to the right flank of hLRRC32KI mice. PIIO-1 was administered (200 μg/mouse, i.p.) every 3 days for a total of 4 treatments starting on day 5. Tumors were collected on day 17. Intracellular stain for 17 cytokine panel was done followed by spectral flow cytometry and analysis of CD45+CD3+CD8+ T cells. G. Cytokine level in panel F indicated by heatmap showing expression intensity of cytokines by each CD8+ T cell cluster. *p<0.05, **p<0.01; Tumor curve analysis was performed using repeated measures two-way analysis of variance (ANOVA). Cluster differences were measured by two-tailed Student's t test.

FIGS. 24A-24D show PIIO-1 potentiates preclinical activity of anti-PD-1 antibody against bladder cancer. A. Experimental scheme. 1×10⁵ MB-49 Bladder Cancer cells were injected s.c. on the right flank of hLRRC32KI mice PIIO-1 (200 μg/mouse, i.p) and anti-PD-1 antibody were delivered (100 μg/mouse, i.p.) every 3 days. PIIO-1 started on day 4 for 6 doses and anti-PD-1 antibody started on day 10 for 4 doses. B. Represented overall survival of mice treated with isotype control antibody (n=5), PIIO-1 (n=6), anti-PD-1 Ab (n=10) or combination of anti-PD-1 Ab and PIIO-1 (n=10). C. Summary of therapeutic efficacy based on complete response. D. PIIO-1 and anti-PD-1 generated better anti-tumor memory responses. Mice rendered tumor-free by indicated treatment were rechallenged with live MB-49 subcutaneously. The overall survival was compared. Tumor-free naïve mice were used as control. *p<0.05; **, p<0.005; ***, p<0.001. Overall survival is analyzed by log-rank (Mantel-Cox) test. Figures B, D were corrected for multiple testing using the Turkey procedure. p-values in all data are presented as mean+SEM.

FIGS. 25A-25E show PIIO-1 attenuates canonical TGFβ pathway in tumor-infiltrating immune cells and rejuvenates anti-tumor immunity in hLRRC32KI mice. A. 1×10⁵ MB-49 Bladder Cancer cells were injected s.c. in the right flank of hLRRC32KI mice. PIIO-1 (200 μg/mouse, i.p.) were administered on day 18 and 20 for 2 doses. Tumors were collected on day 21. TILs were then isolated and stained for intracellular pSMAD2/3 and indicated cell linage markers on the cell surface, followed by flow cytometry analysis. B. Quantification of panel A. 1×10⁵ MB-49 Bladder Cancer cells were injected s.c. on the right flank of hLRRC32KI mice. PIIO-1 (200 μg/mouse, i.p.) were delivered on day 6 and 9 for 2 doses. Tumors were collected on day 10. Single cell suspension and RNA isolation were prepared, and then subjected to bulk RNA sequencing. C. Volcano plot of gene expression. Differential gene expression was shown in red (up) or blue (down) Representative transcripts such as Ccl3, Ccl9, Cxcl14, Cxcl15, Il6 and Tnfrsf25 were indicated. D. Gene set enrichment analysis of differential expression genes between tumors treated with PBS and PIIO-1. E. Comparison of TILs between PBS and PIIO-1 treated tumor based on deconvolution of bulk RNA sequencing data. *p<0.05. **p<0.01; Other data was performed using two-tailed Student's t test, data presented as mean+/−SEM.

FIGS. 26A-26L show PIIO-1 promotes anti-tumor activity that is dependent on CD8+ T cells and CXCR-3. A and B. CD8-dependence of anti-tumor activity. A. Experimental scheme. B. Tumor growth curve of mice treated with indicated conditions (Isotype, n=5; PIIO-1, n=5; anti-CD8, n=3; Combo, n=5). C-F. Anti-tumor activity of PIIO-1 depends on active egress of lymphocytes from the secondary lymphoid tissues. C. Experimental scheme. D. Tumor growth curve of mice treated with indicated conditions (Isotype, n=4; PIIO-1, n=4; FTY720, n=6; Combo, n=6) E. The frequencies of CD8+ and CD4+ T cells in the peripheral blood of indicated groups of mice. F. Absolute number of CD8+ T cells in the tumors. G. Impact of PIIO-1 on CXCR3 expression and number of CD8+ T cells in the draining LNs. MB-49 bearing hLRRC32KI mice were treated with 2 courses of PIIO-1 or ISO, followed by analysis of CXCR3 expression on CD8+ T cells in the draining LN. H-L. Anti-tumor effect of PIIO-1 requires CXCR3. H. Experimental scheme. I. Tumor growth curve of mice treated with indicated conditions (Isotype, n=5; PIIO-1, n=5; FTY720, n=7; Combo, n=7). J. Tumor weight on day 17. K. Absolute number of CD8+ T cells in the dLN. L. Absolute number of CD8+ T cells in tumors. *p<0.05, **p<0.01; Tumor curve analysis was performed using repeated measures two-way analysis of variance (ANOVA). Other data was performed using two-tailed Student's t test. Figures B, D, I were corrected for multiple testing using the Sidak procedure. Data presented as mean+/−SEM.

FIGS. 27A-27F Systemic administration of PIIO-1 to hLRRC32KI mice increases peripheral LN cellularity including CD8+ T cells and their function. A. hLRRC32KI mice were injected i.v. with 200 μg/mouse PIIO-1 or mIgG1 every 48 hours for a total of 3 injections. Mice were sacrificed and peripheral lymph nodes were harvested 24 hours after the 3rd injection of PIIO-1. B. Absolute number of live cells from peripheral lymph nodes. C-E. Flow cytometric analysis of peripheral lymph node examining the frequency of, C. CD3+CD8+ T cells, D. Ki67+CD8⁺ T cells, and E. Foxp3+ regulatory T cells. F. Percentage of IFNγ and TNFα producing CD8⁺ T cells by intracellular staining. N=3 per group, data representative of two independent experiments. Two-tailed Student's t test was used for statistics. Data presented as mean+/−SEM. *p<0.05, **p<0.01.

FIGS. 28A and 28B show GARP expression alters CD8⁺ T cell phenotype in the TME. A. Subcluster analysis of tumor-infiltrating CD8+ T cells in EV vs hGARP over-expressing MB-49 tumor. 1×10⁵ MB-49-EV or hGARP cells were injected into C57BL/6 mice s.c. and tumors were harvested on day 18. UMAP dimension reduction of tumor-infiltrating CD8+ T cells was done after staining with 33 markers and spectral flow cytometry analysis. Shown is the data gated on live CD45+CD3+CD8+ T cells, subsampled on 5000 cells per sample. Unsupervised clustering analysis was performed using FlowSOM algorithm with an elbow method approach for cluster number determination. B. Heatmap of A showing expression levels of indicated markers by each cluster. Cluster difference was measured by two-tailed Student's t test. Data presented as mean+/−SEM. ***p<0.001.

FIGS. 29A-29D show PIIO-1 alters CD8⁺ T cell infiltration and clustering. A. Cell density analysis of tumor-infiltrating CD8+ T cells in MB-49 tumor treated with mIgG1 or PIIO-1. 1×10⁵ MB-49 cells were injected s.c. on the right flank of bLRRC32KI male mice. PIIO-1 or ISO was delivered (200 μg/mouse, i.p.) on days 6 and 9. Tumors were collected on day 10 and multiplex IF analysis was performed on histology samples of the tumors. (Left) Histology samples were stained with CD45, CD8, SMA, DAPI. (Upper right) Shows tumor regions defined for computational analysis. The boundary at α=1 denotes the boundary between the stromal and the tumor region. This boundary was scaled down by a to create additional tumor regions (see supplemental methods for further details). (Lower right) CD8+ T cell density was quantified in the regions defined in (A) for ISO and the PIIO-1 treated. PIIO-1 treatment increased CD8+ T cell density in the intermediate II region compared to ISO. B. Co-dependence of the densities of SMA+ and CD8+ T cells in the interior region defined in (A). Densities obtained from slides from different mice are shown with different symbols. The magnitude of the negative correlation between the SMA+ and CD8+ T cells in the ISO (Corr=−0.86) decreases when the tumor is treated with PIIO-1 (Corr=−0.62). C. Core steps used in the calculation of the two point correlation function where the density of CD8+ T cells in an annular region of radius r and thickness δ corresponding to a CD8+ T cell at the center is calculated (see supplemental methods for further details). D. Variation of the two point correlation function C(r) with the distance r for the CD8+ T cells in the interior (left) and the intermediate II (right) tumor regions for tumors in ISO and PIIO-1 treated mice. Multiple curves in the same color show the data for C(r) obtained from different slides in different mice (ISO or PIIO-1 treated). The data shows that C(r) has larger peaks at r≈7 μm for the PIIO-1 treated compared to ISO in the intermediate II region. This indicates increased grouping of CD8+ T cells within a length scale of 7 μm in the intermediate II region when treated with PIIO-1. Cell density difference measured by Welch's t test. Data presented as mean+/−SEM.

FIGS. 30A-30C show PIIO-1 overcomes resistance to PD-1 blockade in LLC and CMT-167 models and promotes CD8⁺ T cell infiltration. A. Summary of number of mice in each treatment group with uncontrolled tumors (>115 mm2 on day 17). 5×10⁵ LLC cells were injected s.c. on the right flank of hLRRC32KI female mice, followed by treatment with ISO, PIIO-1, PD-1 or combination. Treatments were delivered on day 8 after tumor inoculation and every 3 days thereafter for a total of 4 doses B. Tumor volume 18 days after s.c. injection of 1×10⁵ CMT-167 cells. Mice were treated with 4 injections of indicated antibody (day 8, 11, 14 and 17). C. Frequency of tumor-infiltrating CD8⁺ T cells of day 18 CMT-167 tumors (left-representative flow plots gated on CD45+ cells; right—data quantification). Data from A is analyzed by two-tailed Fisher's exact test. Other data is analyzed by two-tailed Student's t test. All data are presented as mean±SEM. *p<0.05, ****p<0.0001.

FIGS. 31A-31C show PIIO-1 attenuates canonical TGFβ pathway in immune cells and target Tregs primarily in the dLN. FIG. 31A shows 1×10⁵ MB-49 cells were injected s.c. in the right flank of hLRRC32KI male mice. Humanized PIIO-1 (200 μg/mouse, i.p.) was administered on days 18 and 20. dLNs were collected on day 21, then isolated and stained for intracellular pSMAD2/3 with cell linage markers (see supplemental methods for further details), followed by flow cytometry analysis. pSMAD2/3 expression level in cells from dLN was shown. FIG. 31B shows quantification of panel 31A. FIG. 31C shows 1×10⁵ MB-49 cells were injected s.c. in the right flank of hLRRC32KI male mice. Humanized PIIO-1 (200 μg/mouse, i.p.) was administered on day 18. Tumor dLNs, tumor and spleen were collected on day 19. Humanized PIIO-1 was detected by anti-human Fc flow antibody. Humanized PIIO-1 and LAP co-expressed cells were gated and further analyzed for cell identity. Data was performed using two-tailed Student's t test and presented as mean+/−SEM. *p<0.05, **p<0.01.

FIG. 32 shows Anti-CXCR3, with or without anti-GARP antibody PIIO-1 does not alter Treg numbers in the TME. 1×10⁵ MB-49 cells were injected s.c. in the right flank of hLRRC32KI male mice. Humanized PIIO-1 and anti-CXCR3 antibody were administered (200 μg/mouse, i.p.) every 3 days for a total of 4 treatments starting on day 5. Absolute number of Treg cells in the tumor was then quantified by flow cytometry, based on live gating of TILs with the following phenotype: CD45+CD3+CD4+CD25+Foxp3+. Data are presented as mean+/−SEM. No significant difference between groups was observed based on two-tailed Student's t test.

FIGS. 33A and 33B show the characterization of anti-human GARP antibodies for recognition of cell surface GARP and blocking of GARP-LTGFβ interaction. FIG. 33A shows GARP expression on Jurkat-hGARP cell line was detected by flow cytometry with anti-GARP antibodies at indicated concentrations. Geometric mean fluorescence intensity (gMFI) of human GARP was plotted. FIG. 33B shows stable hGARP-expressing Jurkat cell line was incubated with recombinant LTGFβ1 together with isotype control or anti-GARP antibodies at indicated concentrations for 30 min at 37° C. Human LTGFβ1 expression level was detected by flow cytometry.

### DESCRIPTION OF ILLUSTRATIVE EMBODIMENTS

It is demonstrated herein that both membrane-bound and soluble GARP is widely expressed by human cancer cells but less by normal epithelial cells, and the expression of GARP correlates uniformly with an advanced stage of cancer and poor prognosis. Additionally, it was found that GARP itself has a transformation potential, which renders normal mammary gland epithelial cells tumorgenic. It was observed that GARP expression in cancer cells led to increased TGF-β activity, likely due to its ability to concentrate LTGF-β in cis as well as trans, to contribute to cancer aggressiveness and metastasis. GARP expression in the tumor microenvironment promoted the induction of regulatory T cells and thus blunting the function of effector T cells against cancers. However, neutralizing GARP by blockings its ability to bind to TGF-β results in anti-cancer activity even, without chemotherapy. In particular, there are provided here new antibody molecules, the humanized PIIO-1 antibodies HuPIIO-1VH1/L1, HuPIIO-1VH1/L2, HuPIIO-1VH2/L1, HuPIIO-1VH1/L3, HuPIIO-1VH2/L2, HuPIIO-1VH2/L3, HuPIIO-1VH3/L1, HuPIIO-1VH2/L3, HuPIIO-1VH3/L3, HuPIIO-1VH4/L1, HuPIIO-1VH4/L2, HuPIIO-1VH4/L3 and 5c5 antibodies that can effectively bind to and neutralize GARP. Thus, the antibodies of the embodiments can be used in methods for treating cancers and enhancing immune response (e.g., in conjunction with an adoptive T-cell therapy).

While T cell therapy has the potential to treat cancer by recognizing and attacking tumor cells, the tumor microenvironment can evade the immune system through the induction of regulatory T cells which blunt the ability of adoptively transferred effector T cells to control cancer. Accordingly, embodiments of the present disclosure overcomes challenges associated with current technologies by providing methods for the treatment of cancer comprising the combination of a T cell therapy and an anti-platelet agent. In this method, the anti-platelet agent can potentiate the adoptive T cell therapy of tumors as soluble factors secreted from activated platelets have been shown to suppress T cells. For example, it has been shown that platelet-secreted latent TGFβ and GARP can lead to the resistance of cancer cells to adoptive T cell therapy. Thus, anti-platelet factors such as an anti-GARP monoclonal antibody (that can block TGFβ binding) can be used in combination with the T cell therapy to overcome this resistance and treat cancer. In addition, other immunotherapies such as an immune checkpoint inhibitor can be used in combination with the T cell therapy and anti-platelet agent to enhance the immune response.

### I. DEFINITIONS

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%. Most preferred is a composition in which no amount of the specified component can be detected with standard analytical methods.

As used herein in the specification and claims, “a” or “an” may mean one or more. As used herein in the specification and claims, when used in conjunction with the word “comprising”, the words “a” or “an” may mean one or more than one. As used herein, in the specification and claim, “another” or “a further” may mean at least a second or more.

As used herein in the specification and claims, the term “about” is used to indicate that a value includes the inherent variation of error for the device, the method being employed to determine the value, or the variation that exists among the study subjects.

“Treatment” and “treating” refer to administration or application of a therapeutic agent to a subject or performance of a procedure or modality on a subject for the purpose of obtaining a therapeutic benefit of a disease or health-related condition. For example, a treatment may include administration of a pharmaceutically effective amount of an antibody that inhibits the GARP signaling. In another example, a treatment may include administration of a T cell therapy and a pharmaceutically effective amount of an anti-platelet agent (e.g., an antibody that inhibits the GARP signaling).

“Subject” and “patient” refer to either a human or non-human, such as primates, mammals, and vertebrates. In particular embodiments, the subject is a human.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer.

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

An “anti-cancer” agent is capable of negatively affecting a cancer cell/tumor in a subject, for example, by promoting killing of cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer.

The term “antibody” herein is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity.

The term “monoclonal antibody” as used herein refers to an antibody obtained from a population of substantially homogeneous antibodies, e.g., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

The term “unit dose” or “dosage” refers to physically discrete units suitable for use in a subject, each unit containing a predetermined quantity of the therapeutic composition calculated to produce the desired responses discussed above in association with its administration, i.e., the appropriate route and treatment regimen. The quantity to be administered, both according to number of treatments and unit dose, depends on the effect desired. The actual dosage amount of a composition of the present embodiments administered to a patient or subject can be determined by physical and physiological factors, such as body weight, the age, health, and sex of the subject, the type of disease being treated, the extent of disease penetration, previous or concurrent therapeutic interventions, idiopathy of the patient, the route of administration, and the potency, stability, and toxicity of the particular therapeutic substance. For example, a dose may also comprise from about 1 μg/kg/body weight to about 1000 mg/kg/body weight (this such range includes intervening doses) or more per administration, and any range derivable therein. In non-limiting examples of a derivable range from the numbers listed herein, a range of about 5 μg/kg/body weight to about 100 mg/kg/body weight, about 5 μg/kg/body weight to about 500 mg/kg/body weight, etc., can be administered. The practitioner responsible for administration will, in any event, determine the concentration of active ingredient(s) in a composition and appropriate dose(s) for the individual subject.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing, for example, both agents are delivered to a cell in a combined amount effective to kill the cell or prevent it from dividing.

The term “immune checkpoint” refers to a molecule such as a protein in the immune system which provides inhibitory signals to its components in order to balance immune reactions. Known immune checkpoint proteins comprise cytotoxic T-lymphocyte-associated protein 4 (CTLA-4), program cell death protein 1 (PD1) and its ligands programmed death ligand 1 (PD-L1) and programmed death ligand 2 (PD-L2) and in addition LAG-3, lymphocyte activation gene 3 (LAG-3), B- and T-lymphocyte attenuator (BTLA), B7 homolog 3 (B7H3), B7 homolog 4 (B7H4), T-cell immunoglobulin and mucin domain 3 (Tim-3), killer immunoglobulin-like receptor (KIR). The pathways involving LAG3, B- and T-lymphocyte attenuator (BTLA), V-domain Ig suppressor of T cell activation (VISTA), B7H3, B7H4, TIM3, T cell immunoreceptor with Ig and ITIM domains (TIGIT), and KIR are recognized in the art to constitute immune checkpoint pathways similar to the CTLA-4 and PD-1 dependent pathways (see, e.g., Pardoll, 2012, Nature Rev Cancer 12:252-264; Mellman et al., 2011, Nature 480:480-489).

An “immune checkpoint inhibitor” refers to any compound inhibiting the function of an immune checkpoint protein. Inhibition includes reduction of function and full blockade. In particular the immune checkpoint protein is a human immune checkpoint protein. Thus, the immune checkpoint protein inhibitor in particular is an inhibitor of a human immune checkpoint protein. Examples of checkpoint inhibitors include, but are not limited to anti-PD-1 (such as, for example, Nivolumab (BMS-936558 or MDX1106), pembrolizumab, CT-011, MK-3475), anti-PD-L1 (such as, for example, atezolizumab, avelumab, durvalumab, MDX-1105 (BMS-936559), MPDL3280A, or MSB0010718C) such as, for example, PD-L2 (rHIgM12B7), anti-CTLA-4 (such as, for example, Ipilimumab (MDX-010), Tremelimumab (CP-675,206)), anti-IDO, anti-B7-H3 (such as, for example, MGA271, MGD009, omburtamab), anti-B7-H4, anti-TIM3 (such as, for example, TSR-022, MBG453, Sym023, INCAGN2390, LY3321367, BMS-986258, SHR-1702, RO7121661), anti-TIGIT (such as, for example BMS-986207, OMP-313M32, MK-7684, AB-154, ASP-8374, MTIG7192A, or PVSRIPO), anti-BTLA, anti-LAG-3 (such as, for example, BMS-986016, LAG525, MK-4280, REGN3767, TSR-033, BI754111, Sym022, FS118, MGD013, and Immutep).

### II. ANTIBODIES OF THE EMBODIMENTS

In certain embodiments, an antibody or a fragment thereof that binds to at least a portion of GARP protein and inhibits GARP signaling and cancer cell proliferation are contemplated. As used herein, the term “antibody” is intended to refer broadly to any immunologic binding agent, such as IgG, IgM, IgA, IgD, IgE, and genetically modified IgG as well as polypeptides comprising antibody CDR domains that retain antigen binding activity. The antibody may be selected from the group consisting of a chimeric antibody, an affinity matured antibody, a polyclonal antibody, a monoclonal antibody, a humanized antibody, a human antibody, or an antigen-binding antibody fragment or a natural or synthetic ligand. Preferably, the anti-GARP antibody is a monoclonal antibody or a humanized antibody.

Thus, by known means and as described herein, polyclonal or monoclonal antibodies, antibody fragments, and binding domains and CDRs (including engineered forms of any of the foregoing) may be created that are specific to GARP protein, one or more of its respective epitopes, or conjugates of any of the foregoing, whether such antigens or epitopes are isolated from natural sources or are synthetic derivatives or variants of the natural compounds.

Examples of antibody fragments suitable for the present embodiments include, without limitation: (i) the Fab fragment, consisting of V_{L}, V_{H}, C_{L}, and C_{H1} domains; (ii) the “Fd” fragment consisting of the V_{H} and C_{H1} domains; (iii) the “Fv” fragment consisting of the V_{L} and V_{H} domains of a single antibody; (iv) the “dAb” fragment, which consists of a V_{H} domain; (v) isolated CDR regions; (vi) F(ab′)2 fragments, a bivalent fragment comprising two linked Fab fragments; (vii) single chain Fv molecules (“scFv”), wherein a V_{H} domain and a V_{L} domain are linked by a peptide linker that allows the two domains to associate to form a binding domain; (viii) bi-specific single chain Fv dimers (see U.S. Pat. No. 5,091,513); and (ix) diabodies, multivalent or multispecific fragments constructed by gene fusion (U.S. Patent Pub. 20050214860). Fv, scFv, or diabody molecules may be stabilized by the incorporation of disulphide bridges linking the V_{H} and V_{L} domains. Minibodies comprising a scFv joined to a CH3 domain may also be made (Hu et al., 1996).

Antibody-like binding peptidomimetics are also contemplated in embodiments. Liu et al. (2003) describe “antibody like binding peptidomimetics” (ABiPs), which are peptides that act as pared-down antibodies and have certain advantages of longer serum half-life as well as less cumbersome synthesis methods.

Animals may be inoculated with an antigen, such as a GARP extracellular domain (ECD) protein, in order to produce antibodies specific for GARP protein. Frequently an antigen is bound or conjugated to another molecule to enhance the immune response. As used herein, a conjugate is any peptide, polypeptide, protein, or non-proteinaceous substance bound to an antigen that is used to elicit an immune response in an animal. Antibodies produced in an animal in response to antigen inoculation comprise a variety of non-identical molecules (polyclonal antibodies) made from a variety of individual antibody producing B lymphocytes. A polyclonal antibody is a mixed population of antibody species, each of which may recognize a different epitope on the same antigen. Given the correct conditions for polyclonal antibody production in an animal, most of the antibodies in the animal's serum will recognize the collective epitopes on the antigenic compound to which the animal has been immunized. This specificity is further enhanced by affinity purification to select only those antibodies that recognize the antigen or epitope of interest.

A monoclonal antibody is a single species of antibody wherein every antibody molecule recognizes the same epitope because all antibody producing cells are derived from a single B-lymphocyte cell line. The methods for generating monoclonal antibodies (MAbs) generally begin along the same lines as those for preparing polyclonal antibodies. In some embodiments, rodents such as mice and rats are used in generating monoclonal antibodies. In some embodiments, rabbit, sheep, or frog cells are used in generating monoclonal antibodies. The use of rats is well known and may provide certain advantages. Mice (e.g., BALB/c mice) are routinely used and generally give a high percentage of stable fusions.

Hybridoma technology involves the fusion of a single B lymphocyte from a mouse previously immunized with a GARP antigen with an immortal myeloma cell (usually mouse myeloma). This technology provides a method to propagate a single antibody-producing cell for an indefinite number of generations, such that unlimited quantities of structurally identical antibodies having the same antigen or epitope specificity (monoclonal antibodies) may be produced.

Plasma B cells (CD45⁺CD5⁻CD19⁺) may be isolated from freshly prepared rabbit peripheral blood mononuclear cells of immunized rabbits and further selected for GARP binding cells. After enrichment of antibody producing B cells, total RNA may be isolated and cDNA synthesized. DNA sequences of antibody variable regions from both heavy chains and light chains may be amplified, constructed into a phage display Fab expression vector, and transformed into E. coli. GARP specific binding Fab may be selected out through multiple rounds enrichment panning and sequenced. Selected GARP binding hits may be expressed as full-length IgG in rabbit and rabbit/human chimeric forms using a mammalian expression vector system in human embryonic kidney (HEK293) cells (Invitrogen) and purified using a protein G resin with a fast protein liquid chromatography (FPLC) separation unit.

In one embodiment, the antibody is a chimeric antibody, for example, an antibody comprising antigen binding sequences from a non-human donor grafted to a heterologous non-human, human, or humanized sequence (e.g., framework and/or constant domain sequences). Methods have been developed to replace light and heavy chain constant domains of the monoclonal antibody with analogous domains of human origin, leaving the variable regions of the foreign antibody intact. Alternatively, “fully human” monoclonal antibodies are produced in mice transgenic for human immunoglobulin genes. Methods have also been developed to convert variable domains of monoclonal antibodies to more human form by recombinantly constructing antibody variable domains having both rodent, for example, mouse, and human amino acid sequences. In “humanized” monoclonal antibodies, only the hypervariable CDR is derived from mouse monoclonal antibodies, and the framework and constant regions are derived from human amino acid sequences (see U.S. Pat. Nos. 5,091,513 and 6,881,557). It is thought that replacing amino acid sequences in the antibody that are characteristic of rodents with amino acid sequences found in the corresponding position of human antibodies will reduce the likelihood of adverse immune reaction during therapeutic use. A hybridoma or other cell producing an antibody may also be subject to genetic mutation or other changes, which may or may not alter the binding specificity of antibodies produced by the hybridoma.

Methods for producing polyclonal antibodies in various animal species, as well as for producing monoclonal antibodies of various types, including humanized, chimeric, and fully human, are well known in the art and highly predictable. For example, the following U.S. patents and patent applications provide enabling descriptions of such methods: U.S. Patent Application Nos. 2004/0126828 and 2002/0172677, and U.S. Pat. Nos. 3,817,837; 3,850,752; 3,939,350; 3,996,345; 4,196,265; 4,275, 149; 4,277,437; 4,366,241; 4,469,797; 4,472,509; 4,606,855; 4,703,003; 4,742, 159; 4,767,720; 4,816,567; 4,867,973; 4,938,948; 4,946,778; 5,021,236; 5,164,296; 5,196,066; 5,223,409; 5,403,484; 5,420,253; 5,565,332; 5,571,698; 5,627,052; 5,656,434; 5,770,376; 5,789,208; 5,821,337; 5,844,091; 5,858,657; 5,861,155; 5,871,907; 5,969,108; 6,054,297; 6,165,464; 6,365,157; 6,406,867; 6,709,659; 6,709,873; 6,753,407; 6,814,965; 6,849,259; 6,861,572; 6,875,434; and 6,891,024. All patents, patent application publications, and other publications cited herein and therein are hereby incorporated by reference in the present application.

Antibodies may be produced from any animal source, including birds and mammals. Preferably, the antibodies are ovine, murine (e.g., mouse and rat), rabbit, goat, guinea pig, camel, horse, or chicken. In addition, newer technology permits the development of and screening for human antibodies from human combinatorial antibody libraries. For example, bacteriophage antibody expression technology allows specific antibodies to be produced in the absence of animal immunization, as described in U.S. Pat. No. 6,946,546, which is incorporated herein by reference. These techniques are further described in: Marks (1992); Stemmer (1994); Gram et al. (1992); Barbas et al. (1994); and Schier et al. (1996).

It is fully expected that antibodies to GARP will have the ability to neutralize or counteract the effects of GARP regardless of the animal species, monoclonal cell line, or other source of the antibody. Certain animal species may be less preferable for generating therapeutic antibodies because they may be more likely to cause allergic response due to activation of the complement system through the “Fc” portion of the antibody. However, whole antibodies may be enzymatically digested into “Fc” (complement binding) fragment, and into antibody fragments having the binding domain or CDR. Removal of the Fc portion reduces the likelihood that the antigen antibody fragment will elicit an undesirable immunological response, and thus, antibodies without Fc may be preferential for prophylactic or therapeutic treatments. As described above, antibodies may also be constructed so as to be chimeric or partially or fully human, so as to reduce or eliminate the adverse immunological consequences resulting from administering to an animal an antibody that has been produced in, or has sequences from, other species.

Substitutional variants typically contain the exchange of one amino acid for another at one or more sites within the protein and may be designed to modulate one or more properties of the polypeptide, with or without the loss of other functions or properties. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine, tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa.

Proteins may be recombinant or synthesized in vitro. Alternatively, a non-recombinant or recombinant protein may be isolated from bacteria. It is also contemplated that a bacteriim containing such a variant may be implemented in compositions and methods. Consequently, a protein need not be isolated.

It is contemplated that in compositions there is between about 0.001 mg and about 10 mg of total polypeptide, peptide, and/or protein per ml. Thus, the concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein). Of this, about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% may be an antibody that binds GARP

An antibody or preferably an immunological portion of an antibody, can be chemically conjugated to, or expressed as, a fusion protein with other proteins. For purposes of this specification and the accompanying claims, all such fused proteins are included in the definition of antibodies or an immunological portion of an antibody.

Embodiments provide antibodies and antibody-like molecules against GARP, polypeptides and peptides that are linked to at least one agent to form an antibody conjugate or payload. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules that have been attached to antibodies include toxins, therapeutic enzymes, antibiotics, radio-labeled nucleotides and the like. By contrast, a reporter molecule is defined as any moiety that may be detected using an assay. Non-limiting examples of reporter molecules that have been conjugated to antibodies include enzymes, radiolabels, haptens, fluorescent labels, phosphorescent molecules, chemiluminescent molecules, chromophores, luminescent molecules, photoaffinity molecules, colored particles or ligands, such as biotin.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3-6-diphenylglycouril-3 attached to the antibody. Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate.

### III. T CELL THERAPY

Certain embodiments of the present disclosure concern obtaining and administering T cells to a subject as an immunotherapy to target cancer cells. Several basic approaches for the derivation, activation and expansion of functional anti-tumor effector T cells have been described in the last two decades. These include: autologous cells, such as tumor-infiltrating lymphocytes (TILs); T cells activated ex-vivo using autologous DCs, lymphocytes, artificial antigen-presenting cells (APCs) or beads coated with T cell ligands and activating antibodies, or cells isolated by virtue of capturing target cell membrane; allogeneic cells naturally expressing anti-host tumor T cell receptor (TCR); and non-tumor-specific autologous or allogeneic cells genetically reprogrammed or “redirected” to express tumor-reactive TCR or chimeric TCR molecules displaying antibody-like tumor recognition capacity known as “T-bodies”. These approaches have given rise to numerous protocols for T cell preparation and immunization which can be used in the methods of the present disclosure.

### A. T Cell Preparation

In some embodiments, the T cells are derived from the blood, bone marrow, lymph, or lymphoid organs. In some aspects, the cells are human cells. The cells typically are primary cells, such as those isolated directly from a subject and/or isolated from a subject and frozen. In some embodiments, the cells include one or more subsets of T cells or other cell types, such as whole T cell populations, CD4⁺ cells, CD8⁺ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, as described herein, and re-introducing them into the same patient, before or after cryopreservation.

Among the sub-types and subpopulations of T cells (e.g., CD4⁺ and/or CD8⁺ T cells) are naive T (T_{N}) cells, effector T cells (T_{EFF}), memory T cells and sub-types thereof, such as stem cell memory T (TSC_{M}), central memory T (TC_{M}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, one or more of the T cell populations is enriched for or depleted of cells that are positive for a specific marker, such as surface markers, or that are negative for a specific marker. In some cases, such markers are those that are absent or expressed at relatively low levels on certain populations of T cells (e.g., non-memory cells) but are present or expressed at relatively higher levels on certain other populations of T cells (e.g., memory cells). In one embodiment, the cells (e.g., CD8⁺ cells or CD3⁺ cells) are enriched for (i.e., positively selected for) cells that are positive or expressing high surface levels of CD45RO, CCR7, CD28, CD27, CD44, CD127, and/or CD62L and/or depleted of (e.g., negatively selected for) cells that are positive for or express high surface levels of CD45RA. In some embodiments, cells are enriched for or depleted of cells positive or expressing high surface levels of CD122, CD95, CD25, CD27, and/or IL7-Ra (CD127). In some examples, CD8⁺ T cells are enriched for cells positive for CD45RO (or negative for CD45RA) and for CD62L.

In some embodiments, T cells are separated from a PBMC sample by negative selection of markers expressed on non-T cells, such as B cells, monocytes, or other white blood cells, such as CD14. In some aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations.

In some embodiments, CD8⁺ cells are further enriched for or depleted of naive, central memory, effector memory, and/or central memory stem cells, such as by positive or negative selection based on surface antigens associated with the respective subpopulation. In some embodiments, enrichment for central memory T (TC_{M}) cells is carried out to increase efficacy, such as to improve long-term survival, expansion, and/or engraftment following administration, which in some aspects is particularly robust in such sub-populations. See Terakura et al. (2012) Blood. 1:72-82; Wang et al. (2012) J Immunother. 35(9):689-701. In some embodiments, combining T_{CM}-enriched CD8⁺ T cells and CD4⁺ T cells further enhances efficacy.

In some embodiments, the T cells are autologous T cells. In this method, tumor samples are obtained from patients and a single cell suspension is obtained. The single cell suspension can be obtained in any suitable manner, e.g., mechanically (disaggregating the tumor using, e.g., a gentleMACS™ Dissociator, Miltenyi Biotec, Auburn, Calif.) or enzymatically (e.g., collagenase or DNase). Single-cell suspensions of tumor enzymatic digests are cultured in interleukin-2 (IL-2). The cells are cultured until confluence (e.g., about 2×10⁶ lymphocytes), e.g., from about 5 to about 21 days, preferably from about 10 to about 14 days. For example, the cells may be cultured from 5 days, 5.5 days, or 5.8 days to 21 days, 21.5 days, or 21.8 days, such as from 10 days, 10.5 days, or 10.8 days to 14 days, 14.5 days, or 14.8 days.

The cultured T cells can be pooled and rapidly expanded. Rapid expansion provides an increase in the number of antigen-specific T-cells of at least about 50-fold (e.g., 50-, 60-, 70-, 80-, 90-, or 100-fold, or greater) over a period of about 10 to about 14 days, preferably about 14 days. More preferably, rapid expansion provides an increase of at least about 200-fold (e.g., 200-, 300-, 400-, 500-, 600-, 700-, 800-, 900-, or greater) over a period of about 10 to about 14 days, preferably about 14 days.

Expansion can be accomplished by any of a number of methods as are known in the art. For example, T cells can be rapidly expanded using non-specific T-cell receptor stimulation in the presence of feeder lymphocytes and either interleukin-2 (IL-2) or interleukin-15 (IL-15), with IL-2 being preferred. The non-specific T-cell receptor stimulus can include around 30 ng/ml of OKT3, a mouse monoclonal anti-CD3 antibody (available from Ortho-McNeil®, Raritan, N.J.). Alternatively, T cells can be rapidly expanded by stimulation of peripheral blood mononuclear cells (PBMC) in vitro with one or more antigens (including antigenic portions thereof, such as epitope(s), or a cell) of the cancer, which can be optionally expressed from a vector, such as an human leukocyte antigen A2 (HLA-A2) binding peptide, in the presence of a T-cell growth factor, such as 300 IU/ml IL-2 or IL-15, with IL-2 being preferred. The in vitro-induced T-cells are rapidly expanded by re-stimulation with the same antigen(s) of the cancer pulsed onto HLA-A2-expressing antigen-presenting cells. Alternatively, the T-cells can be re-stimulated with irradiated, autologous lymphocytes or with irradiated HLA-A2+ allogeneic lymphocytes and IL-2, for example.

The autologous T-cells can be modified to express a T-cell growth factor that promotes the growth and activation of the autologous T-cells. Suitable T-cell growth factors include, for example, interleukin (IL)-2, IL-7, IL-15, and IL-12. Suitable methods of modification are known in the art. See, for instance, Sambrook et al., Molecular Cloning: A Laboratory Manual, 3^{rd} ed., Cold Spring Harbor Press, Cold Spring Harbor, N.Y. 2001; and Ausubel et al., Current Protocols in Molecular Biology, Greene Publishing Associates and John Wiley & Sons, NY, 1994. In particular aspects, modified autologous T-cells express the T-cell growth factor at high levels. T-cell growth factor coding sequences, such as that of IL-12, are readily available in the art, as are promoters, the operable linkage of which to a T-cell growth factor coding sequence promote high-level expression.

### B. Genetically Engineered Antigen Receptors

The T cell can genetically engineered to express antigen receptors such as engineered TCRs and/or chimeric antigen receptors (CARs). For example, the autologous T-cells are modified to express a T cell receptor (TCR) having antigenic specificity for a cancer antigen. Suitable TCRs include, for example, those with antigenic specificity for a melanoma antigen, e.g., gp100 or MART-1. Suitable methods of modification are known in the art. See, for instance, Sambrook and Ausubel, supra. For example, the T cells may be transduced to express a T cell receptor (TCR) having antigenic specificity for a cancer antigen using transduction techniques described in Heemskerk et al. Hum Gene Ther. 19:496-510 (2008) and Johnson et al. Blood 114:535-46 (2009).

In some embodiments, the T cells comprise one or more nucleic acids introduced via genetic engineering that encode one or more antigen receptors, and genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature (e.g., chimeric).

In some embodiments, the CAR contains an extracellular antigen-recognition domain that specifically binds to an antigen. In some embodiments, the antigen is a protein expressed on the surface of cells. In some embodiments, the CAR is a TCR-like CAR and the antigen is a processed peptide antigen, such as a peptide antigen of an intracellular protein, which, like a TCR, is recognized on the cell surface in the context of a major histocompatibility complex (MHC) molecule.

Exemplary antigen receptors, including CARs and recombinant TCRs, as well as methods for engineering and introducing the receptors into cells, include those described, for example, in international patent application publication numbers WO200014257, WO2013126726, WO2012/129514, WO2014031687, WO2013/166321, WO2013/071154, WO2013/123061 U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov. 2013 April; 3(4):388-398; Davila et al. (2013) PLOS ONE 8(4): e61338; Turtle et al., Curr. Opin. Immunol., 2012 October; 24(5): 633-39; Wu et al., Cancer, 2012 Mar. 18(2): 160-75. In some aspects, the genetically engineered antigen receptors include a CAR as described in U.S. Pat. No. 7,446,190, and those described in International Patent Application Publication No.: WO/2014055668 A1.

In some aspects, the tumor antigen is a human telomerase reverse transcriptase (hTERT), survivin, mouse double minute 2 homolog (MDM2), cytochrome P450 1B1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53 or cyclin (DI). For example, the target antigen is hTERT or survivin. In some aspects, the target antigen is CD38. In other aspects, the target antigen is CD33 or TIM-3. In other aspects, it is CD26, CD30, CD53, CD92, CD148, CD150, CD200, CD261, CD262, or CD362. In some embodiments, the engineered immune cells can contain an antigen that targets one or more other antigens. In some embodiments, the one or more other antigens is a tumor antigen or cancer marker Other antigens include orphan tyrosine kinase receptor ROR1, tEGFR, Her2, L1-CAM, CD19, CD20, CD22, mesothelin, CEA, and hepatitis B surface antigen, anti-folate receptor, CD23, CD24, CD30, CD33, CD38, CD44, EGFR, EGP-2, EGP-4, EPHa2, ErbB2, 3, or 4, FBP, fetal acethycholine e receptor, GD2, GD3, HMW-MAA, IL-22R-alpha, IL-13R-alpha2, kdr, kappa light chain, Lewis Y, L1-cell adhesion molecule, MAGE-A1, mesothelin, MUCI, MUC16, PSCA, NKG2D Ligands, NY-ESO-1, MART-1, gplOO, oncofetal antigen, ROR1, TAG72, VEGF-R2, carcinoembryonic antigen (CEA), prostate specific antigen, PSMA, Her2/neu, estrogen receptor, progesterone receptor, ephrinB2, CD 123, CS-1, c-Met, GD-2, and MAGE A3, CE7, Wilms Tumor 1 (WT-1), a cyclin, such as cyclin A1 (CCNA1), and/or biotinylated molecules, and/or molecules expressed by HIV, HCV, HBV or other pathogens.

### 1. Chimeric Antigen Receptors

In some embodiments, the engineered antigen receptors include chimeric antigen receptors (CARs), including activating or stimulatory CARs, costimulatory CARs (see WO2014/055668), and/or inhibitory CARs (iCARs, see Fedorov et al., Sci. Transl. Medicine, 5(215) (2013). The CARs generally include an extracellular antigen (or ligand) binding domain linked to one or more intracellular signaling components, in some aspects via linkers and/or transmembrane domain(s). Such molecules typically mimic or approximate a signal through a natural antigen receptor, a signal through such a receptor in combination with a costimulatory receptor, and/or a signal through a costimulatory receptor alone.

In some embodiments, CAR is constructed with a specificity for a particular antigen (or marker or ligand), such as an antigen expressed in a particular cell type to be targeted by adoptive therapy, e.g., a cancer marker, and/or an antigen intended to induce a dampening response, such as an antigen expressed on a normal or non-diseased cell type. Thus, the CAR typically includes in its extracellular portion one or more antigen binding molecules, such as one or more antigen-binding fragment, domain, or portion, or one or more antibody variable domains, and/or antibody molecules. In some embodiments, the CAR includes an antigen-binding portion or portions of an antibody molecule, such as a single-chain antibody fragment (scFv) derived from the variable heavy (VH) and variable light (VL) chains of a monoclonal antibody (mAb).

In some aspects, the antigen-specific binding, or recognition component is linked to one or more transmembrane and intracellular signaling domains. In some embodiments, the CAR includes a transmembrane domain fused to the extracellular domain of the CAR. In one embodiment, the transmembrane domain that naturally is associated with one of the domains in the CAR is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CD5, CDS, CD9, CD 16, CD22, CD33, CD37, CD64, CD80, CD86, CD 134, CD137, CD 154. Alternatively, the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain.

The CAR generally includes at least one intracellular signaling component or components. In some embodiments, the CAR includes an intracellular component of the TCR complex, such as a TCR CD3⁺ chain that mediates T-cell activation and cytotoxicity, e.g., CD3 zeta chain. Thus, in some aspects, the antigen binding molecule is linked to one or more cell signaling modules. In some embodiments, cell signaling modules include CD3 transmembrane domain, CD3 intracellular signaling domains, and/or other CD transmembrane domains. In some embodiments, the CAR further includes a portion of one or more additional molecules such as Fc receptor γ, CD8, CD4, CD25, or CD16. For example, in some aspects, the CAR includes a chimeric molecule between CD3-zeta (CD3-Q or Fc receptor γ and CD8, CD4, CD25 or CD16.

### 2. T Cell Receptor (TCR)

In some embodiments, the genetically engineered antigen receptors include recombinant T cell receptors (TCRs) and/or TCRs cloned from naturally occurring T cells. A “T cell receptor” or “TCR” refers to a molecule that contains a variable a and β chains (also known as TCRa and TCRp, respectively) or a variable γ and δ chains (also known as TCRy and TCR5, respectively) and that is capable of specifically binding to an antigen peptide bound to a MHC receptor. In some embodiments, the TCR is in the αβ form. Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules. In some embodiments, a TCR also can contain a constant domain, a transmembrane domain and/or a short cytoplasmic tail (see, e.g., Janeway et al, Immunobiology: The Immune System in Health and Disease, 3rd Ed., Current Biology Publications, p. 4:33, 1997). For example, in some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction. Unless otherwise stated, the term “TCR” should be understood to encompass functional TCR fragments thereof. The term also encompasses intact or full-length TCRs, including TCRs in the αβ form or γδ form.

Thus, for purposes herein, reference to a TCR includes any TCR or functional fragment, such as an antigen-binding portion of a TCR that binds to a specific antigenic peptide bound in an MHC molecule, i.e. MHC-peptide complex. An “antigen-binding portion” or antigen-binding fragment” of a TCR, which can be used interchangeably, refers to a molecule that contains a portion of the structural domains of a TCR, but that binds the antigen (e.g., MHC-peptide complex) to which the full TCR binds. In some cases, an antigen-binding portion contains the variable domains of a TCR, such as variable a chain and variable β chain of a TCR, sufficient to form a binding site for binding to a specific MHC-peptide complex, such as generally where each chain contains three complementarity determining regions.

In some embodiments, the variable domains of the TCR chains associate to form loops, or complementarity determining regions (CDRs) analogous to immunoglobulins, which confer antigen recognition and determine peptide specificity by forming the binding site of the TCR molecule and determine peptide specificity. Typically, like immunoglobulins, the CDRs are separated by framework regions (FRs) (see, e.g., Jores et al., Nat'l Acad. Sci. U.S.A. 87:9138, 1990; Chothia et al., EMBO J. 7:3745, 1988; see also Lefranc et al., Dev. Comp. Immunol. 27:55, 2003). In some embodiments, CDR3 is the main CDR responsible for recognizing processed antigen, although CDR1 of the alpha chain has also been shown to interact with the N-terminal part of the antigenic peptide, whereas CDR1 of the beta chain interacts with the C-terminal part of the peptide. CDR2 is thought to recognize the MHC molecule. In some embodiments, the variable region of the β-chain can contain a further hypervariability (HV4) region.

In some embodiments, the TCR chains contain a constant domain. For example, like immunoglobulins, the extracellular portion of TCR chains (e.g., a-chain, β-chain) can contain two immunoglobulin domains, a variable domain (e.g., Vₐ or Vₚ; typically amino acids 1 to 116 based on Kabat numbering Kabat et al., “Sequences of Proteins of Immunological Interest, U.S. Dept. Health and Human Services, Public Health Service National Institutes of Health, 1991, 5^{th} ed.) at the N-terminus, and one constant domain (e.g., a-chain constant domain or Cₐ, typically amino acids 117 to 259 based on Kabat, B-chain constant domain or Cp, typically amino acids 117 to 295 based on Kabat) adjacent to the cell membrane. For example, in some cases, the extracellular portion of the TCR formed by the two chains contains two membrane-proximal constant domains, and two membrane-distal variable domains containing CDRs. The constant domain of the TCR domain contains short connecting sequences in which a cysteine residue forms a disulfide bond, making a link between the two chains. In some embodiments, a TCR may have an additional cysteine residue in each of the α and β chains such that the TCR contains two disulfide bonds in the constant domains.

In some embodiments, the TCR chains can contain a transmembrane domain. In some embodiments, the transmembrane domain is positively charged. In some cases, the TCR chains contains a cytoplasmic tail. In some cases, the structure allows the TCR to associate with other molecules like CD3. For example, a TCR containing constant domains with a transmembrane region can anchor the protein in the cell membrane and associate with invariant subunits of the CD3 signaling apparatus or complex.

Generally, CD3 is a multi-protein complex that can possess three distinct chains (γ, δ, and ε) in mammals and the ζ-chain. For example, in mammals the complex can contain a CD3γ chain, a CD3δ chain, two CD3ε chains, and a homodimer of CD3ζ chains. The CD3γ, CD3δ, and CD3ε chains are highly related cell surface proteins of the immunoglobulin superfamily containing a single immunoglobulin domain. The transmembrane regions of the CD3γ, CD3δ, and CD3ε chains are negatively charged, which is a characteristic that allows these chains to associate with the positively charged T cell receptor chains. The intracellular tails of the CD3γ, CD3δ, and CD3ε chains each contain a single conserved motif known as an immunoreceptor tyrosine-based activation motif or ITAM, whereas each CD3ζ chain has three. Generally, ITAMs are involved in the signaling capacity of the TCR complex. These accessory molecules have negatively charged transmembrane regions and play a role in propagating the signal from the TCR into the cell. The CD3- and ζ-chains, together with the TCR, form what is known as the T cell receptor complex.

In some embodiments, the TCR may be a heterodimer of two chains a and B (or optionally γ and δ) or it may be a single chain TCR construct. In some embodiments, the TCR is a heterodimer containing two separate chains (α and β chains or γ and δ chains) that are linked, such as by a disulfide bond or disulfide bonds. [0140] In some embodiments, a TCR for a target antigen (e.g., a cancer antigen) is identified and introduced into the cells. In some embodiments, nucleic acid encoding the TCR can be obtained from a variety of sources, such as by polymerase chain reaction (PCR) amplification of publicly available TCR DNA sequences. In some embodiments, the TCR is obtained from a biological source, such as from cells such as from a T cell (e.g., cytotoxic T cell), T-cell hybridomas or other publicly available source. In some embodiments, the T-cells can be obtained from in vivo isolated cells. In some embodiments, a high-affinity T cell clone can be isolated from a patient, and the TCR isolated. In some embodiments, the T-cells can be a cultured T-cell hybridoma or clone. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al. (2009) Clin Cancer Res. 15:169-180 and Cohen et al. (2005) J. Immunol. 175:5799-5808. In some embodiments, phage display is used to isolate TCRs against a target antigen (see, e.g., Varela-Rohena et al. (2008) Nat. Med. 14:1390-1395 and Li (2005) Nat. Biotechnol. 23:349-354. In some embodiments, the TCR or antigen-binding portion thereof can be synthetically generated from knowledge of the sequence of the TCR.

### IV. METHODS OF TREATMENT

Certain aspects of the present embodiments can be used to prevent or treat a disease or disorder associated with GARP signaling. Signaling of GARP may be reduced by any suitable drugs to prevent cancer cell proliferation. Preferably, such substances would be an anti-GARP antibody.

Provided herein, in certain embodiments, are methods for treating or delaying progression of cancer in an individual comprising administering to the individual an effective amount an anti-platelet agent and T cell therapy. Examples of cancers contemplated for treatment include lung cancer, head and neck cancer, breast cancer, pancreatic cancer, prostate cancer, renal cancer, bone cancer, testicular cancer, cervical cancer, gastrointestinal cancer, lymphomas, pre-neoplastic lesions in the lung, colon cancer, melanoma, and bladder cancer.

In some embodiments, the individual has cancer that is resistant (has been demonstrated to be resistant) to one or more anti-cancer therapies. In some embodiments, resistance to anti-cancer therapy includes recurrence of cancer or refractory cancer. Recurrence may refer to the reappearance of cancer, in the original site or a new site, after treatment. In some embodiments, resistance to anti-cancer therapy includes progression of the cancer during treatment with the anti-cancer therapy. In some embodiments, the cancer is at early stage or at late stage.

In some embodiments of the methods of the present disclosure, activated CD4 and/or CD8 T cells in the individual are characterized by γ-IFN producing CD4 and/or CD8 T cells and/or enhanced cytolytic activity relative to prior to the administration of the combination. γ-IFN may be measured by any means known in the art, including, e.g., intracellular cytokine staining (ICS) involving cell fixation, permeabilization, and staining with an antibody against γ-IFN. Cytolytic activity may be measured by any means known in the art, e.g., using a cell killing assay with mixed effector and target cells.

A T cell therapy may be administered before, during, after, or in various combinations relative to an anti-platelet agent. The administrations may be in intervals ranging from concurrently to minutes to days to weeks. In embodiments where the T cell therapy is provided to a patient separately from an anti-platelet agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations, it may be desirable to extend the time period for treatment significantly where several days (2, 3, 4, 5, 6, or 7) to several weeks (1, 2, 3, 4, 5, 6, 7, or 8) lapse between respective administrations.

In some embodiments, the subject can be administered nonmyeloablative lymphodepleting chemotherapy prior to the T cell therapy. The nonmyeloablative lymphodepleting chemotherapy can be any suitable such therapy, which can be administered by any suitable route. The nonmyeloablative lymphodepleting chemotherapy can comprise, for example, the administration of cyclophosphamide and fludarabine, particularly if the cancer is melanoma, which can be metastatic. An exemplary route of administering cyclophosphamide and fludarabine is intravenously. Likewise, any suitable dose of cyclophosphamide and fludarabine can be administered. In particular aspects, around 60 mg/kg of cyclophosphamide is administered for two days after which around 25 mg/m² fludarabine is administered for five days.

In certain embodiments, a T-cell growth factor that promotes the growth and activation of the autologous T cells is administered to the subject either concomitantly with the autologous T cells or subsequently to the autologous T cells. The T-cell growth factor can be any suitable growth factor that promotes the growth and activation of the autologous T-cells. Examples of suitable T-cell growth factors include interleukin (IL)-2, IL-7, IL-15, and IL-12, which can be used alone or in various combinations, such as IL-2 and IL-7, IL-2 and IL-15, IL-7 and IL-15, IL-2, IL-7 and IL-15, IL-12 and IL-7, IL-12 and IL-15, or IL-12 and IL2. IL-12 is a preferred T-cell growth factor.

The T cell therapy and anti-platelet agent may be administered by the same route of administration or by different routes of administration. In some embodiments, the T cell therapy and/or anti-platelet agent is administered intravenously, intramuscularly, subcutaneously, topically, orally, transdermally, intraperitoneally, intraorbitally, by implantation, by inhalation, intrathecally, intraventricularly, or intranasally. An effective amount of the T cell therapy and anti-platelet agent may be administered for prevention or treatment of disease. The appropriate dosage of the T cell therapy and anti-platelet agent be determined based on the type of disease to be treated, severity and course of the disease, the clinical condition of the individual, the individual's clinical history and response to the treatment, and the discretion of the attending physician.

Intratumoral injection, or injection into the tumor vasculature is specifically contemplated for discrete, solid, accessible tumors. Local, regional or systemic administration also may be appropriate. For tumors of >4 cm, the volume to be administered will be about 4-10 ml (in particular 10 ml), while for tumors of <4 cm, a volume of about 1-3 ml will be used (in particular 3 ml). Multiple injections delivered as single dose comprise about 0.1 to about 0.5 ml volumes.

### A. Pharmaceutical Compositions

Where clinical application of a therapeutic composition containing an inhibitory antibody is undertaken, it will generally be beneficial to prepare a pharmaceutical or therapeutic composition appropriate for the intended application. In certain embodiments, pharmaceutical compositions may comprise, for example, at least about 0.1% of an active compound. In other embodiments, an active compound may comprise between about 2% to about 75% of the weight of the unit, or between about 25% to about 60%, for example, and any range derivable therein.

Also provided herein are pharmaceutical compositions and formulations comprising T cell therapy, an anti-platelet agent and a pharmaceutically acceptable carrier.

The therapeutic compositions of the present embodiments are advantageously administered in the form of injectable compositions either as liquid solutions or suspensions, solid forms suitable for solution in, or suspension in, liquid prior to injection may also be prepared. These preparations also may be emulsified.

The active compounds can be formulated for parenteral administration, e.g., formulated for injection via the intravenous, intramuscular, sub-cutaneous, or even intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaceous compositions may be formulated into a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Pharmaceutical compositions and formulations as described herein can be prepared by mixing the active ingredients (such as an antibody or a polypeptide) having the desired degree of purity with one or more optional pharmaceutically acceptable carriers (Remington's Pharmaceutical Sciences 22nd edition, 2012), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

### B. Anti-Platelet Agents

Embodiments of the present methods concern anti-platelet agents. The phrase “anti-platelet agent” refers to any compound which inhibits activation, aggregation, and/or adhesion of platelets, and is intended to include all pharmaceutically acceptable salts, prodrugs e.g., esters and solvate forms, including hydrates, of compounds which have the activity, compounds having one or more chiral centers may occur as racemates, racemic mixtures and as individual diastereomers or enantiomers with all such isomeric forms and mixtures thereof being included, any crystalline polymorphs, co-crystals and the amorphous form are intended to be included.

Non-limiting examples of antiplatelet agents that may be used in the oral dosage forms of the present disclosure include adenosine diphosphate (ADP) antagonists or P₂ Yi₂ antagonists, phosphodiesterase (PDE) inhibitors, adenosine reuptake inhibitors, Vitamin K antagonists, heparin, heparin analogs, direct thrombin inhibitors, glycoprotein IIB/IIIA inhibitors, anti-clotting enzymes, as well as pharmaceutically acceptable salts, isomers, enantiomers, polymorphic crystal forms including the amorphous form, solvates, hydrates, co-crystals, complexes, active metabolites, active derivatives and modifications, pro-drugs thereof, and the like.

ADP antagonists or P₂Y₁₂ antagonists block the ADP receptor on platelet cell membranes. This P₂Y₁₂ receptor is important in platelet aggregation, the cross-linking of platelets by fibrin. The blockade of this receptor inhibits platelet aggregation by blocking activation of the glycoprotein Ilb/IIIa pathway. In an exemplary embodiment, the antiplatelet agent is an ADP antagonist or P₂Yi₂ antagonist. In another exemplary embodiment, the antiplatelet agent is a thienopyridine. In another exemplary embodiment, the ADP antagonist or P₂Yi₂ antagonist is a thienopyridine.

In another exemplary embodiment, the ADP antagonist or P₂Yi₂ antagonist is a member selected from sulfinpyrazone, ticlopidine, clopidogrel, prasugrel, R-99224 (an active metabolite of prasugrel, supplied by Sankyo), R-1381727, R-125690 (Lilly), C-1330-7, C-50547 (Millennium Pharmaceuticals), INS-48821, INS-48824, INS-446056, INS-46060, INS-49162, INS-49266, INS-50589 (Inspire Pharmaceuticals) and Sch-572423 (Schering Plough). In another exemplary embodiment, the ADP antagonist or P₂Yi₂ antagonist is ticlopidine hydrochloride (TICLID™) In another exemplary embodiment, the ADP antagonist or P₂Yi₂ antagonist is a member selected from sulfinpyrazone, ticlopidine, AZD6140, clopidogrel, prasugrel and mixtures thereof. In another exemplary embodiment, the ADP antagonist or P₂Yi₂ antagonist is clopidogrel. In another exemplary embodiment, the therapeutically effective amount of clopidogrel is from about 50 mg to about 100 mg. In another exemplary embodiment, the therapeutically effective amount of clopidogrel is from about 65 mg to about 80 mg. In another exemplary embodiment, the ADP antagonist or P₂Yi₂ antagonist is a member selected from clopidogrel bisulfate (PLA VIX™), clopidogrel hydrogen sulphate, clopidogrel hydrobromide, clopidogrel mesylate, cangrelor tetrasodium (AR-09931 MX), ARL67085, AR-C66096 AR-C 126532, and AZD-6140 (AstraZeneca). In another exemplary embodiment, the ADP antagonist or P₂Yi₂ antagonist is prasugrel. In another exemplary embodiment, the therapeutically effective amount of prasugrel is from about 1 mg to about 20 mg. In another exemplary embodiment, the therapeutically effective amount of clopidogrel is from about 4 mg to about 11 mg. In another exemplary embodiment, the ADP antagonist or P₂Yi₂ antagonist is a member selected from clopidogrel, ticlopidine, sulfinpyrazone, AZD6140, prasugrel and mixtures thereof.

In certain embodiments the anti-platelet agent is clopidogrel or a pharmaceutically acceptable salt, solvate, polymorph, co-crystal, hydrate, enantiomer or prodrug thereof. In another embodiment clopidogrel or pharmaceutically acceptable salt, solvate, polymorph, co-crystal, hydrate, enantiomer or prodrug thereof is a powder.

A PDE inhibitor is a drug that blocks one or more of the five subtypes of the enzyme phosphodiesterase (PDE), preventing the inactivation of the intracellular second messengers, cyclic adenosine monophosphate (cAMP) and cyclic guanosine monophosphate (cGMP), by the respective PDE subtype(s). In an exemplary embodiment, the antiplatelet agent is a PDE inhibitor. In an exemplary embodiment, the antiplatelet agent is a selective cAMP PDE inhibitor, hi an exemplary embodiment, the PDE inhibitor is cilostazol (Pletal™).

Adenosine reuptake inhibitors prevent the cellular reuptake of adenosine into platelets, red blood cells and endothelial cells, leading to increased extracellular concentrations of adenosine. These compounds inhibit platelet aggregation and cause vasodilation, hi an exemplary embodiment, the antiplatelet agent is an adenosine reuptake inhibitor. In an exemplary embodiment, the adenosine reuptake inhibitor is dipyridamole (Persantine™).

Vitamin K inhibitors are given to people to stop thrombosis (blood clotting inappropriately in the blood vessels). This is useful in primary and secondary prevention of deep vein thrombosis, pulmonary embolism, myocardial infarctions and strokes in those who are predisposed. In an exemplary embodiment, the anti-platelet agent is a Vitamin K inhibitor, hi an exemplary embodiment, the Vitamin K inhibitor is a member selected from acenocoumarol, clorindione, dicumarol (Dicoumarol), diphenadione, ethyl biscoumacetate, phenprocoumon, phenindione, tioclomarol and warfarin.

Heparin is a biological substance, usually made from pig intestines. It works by activating antithrombin III, which blocks thrombin from clotting blood. In an exemplary embodiment, the antiplatelet agent is heparin or a prodrug of heparin. In an exemplary embodiment, the antiplatelet agent is a heparin analog or a prodrug of a heparin analog. In an exemplary embodiment, the heparin analog a member selected from Antithrombin III, Bemiparin, Dalteparin, Danaparoid, Enoxaparin, Fondaparinux (subcutaneous), Nadroparin, Parnaparin, Reviparin, Sulodexide, and Tinzaparin.

Direct thrombin inhibitors (DTIs) are a class of medication that act as anticoagulants (delaying blood clotting) by directly inhibiting the enzyme thrombin. In an exemplary embodiment, the antiplatelet agent is a DTI. In another exemplary embodiment, the DTI is univalent. In another exemplary embodiment, the DTI is bivalent. In an exemplary embodiment, the DTI is a member selected from hirudin, bivalirudin (IV), lepirudin, desirudin, argatroban (IV), dabigatran, dabigatran etexilate (oral formulation), melagatran, ximelagatran (oral formulation but liver complications) and prodrugs thereof.

In an exemplary embodiment, the anti-platelet agent is a member selected from aloxiprin, beraprost, carbasalate calcium, cloricromen, defibrotide, ditazole, epoprostenol, indobufen, iloprost, picotamide, rivaroxaban (oral FXa inhibitor) treprostinil, triflusal, or prodrugs thereof.

In certain embodiments, the anti-platelet agent is an antibody or a fragment thereof that binds to at least a portion of GARP protein. As used herein, the term “antibody” is intended to refer broadly to any immunologic binding agent, such as IgG, IgM, IgA, IgD, IgE, and genetically modified IgG as well as polypeptides comprising antibody CDR domains that retain antigen binding activity. The antibody may be selected from the group consisting of a chimeric antibody, an affinity matured antibody, a polyclonal antibody, a monoclonal antibody, a humanized antibody, a human antibody, or an antigen-binding antibody fragment or a natural or synthetic ligand. Preferably, the anti-GARP antibody is a monoclonal antibody or a humanized antibody. Thus, by known means and as described herein, polyclonal or monoclonal antibodies, antibody fragments, and binding domains and CDRs (including engineered forms of any of the foregoing) may be created that are specific to GARP protein, one or more of its respective epitopes, or conjugates of any of the foregoing, whether such antigens or epitopes are isolated from natural sources or are synthetic derivatives or variants of the natural compounds.

Examples of antibody fragments suitable for the present embodiments include, without limitation: (i) the Fab fragment, consisting of V_{L}, V_{H}, C_{L}, and C_{H1} domains; (ii) the “Fd” fragment consisting of the VA and Cat domains; (iii) the “Fv” fragment consisting of the Vt. and V_{H} domains of a single antibody; (iv) the “dAb” fragment, which consists of a V_{H} domain; (v) isolated CDR regions; (vi) F(ab′)2 fragments, a bivalent fragment comprising two linked Fab fragments; (vii) single chain Fv molecules (“scFv”), wherein a V_{H} domain and a V_{L} domain are linked by a peptide linker that allows the two domains to associate to form a binding domain; (viii) bi-specific single chain Fv dimers (see U.S. Pat. No. 5,091,513); and (ix) diabodies, multivalent or multispecific fragments constructed by gene fusion (US Patent App. Pub 20050214860). Fv, scFv, or diabody molecules may be stabilized by the incorporation of disulphide bridges linking the V_{H} and V_{L} domains. Minibodies comprising a scFv joined to a CH3 domain may also be made (Hu et al., 1996).

### C. Additional Therapy

In certain embodiments, the compositions and methods of the present embodiments involve an antibody or an antibody fragment against GARP to inhibit its activity in cancer cell proliferation, in combination with a second or additional therapy. Such therapy can be applied in the treatment of any disease that is associated with GARP-mediated cell proliferation. For example, the disease may be cancer.

In certain embodiments, the compositions and methods of the present embodiments involve a T cell therapy and an anti-platelet agent in combination with at least one additional therapy. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy.

The methods and compositions, including combination therapies, enhance the therapeutic or protective effect, and/or increase the therapeutic effect of another anti-cancer or anti-hyperproliferative therapy. Therapeutic and prophylactic methods and compositions can be provided in a combined amount effective to achieve the desired effect, such as the killing of a cancer cell and/or the inhibition of cellular hyperproliferation. This process may involve contacting the cells with both an antibody or antibody fragment and a second therapy. A tissue, tumor, or cell can be contacted with one or more compositions or pharmacological formulation(s) comprising one or more of the agents (i.e., antibody or antibody fragment or an anti-cancer agent), or by contacting the tissue, tumor, and/or cell with two or more distinct compositions or formulations, wherein one composition provides 1) an antibody or antibody fragment, 2) an anti-cancer agent, or 3) both an antibody or antibody fragment and an anti-cancer agent. Also, it is contemplated that such a combination therapy can be used in conjunction with chemotherapy, radiotherapy, surgical therapy, or immunotherapy.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing, for example, both agents are delivered to a cell in a combined amount effective to kill the cell or prevent it from dividing.

An inhibitory antibody may be administered before, during, after, or in various combinations relative to an anti-cancer treatment. The administrations may be in intervals ranging from concurrently to minutes to days to weeks. In embodiments where the antibody or antibody fragment is provided to a patient separately from an anti-cancer agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations, it may be desirable to extend the time period for treatment significantly where several days (2, 3, 4, 5, 6, or 7) to several weeks (1, 2, 3, 4, 5, 6, 7, or 8) lapse between respective administrations.

In certain embodiments, a course of treatment will last 1-90 days or more (this such range includes intervening days). It is contemplated that one agent may be given on any day of day 1 to day 90 (this such range includes intervening days) or any combination thereof, and another agent is given on any day of day I to day 90 (this such range includes intervening days) or any combination thereof. Within a single day (24-hour period), the patient may be given one or multiple administrations of the agent(s). Moreover, after a course of treatment, it is contemplated that there is a period of time at which no anti-cancer treatment is administered. This time period may last 1-7 days, and/or 1-5 weeks, and/or 1-12 months or more (this such range includes intervening days), depending on the condition of the patient, such as their prognosis, strength, health, etc. It is expected that the treatment cycles would be repeated as necessary.

In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent. The additional therapy may be one or more of the chemotherapeutic agents known in the art.

Various combinations may be employed. For the example below an antibody therapy, or a T cell therapy and anti-platelet agent, is “A” and an anti-cancer therapy is “B”:
A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

### 1. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa, ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin, spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gamma1I and calicheamicin omegaI1); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid, aceglatone, aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine, pentostatin; phenamet; pirarubicin, losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate, irinotecan (e.g., CPT-11); topoisomerase inhibitor RFS 2000, difluorometlhylornithine (DMFO); retinoids, such as retinoic acid; capecitabine; carboplatin, procarbazine, plicomycin, gemcitabien, navelbine, farnesyl-protein transferase inhibitors, transplatinum, and pharmaceutically acceptable salts, acids, or derivatives of any of the above.

### 2. Radiotherapy

Other factors that cause DNA damage and have been used extensively include what are commonly known as y-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

### 3. Immunotherapy

The skilled artisan will understand that additional immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells

Antibody-drug conjugates have emerged as a breakthrough approach to the development of cancer therapeutics. Cancer is one of the leading causes of deaths in the world. Antibody-drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen (Carter et al., 2008; Teicher 2014; Leal et al., 2014). Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment (Leal et al., 2014). As antibody engineering and linker-payload optimization are becoming more and more mature, the discovery and development of new ADCs are increasingly dependent on the identification and validation of new targets that are suitable to this approach (Teicher 2009) and the generation of targeting MAbs. Two criteria for ADC targets are upregulated/high levels of expression in tumor cells and robust internalization.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons α, β, and γ, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints are molecules in the immune system that either turn up a signal (e.g., co-stimulatory molecules) or turn down a signal. Inhibitory checkpoint molecules that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication WO2015016718; Pardoll, Nat Rev Cancer, 12(4): 252-64, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present invention. For example it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 axis antagonists for use in the methods provided herein are known in the art such as described in U.S. Patent Application No. US20140294898, US2014022021, and US20110008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al. (1998) Proc Natl Acad Sci USA 95(17): 10067-10071; Camacho et al. (2004) J Clin Oncology 22(145): Abstract No. 2505 (antibody CP-675206); and Mokyr et al. (1998) Cancer Res 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001014424, WO2000037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WOO 1/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. No. 844,905, U.S. Pat. No. 5,885,796 and International Patent Application Nos. WO1995001994 and WO1998042752; all incorporated herein by reference, and immunoadhesions such as described in U.S. Pat. No. 8,329,867, incorporated herein by reference.

### 4. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well

### 5. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### V. ARTICLES OF MANUFACTURE OR KITS

In various aspects of the embodiments, a kit is envisioned containing therapeutic agents and/or other therapeutic and delivery agents. In some embodiments, the present disclosure contemplates a kit for preparing and/or administering a therapy of the embodiments. The kit may comprise one or more sealed vials containing any of the pharmaceutical compositions of the present embodiments. The kit may include, for example, at least one GARP antibody as well as reagents to prepare, formulate, and/or administer the components of the embodiments or perform one or more steps of the inventive methods. In some embodiments, the kit may also comprise a suitable container, which is a container that will not react with components of the kit, such as an Eppendorf tube, an assay plate, a syringe, a bottle, or a tube. The container may be made from sterilizable materials such as plastic or glass.

In some embodiment, an article of manufacture or a kit is provided comprising adoptive T cells and an anti-platelet agent (e.g., anti-GARP antibody) is also provided herein. The article of manufacture or kit can further comprise a package insert comprising instructions for using the adoptive T cells in conjunction with an anti-platelet agent to treat or delay progression of cancer in an individual or to enhance immune function of an individual having cancer. Any of the adoptive T cells and/or anti-platelet agents described herein may be included in the article of manufacture or kits. In some embodiments, the adoptive T cells and anti-platelet agent are in the same container or separate containers. Suitable containers include, for example, bottles, vials, bags and syringes. The container may be formed from a variety of materials such as glass, plastic (such as polyvinyl chloride or polyolefin), or metal alloy (such as stainless steel or hastelloy). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., a chemotherapeutic agent, and anti-neoplastic agent). Suitable containers for the one or more agent include, for example, bottles, vials, bags and syringes.

The kit may further include an instruction sheet that outlines the procedural steps of the methods set forth herein, and will follow substantially the same procedures as described herein or are known to those of ordinary skill in the art. The instruction information may be in a computer readable media containing machine-readable instructions that, when executed using a computer, cause the display of a real or virtual procedure of delivering a pharmaceutically effective amount of a therapeutic agent.

### VI. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Expression of GARP in Cancer Cells

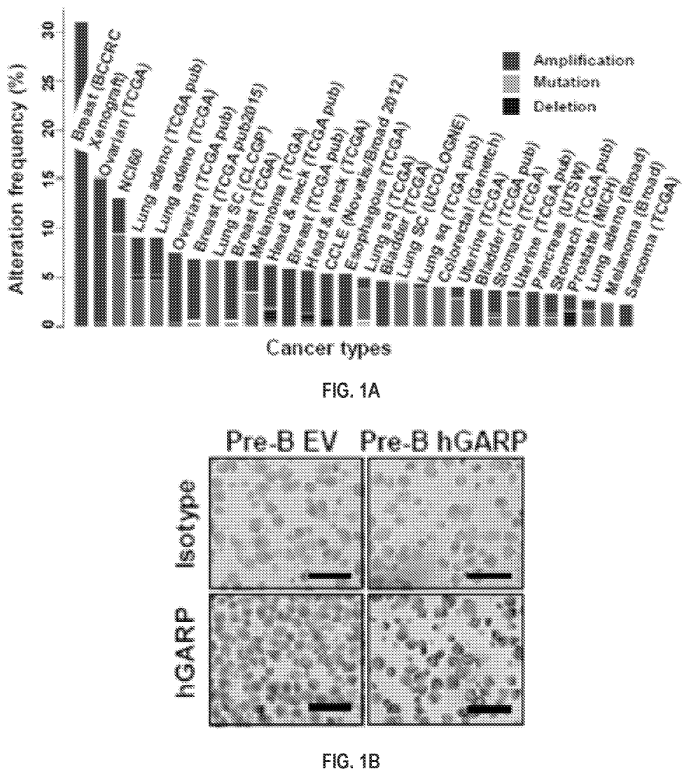

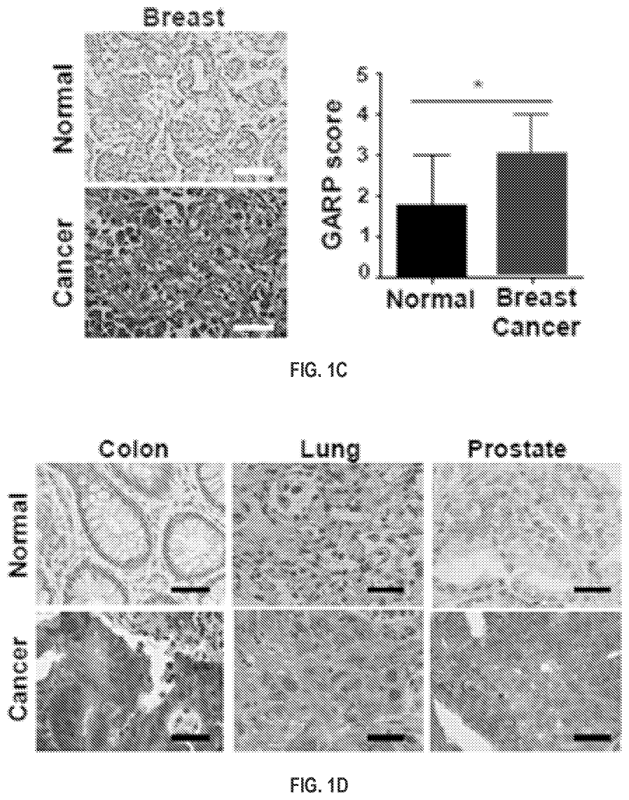

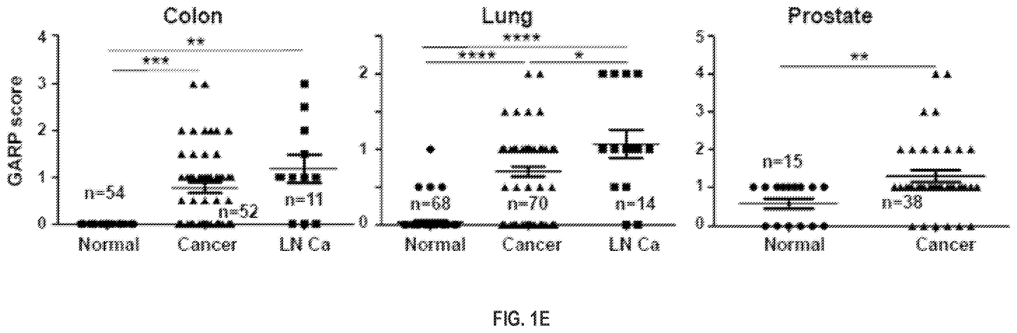

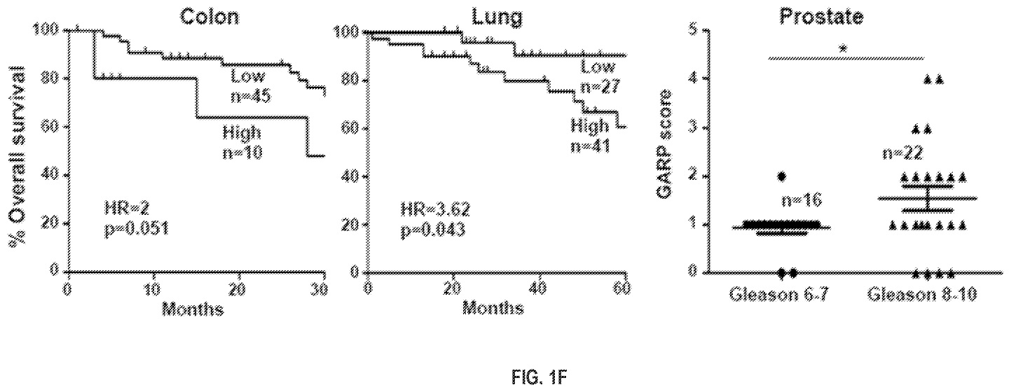

Recent studies, including The Cancer Genome Atlas (TCGA) project, have shown that the GARP gene, LRRC32, is amplified in up to 30% of patients with many human cancer types, including ovarian, lung, breast, and head and neck cancers (FIG. 1A). To examine GARP protein expression, immunohistochemistry (IHC) was performed on a human tumor microarray from archived human tumors and it was subsequently determined whether GARP expression carried any prognostic significance. The specificity of the anti-human GARP antibody was ascertained by its staining of a Pre-B leukemia cell line stably transfected with human GARP (FIG. 1B). Given that LRRC32 was amplified in human breast cancer (Szepetowski et al., 1992), GARP expression was first evaluated in breast cancer patients using IHC. The results were read and scored by a clinical pathologist in a double-blinded fashion. IHC analysis of patient-matched uninvolved breast tissue versus primary breast cancer (n=16) indicated a significant increase of GARP expression on cancer tissues in 9 out of 16 patients (FIG. 1C). By RT-PCR, GARP mRNA expression was increased by ≥2-fold in 28.5% of patients with breast cancer (n=42) compared with normal breast tissues. IHC was then performed on cancer specimens, including 55 colon cancer specimens, $5 adjacent normal tissues, and 11 corresponding lymph nodes, and adjacent normal tissues (FIG. 1D). Normal epithelial specimens showed no significant GARP positivity (FIGS. 1D and 1E). However, the primary cancers (colon and lung) and lymph node (LN) metastatic tissues stained variably positive for GARP (uniformly negative with isotype control antibody) (FIG. 1E). Compared to the undetectable level (defined as 0) in normal tissue, the percentage of GARP positive cells was 26.1% (p=8.6×10⁻⁹) in primary cancer and 25% (p=0.008) in LN metastasis. On a scale of 0 to 4, GARP intensity score ranged between 0 and 3, averaging at 0.78 (p=1.1×10⁻⁸) in primary colon cancers and 1.18 (p=0.003) in LN metastasis (FIG. 1E). Similarly, significantly increased GARP levels were found in primary cancers of the lung and the prostate (FIG. 1E). More importantly, GARP levels correlated inversely with overall survival in patients with colon and lung cancer. regardless of the pathological grade of tumors or lymph node status of the disease (FIG. 1F). High GARP expression also correlated with high Gleason score in prostate cancer (p=0.035) (FIG. 1F). These results demonstrate for the first time that GARP is widely expressed in human cancers, and that the level of expression correlates with disease aggressiveness.

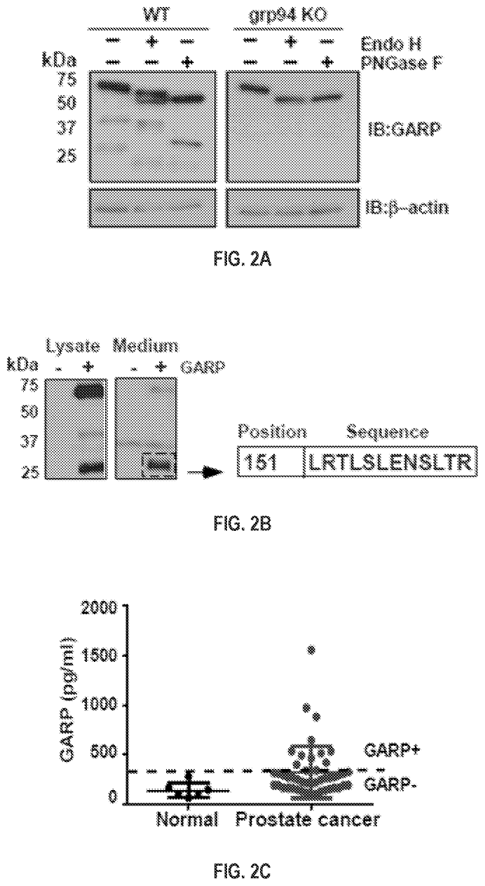

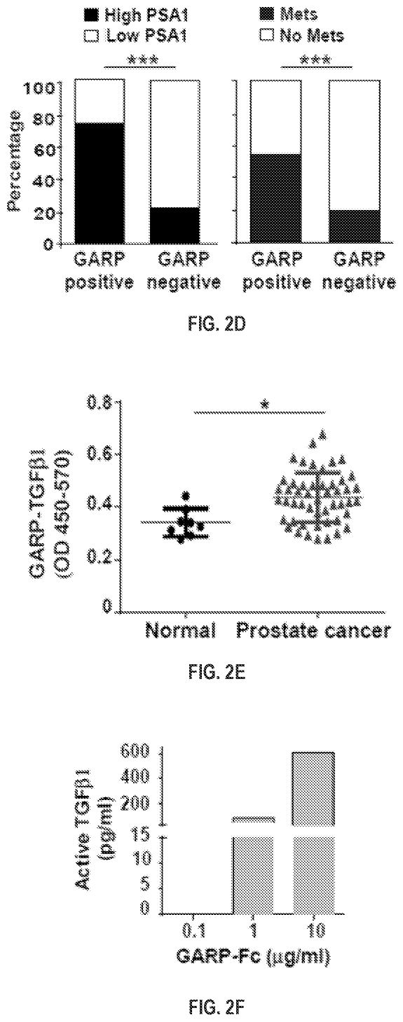

In vitro biochemical studies have established that GARP also exists in a soluble form that is secreted in complex with latent TGF-β1 from Treg cells (Gauthy et al., 2013). It has also been shown that GARP depends on the molecular chaperone grp94 in the endoplasmic reticulum for folding and cell surface expression (Zhang et al., 2015). To determine whether GARP secretion is a Treg cell-specific event or a GARP-intrinsic phenomenon, N-terminal hemagglutinin (HA)-tagged GARP was expressed in murine Pre-B cells with and without grp94, and then GARP expression was analyzed in cell lysates and conditioned media. Three GARP bands were observed only in gp96⁺ (WT) cells: approximately 75, 45, and 30 kDa in molecular weight, respectively (FIG. 2A). The 45 and 30 kDa fragments appeared to be the postranslationally cleaved products of the full-length cell surface GARP (75 kDa) because they were more resistant to Endo H compared with PNGase F and were found in WT, but not grp94 KO cells. If so, the 30 kDa N-terminal GARP fragment should be liberated from the cell surface into the media. Indeed, gel extraction and sequencing of the 30 kDa protein in the media by mass spectrometry confirmed that it was derived from the N-terminal fragment of GARP (FIG. 2B).

It was next determined whether soluble GARP (sGARP) was present in the sera of cancer patients and whether the serum levels of sGARP had any prognostic significance. Sera were collected from male normal controls (n=7) and prostate cancer patients (n=48) and analyzed for GARP by ELISA. It was found that sGARP was present in serum from both normal individuals and from prostate cancer patients (FIG. 2C). Further analysis revealed that higher GARP levels correlated with increased prostate cancer specific antigen (PSA) levels and metastasis (FIG. 2D). Moreover, the presence of the sGARP-TGF-β1 complex was evaluated in the serum of prostate cancer patients and normal controls using a GARP-TGF-β1 sandwich ELISA. As predicted, cancer patients' sera contained higher levels of soluble GARP and TGF-β1 complex than normal subjects (FIG. 2E). To gain insight into the function of soluble GARP, a fusion protein was prepared consisting of the N-terminal extracellular domain of GARP linked to an Fc domain of IgG (GARP-Fc). The construct was expressed in the Chinese hamster ovary (CHO) cells. The GARP fusion protein was then purified from the conditioned medium. As measured by active TGF-β ELISA, a direct association was found between GARP-Fc and active TGF-β1 (FIG. 2F), indicating the presence of GARP-Fc-TGF-β1 complexes.

### Example 2—GARP and TGF-β

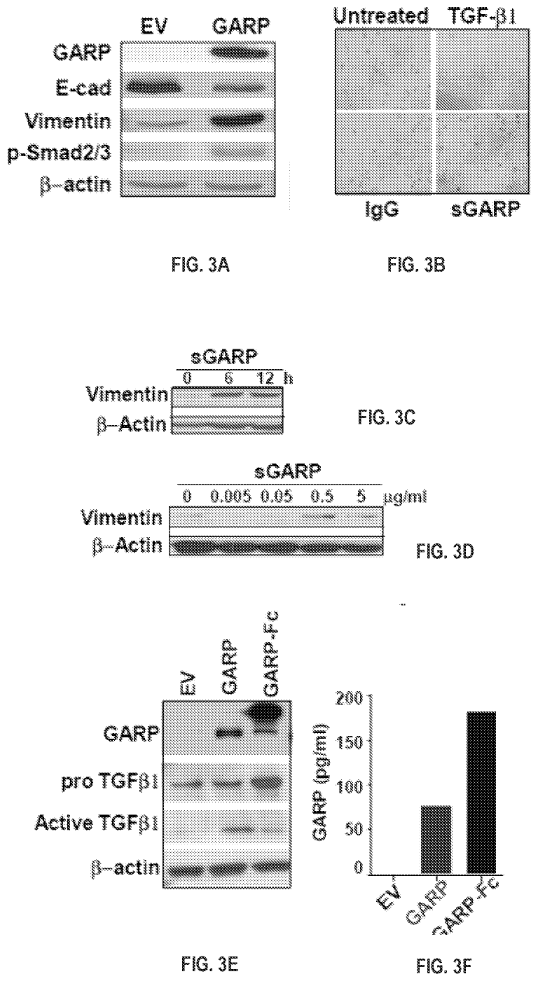

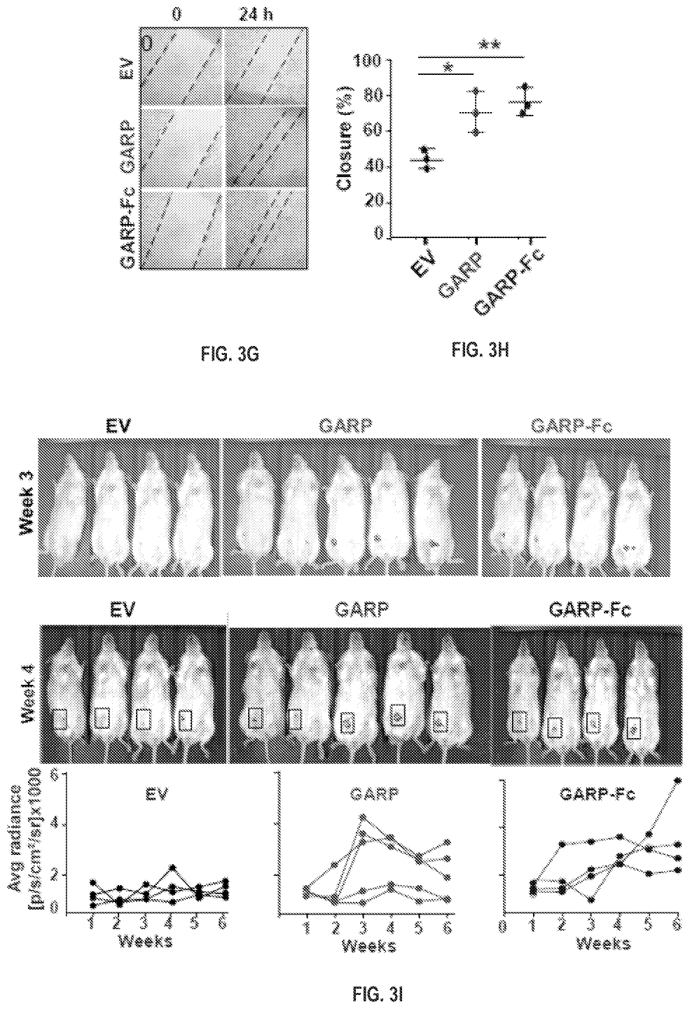

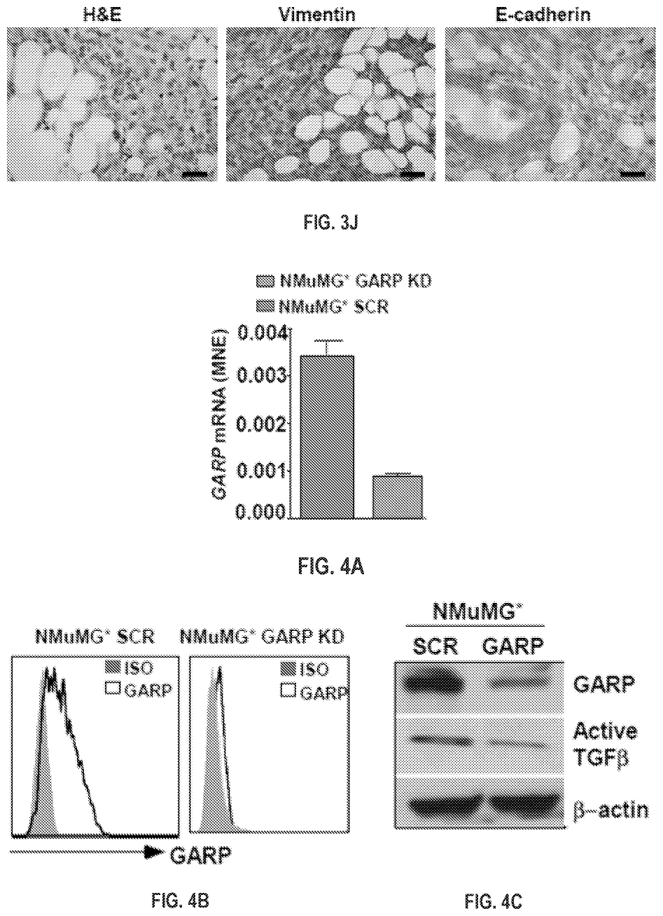

Enforced GARP expression in normal murine mammary epithelial cells upregulates TGF-β expression and drives oncogenesis. In normal murine mammary gland epithelia (NMuMG) cells, TGF-β exerts both a growth inhibitory response and an epithelial-to-mesenchymal cell transition (EMT) response (Xie et al., 2003). As such, NMuMG cells have been extensively utilized to study TGF-β signaling and biology (Xu et al., 2009). Given that GARP regulates the bioavailability of TGF-β. NMuMG cells were used in a bioassay to study the effect of both membrane-bound GARP and soluble GARP on epithelial cells. It was found that stable GARP expression induced Smad-2/3 phosphorylation and expression of vimentin, but downregulated E-cadherin, consistent with increased canonical TGF-signaling (FIG. 3A). Moreover, NMuMG cells stimulated with soluble GARP-Fc changed from their typical polygonal and flattened epithelial cell morphology to a spindle-shaped morphology within 24 hours (FIG. 3B), with an accompanying time- and dose-dependent upregulation of vimentin (FIGS. 3C and 3D). As expected, NMuMG cells stably expressing either GARP or GARP-Fc had higher expression of active TGF-β1 (FIG. 3E) as well as soluble GARP (FIG. 3F), compared to cells transduced with empty vector (EV). An in vitro “scratch” assay was performed to gauge the migratory properties of GARP-expressing cells. The closure rate of the gap (created by scratching the culture plate) was significantly increased with GARP-expressing cells, indicating increased acquired migratory ability (FIGS. 3G and 3H). It was also examined whether enforced GARP expression enabled NMuMG cells to establish tumors in vivo. To this end, female immunodeficient NOD-RagI^{-/-} mice were injected in the fourth mammary fat pad with GARP-expressing NMuMG cells or with EV control cells all of which were also engineered to co-express luciferase. By in vivo imaging of the bioluminescence, it was found that the bioactive mass formed only in mice that received GARP⁺ or GARP-Fc⁺NMuMG, but not in mice receiving EV transduced cells (FIG. 3I). The tumor formation by GARP-expressing cells was confirmed by histology (FIG. 3J). Collectively, these results demonstrate that GARP has a transforming property via upregulation of TGF-β, identifying GARP as a potential novel oncogene.

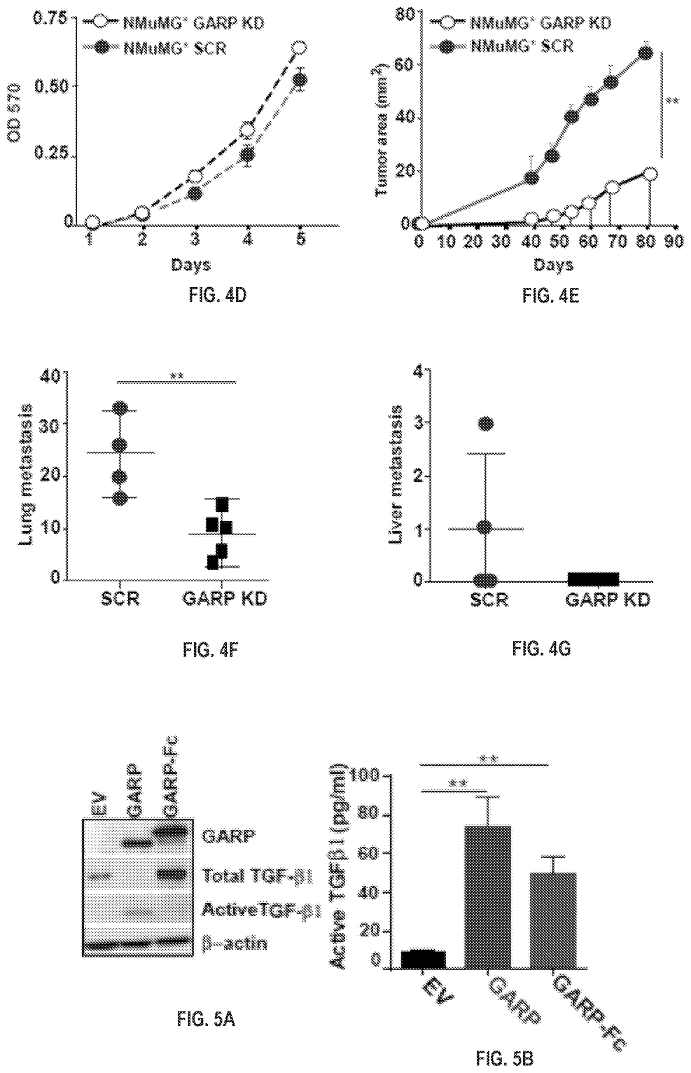

Silencing GARP delays tumor growth. A variant of the normal murine mammary gland epithelial cell line (NMuMG*), in which an RNA-binding protein hnRNPE1 is knocked down by RNA interference, was recently described as being capable of forming tumors in nude mice (Howley et al., 2015). Intriguingly, it was found that these cells expressed a significant level of endogenous GARP (FIGS. 4A-4C), raising the possibility that heightened TGF-β biogenesis, in addition to the silencing of the TGF-β-mediated translation repression complex, drives mammary cancer in this model. To test this hypothesis, short hairpin RNA (shRNA) knock down (KD) of GARP was performed in the NMuMG* cells (FIGS. 4A-4C). GARP silencing did not affect the in vitro proliferation of NMuMG* cells as determined by MTT assay (FIG. 4D). Remarkably, silencing of GARP alone in the NMuMG* cells significantly attenuated their growth in vivo (FIG. 4E). Further, the ability of these GARP KD cells to metastasize to the lungs and liver was compromised (FIGS. 4F and 4G).

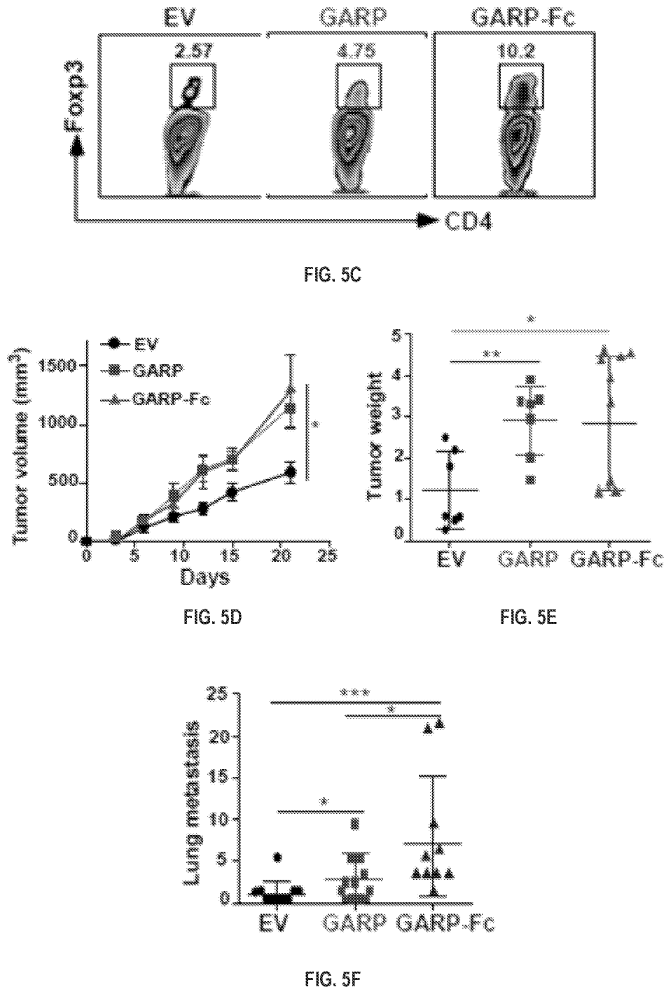

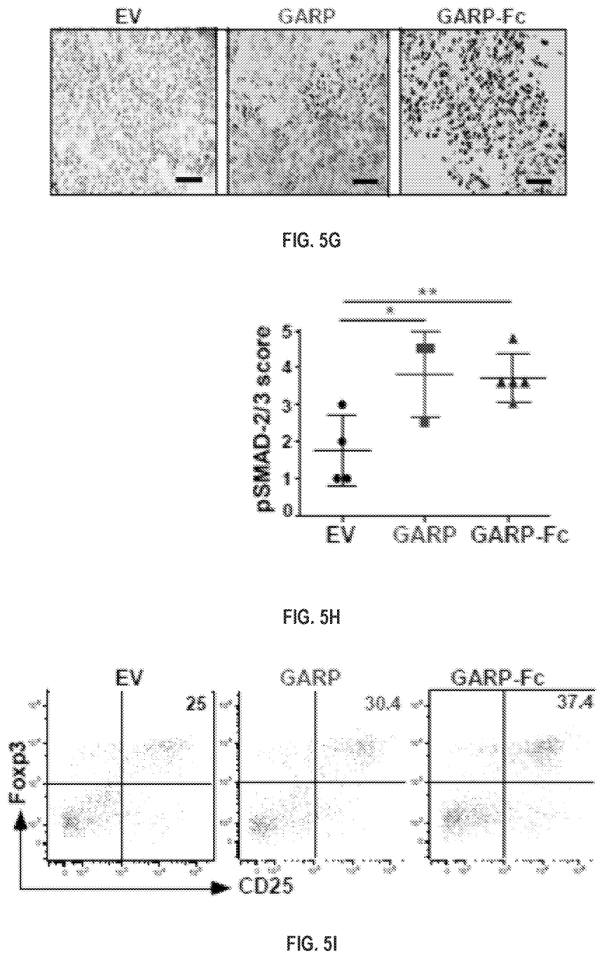

GARP upregulation in murine mammary cancer cells promotes TGF-β activation, tumor growth, metastasis and immune tolerance. LRRC32 was initially described in breast cancer as a frequently amplified gene (Ollendorff et al., 1994), and TGF-β signaling has been shown to promote breast cancer invasion and metastasis (Massague, 2008; Padua et al., 2008; Siegel et al., 2003). However, an under-studied aspect of TGF-β biology in cancer is the cancer-extrinsic role of TGF-β via modulating the host immune response (Li and Flavell, 2008). Thus, the impact of GARP on cancer growth and metastasis in a syngeneic immune-sufficient setting was examined in the highly aggressive and metastatic 4Tl mammary carcinoma model in BALB/c mice (Pulaski and Ostrand-Rosenberg, 2001). Similar to the NMuMG system, the over-expression of GARP or GARP-Fc in 4T1 cells led to increased production of active TGF-β (FIGS. SA and SB). One of the key mechanisms by which TGF-β inhibits tumor-specific immunity is via the induction of Foxp3⁺ Tregs. To this end, purified naïve CD4⁺ T cells were cultured in vitro with conditioned media from 4T1-GARP, 4T1-GARP-Fc and empty vector (EV) control cells in the presence of polyclonal T cell activators for 3 days. The conditioned media from GARP-expressing cells was 2- to 3-fold more efficient at inducing Treg differentiation compared to media from control cells (FIG. 5C). 4T1-EV, 4T1-GARP and 4T1-GARP-Fc cells were injected orthotopically in the fourth right mammary fat pad of 6-8 weeks old female BALB/c mice. It was found that GARP-expressing cells were more aggressive, as indicated by both increased growth kinetics of the primary tumor (FIGS. 5D and SE) and increased lung metastasis (FIG. 5F) It was also found that this aggressiveness correlated with enhanced TGF-β signaling in the tumor microenvironment as determined by increased p-Smad-2/3 in cancer cells (FIGS. 5G and 5H), as well as by expansion of tolerogenic Treg cells (FIGS. 5I and 5J).

### Example 3—Melanoma Studies

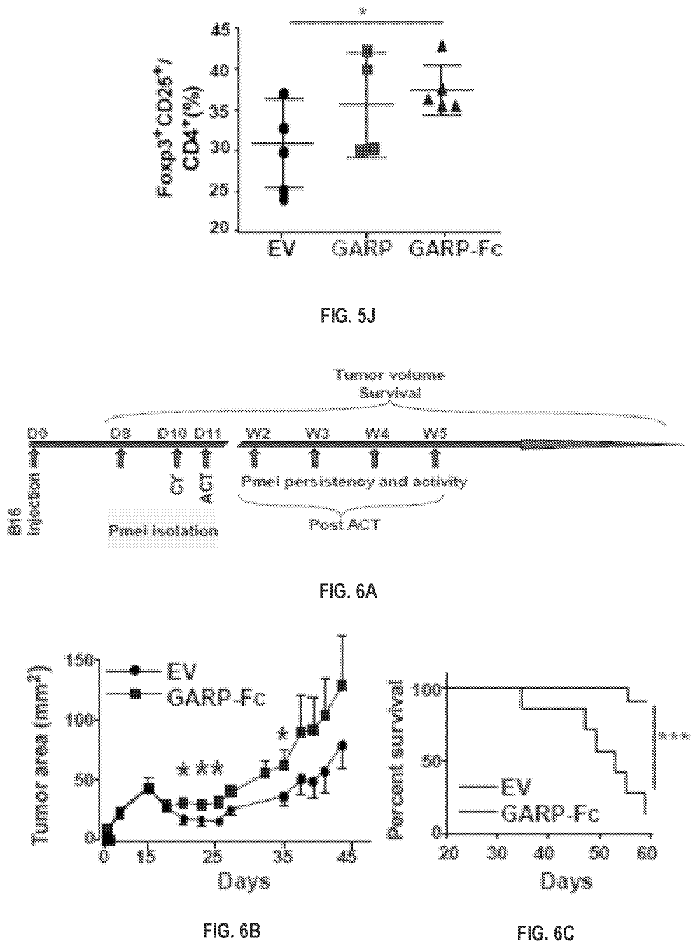

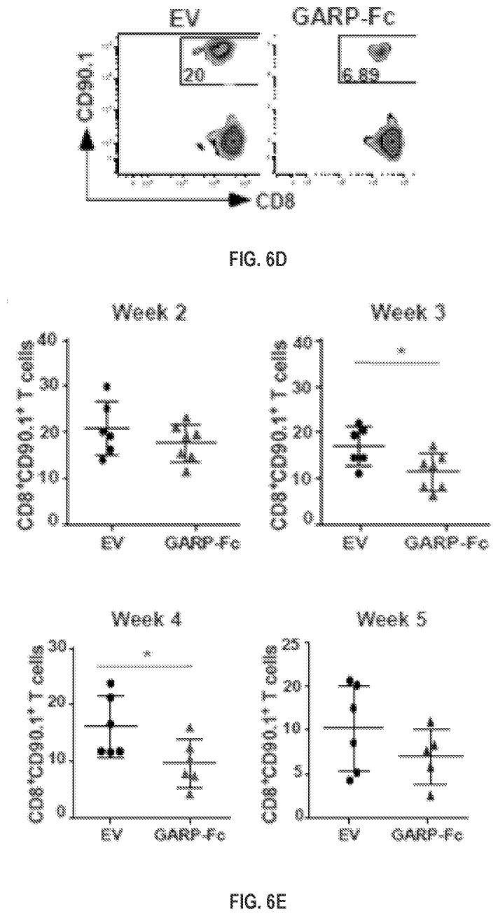

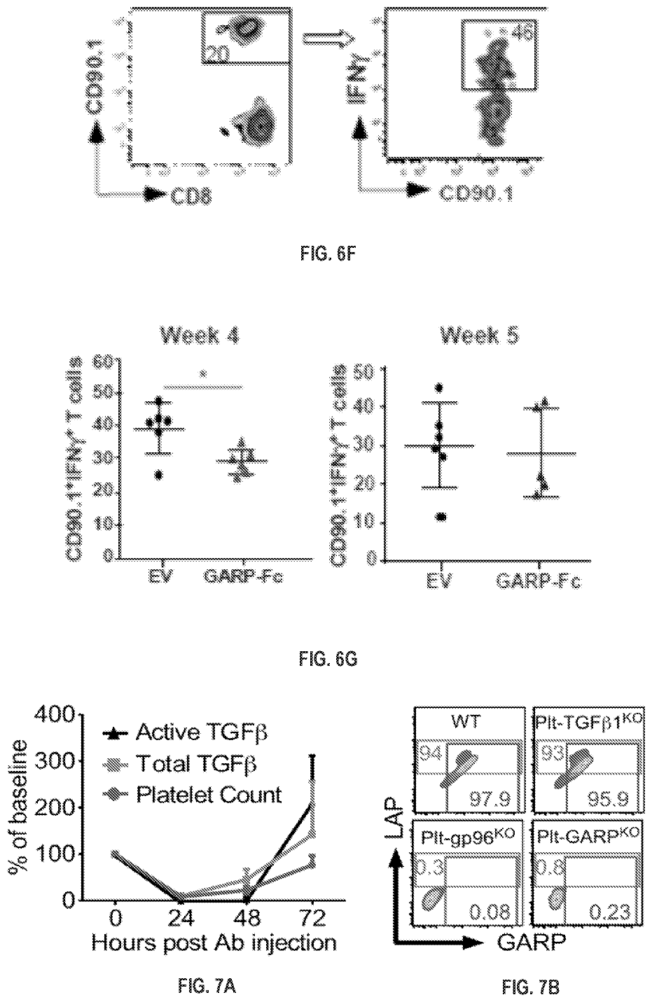

The studies in the 4Tl tumor model prompted the question of whether GARP exerts an inhibitory effect on the function of tumor-specific T cells. To address this possibility, a B16 melanoma model with a defined antigen specificity was utilized along with CD8⁺ T cell receptor (TCR) transgenic mice (Pmel) with T cells specific for the melanoma-associated antigen gp100 (Muranski et al., 2008; Overwijk et al., 2003). B16-F1 cells were prepared with or without GARP-Fc, and then injected subcutaneously in C57BL/6 mice. The tumor bearing mice were then lymphodepleted with cyclophosphamide (CY) before adoptive cell transfer (ACT) of ex-vivo activated Pmel cells (Rubinstein et al., 2015) (FIG. 6A). It was found that expression of GARP-Fc by B16 cells led to increased resistance to ACT (FIGS. 6B and 6C), which was associated with reduced numbers of antigen-specific Pmel cells in the recipient mice, particularly during the first four weeks of tumor growth when the tumor surface area was less than 100 mm² (FIGS. 6D and 6E). Similarly, the ability of Pmel CD8⁺ T cell cells to produce IFNγ in response to antigen stimulation was also impaired in mice bearing GARP-Fc⁺ B16 melanoma (FIGS. 6F and 6G).

### Example 4—GARP as a Novel Therapeutic Target in Cancer

The studies described herein have demonstrated that GARP is aberrantly expressed in multiple human cancers, and that GARP expression in murine tumors is associated with increased TGF-β bioavailability, cancer aggressiveness, and T cell tolerance. It was next determined whether GARP could serve as a novel therapeutic target in cancer, using an antibody-based strategy. For the generation of anti-GARP monoclonal antibodies (mAbs), mice were immunized with recombinant human GARP, followed by boosting with irradiated whole myeloma SP2/0 cells stably expressing human GARP, with the aim of generating mAbs against GARP that were conformation-specific.

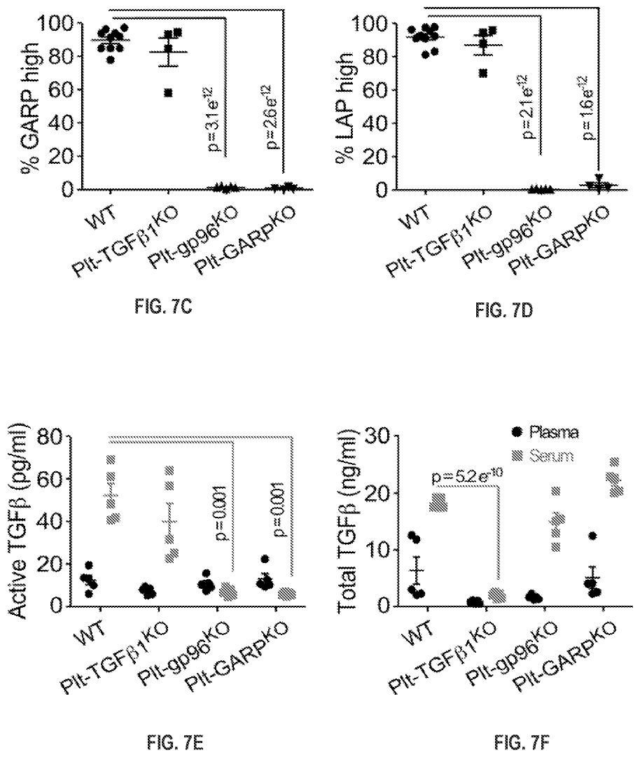

Platelets not only produce and store high levels of TGFβ intracellularly, but also are the only cellular entity known so far that constitutively expresses cell surface docking receptor GARP for TGFβ. Thus, platelets may contribute to the systemic levels of TGFβ via active secretion as well as GARP-mediated capturing from other cells or the extracellular matrix. To what extent and how platelets contribute to the physiological TGFβ pool were addressed. Baseline sera were obtained from wild type (WT) mice followed by administration of a platelet depleting antibody. These mice were sequentially bled and serum TGFβ was quantified by ELISA. Depletion of platelets resulted in a complete loss of active and total TGFβ, which rebounded effectively as soon as platelet count recovered (FIG. 7A). These experiments demonstrate that platelets contribute dominantly to the circulating TGFβ level.

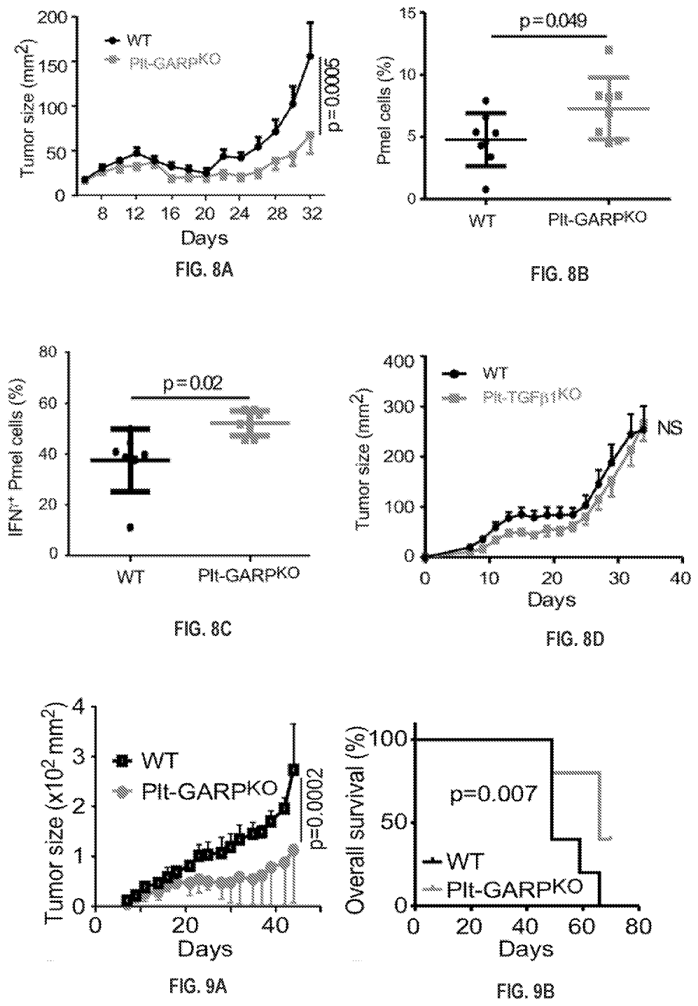

The biology of platelet-derived TGFβ in cancer immunity was experimentally addressed by focusing on the role of platelet GARP in the production of active TGFβ. In addition to platelet-specific Hsp90b1 KO mice, two additional mouse models were generated. One with selective deletion of GARP in platelets (Pf4-cre-Lrrc32flox/flox, or Plt-GARPKO) and another with platelet-restricted knockout of TGFβ1 (Pf4-cre-Tgfb1flox/flox or Plt-Tgfβ1KO). As gp96 is also an obligate chaperone for GARP, platelets from neither Plt-gp96KO mice nor Plt-GARPKO mice expressed cell surface GARP-TGFβ complex. Platelets from Plt-Tgfβ1KO mice, however, expressed similar levels of surface GARP-TGFβ1 complex when compared with WT platelets (FIGS. 7B-8D), indicating that the GARP-TGFβ1 complex can be formed without autocrine TGFβ1.

The levels of active and latent TGFβ were then measured in the plasma and sera of WT and knockout mice (FIGS. 7E-F). In WT mice, active TGFβ was elevated in serum compared to plasma, indicating a role for platelets and/or the coagulation cascade in TGFβ activation (FIG. 7E). Importantly, Plt-gp96KO and Plt-GARPKO mice had very little active TGFβ in their sera, confirming the importance of platelet-intrinsic GARP in converting latent TGFβ to the active form. In contrast, the serum level of active TGFβ in Plt-Tgfβ1KO mice was comparable to that of WT mice (FIG. 7E), indicating that platelets are capable of activating TGFβ from non-platelet sources in a trans fashion. Significantly, the total latent TGFβ level in the serum is only reduced in Plt-Tgfβ1KO mice but not Plt-gp96KO or Plt-GARPKO mice (FIG. 7F). Collectively, these data indicate that platelet-intrinsic GARP is the most important mechanism in the activation of TGFβ systemically. This experiment also categorically confirmed that serum but not plasma level of active TGFβ reflects exclusively platelet activation.

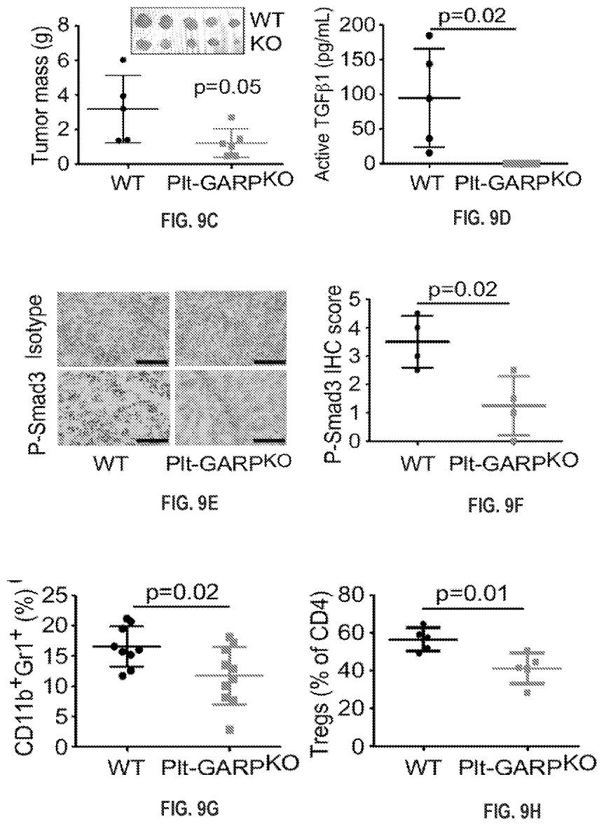

It is hypothesized that platelet-specific GARP play critically negative roles in anti-tumor T cell immunity. This hypothesis was addressed by comparing the efficacy of adoptive T cell therapy of melanoma in WT, Plt-Tgfβ1KO and Plt-GARPKO recipient mice (FIG. 8). B16-F1 melanomas were established in either WT or KO mice, followed by lymphodepletion with cyclophosphamide (Cy) on day 9, and the infusion of ex vivo activated Pmel T cells on day 10 (FIG. 8A). Tumors were controlled much more efficiently in the Plt-GARPKO mice compared with WT mice (FIG. 8A). This was associated with enhanced persistence (FIG. 8B) and functionality of Pmel cells in the peripheral blood of Plt-GARPKO mice (FIG. 8C). In stark contrast, Plt-Tgfβ1KO mice, whose platelets express GARP and remain capable of activating TGFβ, did not have improved control of tumors (FIG. 8D). The generality of these findings was next studied in the MC38 colon carcinoma system given that the growth of this transplantable tumor in syngeneic mice undergoes both CD4 and CD8-mediated immune pressure. The growth of MC38 was significantly diminished in Plt-GARPKO mice compared to WT mice (FIGS. 9A-9C). The MC38-bearing Plt-GARPKO mice had reduced serum levels of active TGFβ (9D). More importantly, staining for p-Smad2/3 (p-Smad2/3) in MC38 tumor sections demonstrated a remarkable attenuation of TGFβ signaling in MC38 cells in Plt-GARPKO mice (FIGS. 9E and 9F). This was associated with reduction of both systemic myeloid-derived suppressor cells (FIG. 9G) and tumor-infiltrating regulatory T cells in Plt-GARPKO mice (FIG. 9H). Taken together, this demonstrates that platelets are the commanding source of TGFβ activity in the tumor microenvironment and they exert potent immunosuppressive effects on anti-tumor immunity via GARP-TGFβ.

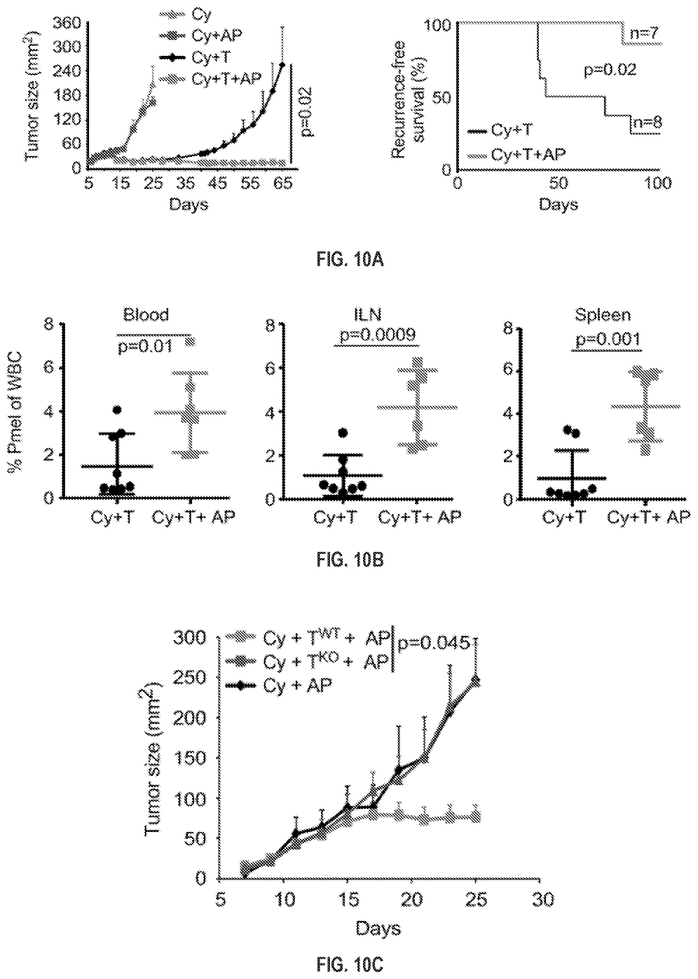

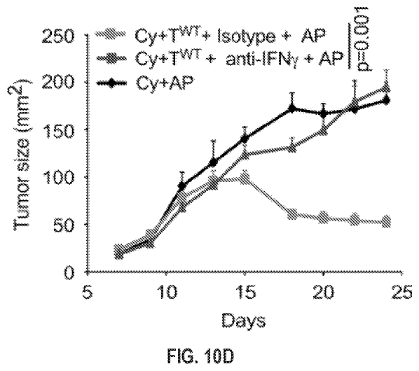

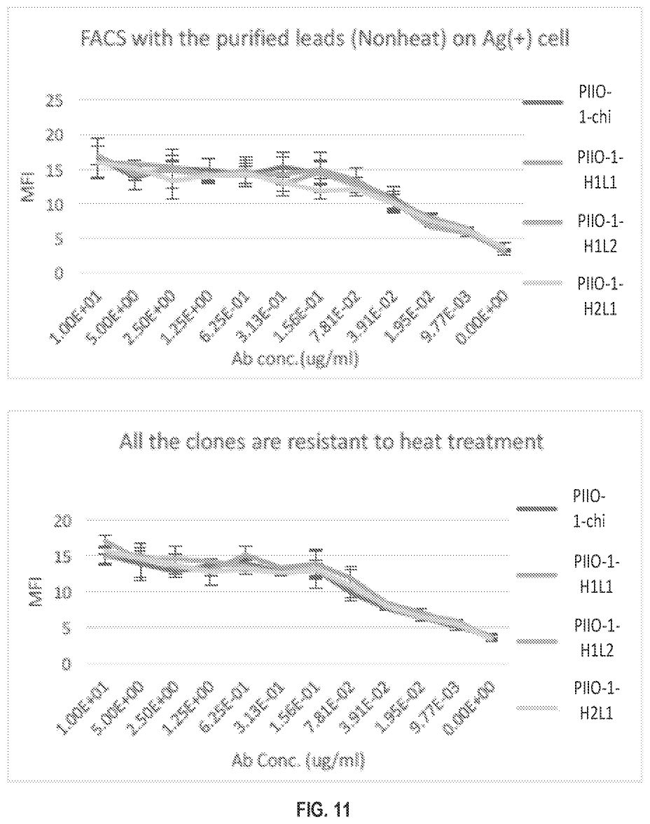

To establish the clinical relevance of the suppressive effect of platelets on anti-tumor immunity, the impact of platelets on immunotherapy was addressed pharmacologically. B16-F1 melanomas were established in C57BL/6 mice after subcutaneous injection on day 0, followed by lymphodepletion with Cy on day 7, and infusion of ex vivo primed Pmel cells on day 8, along with anti-platelet (AP) agents: aspirin and clopidogrel. Aspirin and clopidogrel inhibit platelet activation by blocking cycloxyenase and ADP receptors, respectively. Cy alone failed to control tumors, and the additional AP also had no anti-tumor effects in this model (FIG. 10A, left panel). Melanoma was controlled well with T cells plus Cy for about one month, but most mice eventually relapsed. In contrast, anti-platelet agents plus adoptive T cell transfer were highly effective against B16-F1 with relapse-free survival of most mice beyond 3 months (FIG. 10A, right panel). As a further proof, antigen-specific T cells were sustained at higher numbers in the blood, inguinal lymph nodes (ILNs) and spleens of mice receiving concurrent anti-platelet therapy and ACT (FIG. 10B). Importantly, antiplatelet agents conferred no benefit when the transferred T cells lacked IFNgamma (FIG. 10C) or when anti-IFNgamma neutralization antibodies were administered (FIG. 10D), demonstrating that the effects of anti-platelet agents were immune-mediated.

### Example 5—Materials and Methods

Cell lines and mice. Pre-B cell line (70Z/3) was a kind gift from Brian Seed (Harvard University) (Randow and Seed, 2001). The 4Tl mouse mammary epithelial cell cancer line, wild-type (WT) normal murine mammary gland epithelial cells (NMuMG) and NMuMG* subline with silencing of hnRNP E1. B16-F1 and 293 FT cell lines were purchased from ATCC.

6-8 weeks old female BALB/c, C57BL/6J, NOD-Rag^{-/-}, NSG breeder pairs (NOD Scid Gamma) and Pmel 1 T cell receptor (TCR) transgenic (Tg) mice were purchased from The Jackson Laboratory (Bar Harbor, ME USA). All animal experiments involving mice were approved by Medical University of South Carolina's Institutional Animal Care and Use Committee, and the established guidelines were followed. Control and treated mice were co-housed, and 6-8 weeks old female age-matched mice were used in all experiments.

Tissue microarrays and human serum. All human tumor microarrays (TMAs) were made out of formalin-fixed, paraffin embedded tissues. Colon, lung and one of two breast cancer TMAs were developed from specimens collected at the Medical University of South Carolina (MUSC; Charleston, SC). Each patient specimen in these TMAs was represented in two cores on the slide and each core measured 1 mm in diameter. TMAs for breast and prostate cancers were purchased commercially from Imgenex, Inc (San Diego, CA). These patient specimens were available in a single core of 2 mm in diameter. Clinical and demographic information were obtained from the Cancer Registry of the Hollings Cancer Center at MUSC or provided by the commercial source. This study was approved by the Institutional Review Board (IRB) at MUSC.

Immunohistochemistry (IHC). The mouse anti-human GARP antibody used in this study (ALX-804-867-C100, Enzo Life Sciences) was first tested by Western blot in untransfected and hGARP-transfected Human Embryonic Kidney (HEK)-293 cells and by IHC using hGARP-transfected and control vector-transfected mouse Pre-B leukemic cells 70Z/3. Both analyses demonstrated specificity of the antibody and dilutions used from 1:250 (colon cancer) to 1:60 (all other cancers).

TMA slides were baked for 2 h at 62° C., followed by de-paraffinization in xylenes and rehydration. Antigen retrieval was then performed by boiling in citrate buffer (pH=6.0) for 30 min in a steamer. Slides were incubated in 3% H₂O₂ in dH₂O for 7 min and non-specific binding was blocked by 2% normal horse serum for 2 h at room temperature. Samples were incubated with anti-h-GARP antibody at 4° C. for 16 h, followed by secondary antibody (Vectastain ABC Kit) and development using DAB substrate (Vector Labs SK-4100). Staining was specific to the cytoplasm and cell membrane, with negative nuclear staining.

For mouse IHC, primary tumors and lungs were isolated. Tumor tissue was either placed into OCT media for fresh frozen sections or fixed in 4% paraformaldehyde overnight for fixed sections. For hematoxylin and eosin (H&E) analysis of the tumor and lungs, fixed tissue was incubated in 70% ethanol overnight prior to paraffin embedding, and then cut for H&E staining. For p-Smad-2/3 on fresh frozen tumor sections, 5 μm sections were fixed with 4% paraformaldehyde followed by incubation with 3% H₂O₂. To minimize nonspecific staining, sections were incubated with the appropriate animal serum for 20 min at room temperature, followed by incubation with primary anti-p-Smad-2/3 (EP823Y; Abcam) overnight at 4° C. Standard protocol of anti-rat Vectastain ABC Kil (Vector Labs) was followed.

The staining intensity of GARP and pSmad-2/3 was graded by a board-certified pathologist (S.S.) with the sample identity blinded (0; negative; 1: faint; 2: moderate; 3: strong but less intense than 4; and 4: intense). Percentage of positive cells per patient sample in the TMA was also calculated, in TMAs where specimens where spotted in duplicates, the average of both cores was used as the representative value. Student t-test was implemented to compare categorical variables like normal versus cancer or different disease stages or categories. Kaplan-Meier analysis for correlation of GARP with survival was performed using X-tile software (Camp et al., 2004) Population characteristics were tested for statistically significant differences between low and high GARP expressers using Chi-squared test.

Immunofluorescence analysis. Fresh frozen tumor cryosections (5 μm) were air-dried, fixed in acetone for 10 min and then incubated with

Phycoerythrin conjugated anti-CD31 antibody (1:50). Vessel density was determined by calculating the area of CD31 staining using an ImageJ v1.34 software program (NIH) after imaging on an Olympus fluorescent microscope.

GARP knockdown by lentivirus-expressed short hairpin RNA. A lentivirus vector-expressing short hairpin RNA (shRNA) targeting the mouse GARP transcript was purchased from Sigma-Aldrich (St. Louis, MO). Ecotropic GARP shRNA and control scrambled lentiviral shRNA particles were produced in HEK293FT cells. To knock down GARP in NMuMG* cells, the cells were transduced with lentiviral supernatants targeting GARP and scrambled control. The knockdown efficiency was assessed by RT-PCR (Applied Biosystems Step-One Plus) and flow cytometry (BD Verse) using an anti-mouse GARP antibody (eBioscience).

Generation of GARP-expression vectors. GARP was amplified by PCR and subcloned between the BglII and HpaI sites in a MigR1 retroviral vector. A cDNA construct for expression of the recombinant GARP-Fc fusion protein was generated by joining the extracellular domain of GARP sequence to the sequence encoding the Fc portion of murine IgG2a constant region. The Fc sequence was amplified by PCR from the phCMV1 vector and GARP was amplified using PCR from MigR1 retroviral vector. The two fragments were ligated and cloned into the MigR1 retroviral expression vector. Ecotropic GARP and GARP-Fc retroviral particles were packaged into the Pheonix-Ecotropic cells. Virus propagation and transduction of Pre-B cells, 4TI cells and NMuMG* cells were based on the established protocols (Wu et al., 2012; Zhang et al., 2015). Cells were stably selected by culturing in presence of blasticidin 48 h post transduction for at least 72 h.

Purification of GARP-Fc. For purification of GARP-Fc protein GARP-Fc, MigR1 vector was transfected into Chinese hamster ovary (CHO) cells using Lipofectamine 2000 (Invitrogen) according to the manufacturer's instructions. Stably transfected clones were selected by blasticidin (5 μg/ml) and protein expression was quantified by SDS-PAGE and Western blot under reducing conditions using anti-mouse GARP and anti-mouse Fc antibody. Recombinant GARP-Fc was purified from cell culture supernatants by protein A affinity chromatography (GE Health).

Generation and characterization of anti-GARP antibody Four BALB/c mice were immunized with recombinant human GARP (R&D Systems, Minneapolis, MN) with Freund's complete adjuvant, followed by boosting with SP2/0 cells stably expressing human GARP for 2-3 times. Splenic B cells from mice with high anti-GARP antibody titers were fused to SP2/0 cells in the presence of polyethylene glycol. Hybridomas were selected in HAT medium and cloned by limiting dilution assay. The specificity of antibody was screened and determined by ELISA and flow cytometry using 70Z/3 cells stably transduced with empty vector (70Z/3-EV) and overexpression of human GARP (70Z/3-GARP).

Protein extraction, immunoprecipitation, and Western blot analysis. Cells were harvested by trypsin-EDTA when necessary, washed in PBS, and lysed on ice in radio-immunoprecipitation assay (RIPA) lysis buffer in the presence of a protease inhibitor cocktail (Sigma-Aldrich). Nuclear-free protein lysate was quantified by Bradford assay (Bio-Rad), and an equal amount of lysate was analysed by SDS-PAGE and Western blot under reducing conditions using anti-mouse GARP (AF6229; R&D system), anti-mouse Vimentin (D21H3; Cell signaling), anti-mouse E-Cadherin (24E10; Cell Signaling) and anti-mouse p-Smad-2/3 (EP823Y; Abcam).

Cell proliferation and in vitro wound healing assay. NMuMG cells (4×10⁵) were starved overnight in serum free DMEM (Corning cellgro). Starved cells were cultured at the indicated times with GARP-Fc in 2% FBS DMEM. To measure cell proliferation, 2.5×10⁴ cells were seeded in a 96-well plate in complete medium (DMEM, 10% FCS, 1% penicillin-streptomycin) and incubated overnight. Proliferation was determined with 3-[4,5 dimethylthiazol-2-yl]-2,5-diphenyltetrazolium bromide (MTT), which was added to the cells at the indicated times and incubated for an additional 3 h at 37° C. The medium was then removed and mixed with 100 μl of DMSO for 15 minutes by shaking. Absorbance at 570 nm was then measured using a plate reader. The cell migration was measured by the wound-healing assay: at 100% confluence, two parallel wounds were made using a 1 ml pipette tip. Migration was assessed after 24, 48 and 72 hours and quantification of wound closure was measured using the ImageJ software (NIH).

4T1 Tumor model and GARP antibody therapy. Female BALB/c mice, 6-8-week old were inoculated in the fourth mammary fat pad subcutaneously (s.q.) with 5×10⁵ cells (4T1 EV, 4Tl GARP, or 4T1 GARP-Fc). Tumor growth was monitored three times per week with a digital vernier caliper and tumor volume was calculated using the following formula: tumor volume (mm³)=[(width)²×length]/2. In GARP antibody therapy experiments, beginning at 3 days post-tumor inoculation, anti-GARP antibody or polyclonal isotype-controlled antibody (0.1 mg/mouse in 0.1 mL PBS; three times per week) were administered intraperitoneally (i.p.) into mice. For combination therapy with cyclophosphamide (CY) and antibody, mice were treated with one injection of CY (4 mg/mouse) 3 days post-tumor inoculation in addition to the antibody treatment. At end-point, mice were sacrificed and the primary tumor, draining LNs, spleen, lungs and liver were isolated. Tumor infiltrated lymphocytes were isolated by Collagenase D (Sigma) digestion followed by Histopaque-1083 (Sigma) mediated density separation.

B16-F1 tumor model and adoptive T cell therapy (ACT). Three groups (B16 EV, and B16 GARP-Fc; n=5-7 each group) of 6-8-week old female C57BL/6J mice were inoculated s.q. in the right flank using 2.5×10⁵ cells and, when specified, treated with one intra-peritoneal injection of CY (4 mg/mouse) a day prior to adoptive T cell therapy. To obtain gp100-specific T cells, the splenocytes from Pmel TCR transgenic female mouse were stimulated with hgp 100 (25-33 epitope, 1 μg/ml, American peptide Company) and mouse IL-12 (10 ng/ml, Shenandoa) for 3 days ACT was done via tail vein injection of 2×10⁶ activated Pmel T cells per recipient mouse a day after injection of CY. Primary tumor growth was monitored 3 times per week with vernier calipers. Peripheral adoptively transferred Pmel cells were monitored at 2, 3, 4, and 5 weeks after ACT. Ex-vivo Pmel IFN-γ production was assess stimulating Pmel cells for 3 h in presence of hgp100 and brefeldin A (BFA) at 37° C. and analyzed by flow cytometry.

NMuMG tumor model. Female NOD-Rag-1^{-/-} (n=5 each group; 6-8 week-old) mice were inoculated in the fourth and left mammary fat pad subcutaneously using 5×10⁵ cells (NMuMG*-EV, GARP knockdown NMuMG*). Animals were weighed and tumors measured weekly. At endpoint, primary tumors, lungs and livers were harvested. In another experiment, female NOD-RagI^{-/-} mice (n=4-5 each group; 6-8 week-old) were inoculated in the fourth left mammary fat pad subcutaneously with 5×10⁵ cells (NMuMG-GARP-Luc, NMuMG-GARP-Fc-Luc or NMuMG-Luc cells). In vivo luciferase imaging was evaluated weekly as follows: mice were intraperitoneally injected with D-luciferin (Perkin Elmer) at a dose of 150 mg/kg per mouse and anesthetized. Bioluminescence images were then acquired using Xenogen IVIS imaging system. Bioluminescence signal was quantified as photon flux (photons/s/cm²) in defined regions of interest using Living Image software (Xenogen).

TGF-β1, GARP, and GARP-TGF-β1 analysis. Active TGF-β1, total TGF-β1, and soluble GARP were measured in human and mouse serum using TGF-β1 and GARP ELISA kits (BioLegend, San Diego, CA) according to the manufacturer's protocols. To measure GARP-TGF-β1 complex by ELISA, 96-well plates were coated with TGF-β1 capture antibody according to the manufacturer's instructions (BioLegend, San Diego, CA). Samples were incubated for 2 h at room temperature followed by the incubation with the anti-hGARP detection antibody developed in our lab for another 2 h.

For MFB-F11 functional assay, MFB-F11 cells (a kind gift from Tony Wyss-Coray, Stanford University) were cultured in DMEM with 10% FBS and 1% penicillin/strepomycin. 2×10+ cells were seeded per well and incubated overnight. Prior to addition of diluted serum or tumor supernatant, cells were serum starved for 2-3 hours. Diluted serum or tumor supernatant samples were incubated for 24 hours, followed by analysis using QUANTI-Blue Medium (InvivoGen, San Diego, CA) (Tesseur et al., 2006).

Statistical Analysis. In TMAs where specimens were spotted in duplicate, the average of both cores was used as the representative value. Student t-test was implemented to compare categorical variables such as normal versus cancer or different disease stages or categories. Kaplan-Meier analysis for correlation of GARP with survival was performed using X-tile software (Camp et al., 2004). Population characteristics were tested for statistically significant differences between low and high GARP expressers using Chi-squared test. Tumor curve analysis was performed using 2-way analysis of variance (ANOVA), all other experiments were analyzed using Two-tailed Student T-test with GraphPad Prism. All data are presented as mean+SEM. P values less than 0.05 were considered to be statistically significant.

### Example 6—Humanization Report for Antibody 4D3

Computational modeling. MAb 4D3 Fv homology model was built up by using pdb 1KC5 as model structure and humanization design was double checked with another hetero model built up on pdb IMCP and pdb 32C2.

Backmutation design rule. During the humanization process, mouse CDRs were grafted into the human framework acceptor, residues in human framework which are different from those in mouse framework were studied. Backmutations from human residue to mouse residue were designed based on the following rule:

If a new contact (ironical interaction, hydrogen bond, hydrophobic interaction) is created between this human residue to mouse Fv CDR residue, canonical residue, interface residue or vernier residue, this human residue needs to be back-mutated to mouse residue. If an old contact (ironical interaction, hydrogen bond, hydrophobic interaction) between a mouse residue and canonical residue, interface residue or vernier residue is lost when a human residue replacing a mouse residue, this human residue needs to be back mutated to mouse residue. Replacement of mouse canonical residue, interface residue or Vernier residue with human residue should be carefully studied and usually avoided.

Schrodinger surface analysis data. Schrodinger surface analysis of mouse MAb 4D3 Fv and huVHv1VLv1 was performed. Only if an aggregation problem is observed in humanization leads from bench work in the future, the surface analysis data is further studied in order to fix the problem.

Schrodinger post-translational modification data. Schrodinger post-translational modification of mouse MAb4D3 Fv and huPIIO-1VH1VL1 (data from the humanized version with the highest humanization percentage) was performed. Only typical PTM motif with side chain has >50% accessibility to 3D surface are ranked as “high risk” residues (e.g., “NG” is typical deamidation site, “QG” is non-typical). These kinds of PTMs were found in the huMAbPIIO-1VH1VL1 PTM analysis files.

T-cell epitope, B cell epitope and MHC II epitope study. All potential T-cell epitope, B cell epitope, MHC II epitope and antigenicity epitopes predicted by Protean 3D in the framework of the highest humanized version PIIO-1VH1VL1, which contain backmutations, were listed. Those framework epitopes contain backmutations. Removal of these backmutations may lead to loss of affinity and/or developability.

Ranking humanized candidates. Four humanized VHs and 3 VLs were designed, resulting in 12 VH/VL combination. IgG protein of the 12 humanized leads and the wild-type HCL64 clone were produced in small scale, and the unpurified culture supernatant was used in the following ELISA assay.

TABLE A
FACS analysis with Ag(+) cell

| | FACS with Ag(+) cell (MFI) | | | | | | | | | | | | |
|---|---|---|---|---|---|---|---|---|---|---|---|---|---|
| Ab conc.(ug/ml) | H1L1 | H2L1 | H3L1 | H4L1 | H1L2 | H2L2 | H3L2 | H4L2 | H1L3 | H2L3 | H3L3 | H4L3 | Chimeric |
| 5 | 26.39 | 26.79 | 26.72 | 27.31 | 26.06 | 26.24 | 26.45 | 26.32 | 26.34 | 26.46 | 26.25 | 26.71 | 26.68 |
| 1 | 25.76 | 24. [symbol] | 25.1 | 26.18 | 25.22 | 2 [symbol] .86 | 24.87 | 24.87 | 25.74 | 25.64 | 25.34 | 26.26 | 26.09 |
| 0.2 | 23.93 | 23.3 | 23.95 | 24.84 | 22.19 | 23.21 | 22.73 | 22.41 | 22.85 | 23.37 | 21.95 | 25.04 | 23.27 |
| 0 | 9.91 | 9.91 | 9.91 | 9.91 | 9.91 | 9.91 | 9.91 | 9.91 | 9.91 | 9.91 | 9.91 | 9.91 | 9.91 |

[symbol]

indicates data missing or illegible when filed

Binding test on FACS with Culture Supernatant. The FACS result showed that all the humanized leads kept the similar binding capacity to the wild-type 4D3 chimeric clone. The plateau MFI is relatively low. To double check the binding, the FACS binding assay was repeated. To quickly evaluate the thermostability of the humanization leads, the inventor treated the supernatant sample under 70° C. for 5 mins, then use the sample to repeat the FACS assay with the same conditions. The results show that both the 4D3 chimeric clone and all the designed humanization clones are totally resistant to the heating treatment.

TABLE B
Preliminary thermostability test

| | FACS with Ag(+) cell and Heated supernatant sample (MFI) | | | | | | | | | | | | |
|---|---|---|---|---|---|---|---|---|---|---|---|---|---|
| Ab conc.(ug/ml) | H1L1 | H2L1 | H3L1 | H4L1 | H1L2 | H2L2 | H3L2 | H4L2 | H1L3 | H2L3 | H3L3 | H4L3 | Chimeric |
| 5 | 26.24 | 26.25 | 25.69 | 26.85 | 25.68 | 26.09 | 26.51 | 25.09 | 26.32 | 26.39 | 25.77 | 26.97 | 2 [symbol] .35 |
| 1 | 25.2 [symbol] | 25.12 | 25.0 [symbol] | 25.35 | 24. [symbol] 6 | 24.84 | 24.64 | 25.02 | 25.41 | 25.41 | 24.59 | 25.83 | 24.68 |
| 0.2 | 23.9 [symbol] | 23.02 | 23.59 | 24.25 | 21.82 | 22.61 | 22.71 | 21.74 | 22.97 | 23.19 | 22.14 | 24.17 | 22. [symbol] 9 |
| 0 | 9.91 | 9.91 | 9.91 | 9.91 | 9.91 | 9.91 | 9.91 | 9.91 | 9.91 | 9.91 | 9.91 | 9.91 | 9.91 |

[symbol]

indicates data missing or illegible when filed

No clone showed any non-specific binding on the Ag(−) cell.

TABLE C
FACS Analysis with Ag(−) cell

| | FACS with Ag(−) cell (MFI) | | | | | | | | | | | | |
|---|---|---|---|---|---|---|---|---|---|---|---|---|---|
| Condition | H1L1 | H2L1 | H3L1 | H4L1 | H1L2 | H2L2 | H3L2 | H4L2 | H1L3 | H2L3 | H3L3 | H4L3 | Chimeric |
| Non-Heating | 3.31 | 3.23 | 3.28 | 3.11 | 3.24 | 3.24 | 3.37 | 3.26 | 3.22 | 3.18 | 3.16 | 3.27 | 3.37 |
| Heating | 3.36 | 3.29 | 3.4 | 3.25 | 3.35 | 3. [symbol] 6 | 3.26 | 3.29 | 3.33 | 3.35 | 3.29 | 3.36 | 3.36 |

All the antibody sample has a concentration of 5 ug/ml. [symbol]
indicates data missing or illegible when filed

Conclusion. Since all the designed humanization clones are very similar in specific binding and thermo-stability assays, the inventor selected clone VH1VL1, VH1VL2, VH2VL1 for more tests mainly based on humanization percentage (VH1>VH2>VH3>VH4, VL1>VL2>VL3).

Methods—transient transfection. Synthesize the wild-type 4D3 (chimeric) and humanized VH/VL DNA. Transient transfect Expi293 cell with different VH/VL combination. Three days-post transfection, collect the culture supernatant, measure the IgG level using ProteinA sensor on Gator (similar to Octet, ProbeLife at Palo Alto, CA) and amended with ELISA measurement.

Methods—FACS analysis. Cell preparation: Cell were harvested and washed with PBS+2% FBS for one time. Cell density was adjusted to 1.5E6/mL in PBS/2% FBS. Cell were added to 96 U-bottom well plates at 100 μL/well. Antibody samples preparation: Antibody in supernatant was adjusted to 10 μg/mL using PBS+2% FBS, with serial 5 times dilution performed to obtain 3 concentrations of antibody solution. The blank was PBS+2% FBS. Antibody solutions were placed into 96 U-bottom well plates at 100 μL/well in the same arranging pattern as the cell preparation. Incubation: The antibody samples (100 μl) were mixed with the cells (100 μl), and incubated at RT for 1 h, then centrifuged the plate for 3 min at 1000 rpm (swing bucket). The supernatant was pipetted and the cells were washed with PBS+2% FBS for one time. Incubation with the secondary antibody: Cy3-Conjugated AffiniPure Goat Anti-Human IgG was diluted 250× by PBS+2% FBS, and added to the 96 U-bottom well plate with 100 μl/well, then incubated at RT for 30 min. After that, the plate was centrifuged at 1000 rpm for 3 min and the supernatant was pipetted out. The cell was washed with PBS+2% FBS twice. Cells were re-suspended in 200 μl PBS+2% FBS and analyzed on FACS. The MFI of total live cells were used as binding signal.

Methods—heat treatment. Culture supernatant was serial diluted to the indicated concentration of IgG with cell media and heated at 70° C. for 5 mins on a PCR machine, then quickly cooled down to room temperature.

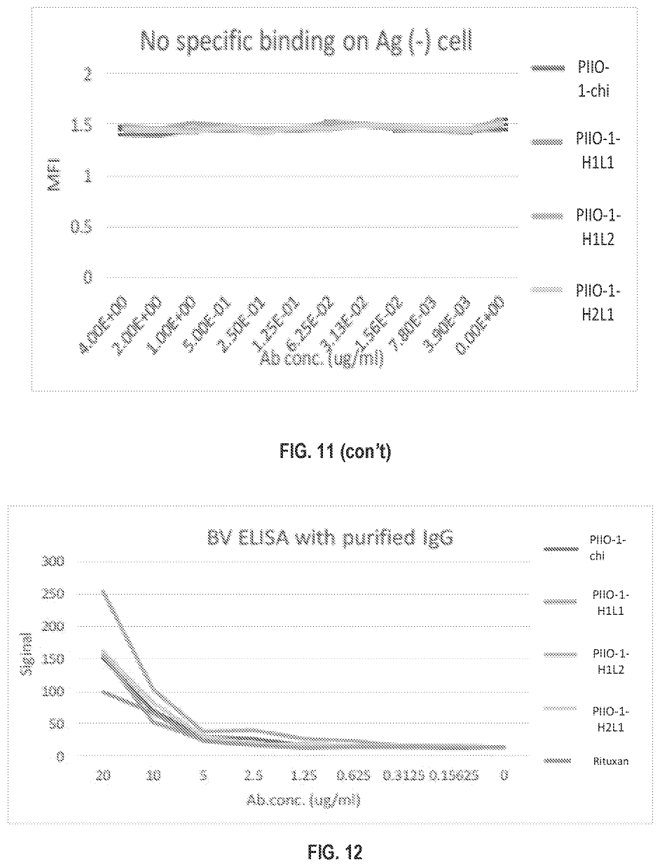

Characterizing selected humanization leads with purified IgG. Based on the culture supernatant results, the inventor picked three humanized leads VH1VL1, VH1VL2, and VH2VL1. Expi 293 cells were co-transfected with VH and VL plasmid DNA of each of the selected leads and IgG was purified for each candidate. FACS analysis was repeated with the purified antibody to compare the humanized leads with the wild-type chimeric in specific binding capacity. Preliminary assays were conducted to compare their thermo-stability and non-specific binding. The results are shown in FIG. 11.

Conclusion. With the purified IgG antibodies, the results confirmed that the selected candidates have very similar binding affinity. Under treatment of 70° C. for 5 minutes, all of three leads showed similar binding ability compared with chimeric antibody.

Methods—FACS analysis. Cell preparation: Cell were harvested and washed with PBS+2% FBS once. Cell density was adjusted to 1.5E6/mL in PBS/2% FBS. The cells were added to 96 U-bottom well plate at 100 μl/well. Antibody samples preparation: Antibody concentration was adjusted to 20 μg/mL using PBS+2% FBS, then serial dilutions were performed to achieve different concentrations of antibody solution. The blank was PBS+2% FBS, and antibody solutions were placed into 96 U-bottom well plate with 100 μl/well using the same pattern as for the cell preparation. Incubation: The antibody samples (100 μl) were mixed with the cells (100 μl) and incubated at RT for 1 hr. Then the plates were centrifuged for 3 min at 1000 rpm (swing bucket). The supernatant was pipetted out and the cells were washed once with PBS+2% FBS. Incubation with the secondary antibody: Cy3-Conjugated AffiniPure Goat Anti-Human IgG was diluted 250× by PBS+2% FBS, added to the 96 U-bottom well plate at 100 μl/well and then incubated at RT for 30 min. After that, the plate was centrifuged at 1000 rpm for 3 min and the supernatant was pipetted out. The cells were washed twice with PBS+2% FBS twice. FACS detection: The cells were resuspended with 200 μL PBS+2% FBS and then analyzed by FACS.

Methods—heat treatment. Antibody was heated at 70° C. for 5 mins on a PCR machine, then quickly cooled down to room temperature.

Affinity measurement. Label-free kinetic binding assay was performed on Gator (similar to Octet). The results show that the 3 humanization leads (H1L1, H1L2, H2L1 also referred to herein as VH1VL1, VH1VL2, and VH2VL1, respectively) have the same KD value to the chimeric.

TABLE D
Analyzed kinetic binding data

| Sensor | Antibody | kobs | koff | kon | KD | Rmax | Req | Response |
|---|---|---|---|---|---|---|---|---|
| CH1 | Chimeric | 0.013 | 0.000812 | 9.74E+04 | 8.34E−09 | 0.892 | 0.836 | 0.828 |
| CH2 | H1L1 | 0.0134 | 0.00081 | 1.01E+05 | 8.05E−09 | 0.791 | 0.743 | 0.737 |
| CH3 | H1L2 | 0.0133 | 0.000813 | 9.98E+04 | 8.15E−09 | 0.885 | 0.831 | 0.824 |
| CH4 | H2L1 | 0.0133 | 0.000806 | 1.00E+05 | 8.03E−09 | 0.756 | 0.71 | 0.703 |

Methods. Affinity was measured on Gator (ProbeLife, Palo Alto). In brief, the purified IgG sample was diluted in K buffer at 2 μg/ml, and the antigen was diluted at 5 μg/ml. The antibody-loaded anti-human Fc probes were dipped in antigen wells for 5 mins and then moved to K buffer wells for 5 mins. In the whole process, the sample plate was shaken at 1,000 rpm. The data analysis was carried out using Gator software (ProbeLife).

Evaluation of the humanized lead's non-specific binding to “sugar, lipid and protein”. Baculovirus (BV) ELISA was employed as a preliminary assay to evaluate antibody's potential nonspecific binding risk. The results are shown in FIG. 12 and Table E below.

TABLE E
Raw BV binding data

| | BV ELISA with purified IgG | | | | |
|---|---|---|---|---|---|
| Ab. Conc. | 4D3 | PIIO- | PIIO- | PIIO- | |
| (ug/ml) | chimeric | 1VH1VL1 | 1VH1VL2 | 1VH2VL1 | Rituxan |
| 20 | 150.9 | 157.2 | 253.4 | 161.0 | 98.8 |
| 10 | 69.9 | 52.7 | 102.3 | 82.7 | 66.0 |
| 5 | 30.3 | 24.9 | 38.1 | 30.8 | 22.7 |
| 2.5 | 26.4 | 18.3 | 39.6 | 17.8 | 17.0 |
| 1.25 | 16.8 | 16.0 | 26.6 | 18.3 | 13.0 |
| 0.625 | 15.7 | 14.7 | 23.5 | 15.6 | 13.7 |
| 0.3125 | 14.2 | 16.2 | 15.4 | 14.6 | 13.7 |
| 0.15625 | 14.3 | 15.0 | 15.6 | 14.5 | 12.1 |
| 0 | 14.0 | 14.0 | 14.0 | 14.0 | 14.0 |

Conclusion and Summary. In Baculovirus (BV) ELISA, the inventor used Rituxan Mab as reference. If an antibody shows weaker BV-binding than Rituxan, it will be class no non-specific binding issue. With purified antibodies, the results show that chimeric, VH1VL1, VH2VL1 and Rituxan have similar binding, and clone VH1VL2 has a bit higher signal. Humanized clones VH1VL1, VH1VL2, and VH2VL1 (also referred to herein as H1L1, H1L2, and H2L1, respectively) are very similar in specific binding, preliminary thermostability, and non-specific binding assay (BV ELISA). In the BV ELISA assay, clone VH1VL2 has a bit higher signal than the other clones, but none of the clones show any non-specific binding on Ag(−) cells (see Experiments 1.1.1 and 2.1.1). If a clone only has binding on BV, but not on 293 cells, it will be considered as having a low risk of non-specific binding. Thus, clone VH1VL1, VH1VL2, and VH2VL 1 can be the candidates for further assessment.

Methods—Baculovirus ELISA. Plates are coated with 50 μl of 1:500 diluted Baculovirus sample in PBS in each well. Plates are kept at 4° C. for overnight. The plates are washed with 300 μL of wash buffer ×3 and 200 μl blocking buffer (1% BSA) is added at RT for 60 min. Plates are washed with 300 μl of wash buffer ×3 and 100 μl of diluted Abs are added at different concentrations/well, followed by RT incubation for 1 hr. Plates are washed with 300 μl of wash buffer ×6 and 100 μl of 1:5000 HRP conjugated second Ab in PBS is added followed by RT incubation for 1 hr. The plates are washed with 300 μl of wash buffer ×6. Developing buffer is added and the plate is read.

Developability Analysis for PIIO-1 humanization leads. Antibodies were analyzed for thermostability by DSF/SLS. Samples were submitted to the UNcle system (Unchained Labs) for analysis. A temperature ramp of 1° C./min was performed with monitoring from 20° C. to 95° C. for DSF and SLS. UNcle measures SLS at 266 nm and 473 nm. Tm and Tagg were calculated and analyzed by using the UNcle Analysis Software. DSF: Differential scanning fluorimetry; SLS: Static light scattering; Tm: Melting temperature; Tagg 266: Thermal aggregation when SLS at 266 nm; Tagg 473: Thermal aggregation when SLS at 473 nm. A summary is shown below in Table F:

TABLE F

| | DSF (° C.) | | | | SLS (° C.) |
|---|---|---|---|---|---|
| Sample | Tm1 | TM2 | Tm3 | Tagg 266 | Tagg 473 |
| 4D3 chimeric | 72.0 | 81.0 | | 75.0 | 75.4 |
| huPIIO-1VH1VL1 | 70.2 | 83.7 | | 74.2 | 74.9 |
| huPIIO-1VH1VL2 | 70.0 | 76.7 | 87.5 | 78.8 | 79.3 |
| huPIIO-1VH2VL1 | 69.9 | 83.2 | | 73.7 | 74.7 |

IgG is a multi-domain structure and each domain has its own melting Temperature (Tm). CH2 domain usually has Tm of ˜70° C. in PBS, while CH3 is more stable, exhibiting a Tm of about 80° C. Fabs have Tm in a wide range, generally about 50-85° C., due to large sequence variation. Therefore, the Tm values measured by various analytical techniques are usually “apparent” transition temperatures rather than the real Tm for each domain. In the case of whole IgG, there are often 2-3 Tm values in DSF measurement, presenting some challenge in determining which Tm represents which domain.

All the huPIIO-1 clones measured have two or three Tms and it is very likely that the higher one (Tm2) represents CH3, while Tm1 represents Fab+CH2. The DSF results show that the three huPIIO-1 candidates have similar thermos-stability to the 4D3 wild-type clone.

Tagg is the temperature at which SLS starts to detect aggregation particles. Tagg266 measures SLS at 266 nm, which is more sensitive and suitable to detect smaller aggregation particles. Tagg473 measures SLS at 473 nm and is better to detect larger particles.

All the huPIIO-1 candidates have somewhat different Taggs value as compared to the 4D3 wild-type clone, meaning that the candidates have similar aggregation potential as wild-type clone.

Aggregation potential by DLS analysis. DLS was performed on UNcle system (Unchained Labs). DLS was measured at 25° C. Data was calculated and analyzed using UNcle Analysis Software. DLS: Dynamic light scattering PDI: Polydispersity index, PDI=(standard deviation/mean hydrodynamic radius). A summary of the results in shown in Table G below:

TABLE G
DLS Results Summary

| | Peak 1 | | | Peak 2 | |
|---|---|---|---|---|---|
| | Mode | | | Mode | |
| | Diameter | Mass | | Diameter | Mass |
| Sample | (nm) | (%) | PDI | (nm) | (%) |
| 4D3 chimeric | 10.41 | 99.94 | 0.250 | N/A | 0 |
| huPIIO-1VH1VL1 | 9.68 | 99.95 | 1.228 | N/A | 0 |
| huPIIO-1VH1VL2 | 9.68 | 99.94 | 0.361 | N/A | 0 |
| huPIIO-1VH2VL1 | 9.68 | 100 | 0.141 | N/A | 0 |

Dynamic Light Scattering (DLS) is used to detect aggregation in the antibody sample. “Mode diameter” is protein particle diameter and “mass percentage” is the amount of each size fraction in percentage. “PDI” is Polydispersity Index; the higher this index, the more polydispersity in the sample is. As shown in the above table, VH1VL2 and VH2VL 1 have similar or better PDI compared with WT, and PDI for VH1VL1 is slight worse than WT. Peak 1 is the major peak and represents the IgG monomer. The inventor takes “Peak 1 mass percentage” and “PDI” value into consideration in selecting a lead. VH1VL2 and VH2VL1 have very similar Peak1 mass percentage and PDI value to the chimeric clone.

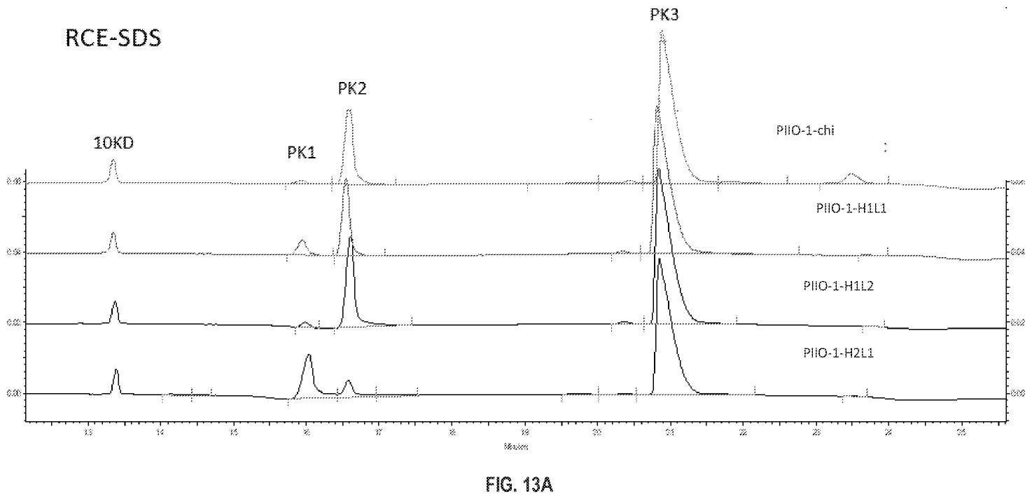

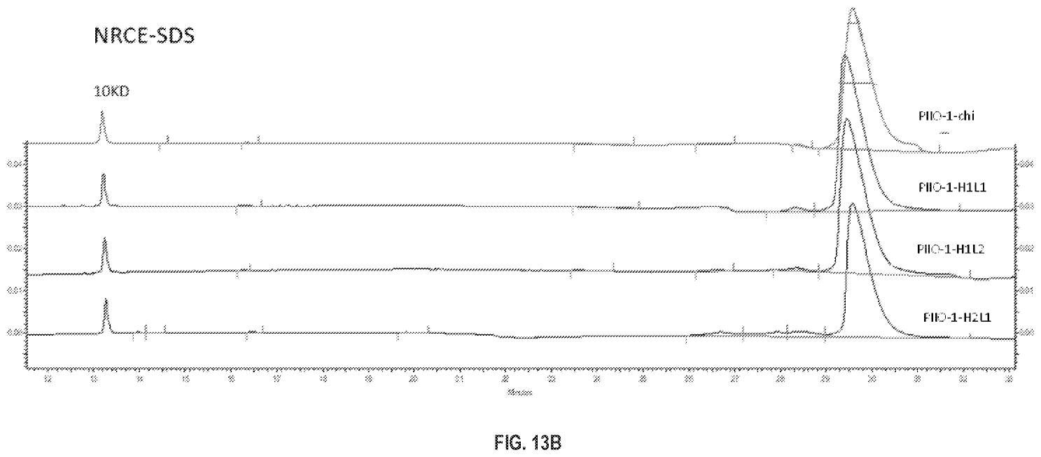

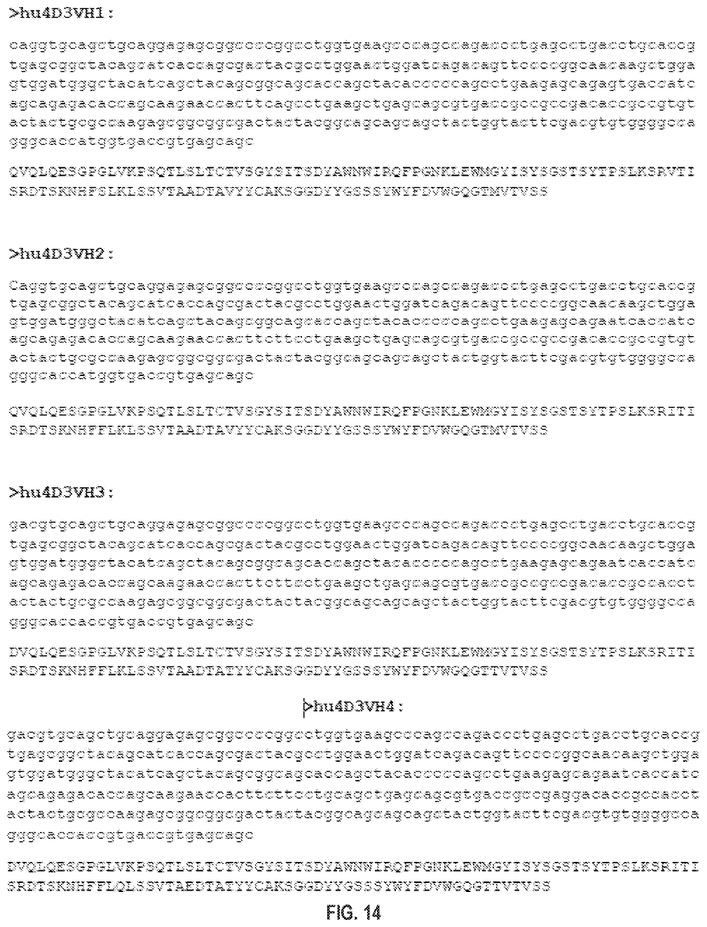

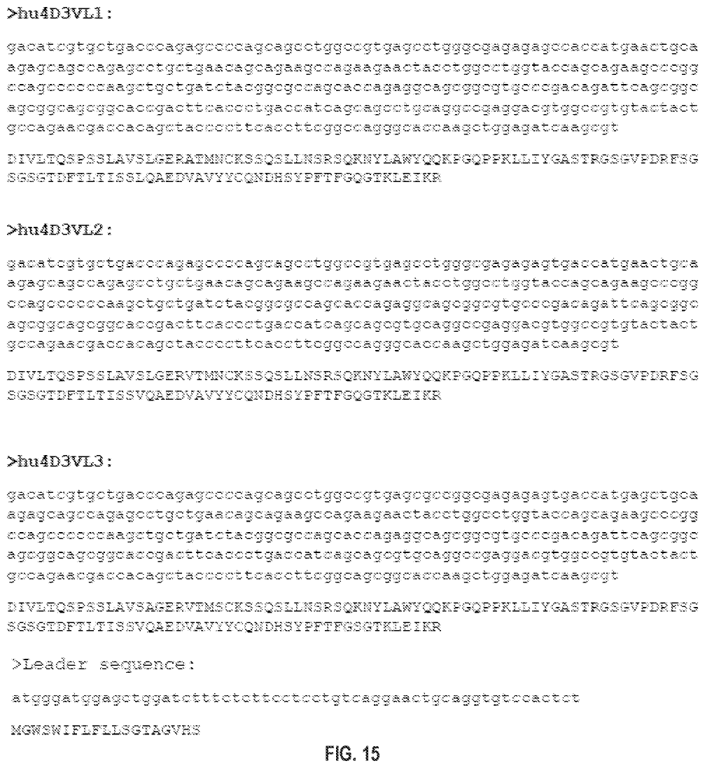

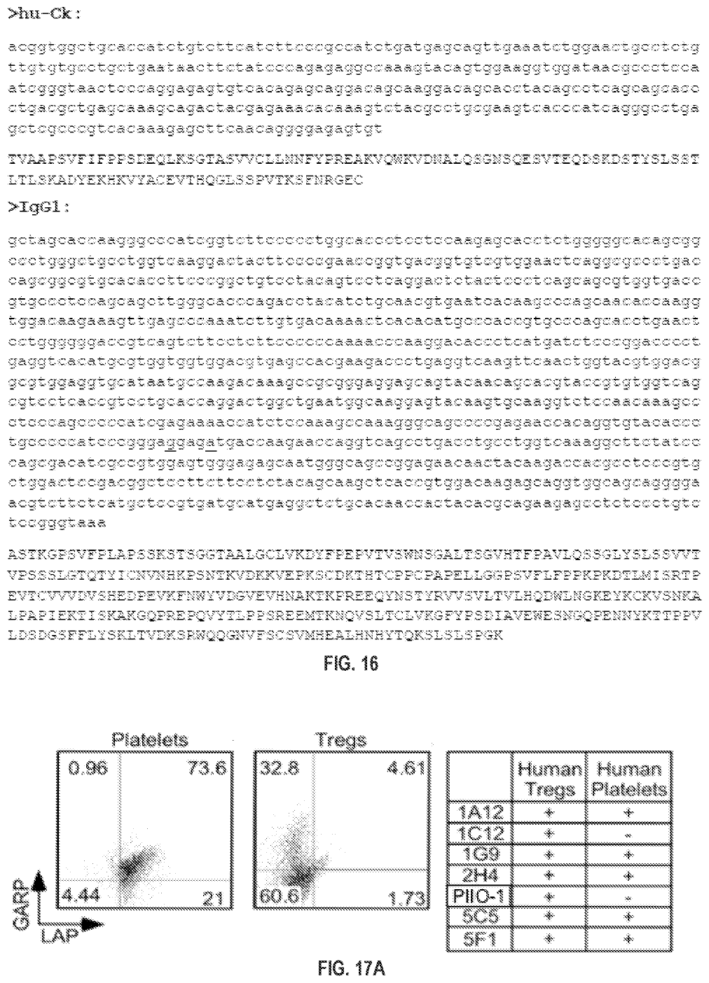

Analysis of the heterogeneity by Capillary Electrophoresis (CE). The sample was prepared in reducing and non-reducing labeling buffer before being submitted to the CE analysis. A summary of the Reducing-CE-SDS and Non-Reducing-CE-SDS results are shown in FIG. 13A/Table H and FIG. 13B/Table I, respectively:

TABLE H

| | 10KD | | PK#1 | | PK#2 | | PK#3 | |
|---|---|---|---|---|---|---|---|---|
| | Moving | PK#1 | Moving | PK#2 | Moving | PK#3 | Moving | OTHER |
| Sample | Time | (%) | Time | (%) | Time | (%) | Time | (%) |
| 4D3 | 13.353 | 1.3 | 15.925 | 24.9 | 16.592 | 65.5 | 20.883 | 8.2 |
| chimeric | | | | | | | | |
| huPIIO- | 13.355 | 5.6 | 16.95 | 23.2 | 16.55 | 70.4 | 20.808 | 0.9 |
| 1VH1VL1 | | | | | | | | |
| huPIIO- | 13.375 | 1.4 | 15.983 | 29.1 | 16.608 | 68.7 | 20.833 | 0.9 |
| 1VH1VL2 | | | | | | | | |
| huPIIO- | 13.392 | 21.7 | 16.033 | 6.9 | 16.575 | 68.7 | 20.85 | 2.7 |
| 1VH2VL1 | | | | | | | | |

TABLE I

| | 10KD | | | |
|---|---|---|---|---|
| | Moving | Main | Main Peak | OTHER |
| Sample | Time | Peak (%) | Moving Time | (%) |
| 4D3 chimeric | 13.187 | 98.7 | 29.575 | 1.5 |
| huPIIO-1VH1VL1 | 13.212 | 97.6 | 29.408 | 2.4 |
| huPIIO-1VH1VL2 | 13.241 | 97.6 | 29.442 | 2.4 |
| huPIIO-1VH2VL1 | 13.267 | 89.1 | 29.567 | 10.8 |

Compared with the chimeric 4D3 clone, the humanized clone VH1VL2 has very similar binding affinity, thermostability (heat treatment), purity in CE, and aggregation potential in DLS assay. In DLS, VH1VL2 has a slightly higher PDI than that of the chimeric clone, but it also has significant better Tagg266 and Tagg473 in SLS assay (78.8 vs 75.0, 79.3 vs 75.4), suggesting that VH1VL2 has very low aggregation risk.

### Example 7—Antibodies Against GARP (LRRC32)

TABLE J
GARP monoclonal antibody clones
Amino acid sequence

| | | | |
|---|---|---|---|
| mAb | CDR1 | CDR2 | CDR3 |

Heavy Chain Sequence

| CDRs for | GYSITSDYA | ISYSGST | AKSGGDYYGSSSY |
|---|---|---|---|
| humanized | (SEQ ID NO: 1) | (SEQ ID NO: 2) | WYFDV |
| PIIO-1 | | | (SEQ ID NO: 3) |
| antibodies | | | |
| HuPIIO- | QVQLQESGPGLVKPSQTLSLTCTVSGYSITSDYAWNWIRQFPGNKLEW | | |
| 1VH1 | MGYISYSGSTSYTPSLKSRVTISRDTSKNHFSLKLSSVTAADTAVYYC | | |
| | AKSGGDYYGSSSYWYEDVWGQGTMVTVSS | | |
| | (SEQ ID NO: 20) | | |
| HuPIIO- | QVQLQESGPGLVKPSQTLSLTCTVSGYSITSDYAWNWIRQFPGNKLEW | | |
| 1VH2 | MGYISYSGSTSYTPSLKSRITISRDTSKNHFFLKLSSVTAADTAVYYC | | |
| | AKSGGDYYGSSSYWYFDVWGQGTMVTVSS | | |
| | (SEQ ID NO: 21) | | |
| HuPIIO- | DVQLQESGPGLVKPSQTLSLTCTVSGYSITSDYAWNWIRQFPGNKLEW | | |
| 1 VH3 | MGYISYSGSTSYTPSLKSRITISRDTSKNHFFLKLSSVTAADTATYYC | | |
| | AKSGGDYYGSSSYWYEDVWGQGTTVTVSS | | |
| | (SEQ ID NO: 19) | | |
| HuPIIO- | DVQLQESGPGLVKPSQTLSLTCTVSGYSITSDYAWNWIRQFPGNKLEW | | |
| 1VH4 | MGYISYSGSTSYTPSLKSRITISRDTSKNHFFLQLSSVTAEDTATYYC | | |
| | AKSGGDYYGSSSYWYFDVWGQGTTVTVSS | | |
| | (SEQ ID NO: 18) | | |
| m5c5 | GFTFSNYV (SEQ | ISSGGSYT | ARGYDNGDYVMDY |
| | ID NO: 9) | (SEQ ID NO: 10) | (SEQ ID NO: 11) |

EVKLVESGGGSVKPGGSLKLSCAASGFTFSNYVMSWVRQTPEKRLEW
VATISSGGSYTYYPDSVKGRLTISRDNAKNTLYLQMSSLRSEDTAMYY
CARGYDNGDYVMDYWGQGTSVTVSS (SEQ ID NO: 12)

Light Chain Sequence

| CDRs for | QSLLNSRSQKNY | GAS | QNDHSYPFT |
|---|---|---|---|
| humanized | (SEQ ID NO: 5) | (SEQ ID NO: 6) | (SEQ ID NO: 7) |
| PIIO-1 | | | |
| antibodies | | | |
| HuPIIO- | DIVLTQSPSSLAVSLGERATMNCKSSQSLLNSRSQKNYLAWYQQKPGQ | | |
| 1VL1 | PPKLLIYGASTRGSGVPDRESGSGSGTDFTLTISSLQAEDVAVYYCQN | | |
| | DHSYPFTFGQGTKLEIKR (SEQ ID NO: 23) | | |
| HuPIIO- | DIVLTQSPSSLAVSLGERVTMNCKSSQSLLNSRSQKNYLAWYQQKPGQ | | |
| 1VL2 | PPKLLIYGASTRGSGVPDRFSGSGSGTDFTLTISSVQAEDVAVYYCQN | | |
| | DHSYPFTFGQGTKLEIKR (SEQ ID NO: 24) | | |
| PIIO- | DIVLTQSPSSLAVSAGERVTMSCKSSQSLLNSRSQKNYLAWYQQKPGQ | | |
| 1VL3 | PPKLLIYGASTRGSGVPDRFSGSGSGTDFTLTISSVQAEDVAVYYCQN | | |
| | DHSYPFTFGSGTKLEIKR (SEQ ID NO: 22) | | |
| m5c5 | ESVDTYGNSF | RAS | QQTNEHPPT |
| | (SEQ ID NO: 13) | (SEQ ID NO: 14) | (SEQ ID NO: 15) |

DIVLTQSPASLAVSLGQRATISCRASESVDTYGNSFMHWYQQIPGQPPK
VLIYRASNLESGIPARFSGSGSRTDFTLTINPVEAGDVATYYCQQTNEH
PPTFGGGTKLEIK (SEQ ID NO: 16)

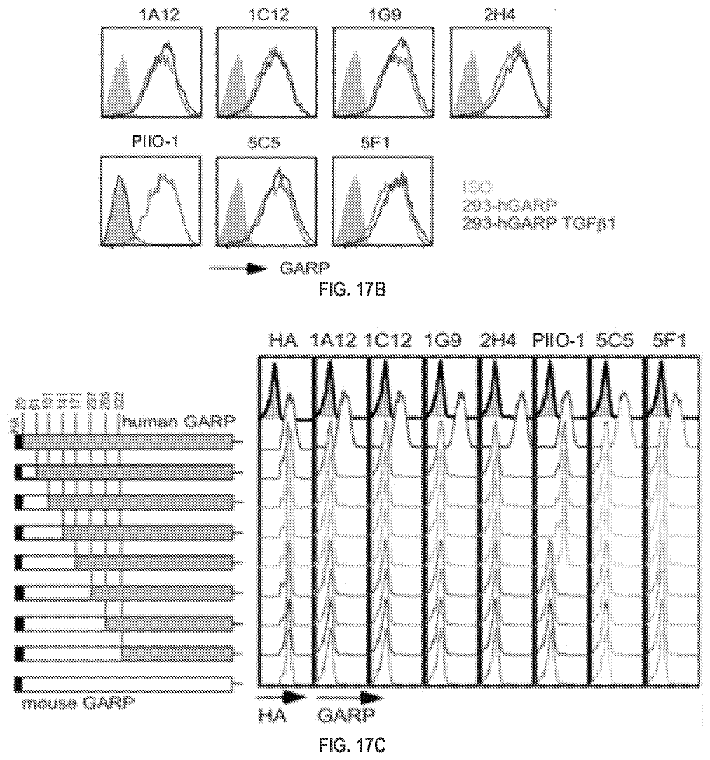

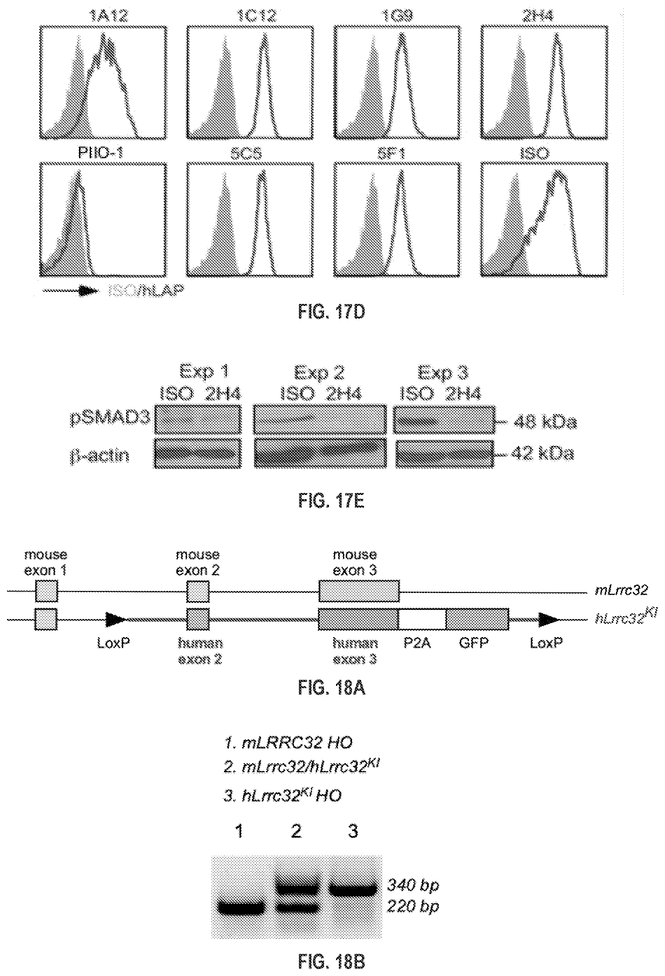

One of the goals of the Ohio State University (OSU) SPORE in Lung Cancer is to generate novel cancer therapeutics targeting GARP. Anti-human GARP antibodies were generated by immunizing mice with recombinant human GARP (hGARP) and boosting with irradiated SP2/0 myeloma cells stably made to express hGARP. Confirmation of antigen specificity of the multiple clones generated was performed by flow cytometry (FIG. 17A). Of the seven clones reported here, all recognized hGARP on Tregs while only five clones (excluding clones 1C12 and huPIIO-1) recognized hGARP on platelets. To further characterize antibody function, we tested whether the clones recognize free GARP or GARP-TGFβ complex (GARP-LAP).

Using an overexpression system with 293 cells, we found that only clone huPIIO-1 recognized free GARP, while the other antibodies recognized both free GARP and the GARP-LAP complex (FIG. 17B). Given the antibody's low affinity for mouse GARP (mGARP), we utilized a series of HA-tagged mGARP/hGARP chimeras to map the epitopes of theses clones. We found that huPIIO-1 was the only one that recognized aa 171-297 (FIG. 17C). Importantly, huPIIO-1 was also the only one that blocks binding of exogenous human LTGFβ-1 (huLTGFβ1) to surface GARP (FIG. 17D). Thus, we have generated and validated a library of anti-GARP antibodies to further test the capacity of GARP as a bona fide immune-oncology target

### Example 8—Generation of GARP Humanized Mice

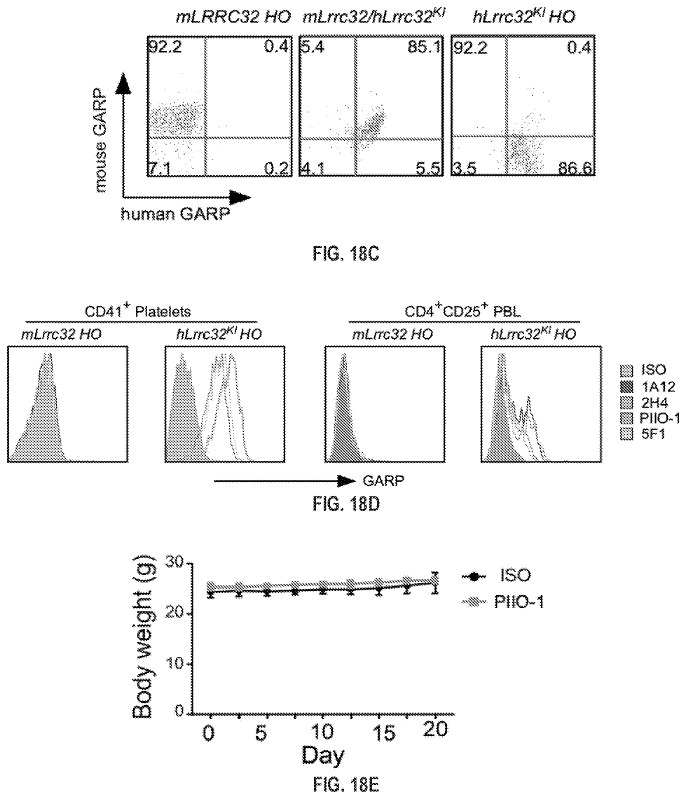

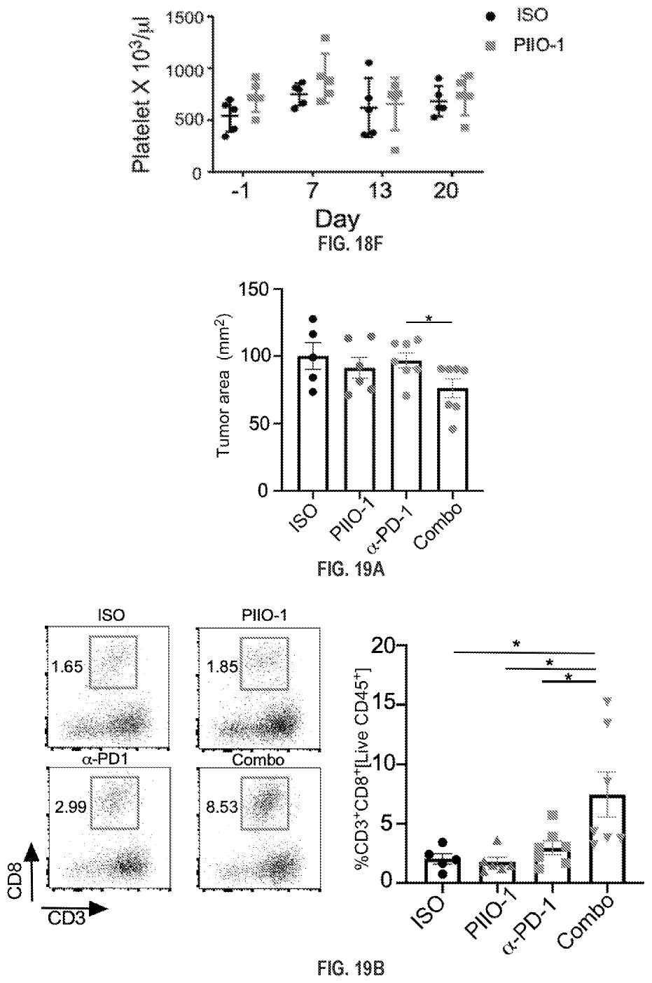

Most of the anti-GARP antibodies recognize human but not mouse GARP. In order to facilitate the translational effort, we have generated a human GARP knockin (hLrrc32^{KI}) mouse (FIG. 18A-C). We confirmed that humanized 4D3 (huPIIO-1) recognizes GARP on Tregs but not on platelets (FIG. 18D). Moreover, we found that i.v. administration of huPIIO-1 is well tolerated without causing significant thrombocytopenia and overt toxicity (FIG. 18E-F). These findings validate huPIIO-1 as a strong candidate for clinical development and provides a system to study the underlying mechanism of action for this drug.

### Example 9—huPIIO-1 has an Immune Modulatory Activity in Lrrc32-Humanized Mice

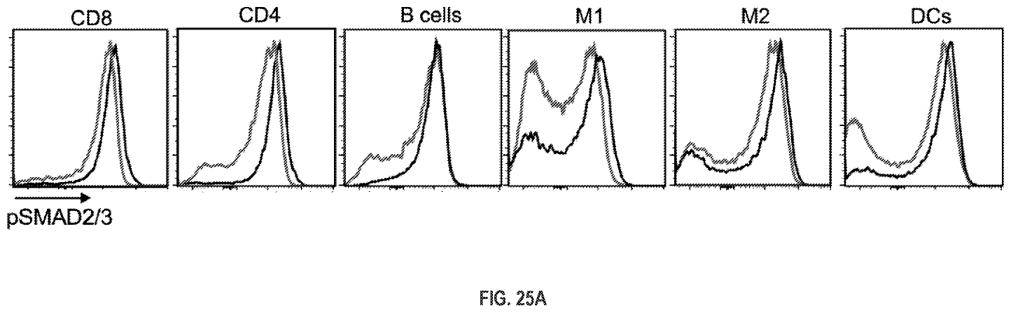

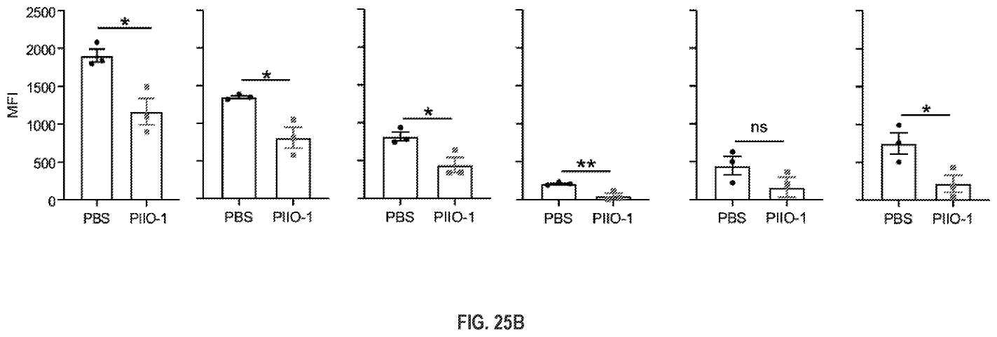

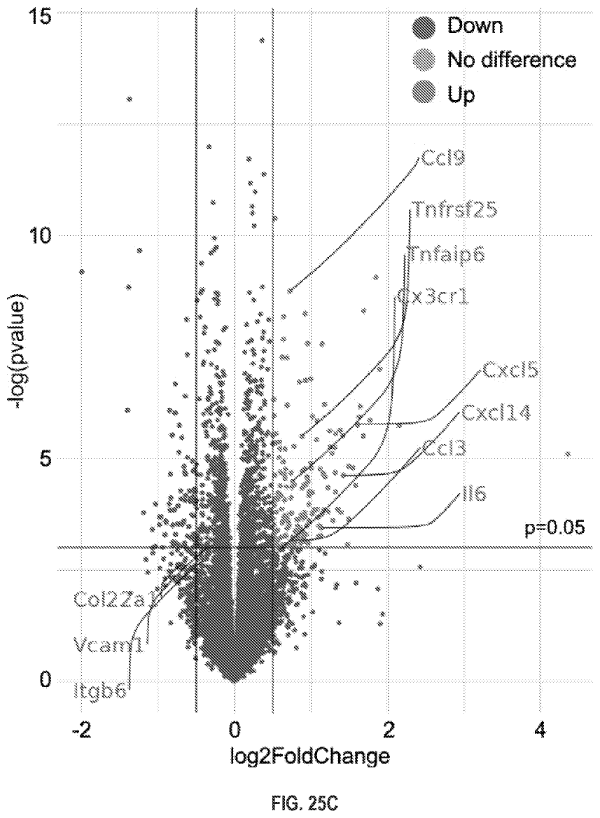

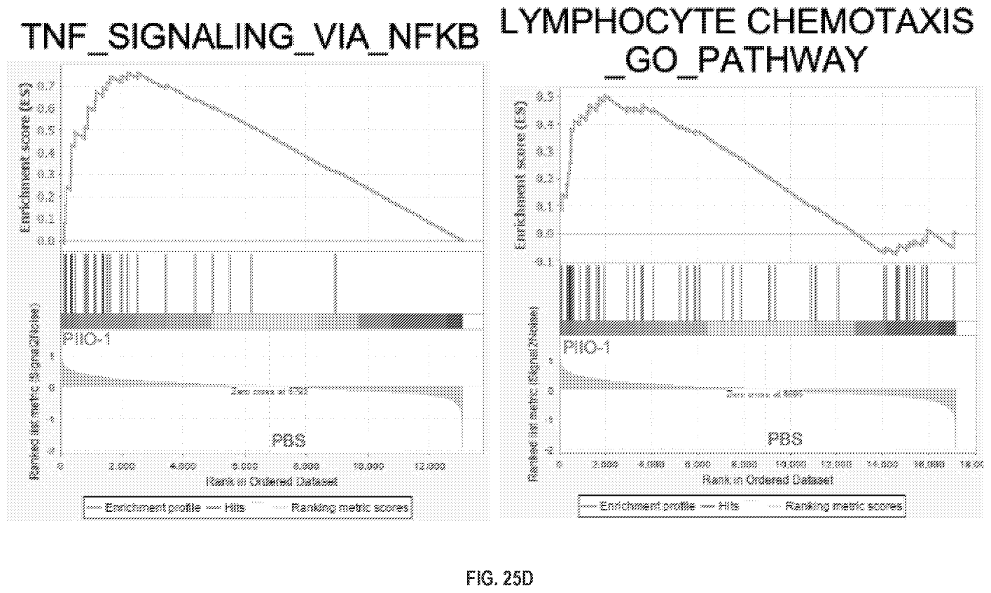

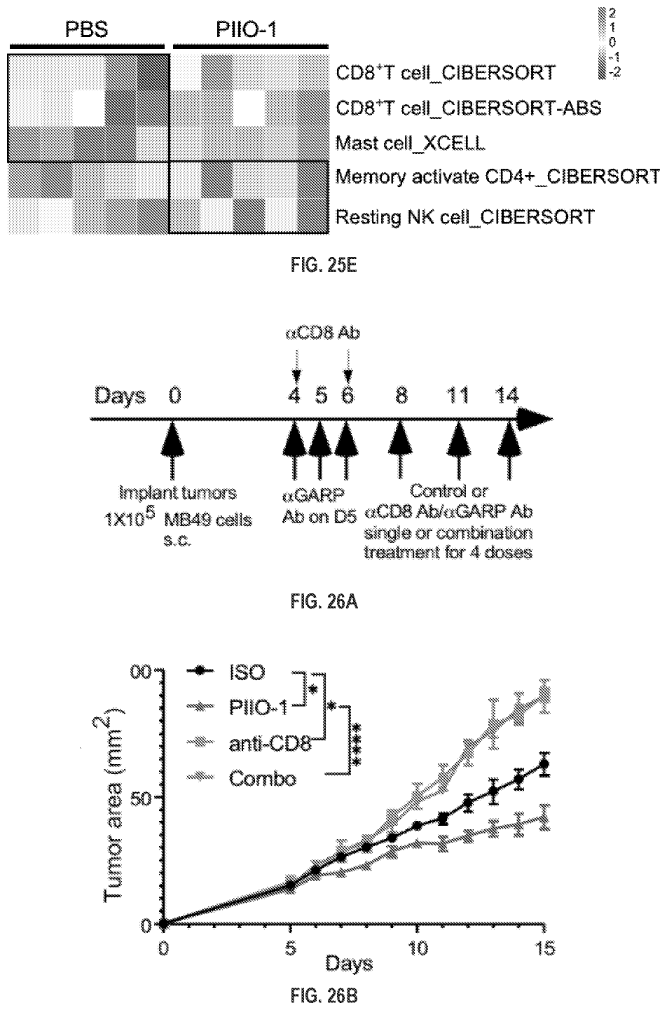

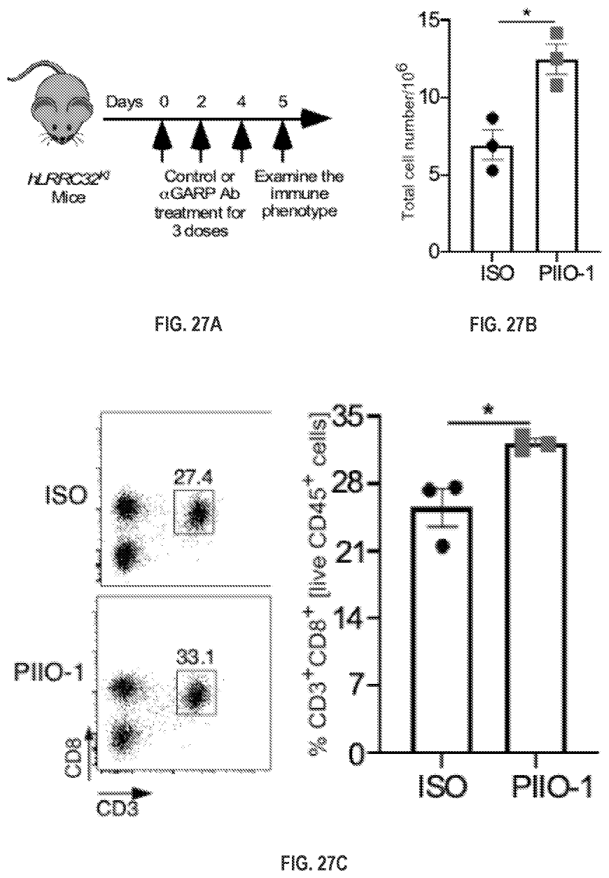

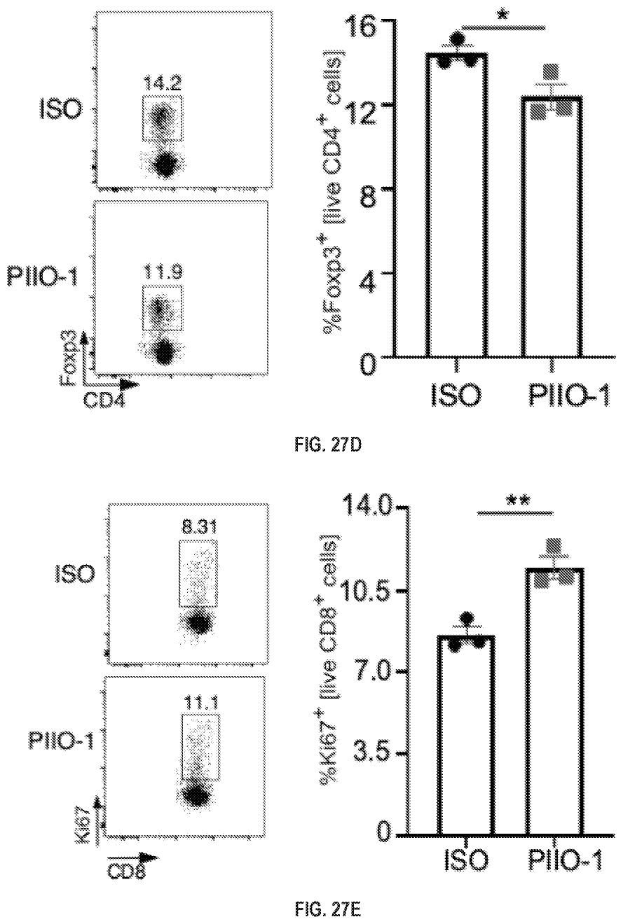

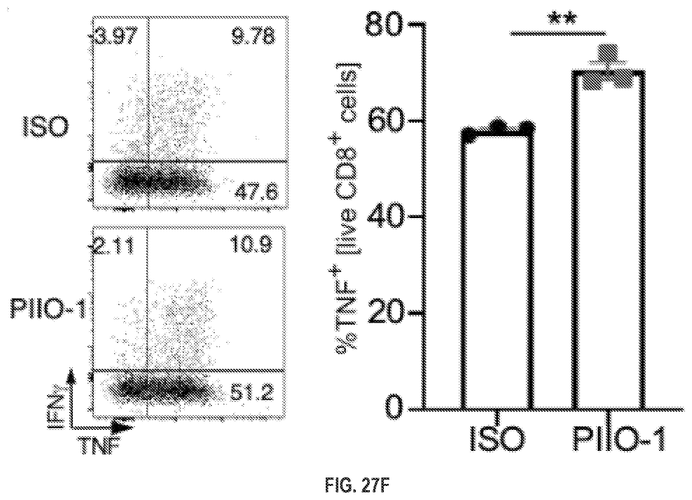

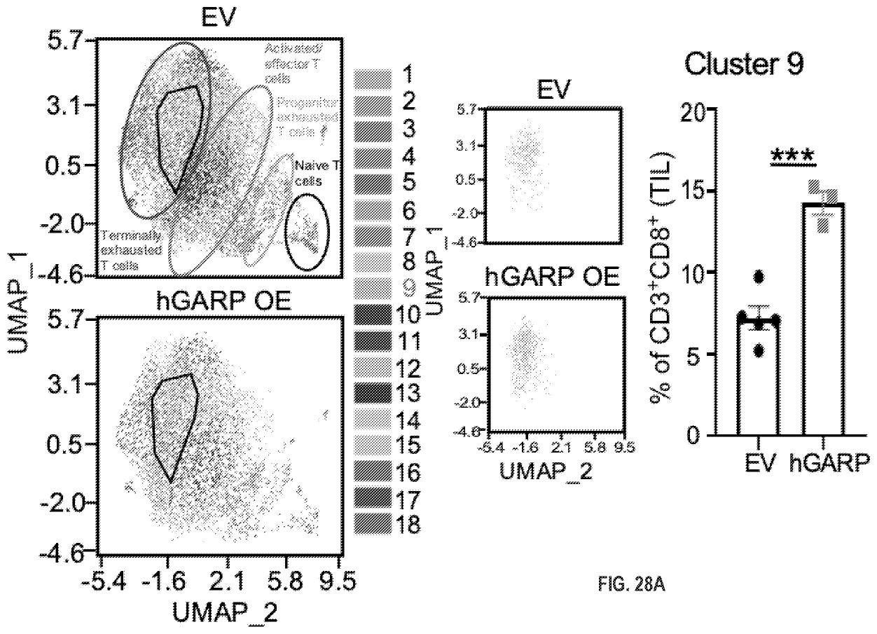

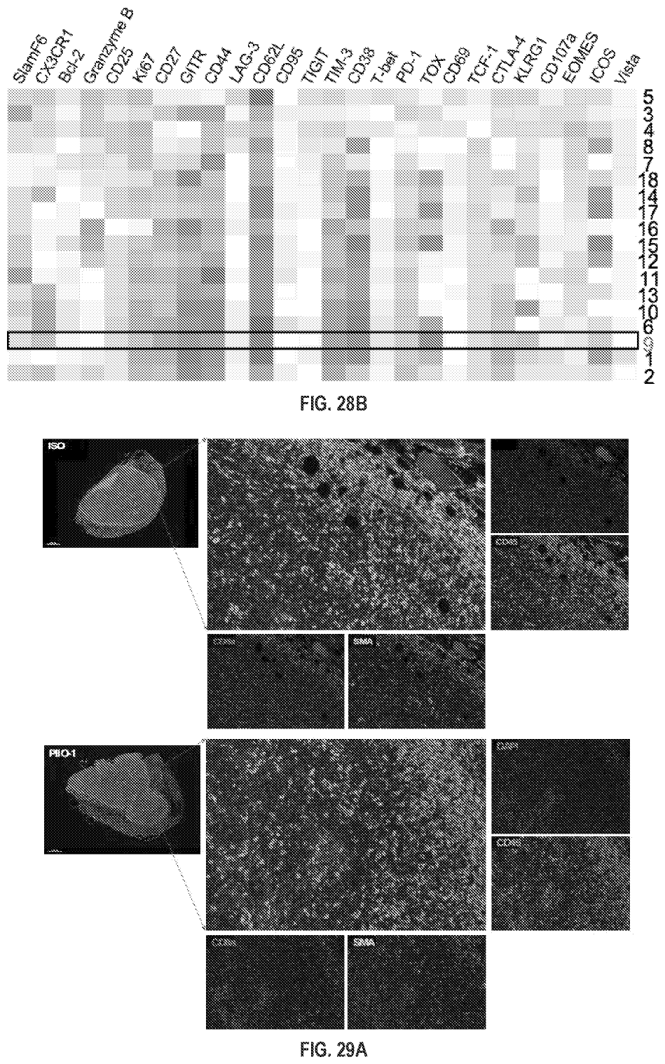

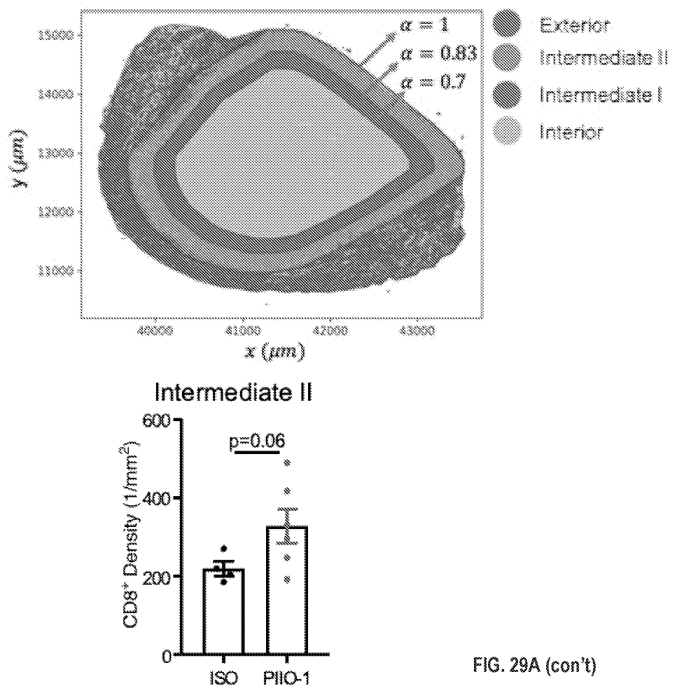

We next utilized the hGARP-mice to determine if anti-GARP antibody huPIIO-1 could modulate the host immune response. Two experiments were performed. First, tumor-free hGARP-mice were injected i.v. with huPIIO-1 or IgG1 (200 μg for 3 doses every 2 days) followed by immune phenotyping. We observed higher cellularity in the peripheral lymph nodes (pLN) of huPIIO-1 treated mice, which was associated with increased frequency of CD8⁺ T cells (FIG. 27A-B). We also observed elevated Ki67 in CD8⁺ T cells indicating the enhanced cellularity was due to increased proliferation (FIG. 27E). In addition, we noted a modest but significant decrease in the frequency of Tregs in the pLN (FIG. 27D). Second, hGARP-mice were injected s.c. with MB-49 bladder cancer cells made to express hGARP, followed by treatment with huPIIO-1. We then examined the activity of TGFβ by intracellular staining of pSMAD2/3 in tumor-infiltrating immune cells. We found that huPIIO-1 was able to dampen TGFβ activity from all immune cell subsets examined including T, B cells, M1, M2 macrophages and dendritic cells in the TME (FIG. 25). Taken together, we conclude that huPIIO-1 has immune modulating activities, likely through blocking the ability of GARP to bind and activate LTGFβ.

### Example 10—huPIIO-1 Monotherapy Facilitates CD8⁺ T Cell Recruitment into the TME and Confers Single Agent Activity Against Cancer in Lrrc32 Humanized Mice

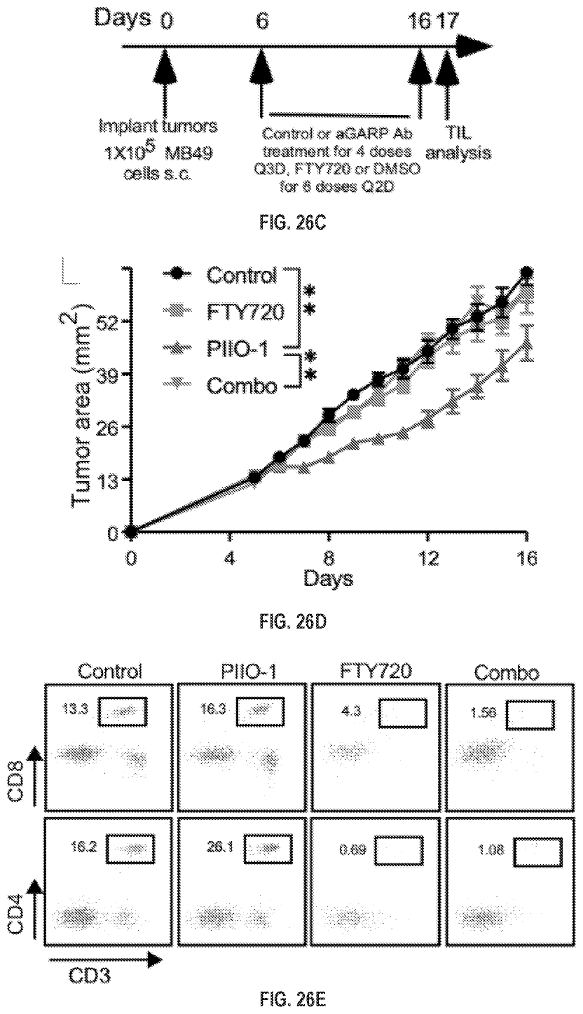

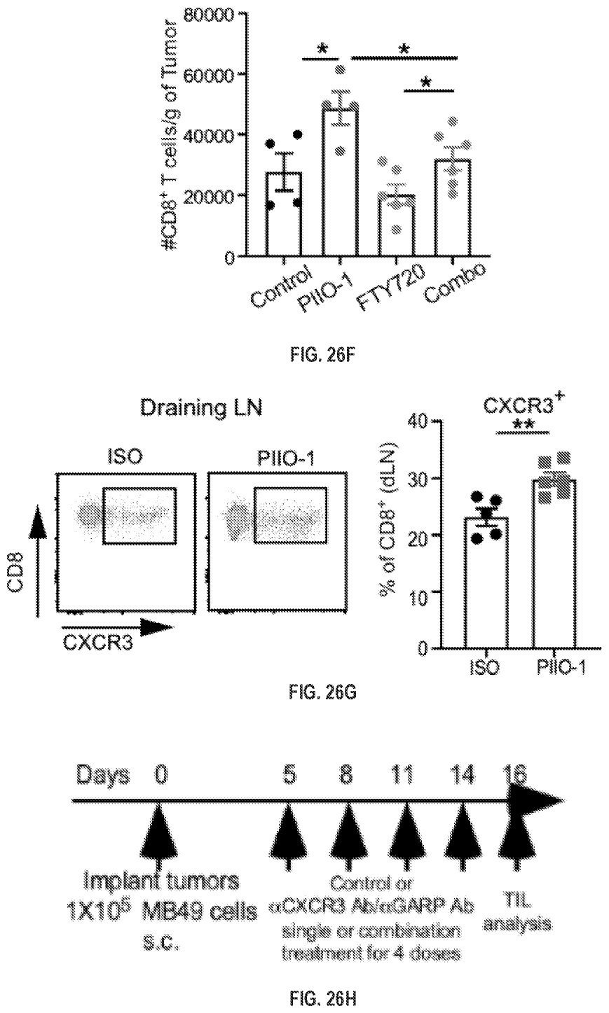

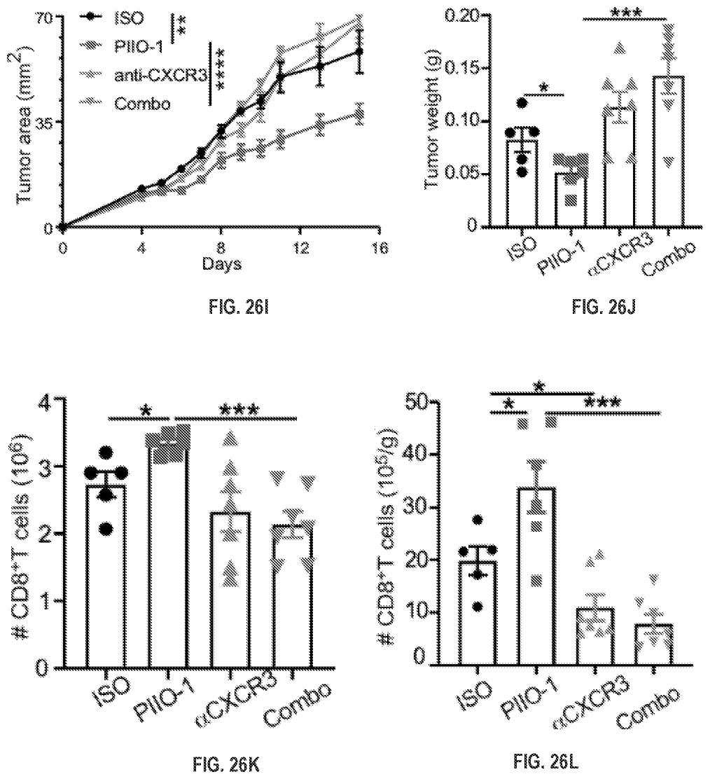

Recent studies demonstrated that TGFβ pathway not only attenuates T cell effector function but also blocks CD8⁺ T cell trafficking into the TME by specifically suppressing CXCR3 expression. We next addressed if huPIIO-1 could induce CXCR3 expression on CD8⁺ T cells and therefore contribute to anti-tumor activity (FIG. 26H). We found that huPIIO-1 has a significant single agent activity against MB49 (FIG. 26I-J), which is associated with increased CD8⁺ T cells in the draining LNs (FIG. 26K), and the TME (FIG. 26L). To examine the contribution of CXCR3 in the process, we blocked CRCX3 with an antagonistic antibody during huPIIO-1 treatment. We observed that this antibody abolished the anti-tumor efficacy of huPIIO-1 completely (FIG. 26I-J). Mechanistically, anti-CXCR3 blocked the CD8⁺ T cell recruitment both in the dLN and TME (FIG. 26K-L). Collectively, the data indicate that huPIIO-1 promotes T cell trafficking via CXCR3 by removing TGFβ from TME.

### Example 11—Potential of Anti-GARP Antibody huPIIO-1 to Overcome Resistance to PD-1 Blockade in Lung Cancer

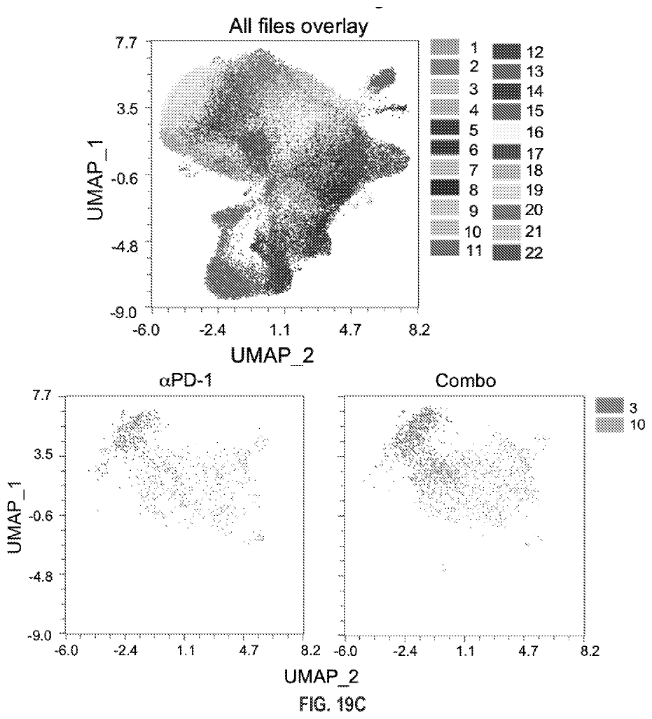

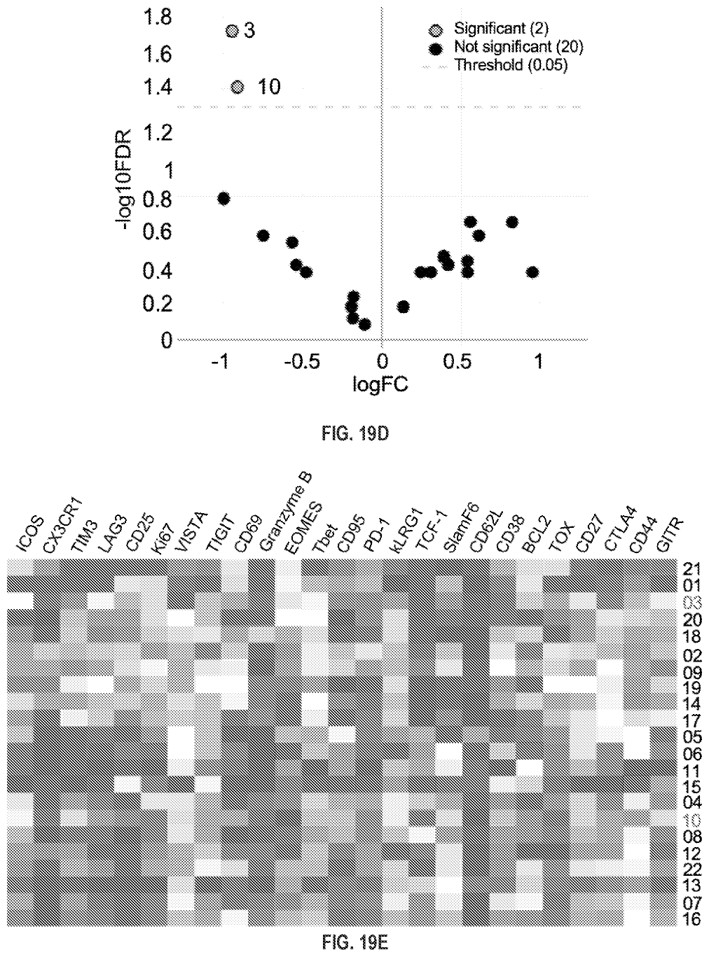

Mechanistically, PD-1 blockade works primarily by targeting the progenitor exhausted population of CD8⁺ T cells in the TME (TCF-1 expressing, SlamF6 expressing, PD-1 intermediate to low expressing). These cells deliver the proliferative burst following treatment resulting in increased differentiation to the terminal exhausted population, which is responsible for tumor clearance. As the monotherapy data showed, huPIIO-1 significantly modulated CD8⁺ T cells both in the TME and in the draining LN. Thus, we hypothesized GARP expression can contribute to PD-1/L1 ICB resistance. To address this hypothesis, we first mined the POPLAR database which compared PD-L1 blockade Atezolizumab with chemotherapy (i.e., docetaxel) for the platinum resistant advanced NSCLC. In this multi-center international phase II trial, bulk RNAseq data from pre-treatment tumors were available for 86 patients enrolled primarily in the US sites. We divided these patients into GARP high and low expression group with median expression level of LRRC32 transcript as the cutoff. We found that Atezolizumab treatment benefits more for patients with low GARP expression in both overall survival (HR=1.89, p=0.086; n=22 in Atezolizumab group vs n=21 in docetaxel group) and disease control rate (68.2% in Atezolizumab group vs 9.5% in docetaxel group, p=0.047703). In patients with high GARP expression, Atezolizumab (n=21) and docetaxel (n=23) did not demonstrate any difference in either OS (p=0.9655) or disease control rate. Although explorative in nature, this data indicates that high GARP expression can contribute to PD-L1 ICB resistance. To further address this hypothesis, we tested the combination therapy of anti-GARP antibody huPIIO-1 and PD-1 blockade in murine Lewis Lung Carcinoma (LLC) and CMT-167 lung cancer models, both of which are considered immunologically cold tumors with resistance to single agent PD-1 ICB. To do so, the human GARP KI mice were challenged with LCC followed by treatment with 200 μg/mouse of Isotype, huPIIO-1, anti-PD-1 antibody, or a combination of huPIIO-1 and PD-1 every 3 days starting on day 8 post tumor challenge for a total of 4 injections. We found that the combination of huPIIO-1 and PD-1 blockade had the greatest effect in slowing LLC growth when compared to the monotherapy treatments (FIG. 36A). While endpoint TIL analysis found that all groups receiving PD-1 blockade demonstrated a reduction in the frequency of CD8⁺ TCF-1⁺ T cells, it was only the combination group that was associated with a subsequent increase in TOX expression. These data are intriguing for two reasons: 1) TOX is known to be associated with and required for terminal T cell exhaustion, as well as generation of memory T cells, and 2) TOX expression has been shown to be downstream of TCR stimulation. Importantly, recent work has demonstrated a direct role for TGFβ signaling in suppressing the antitumor CD8⁺ T cells response by raising the threshold for TCR activation. Thus, these data indicate that huPIIO-1 can improve PD-1 blockade response by increasing the differentiation of progenitor exhausted cells via enhancing TCR stimulation. Finally, we also confirmed that huPIIO-1 can overcome anti-PD-1 resistance in CMT-167. The activity correlates significantly with increased CD8⁺ T cell populations in the TME (FIG. 19A-19B). Additionally, we analyzed the CD8⁺ TIL dynamics with spectral flow cytometry (Cytek Aurora) using the established T cell panel (CD45, CD3, CD8, CD4, Foxp3, CD69, CD25, PD-1, Tim3, Slamf6, TOX, Tef-1, CD44, CD62L, CTLA4, Lag-3, Klrg1, T-bet, Ki-67, GARP, EOMES, Vista, TIGIT, CX3CR1, ICOS, CXCR3, OX40, CD28, GITR, CD101, CD95, and Granzyme B. We found that combination therapy led to significant increase of two CD8⁺ T cell clusters (FIG. 19C-19E), reflecting newly activated effector progenitors (cluster #10: CD44⁺Tox⁻PD-1⁻GZMB⁻Vista⁺Tigit^{low}) and Teff-like cells (cluster #3: CD44⁺Tox⁻ Tcf1⁻ PD-1⁻GZMB⁺⁺Vista⁺Tigit^{low}).

All of the methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### Example 12 High LRRC32-TGFβ Expression in Human Cancers Correlates with Unfavorable TME and Poorer Clinical Response to ICB

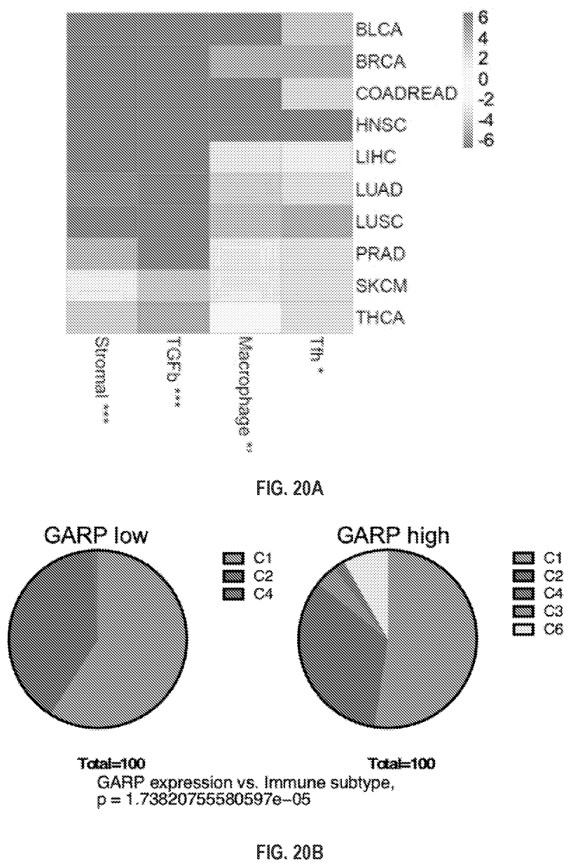

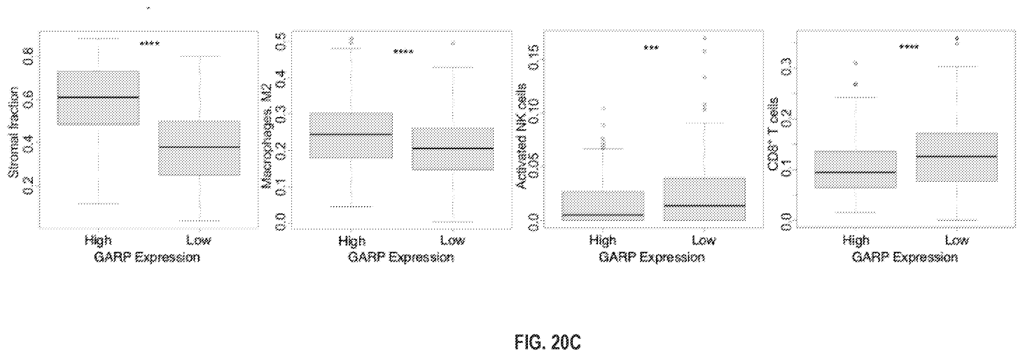

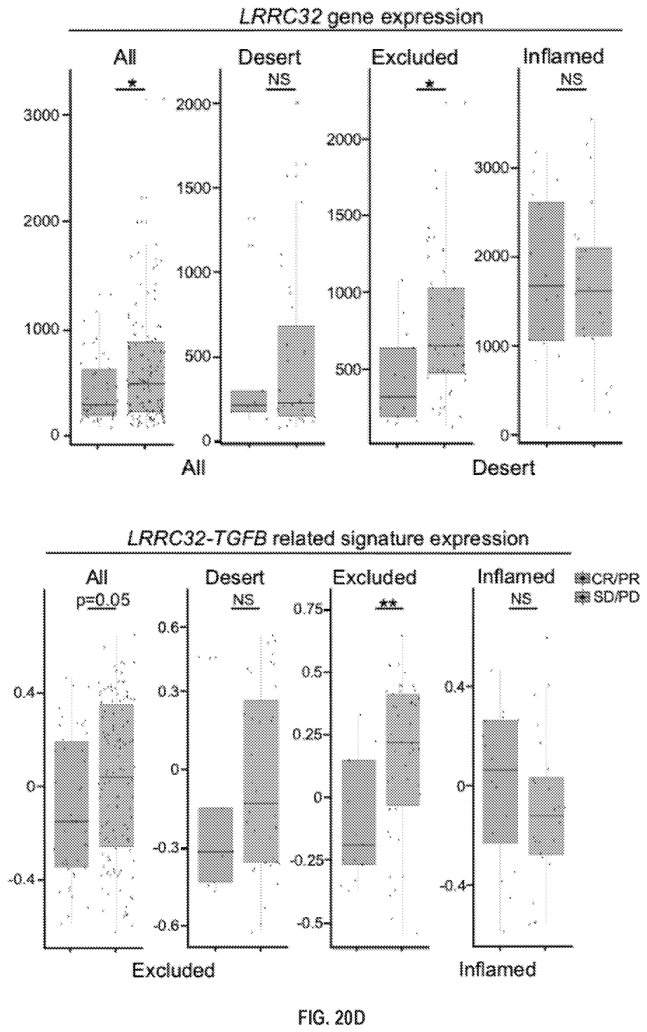

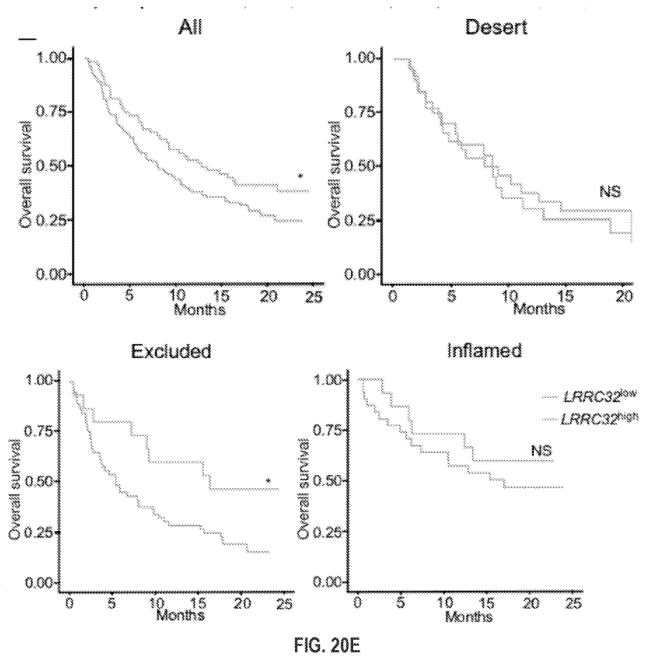

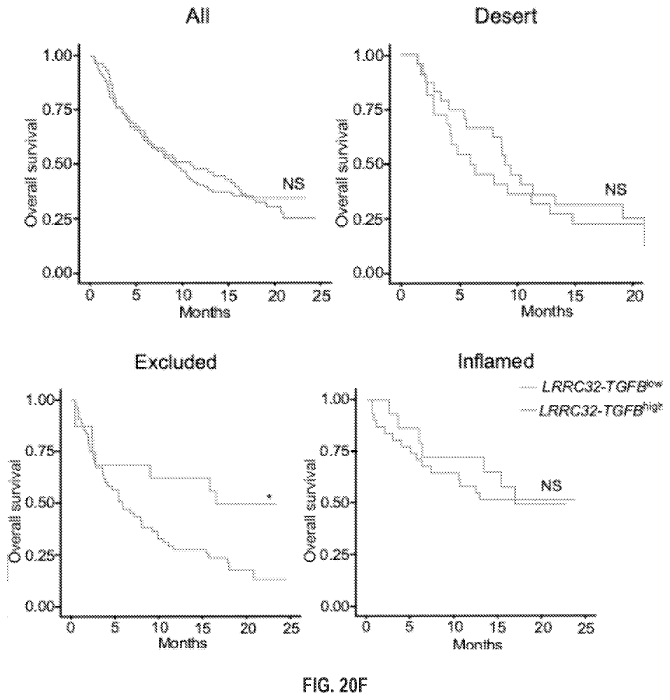

To understand the immunological and clinical implication of GARP expression in cancer, we first mined The Immune Landscape of Cancer database, which developed a global immuno-profiling classification by the bulk transcriptomic analysis of over 10,000 patients from TCGA. The wound healing classification (C1) reflects an induced expression of genes related to angiogenesis. The interferon γ (IFNγ) dominant classification (C2) contains a highest population of type 1 macrophages (M1) and CD8⁺ T cells, with high T cell receptor (TCR) density. Increased T helper (Th) 17 and Th 1 related genes, reduced tumor cell proliferation were included in the inflammatory classification (C3). A low Th1/high type 2 macrophage (M2) response phenotype characterized the lymphocyte depleted classification (C4). The immunologically quiet classification (C5) shows the lowest lymphocyte infiltration and highest M2 response. The TGFβ dominant classification (C6) represents tumors with the highest TGFβ gene signature. In 10 common types of solid tumors including bladder and breast cancers, we found that GARP expression positively correlated with tumors rich for stromal, TGFβ, and macrophage signatures and negatively with tumors with T follicular helper (Tfh) signatures, memory B cells, plasma cells, and activated dendritic cells (DCs) (FIG. 20A). The negative correlation between GARP expression and immune cells such as Tfh, B cells, plasma cells, and activated DCs suggested that GARP-rich TME is unfavorable for the generation of tertiary lymphoid structure (TLS) in the tumors, although this conclusion requires further histological study. Within the lung squamous cell carcinoma cohort, GARPhigh tumors had greater TGFβ dominant immune signatures and lower activated NK cells, CD8+ T cells and IFNγ signatures compared to GARPlow tumors (FIG. 20B-C). We next evaluated the significance of LRRC32 expression and LRRC32-TGFβ related signatures on patients' responsiveness to immunotherapy in metastatic urothelial cancer (mUC). We defined the LRRC32-TGFβ related signature using genes involved in the TGFβ activation process such as αV integrins. LRRC32 expression and LRRC32-TGFB related signatures are higher in patients who did not respond to anti-PD-L1 ICB (atezolizumab) (FIG. 20D). Elevated LRRC32 gene signature expression was predominantly observed in immune-excluded tumors, and we found that high LRRC32 expression (FIG. 20E) and high LRRC32-TGFB related gene signature (FIG. 20F) significantly correlated with worse overall survival in these patients. Therefore, we conclude that high LRRC32-TGFB expression in human cancers correlates with an unfavorable TME and poorer clinical response to anti-PD-L1 ICB, and that GARP is a biologically relevant target for cancer immunotherapy.

### Example 13: Anti-GARP Antibody PIIO-1 Blocks the Formation of GARP-LTGFβ1 Complex

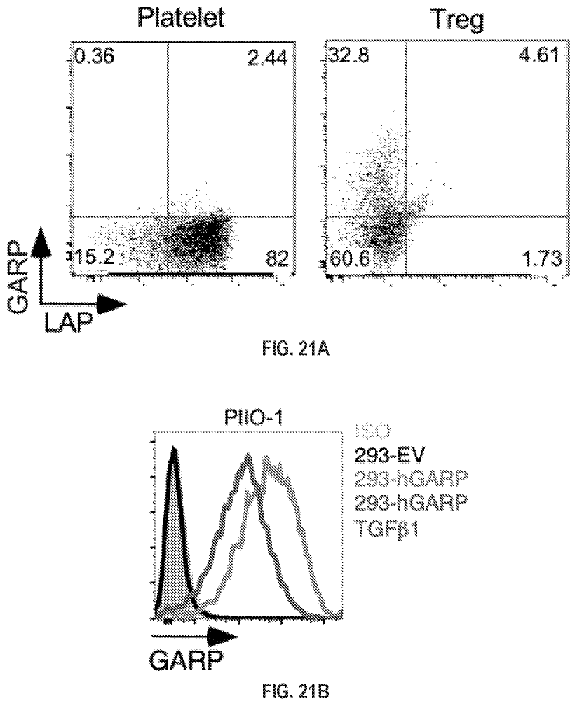

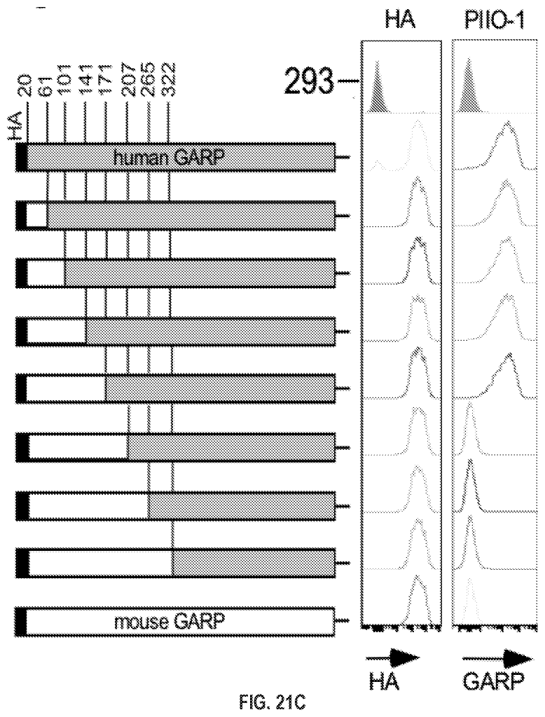

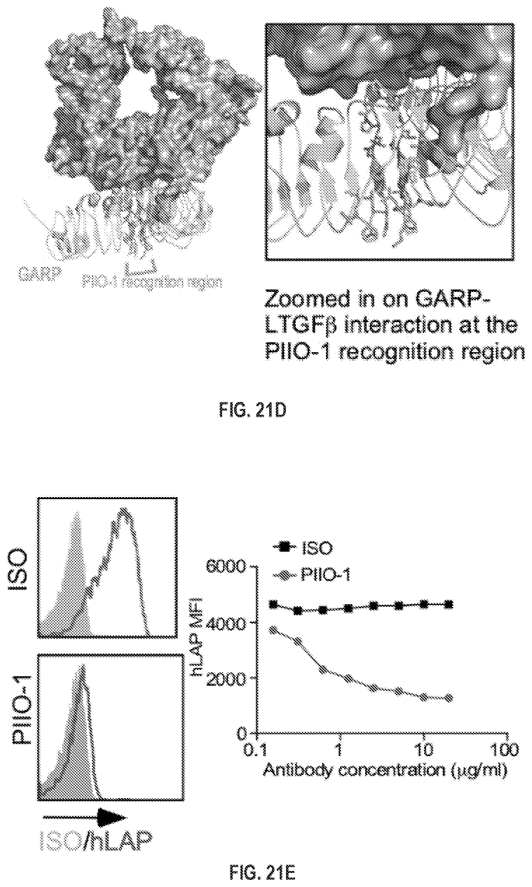

To generate anti-GARP monoclonal antibodies (mAbs), mice were immunized with recombinant hGARP, followed by boosting with irradiated whole myeloma hGARP-expressing SP2/0 cells. We characterized the binding of seven antibodies recognizing hGARP using flow cytometry. While all clones recognized hGARP on Tregs, only one failed to recognize hGARP on platelets (PIIO-1; FIG. 17A). GARP is known to exist biochemically in three major forms: ligand-free membrane-bound GARP; membrane-bound GARP-LTGFβ complex; and soluble GARP (released after proteolytic cleavage). Tregs express both ligand-free and complexed GARP on their cell surface, whereas platelets only express the complexed form. Since PIIO-1 can only recognize GARP on Tregs but not platelets, we can infer that it binds the ligand-free form of GARP (FIG. 21A). To confirm this prediction, we used HEK293FT cells transfected with plasmids expressing hGARP with or without TGFβ1 to create cells expressing either ligand-free GARP (293-hGARP) or the GARP-LTGFβ complex (293-hGARP-TGFβ1) (FIG. 21B and FIG. 17B). We generated a series of HA-tagged murine GARP (mGARP)/hGARP chimeras through standard PCR-based cloning techniques and determined that PIIO-1 binds an epitope corresponding to amino acids 171-207 on hGARP (FIG. 21C), which is the known site for LTGFβ binding (FIG. 21D). Using a competition binding assay, we found that LTGFβ1 blocked PIIO-1 binding to GARP (FIG. 17D). Importantly, we found that the expression of cell surface latency-associated peptide (LAP) decreases in the presence of PIIO-1 in a dose-dependent manner, indicating that PIIO-1 prevents complex formation between GARP and the exogenous LTGFβ1 [half-maximal inhibitory concentration (IC50)=653.4 ng/ml] (FIG. 21E). In summary, we generated a unique monoclonal antibody that specifically binds to ligand-free GARP at the LTGFβ1 binding site and blocks the formation of the GARP-LTGFβ1 complex. This antibody specifically targets GARP on Tregs and other cells expressing the ligand-free form of GARP but does not recognize the TGFβ-GARP complex on platelets.

### Example 14: Targeting GARP on Tumor Cells Enhanced PD-1 Blockade Efficacy in TNBC

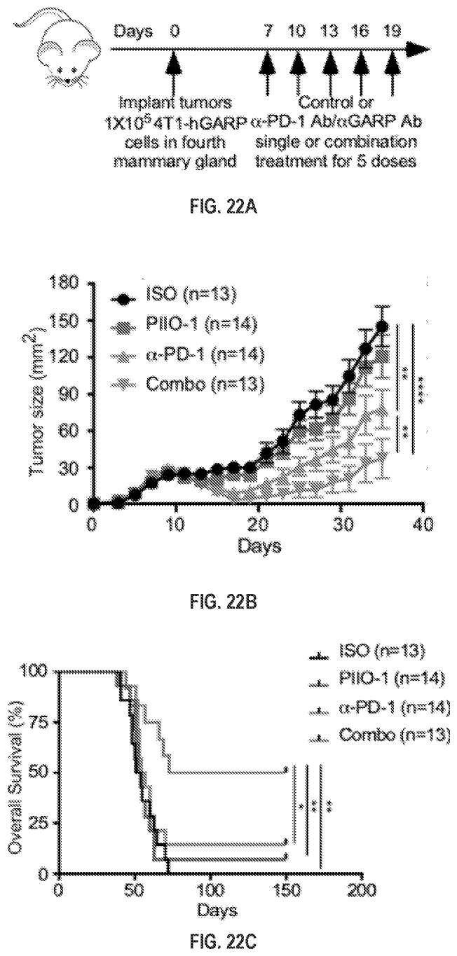

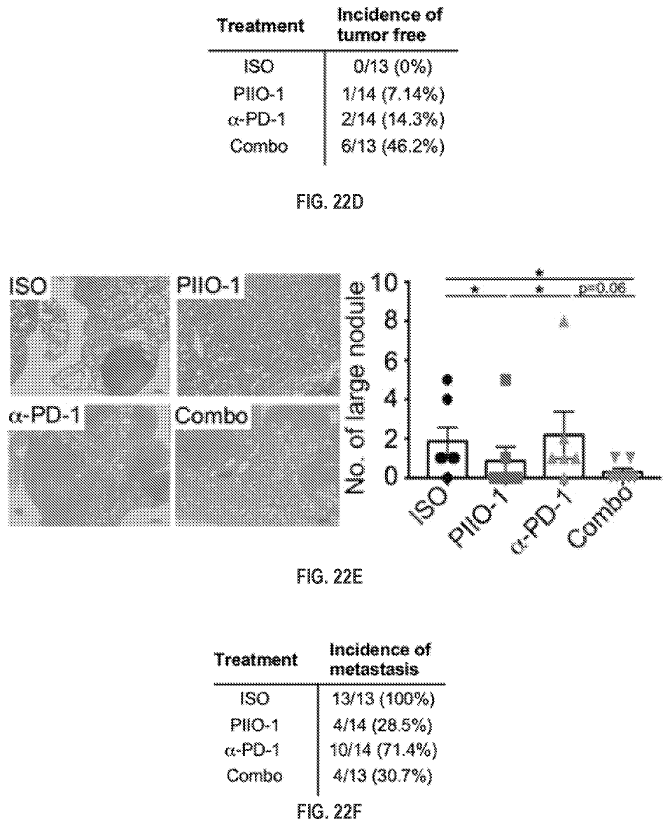

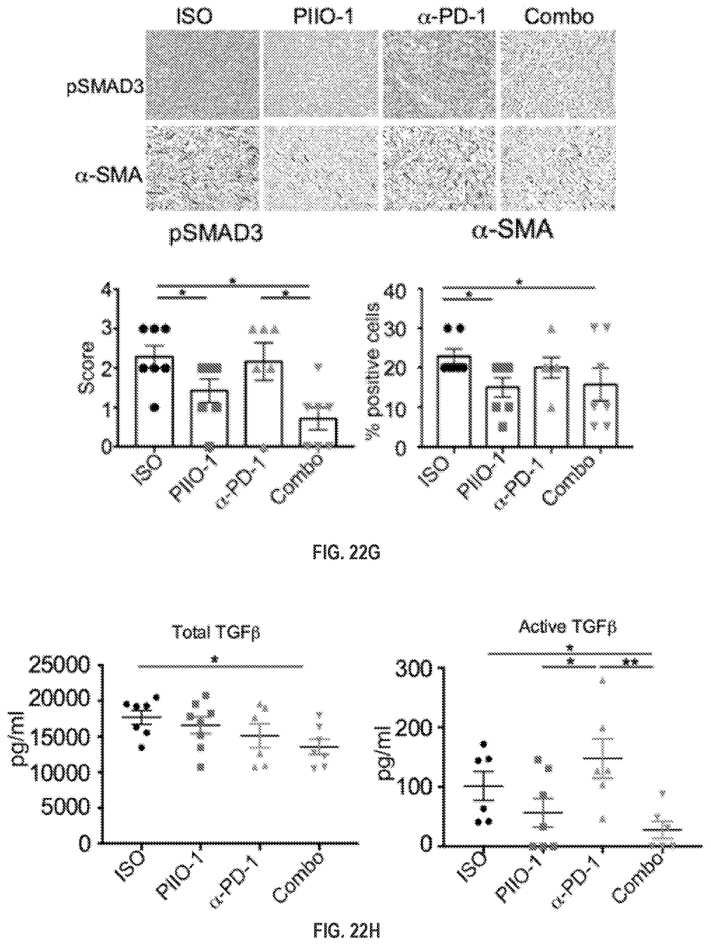

Since PHO-1 can bind GARP on Tregs, we next addressed whether combining PIIO-1 with anti-PD-1 ICB could augment efficacy by shifting an unfavorable TME towards a phenotype sensitive to immunotherapy. We implanted 4TI murine triple negative mammary gland cancer cells that stably express hGARP (4TI-hGARP) orthotopically into BALB/c mice. Mice with established tumors (day 7) were treated with single or combination therapies of PIIO-1 (200 μg/mouse) and anti-PD-1 (150 μg/mouse) every three days (experimental schema in FIG. 22A). Combination therapy slowed tumor growth and prolonged overall survival, resulting in complete response in 46% of mice treated with both PIIO-1 and anti-PD-1 (FIG. 22B-D). Furthermore, lung metastasis in mice treated with PIIO-1 (either single agent or combination therapy) were significantly reduced (FIG. 22E-F). To assess the impact of PIIO-1 on TGFβ downstream signaling in the TME, we stained tumors collected at endpoint for phosphorylated SMAD3 (pSMAD3) and α-smooth muscle actin (α-SMA). SMAD3 is phosphorylated upon TGFβ activation; «-SMA, a marker of cancer-associated fibroblasts (CAFs), is induced upon TGFβ activation. CAFs contribute to primary therapeutic resistance and are an emerging target for cancer immunotherapy. We found that both pSMAD3 and α-SMA were reduced in the TME after PIIO-1 treatment (FIG. 22G), suggesting that local TGFβ signaling was effectively blunted. Both total and active TGFβ were decreased in circulation following combination treatment (FIG. 22H). Finally, mice that experience complete response following combination treatment (FIG. 22C) were completely protected against rechallenge from the wild type 4T1 (4T1-WT) tumor cells, demonstrating that PIIO-1 promotes anti-tumor memory response (FIG. 22I). Taken together, these results demonstrate that combining PIIO-1 with anti-PD-1 leads to enhanced anti-tumor efficacy and anti-tumor memory, and this is likely mediated by a down-regulation of TGFβ activity in the TME.

### Example 15: Targeting TGFβ-GARP Signaling Modulates Immune Homeostasis and Promotes the Differentiation of Anti-Tumor Effector CD8+ T Cells in the TME

Next, we generated a hLRRC32KI mouse wherein the extracellular domains of mouse GARP are replaced with the corresponding human GARP domains in the germline (FIG. 18A-C). This model allows us to assess pre-clinical safety and efficacy of PIIO-1. In the platelets of hLRRC32KI mice, hGARP associates with mouse LAP as effectively as mGARP (FIG. 18D). IP injections of PIIO-1 were well tolerated without causing significant thrombocytopenia or overt toxicity (FIG. 18E-F). All PIIO-1 treated mice were found to have stable weight for at least 20 days without evidence of cardiac failure such as fluid overload and shortness of breath clinically. To assess the impact of PIIO-1 on the immune compartment in non-tumor bearing hLRRC32KI mice, we injected PIIO-1 or mIgG1 (200 μg/mouse each) i.v. every two days for three treatments, followed by tissue harvest, single cell isolation, and immune phenotyping (FIG. 27A). PIIO-1 treatment was associated with increased cellularity of peripheral lymph nodes (pLNs) and elevated frequency of CD8+ T cells (FIG. 27B-C). In addition, we saw reduced Tregs in the pLNs following PIIO-1 treatment (FIG. 27D), consistent with TGFβ's known role in inducing and maintaining Treg lineage. Corresponding to attenuated Treg function and reduced active TGFβ, PIIO-1 increased Ki67 expression and tumor necrosis factor α (TNFα) production by CD8+ T cells in pLNs (FIG. 27E-F). No difference in immune cell composition was observed in other organs, such as spleen, thymus, mesenteric lymph node (mLN) or peripheral blood.

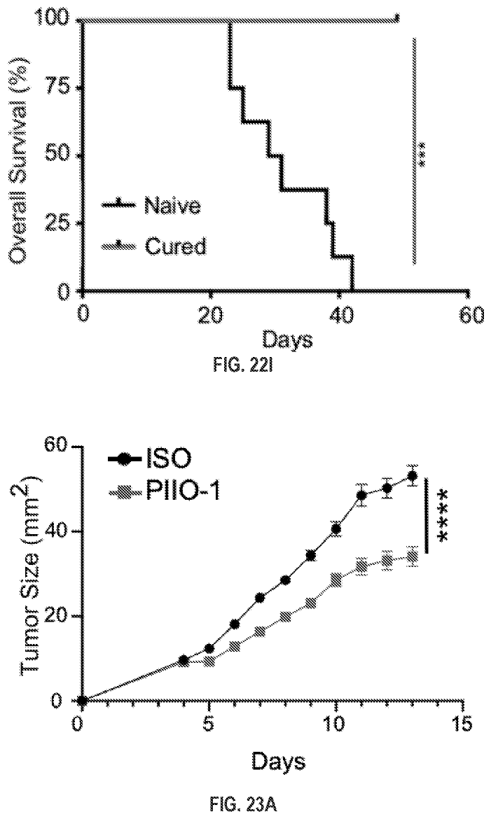

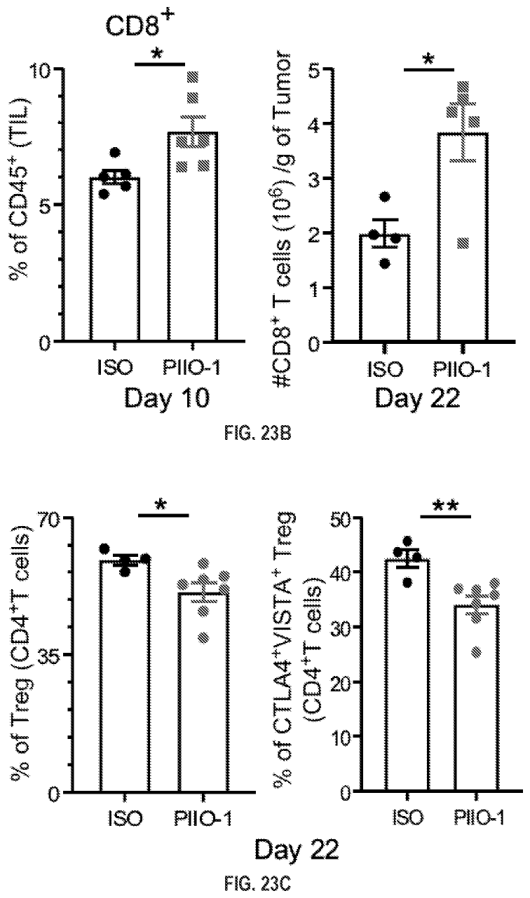

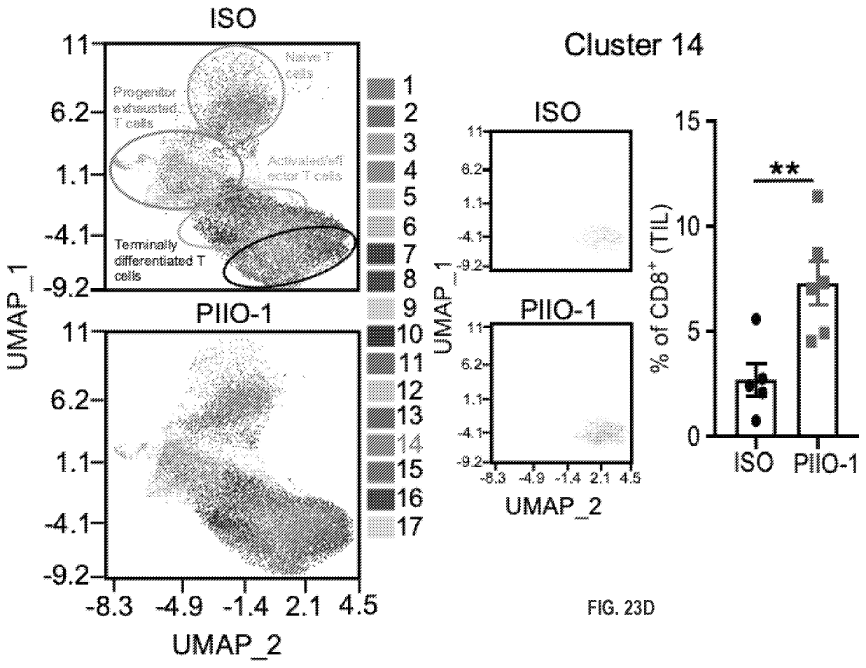

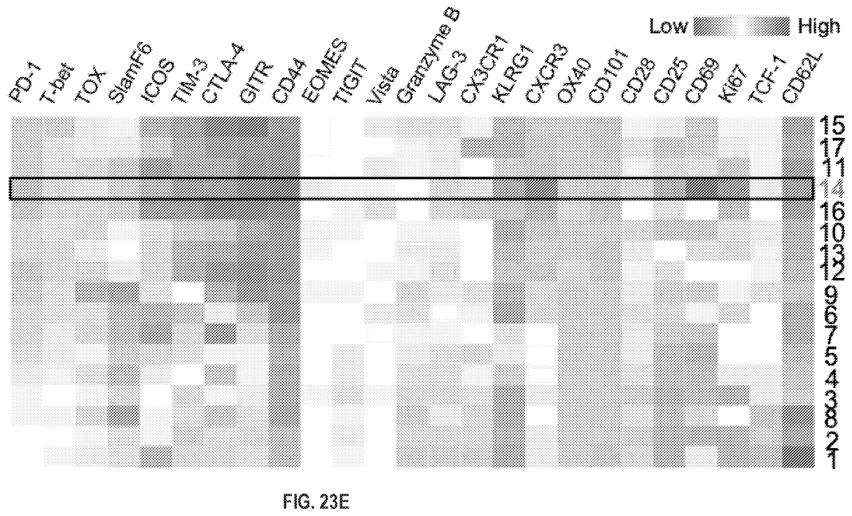

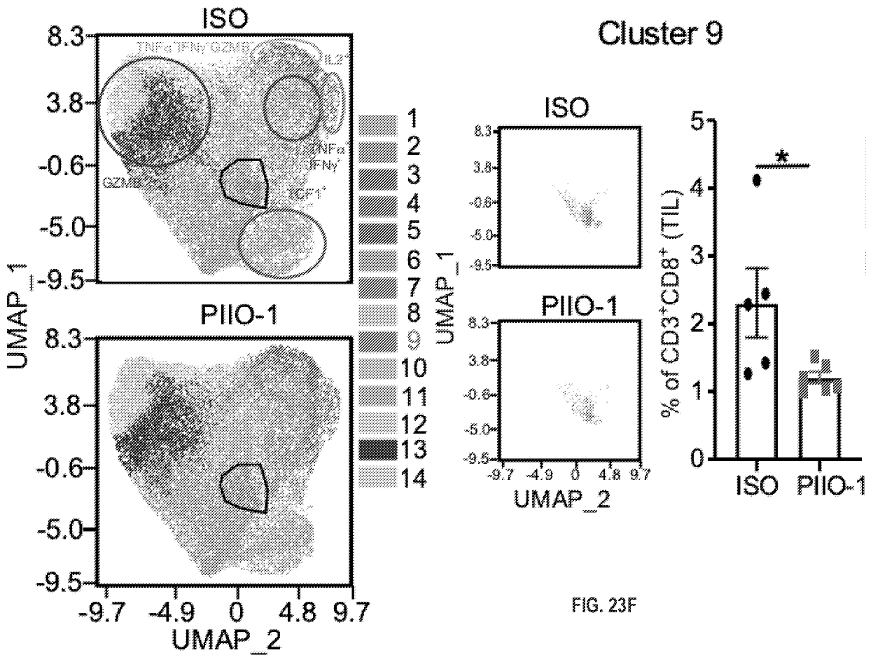

Next, we implanted hLRRC32KI mice s.c. with MB-49 murine urothelial carcinoma, an immunologically “lukewarm” tumor that only partially responds to anti-PD-1 therapy. Starting four days after the tumor implantation, PIIO-1 or mIgG1 was administered i.p. every three days for four total treatments. PIIO-1-treated mice showed a significant delay in tumor growth (FIG. 23A). Since murine MB-49 does not express human GARP, this observed anti-tumor activity must be attributed to an increased anti-tumor immune response. Thus, in a separate experiment, we treated day 6 MB-49 tumors every three days with PIIO-1 or mIgG1 for two (short-term) or six (longer-term) total doses. We harvested tumors 24 hours after final treatment, and isolated tumor-infiltrating lymphocytes (TILs) for analysis by high dimensional spectral flow cytometry. Short term PIIO-1 increased the frequency of CD8+ T cells in the TME (FIG. 23B, left), and this effect was augmented following longer-term treatment (FIG. 23B, right). Longer-term exposure to PIIO-1 also decreased both Treg frequency (FIG. 23C, left) and suppressive function, as indicated by downregulation of CTLA4 and VISTA (FIG. 23C, right).

To examine the effect of PIIO-1 on CD8+ T cells in the TME at the single cell level, we used a 33-marker T cell exhaustion panel for high dimensional spectral flow. We performed dimension reduction using the Uniform Manifold Approximation and Projection (UMAP) approach, which allowed the data to be displayed in two dimensions (FIG. 23D-E). We then performed unsupervised clustering analysis using FlowSOM to partition the data and allow for differential expression analysis between groups. This analysis identified 17 distinct clusters, one of which (cluster 14) was significantly enriched in CD8+ T cells from PIIO-1-treated mice. Cluster 14 displayed elevated expression of activation markers including LAG-3, CD44, GITR, TIM-3, and PD-1 (FIG. 23D-E), but not TOX, a transcription factor associated with terminal exhaustion. Our data supports the hypothesis that PIIO-1 induces CD8⁺ T cell effector differentiation (FIG. 23D, orange circled population in UMAP) and blocks T cell exhaustion. Indeed, with prolonged PIIO-1 treatment (starting on day 5 for 4 doses), there was a decrease in a terminally exhausted population (cluster 9), as indicated by its TOXhigh status with little or no effector cytokine production (including IL-2, IL-21, TNFα, IFNγ and others; FIG. 23F-G). Taken together, our data suggest a multifaceted effect of PIIO-1, wherein it simultaneously shifts immunologically lukewarm tumors towards a pro-inflammatory state with increased CD8⁺ T cell infiltration, while promoting activation and preventing terminal exhaustion of these TILs. To support these results, we found that enforced expression of hGARP in MB-49 cells (MB-49-hGARP) resulted in higher frequency of tumor-infiltrating CD8+ T cells with an exhausted phenotype compared to empty vector (EV) transfected MB-49 (cluster 9; FIG. 28A-B).

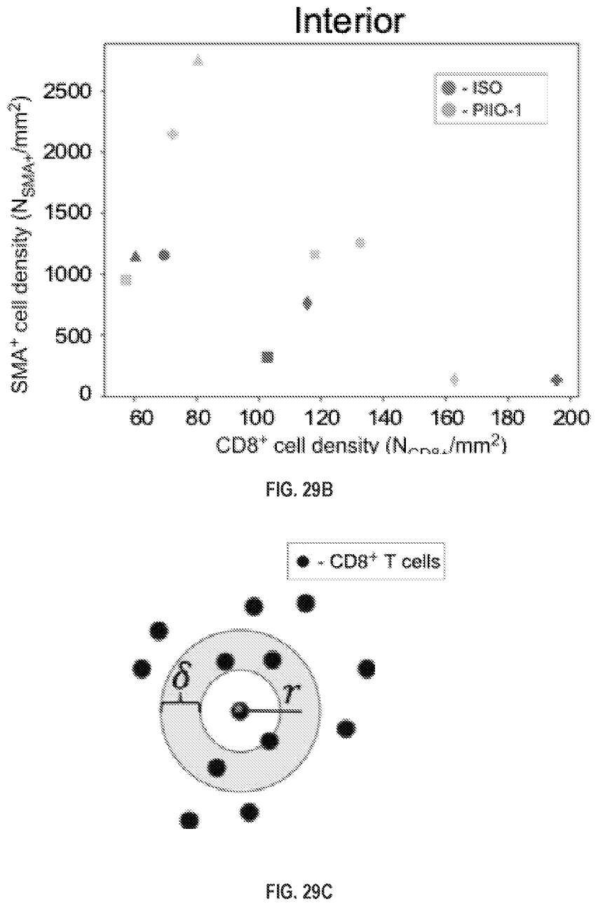

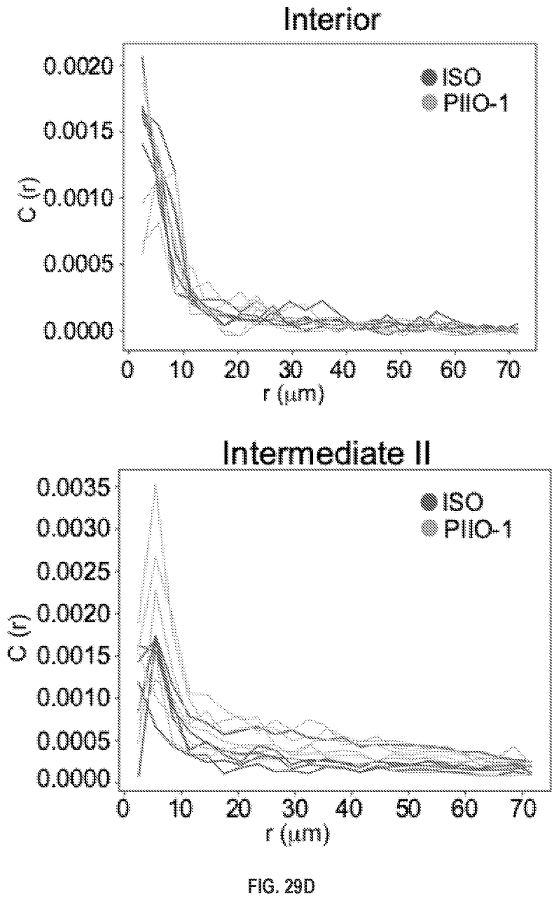

Next, we analyzed MB-49 tumors spatially using multiplex IF imaging. Tumors were stained with CD45, CD8, α-SMA and partitioned into tumor interior, intermediate I, intermediate II and exterior regions. CD8⁺ T cell density increased in the intermediate II region indicating enhanced intratumoral infiltration after PIIO-1 treatment (FIG. 29A). In the interior regions of mIgG1 treated tumors, α-SMA+ cell density negatively correlated with CD8⁺ T cell density. PIIO-1 treatment decreased the magnitude of this negative correlation (FIG. 29B), suggesting that blocking the GARP-TGFβ axis decreased stromal formation and increased T cell infiltration. By applying spatial two-point correlation analysis, we found that CD8⁺ T cells co-localize more frequently in both the interior and intermediate II regions of PIIO-1 treated tumors, compared to controls (FIG. 29C-D). In summary, treatment of MB-49 with PIIO-1 alters CD8⁺ T cell intratumoral infiltration kinetics and mediates functional and spatial changes to their phenotype.

### Example 16: Anti-GARP Antibody Enhances Anti-PD-1 ICB Against GARP-Negative Tumors

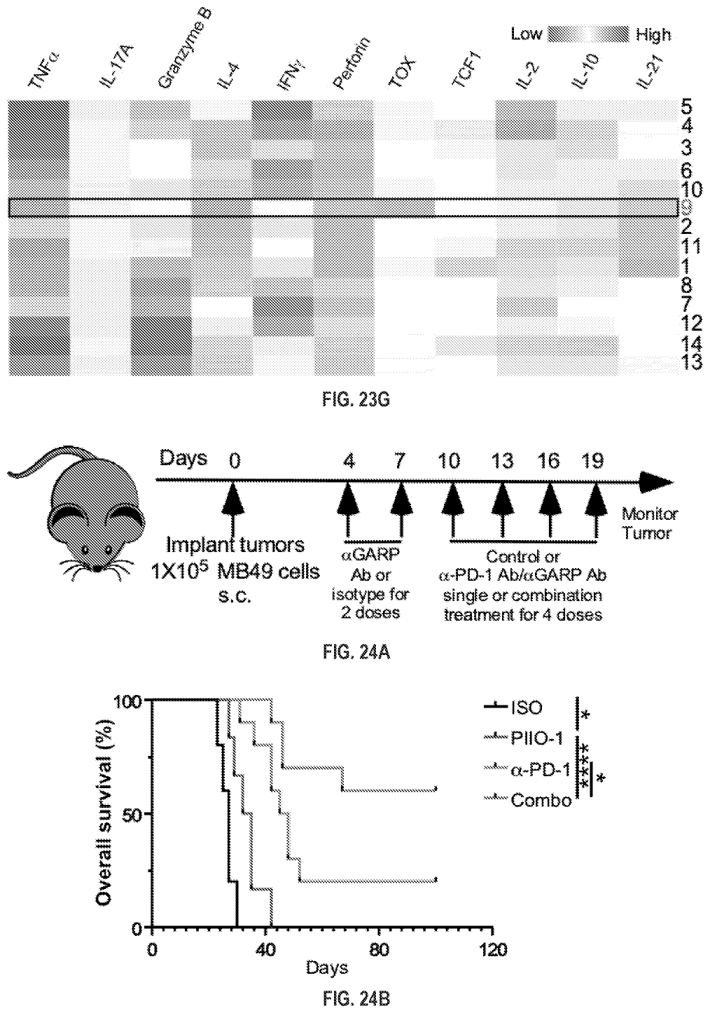

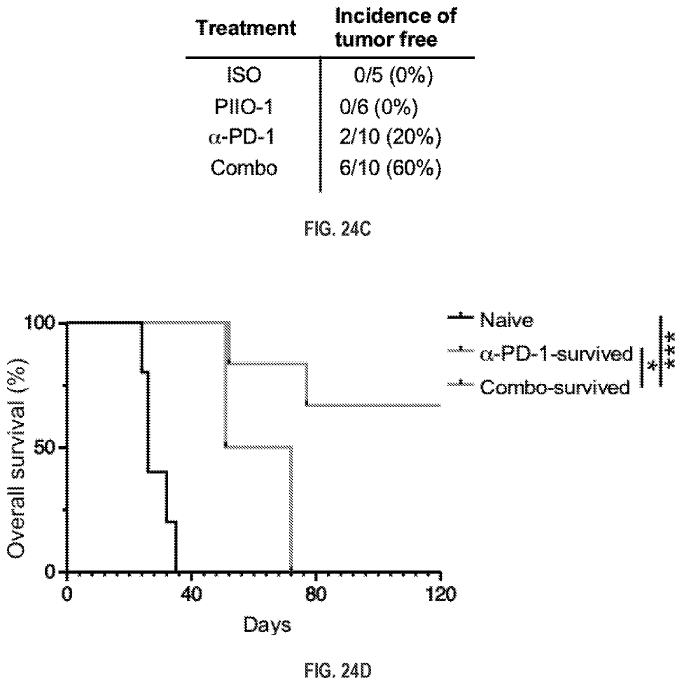

Mechanistically, PD-1 blockade targets progenitor exhausted CD8⁺ T cells in the TME, which persistently express TCF-1 and SlamF6 with low levels of PD-1 and TIM-3. These cells undergo a robust proliferation following anti-PD-1 treatment resulting in differentiation towards an effector phenotype, which induces tumor clearance. Since PIIO-1 monotherapy significantly reduced CD8⁺ T cell exhaustion in the TME, we evaluated whether it could potentiate the anti-tumor activity of anti-PD-1 ICB. We treated hLRRC32KI mice bearing subcutaneous day 4 MB-49 tumors sequentially using PIIO-1 (200 μg/mouse; six doses) and anti-PD-1 antibody (100 μg/mouse; four doses started day 10) (FIG. 24A) While single agent PIIO-1 modestly prolonged overall survival compared to control (FIG. 24B), combination therapy with anti-PD-1 resulted in complete tumor response in 60% of mice (FIG. 24C). Finally, when we rechallenged cured mice with MB-49 cells, those mice that previously received combination therapy had better anti-tumor memory function (FIG. 24D), indicating that PIIO-1 impacts favorably the generation of anti-tumor immunological memory.

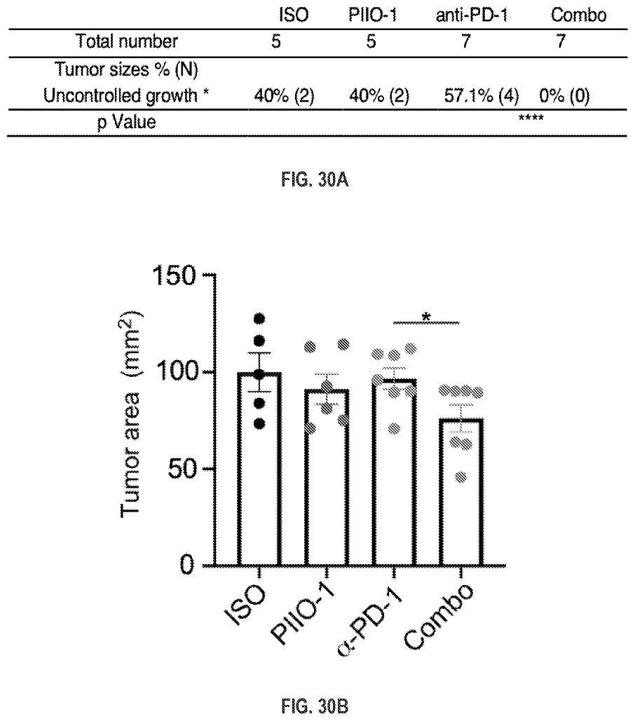

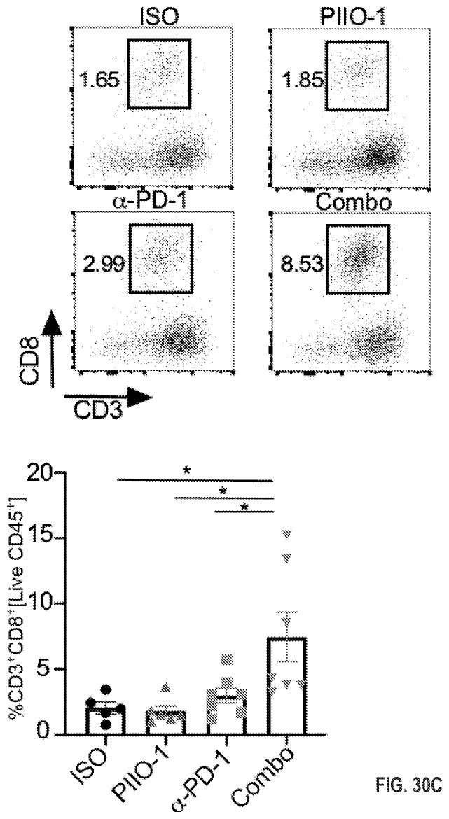

We also tested PIIO-1 and anti-PD-1 combination therapy against murine Lewis Lung Carcinoma (LLC) and CMT-167 lung cancer models, both of which are immunologically cold tumors and are resistant to anti-PD-1 ICB. Day 8 LLC tumors in hLRRC32KI mice were treated every three days with single or combination therapy (PIIO-1 200 μg+anti-PD-1 100 μg; four doses total). The combination of PIIO-1 and anti-PD-1 was most effective in slowing LLC growth when compared to anti-PD-1 monotherapy (FIG. 30A). These results were recapitulated in the CMT-167 model wherein adding PIIO-1 overcame the anti-PD-1 resistance seen in CMT-167, which correlated with increased CD8+ T cells in the TME (FIG. 30B-C).

### Example 17: Humanized PIIO-1 Blunts Canonical TGFβ Signaling in Tumor-Infiltrating Immune Cells and Promotes Pro-Inflammatory TME

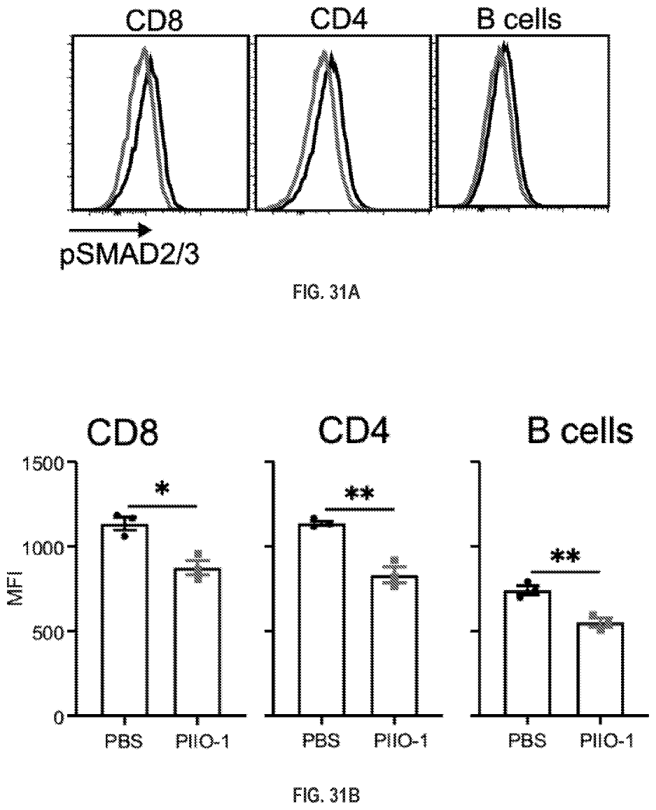

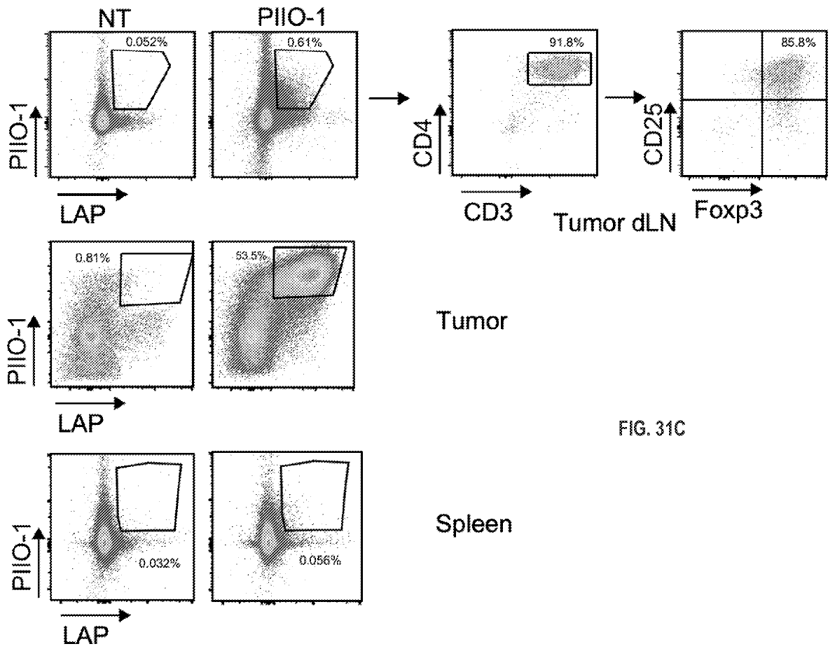

We next humanized PIIO-1 by fusing its complementarity determining regions (CDR) of the variable domains with the remainder of the chain from human IgG4. The humanized PIIO-1 has identical affinity to the parental antibody for human GARP (Kd, 1-3 nM) and it had similar mono-agent anti-tumor efficacy in MB-49 tumor model. Moreover, PIIO-1 treatment of MB49-bearing tumors resulted in decreased pSMAD2/3 signaling in major tumor-infiltrating immune cell subsets including T, B cell, macrophages, and DCs (FIG. 25A-B), as well as T and B cells in the dLN (FIG. 31A-B). Interestingly, on a per cell basis, tumor infiltrating CD8⁺ T cells had the highest TGFβ signaling activity indicated by pSMAD level (FIG. 25B). To determine the immune cell target of PIIO-1, we injected it into tumor bearing hLRRC32KI mice. Twenty-four hours later, tumors, dLNs, and spleens were harvested, and single cell suspensions were analyzed for cell surface binding of PIIO-1. We found that PIIO-1 only recognizes cells in the tumor and the dLN but not in the spleen (FIG. 31C). Tregs were the major cell population that bound PIIO-1 in the dLN (FIG. 31C). The preferential targeting of PIIO-1 to tumors and the dLNs, but not the spleen underscores the favorable biodistribution of this antibody.

Anti-tumor function of cytotoxic CD8⁺ T cells requires lytic function as well as pro-inflammatory cytokine production (e.g., TNFα and IFNγ). In addition, TGFβ is known to dampen CD8⁺ T cell function and migration into the TME. To gain further insight into the mechanism of action of PIIO-1, we performed bulk transcriptome analysis of day 10 MB-49 tumors in hLRRC32KI mice treated with PBS or PIIO-1 on days 6 and 9. mRNA expression analysis revealed that the transcripts of pro-inflammatory cytokines (e.g., Tnf super family, Il6) and chemokines (e.g., Ccl3, Ccl9, Cxcl14, Cxcl15) were increased in the PIIO-1-treated tumors (FIG. 25C), consistent with the ability of PIIO-1 to induce a proinflammatory TME. GSEA showed a similar picture especially with increased TNF-NFκB signaling as well as lymphocyte chemotaxis in PIIO-1-treated tumors (FIG. 25D). The deconvolution analysis of tumor bulk mRNA sequencing data demonstrated enrichment of CD8⁺ T cells, mast cells and activated NK cells in the TME after PIIO-1 administration (FIG. 25E). TGFβ can block mast cell activation through inhibiting its expression of high affinity IgE receptor (FcεRI). In summary, we conclude that treatment with single agent PIIO-1 remodels an immunosuppressive TME and shifts toward improved immune fitness with a rich pro-inflammatory cytokine milieu and abundance of effector lymphocytes.

### Example 18: Humanized PHIO-1 Enhanced Anti-Tumor Immunity by Facilitating CD8⁺ T Cell Recruitment into Tumors Through CXCR3

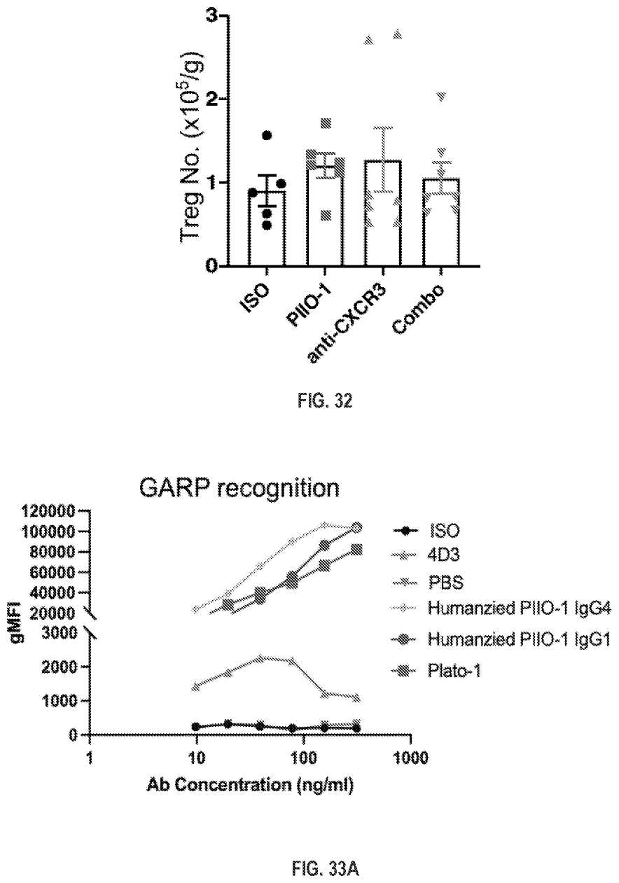

We next addressed the roles of CD8+ T cells in the protective immunity elicited by PIIO-1 and the potential underlying mechanisms. Depleting CD8⁺ T cells completely ablated the anti-tumor effects of PIIO-1 against MB-49 tumors (FIG. 26A-B), underscoring the importance of CD8+ T cells in PIIO-1-mediated tumor control. To determine if anti-tumor immunity is dependent on continuing migration of activated T cells from the dLNs to the tumor, we blocked T cell egress from dLNs with SIP receptor agonist FTY720 (FIG. 26C). We found that FTY20 abrogated the anti-tumor efficacy of PIIO-1 and effectively blocked T cell infiltration (FIG. 26D-F), indicating that expansion of pre-existing CD8⁺ T cells in the TME alone was unlikely a contributing factor for the PIIO-1 anti-tumor activity. Consistent with chemokine-mediated CD8⁺ T cell migration, we found that the CXCR3+CD8+ T cell population was enriched in the dLN after PIIO-1 administration (FIG. 26G), likely due to attenuated TGFβ signaling. Blocking CXCR3 during PIIO-1 treatment (FIG. 26H) completely abolished the anti-tumor activity of PIIO-1 (FIG. 26I-J), which correlated with reduced CD8+ T cell (and not Treg) recruitment to the TME (FIG. 26K-L and FIG. 32). Collectively, by blocking TGFβ activation within the TME, PIIO-1 promotes anti-tumor CD8+ T cell immunity, in part through increased CXCR3-dependent T cell trafficking into the tumor.

### Discussion

A key challenge in the field of immuno-oncology is primary and adaptive immune resistance to ICB seen in the majority of patients with cancer, including those with pancreatic cancer, ovarian cancer and most TNBCs. One underlying mechanism of primary and acquired ICB resistance in advanced malignancies relates to the accumulation of active TGFβ in the TME, which drives immune dysfunction by multiple mechanisms such as inducing Tregs, excluding and inhibiting the function of effector CD8+ T cells, and limiting effector T cell migration into the TME. However, targeting TGFβ has proven difficult to do for the treatment of human diseases due to pleotropic functions that are highly context dependent. Using a GARP-specific monoclonal antibody that blocks LTGFβ binding to Tregs, tumor cells and other cell types in the TME without affecting circulating platelets, we have accomplished tumor-selective targeting of the GARP-TGFβ pathway, as well as anti-tumor activity in multiple pre-clinical tumor models

PIIO-1 offers advantages over other technologies that attempt to drug the TGFβ pathway. It only targets GARP-expressing cells, which are primarily found in the TME, unlike agents that block TGFβ systemically such as anti-TGFβ antibodies and small molecule inhibitors against TGFβ signaling receptors. It differs from existing anti-GARP antibodies such as ABBV-151 under clinical evaluation in several aspects. First, PIIO-1 binds to ligand-free GARP and blocks the binding of GARP to all LTGFβ isoforms. Second, platelets express abundant GARP-LTGFβ1 complex due to their high levels of autocrine LTGFβ1. Antibodies targeting the GARP-LTGFβ1 complex (such as ABBV-151) pose a potential risk for platelet-related side effects, the unique epitope targeted by PIIO-1 (free GARP) ablates this risk. Third, the preferential targeting of PIIO-1 to tumors and dLNs underscores the favorable biodistribution of PIIO-1 over ABBV-151, which likely distributes non-selectively in the peripheral blood, bone marrow, and spleen.

PIIO-1 monotherapy successfully modulated the TME by reducing active TGFβ signaling and associated stromal formation, and enhanced accumulation of effector CD8+ T cells within the tumor. Furthermore, combination of PIIO-1 and anti-PD-1 therapy showed robust anti-tumor activities against GARP-tumors in humanized GARP knock-in mice. Mechanistic studies uncovered several intriguing biological insights related to the roles of GARP in the TME. The increased migration of CD8+ T cells to the TME in response to PIIO-1 is perhaps expected since there was evidence for reduced stromal formation and therefore less immune exclusion. Migration was likely also supported by increased chemokine production in the TME and the ability of TGFβ1 to suppress expression of CXCR3 on CD8+ T cells. We confirmed that PIIO-1 promotes CXCR3+ CD8+ T cells in the tumor dLNs. Interestingly, we found that CXCR3 is not required for Tregs to migrate into the TME. Therefore, increased CD8+ T cell migration over Tregs into the TME shall translate into reduction of Tregs proportionally, which appeared to be indeed the case. Importantly, PIIO-1 treatment curtailed CD8+ T cell exhaustion. Using a chronic viral infection model, Gabriel et al. recently reported that TGFβ1 maintains progenitor exhausted T cells via suppressing mTOR activity, eventually leading to a more terminally exhausted CD8+ T cell state. In our study, we used single cell high dimensional flow cytometry to demonstrate that PIIO-1 treatment significantly blocked formation of terminally exhausted CD8+ T cells in the TME, as indicated by high TOX expression and little-to-no expression of effector cytokines. Thus, by blocking active TGFβ production within the TME and dLNs, PIIO-1 augments CD8+ T cell biology in two ways-first, it promotes priming and migration of antigen-specific T cells in the dLNs, and second, it attenuates CD8+ T cell exhaustion in the TME.

Platelets are the major source of active TGFβ through GARP-mediated latent TGFβ maturation. Since PIIO-1 does not block platelet GARP-LTGFβ axis, we came to the conclusion that targeting GARP in the non-platelet compartment is sufficient to induce anti-tumor activity. Alternatively, extravasated tumor-infiltrating platelets, unlike circulating platelets, may also be a target of PIIO-1; this hypothesis is under active investigation using tissue-based spatial technology.

In conclusion, we generated, humanized and characterized a unique anti-GARP antibody that blocks activation of all isoforms of LTGFβ in the TME. Using our human LRRC32 knock-in mice and multiple preclinical tumor models, we demonstrated the potential drugability of the GARP-LTGFβ pathway for cancer immunotherapy. By doing so, we unraveled several new biological aspects of GARP, including how it contributes to immune exclusion, ICB resistance, CD8+ T cell exhaustion, and CD8+ T cell migration into the TME. Thus, PIIO-1 warrants further clinical development as a promising immunotherapeutic agent against advanced cancers with ICB resistance, both as a monotherapy and in combination with ICBs.

### Methods

### The Cancer Genome Atlas (TCGA) Database Analysis

LRRC32 expression values were obtained from TCGA using RNA-seq data available in the cBioPortal database and further integrated with the Immune Landscape of Cancer data using patient IDs. Comparison of each parameter in the Immune Landscape of Cancer between the top ⅓ (LRRC32 high) vs. the bottom ⅓ expression groups (LRRC32 low) was implemented by an independent t-test.

Generation of Anti-Human GARP (hGARP) Antibodies

The generation of anti-hGARP antibody has been described. BALB/c mice was immunized with recombinant human GARP (R&D Systems) in Freund's complete adjuvant and followed by boosting with SP2/0-hGARP cells for 2-3 times. Splenic B cells with high anti-GARP antibody titers from the immunized mice were fused to SP2/0 cells in the presence of polyethylene glycol. Hybridoma selection was done in HAT medium and cloning was done by limiting dilution assay.

### LAP Competition Binding Assay

1×105 Jurkat-hGARP cells were incubated with 400 ng human recombinant LTGFβ1 (R&D) and murine IgG1 isotype control (mIgG1) or anti-GARP antibodies at indicated concentration for 30 min at 37° C. Cells were washed with PBS twice and flow cytometry was performed using an anti-LAP antibody (eBioscience) to determine cell surface expression.

### In Vivo Models

hLRRC32KI mice received mIgG1 or PIIO-1 (200 μg) intravenously (i.v.) every other day for three treatments. Indicated organs were collected on day 5. The single cell suspension was prepared, followed by staining and flow cytometry analysis.

TNBC Model. 4T1-hGARP (1×105 cells) was injected into the fourth mammary fat pad of 6-8 weeks old female BALB/c mice. Antibodies were given intraperitoneally (i.p.) at day 7 post tumor injection and continued once every three days for 5 injections. Critical parameters were measured include tumor growth, body weight, survival time to the point of necessary euthanasia, lung metastasis, TGFβ level in the sera. To study anti-tumor memory response, mice with complete rejection of the tumors were then rechallenged with 4T1-WT (5×10⁵ cells), followed by close monitoring of tumor growth and overall survival time.

Bladder Cancer Model. MB-49 (1×10⁵ cells) was injected subcutaneously (s.c.) on the right flank of hLRRC32KI male mice. mIgG1 or PIIO-1 were given i.p. every three days on indicated days. Indicated tissues were then collected 24 hours after the last treatment. To study the efficacy of combination therapy, PIIO-1 (200 μg) and anti-PD-1 antibody were delivered (100 μg) every 3 days i.p. post MB-49 injection. PIIO-1 started on day 4 for 6 doses and anti-PD-1 antibody started on day 10 for 4 doses. Tumors were monitored daily. Mice which rejected tumor completely in indicated groups were then rechallenged with MB-49 (1×10⁵) s.c. Tumor growth and overall survival time were monitored.

MB-49 (1×10⁵ cells) were injected s.c. on the right flank of hLRRC32KI male mice. Anti-CD8a antibody (200 μg, i.p) was delivered on day 4, 6, 8, 11 and 14. PIIO-1 was given at 200 μg, i.p. on day 5, 8, 11 and 14. Tumor growth was monitored. To study the roles of T cell migration in the anti-tumor activity, FTY720 (2 mg/kg) was given on day 6 every two days for 6 doses. PIIO-1 was delivered (200 μg, i.p) on day 6, 9, 12 and 15. Experiment ended on day 17 with tumors and other organs analyzed. The roles of CXCR3 were also evaluated in the MB-49 model, with blocking anti-CXCR3 antibody and PIIO-1 (200 μg, i.p. each) given on day 5 post MB-49 injection every three days for 4 treatments Tumor growth was then monitored, with end-of-experiment analysis performed on day 16.

Tumor sizes were measured by longest width and length in mm and reported as tumor areas (width×length). For 4T1, LLC1, CMT167 tumor models, treatment was started when tumor area was around 30 mm2 (≈75 mm3 tumor volume) and for MB-49 model, treatment began when tumor area was around 12-24 mm2 (≈18-48 mm3)

### High Dimensional Flow Cytometry Analysis, Multi-Plex Immunofluorescence (IF) Microscopy

Antibody staining and high dimensional spectral flow cytometry analysis (Cytek) was performed. Multi-plex IF was done using Vectra Polaris. Detailed methods including spatial analysis were provided in the supplemental file.

### RNA-Seq Alignment, Preprocess, and Analysis

Sequencing was outsourced to Macrogen and performed on an Illumina Hiseq6000. Reads were aligned to the GRCm38 reference using the Hisat2 (v.2.0.5), and read counts were determined with the featureCounts (v1.5.0-p3) software. Raw read counts were used for DEGs analysis based on the DESeq2 package. The enrichment analyses of GO terms were performed via the R package clusterProfiler (v.3.18.0). Gene Set Enrichment Analyses (GSEA) (v.4.0.3) was implemented for enrichment analysis and visualization. The deconvolution was performed using TIMER 2.0. Detailed methods were provided in the supplemental file.

### Statistical Analysis

The Student's t-test was implemented to compare continuous variables between two groups such as control versus treatment. Kaplan-Meier curves were used to visualize different groups' survival and the log rank test was to quantify significance. Tumor curve analysis was performed using repeated measures analysis of variance (ANOVA). All data are presented as mean+SEM. P-values less than 0.05 were statistically significant. Turkey or Sidak procedures was used for multiple testing correction.

### Mice

Wild type C57BL/6 (strain #00064) and BALB/c (strain #000651) mice were purchased from Jackson Laboratory (Bar Harbor, ME). hLRRC32KI mice in C57BL/6 background was generated by Ingenious targeting laboratory (Ronkonkoma, NY). Age- and sex-matched mice were used for all the in vivo experiments. All experimental animals were 6-11 weeks old.

### Cell Lines and Mice

Jurkat, 4T1, MB-49 with hGARP overexpression were used. Cancer cells were authenticated by gene expression analysis, in vivo growth and histology. MB-49 urothelial carcinoma cell line was kindly provided by Dr. Xue Li (Cedars-Sinai Medical Center, Los Angeles, CA). 293 FT and LLC1 lines were purchased from ATCC (Manassas, VA). CMT-167 cell line was obtained from Sigma (St. Louis, MO). All cell lines were tested to be free of Mycoplasma by PCR. For all the in vivo tumor experiment, tumor cells were used within the first four passages of the culture.

### Generation of Human/Mouse GARP-Expression Vectors

Human and mouse GARP was amplified by PCR and subcloned between the BglII and HpaI sites in a MigR1 retroviral vector.

For chimeric construction, we used the following
primers:
20-60 Forward:
(SEQ ID NO: 38)
GCTCTCTACTTGTCCGGGAACCAACTGCGGAGTATCCTGGCCTCACCC
20-60 reverse:
(SEQ ID NO: 39)
GGGTGAGGCCAGGATACTCCGCAGTTGGTTCCCGGACAAGTAGAGAGC
61-100 forward:
(SEQ ID NO: 40)
CAGGCCCTGCCCTACCTGGAGCACCTCAGCCTGGCTCACAACCGGCTG
61-100 reverse:
(SEQ ID NO: 41)
CAGCCGGTTGTGAGCCAGGCTGAGGTGCTCCAGGTAGGGCAGGGCCTG
101-140 forward:
(SEQ ID NO: 42)
AACAGCCTGCATGGCAATCTGGTGGAGCGGCTGCTGGGGGAGGCACCC
101-140 reverse:
(SEQ ID NO: 43)
GGGTGCCTCCCCCAGCAGCCGCTCCACCAGATTGCCATGCAGGCTGTT
141-170 forward:
(SEQ ID NO: 44)
CGCCTGGCACGCCACACCTTCTGGGACATGCCTGCGCTGGAGCAGCTT
141-170 reverse:
(SEQ ID NO: 45)
AAGCTGCTCCAGCGCAGGCATGTCCCAGAAGGTGTGGCGTGCCAGGCG
171-207 forward:
(SEQ ID NO: 46)
ACTCACCTCAATCTCTCCAGAAACTCCCTCACCTGCATCTCCGACTTC
171-207 reverse:
(SEQ ID NO: 47)
GAAGTCGGAGATGCAGGTGAGGGAGTTTCTGGAGAGATTGAGGTGAGT
208-265 forward:
(SEQ ID NO: 48)
TTCCCTGACCTGGCCGTGTTCCCGAGACTCATCTACCTGAACTTGTCC
208-265 reverse:
(SEQ ID NO: 49)
GGACAAGTTCAGGTAGATGAGTCTCGGGAACACGGCCAGGTCAGGGAA
266-322 forward:
(SEQ ID NO: 50)
AATGAGATCGAACTGGTCCCTGCTAGCTTTCTTGAGCACCTGACCTCC
266-322 reverse:
(SEQ ID NO: 51)
GGAGGTCAGGTGCTCAAGAAAGCTAGCAGGGACCAGTTCGATCTCATT

All constructs were subcloned into MigR1 retroviral vector for retrovirus production. The efficiency of mutagenesis was assessed by DNA sequencing. Chimeric constructions were transfected into 293 FT cells and the cells with desired expression level of the construct were selected by FACS sorting.

### In Vivo Murine Tumor Model

LLC1 tumor cells (5×105) or CMT-167 cells (1×105) were injected s.c. on the right flank of hLRRC32KI female mice. Mice were given PIIO-1 (200 μg), anti-PD-1 (100 μg) or combination of both on day 8 every three days for 4 treatments. Tumor growth was monitored, and tissues were collected on day 18. Flow cytometry were analyzed at the end point of the experiment.

MB-49-hGARP or -EV tumor cells (1×105) were injected s.c. in the right flank of C56BL/6 male mice. Tumors were harvested on day 18. The single cell suspension was prepared, stained with the proper antibodies, followed by flow cytometry analysis.

### Tissue Digestion, Cell Isolation and Flow Cytometry

Thymus, spleen, mesenteric lymph nodes (mLN), and peripheral lymph nodes (pLN), were dissociated into a single-cell suspension and RBC lysis buffer (Biolegend) was used to remove red blood cells. To isolate tumor, tissues were dissected and incubated for 20 minutes at 37° C. with collagenase D (1 mg/mL; Roche), dispase (0.05 U/mL; Worthington), and DNase I (100 mg/mL; SigmaAldrich). Digested tissue was then filtered through a 40-μm nylon strainer (VWR). Blood cells were removed with RBC lysis buffer (Biolegend). Cell suspension was washed by PBS.

For flow cytometry staining, cells were washed twice in FACS buffer and FcR blocking was applied 10 minutes at 4° C. Live/dead staining was performed for 10 minutes at 4° C. with Fixable Viability Dye (Affymetrix) or live/dead blue (Thermofisher) before staining with the surface antibody (described below) mix for 30 minutes at 4° C. in FACS buffer. For intracellular staining, Foxp3/Transcription Factor Staining Buffer Set (eBioscience) was used according to the manufacturer's protocol. Cells were then incubated with antibodies for 1-3 hours in permeabilization buffer. Cells for cytokine production assessment were stimulated in T cell medium with anti-CD3 (1 μg/ml)/CD28 (5 μg/ml) for 5 hours at 37° C. then followed with FACS staining. Samples were analyzed immediately on BD FACSDiva, Fortessa or Cytek Aurora, and data analysis was performed using FlowJo (Tree Star) or OMIQ software.

For pSMAD2/3 staining, tissues were meshed in the fixation buffer (Invitrogen) for 30 minutes and filtered through a 40-μm nylon strainer (VWR). Cell suspensions were permeabilized in the perm buffer at room temperature (RT) for 30 minutes. Cell surface markers were stained at RT in FACS buffer for 1 hour. pSMAD2/3 and Foxp3 were stained overnight at 4° C. in FACS buffer. Flow cytometry was performed immediately using Cytek Aurora.

### Immune Phenotyping Panel:

Anti-CD45 (Clone 30-F11, Brilliant Violet 510, BioLegend), anti-CD3 (Clone 17A2, BUV737, BD Biosciences), anti-CD8a (Clone 53-6.7, BUV496, BD Biosciences), anti-CD4 (Clone RM4-5, APC/Fire™ 810, BioLegend), anti-Foxp3 (Clone FJK-16s, eFluor450, Invitrogen), anti-CD25 (Clone PC61.5, Super Bright 600, Invitrogen), anti-CD11b (Clone M1/70, Alexa Fluor 532, Invitrogen), anti-F4-80 (Clone T45-2342, BUV395, BD Horizon), anti-CD11c (Clone N418, Brilliant Violet 750, BioLegend), anti-MHC-II (Clone M1/42, BUV615, BD Biosciences), anti-NK-1.1 (Clone PK136, Brilliant Violet 570, BioLegend), anti-Ly-6C (Clone HK1.4, Brilliant Violet 605, BioLegend), anti-Ly-6G (Clone 1A8-Ly6g, Super Bright 436, Invitrogen), anti-CD103 (Clone 2E7, Brilliant Violet 711, BioLegend), anti-PD-1 (Clone J43, FITC, Invitrogen), anti-PD-L1 (Clone B7-H1, Brilliant Violet 421, BioLegend), anti-CD206 (Clone MR6F3, APC-eflour780, Invitrogen), anti-CD38 (Clone 90/CD38, PE/Cyanine7, BioLegend), anti-Arginase 1 (Clone AlexF5, Alexa Fluor 700, Invitrogen), anti-CD64 (Clone X54-5/7.1, APC, BioLegend), XCR1 (Clone ZET, PerCP/Cyanine5.5, BioLegend), anti-CD172 (Clone P84, PE/Dazzle™ 594, BioLegend), anti-CD19 (Clone 6D5, Spark NIR™ 685, BioLegend), anti-CD24 (Clone M1/69, BV480, BD Biosciences);

### T Cell Exhaustion Panel:

Anti-CD45 (Clone 30-F11, Brilliant Violet 510, BioLegend), anti-CD3 (Clone 17A2, BUV737, BD Biosciences), anti-CD8a (Clone 53-6.7, BUV496, BD Biosciences), anti-CD4 (Clone RM4-5, APC/Fire™ 810, BioLegend), anti-Foxp3 (Clone FJK-16s, eFluor450, Invitrogen), anti-CD25 (Clone PC61.5, Super Bright 600, Invitrogen), anti-TOX (Clone REA473, PE, Miltenyi Biotec), anti-CD44 (Clone IM7, BUV611, Invitrogen), anti-CD62L (Clone MEL-14, Brilliant Violet 421, BioLegend), anti-Slamf6 (Clone 13G3-19D, APC, Invitrogen), anti-PD-1 (Clone J43, APC-cflour780, Invitrogen), anti-Tim3 (Clone RMT3-23, Brilliant Violet 711, BioLegend), anti-Lag3 (Clone C9B7W, BUV 805, BD Biosciences), anti-Klrg1 (Clone 2F1, Pacific Orange, Invitrogen), anti-CD27 (Clone LG.3A10, BUV563, BD Biosciences), anti-CD38 (Clone 90/CD38, Brilliant Violet 750, BD Biosciences), anti-ICOS (Clone 7E.17G9, Super Bright 436, Invitrogen), anti-CD69 (Clone H1.2F3, PE/Cyanine7, BioLegend), anti-TIGIT (Clone 1G9, Brilliant Violet 650, BD Optibuild), anti-GITR (Clone MIH44, BUV615, BD Biosciences), anti-CTLA4 (Clone UC10-4B9, PE/Dazzle™ 594, BioLegend), anti-CD95 (Clone Jo2, BV480, BD Biosciences), anti-Ki67 (Clone B56, BUV395, BD Biosciences), anti-Tcf1 (Clone C63D9, PE/Cyanines, Cell Signaling Technology), anti-Bel-2 (Clone BCL/10C4, Alexa Fluor 647, BioLegend), anti-Granzyme B (Clone QA16A02, Alexa Fluor 700, BioLegend), anti-T-bet (Clone 04-46, Brilliant Violet 786, BD Biosciences);

### Cytokine Panel:

Anti-CD45 (Clone 30-F11, Brilliant Violet 510, BioLegend), anti-CD3 (Clone 17A2, BUV737, BD Biosciences), anti-CD8a (Clone 53-6.7, BUV496, BD Biosciences), anti-CD4 (Clone RM4-5, APC/Fire™ 810, BioLegend), anti-Foxp3 (Clone FJK-16s, eFluor450, Invitrogen), anti-CD11b (Clone M1/70, Alexa Fluor 532, Invitrogen), anti-TOX (Clone REA473, PE, Miltenyi Biotec), anti-Tcf1 (Clone C63D9, PE/Cyanine7, Cell Signaling Technology), anti-TNFα (Clone MP6-XT22, Percp-eflour 710, Invitrogen), anti-IFNγ (Clone XMG1.2, Brilliant Violet 786, BD Biosciences), anti-Granzyme B (Clone QA16A02, Alexa Fluor 700, BioLegend), anti-Perforin (Clone eBioOMAK-D, FITC, Invitrogen), anti-IL-2 (Clone JES6-SH34, PE-eflu610, Invitrogen), anti-IL-4 (Clone 11B11, BV605, BD Horizon), anti-IL-10 (Clone JES5-16E3, APC, Invitrogen), anti-IL-17A (Clone TC11-18H10, APC-Cy7, BD Pharmingen), anti-IL-21 (Clone FFA21, eFluor660, Invitrogen);

### Phospho-Flow Panel:

Anti-CD45 (Clone 30-F11, Brilliant Violet 510, BioLegend), anti-CD3 (Clone 17A2, BUV737, BD Biosciences), anti-CD8a (Clone 53-6.7, BUV496, BD Biosciences), anti-CD4 (Clone R.M4-5, APC/Fire™ 810, BioLegend), anti-Foxp3 (Clone FJK-16s, eFluor450, Invitrogen), anti-CD25 (Clone PC61.5, Super Bright 600, Invitrogen), anti-CD11b (Clone M1/70, Alexa Fluor 532, Invitrogen), anti-F4-80 (Clone T45-2342, BUV395, BD Horizon), anti-CD11c (Clone N418, Brilliant Violet 750, BioLegend), anti-MHC-II (Clone M1/42, BUV615, BD Biosciences), anti-NK-1.1 (Clone PK136, Brilliant Violet 570, BioLegend), anti-Ly-6C (Clone HK1.4, Brilliant Violet 605, BioLegend), anti-Ly-6G (Clone 1A8-Ly6g, Super Bright 436, Invitrogen), anti-CD206 (Clone MR6F3, APC-eflour780, Invitrogen), anti-CD19 (Clone 6D5, Spark NIR™ 685, BioLegend), anti-pSMAD2/3 (Clone O72670, PE, BD Pharminen)

### Multiplex Immunofluorescence Analysis

The samples were outsourced to Fred Hutch for the IF staining and the method was provided by Fred Hutch. Formalin-fixed paraffin-embedded tissues were sectioned at 4 microns and baked for 1 h at 60° C. The slides were dewaxed by using dewax solution (Leica). Antigen retrieval (Bond Wash Solution) was applied at 100° C. for 20 mins. 3% H₂O₂ was used for endogenous peroxidase blocking for 5 mins followed by incubating 10% normal mouse serum in TCT buffer (0.05M Tris, 0.15M NaCl, 0.25% Casein, 0.1% Tween 20, pH 7.6) for 10 mins. CD45 Ica antibody was applied for 1 h and the secondary antibody was stained for 10 mins. Then, the tertiary TSA-amplification reagent was applied (PerkinElmer OPAL fluor) for 10 mins. After secondary and tertiary application, a high stringency wash was performed by using high-salt TBST solution (0.05M Tris, 0.3M NaCl, and 0.1% Tween-20, pH 7.2-7.6). Polymer HRP as secondary was indicated in the table (Leica). See Table K below.

TABLE K

| | | Clone/ | Manufacturer/ | | | Opal |
|---|---|---|---|---|---|---|
| Position | Antibody | Host | Catalog Number | Dilution | Secondary | Dye |
| 1 | cd45 1ca | Rabbit | Abcam | 1:3000 | PowerVision | Opal |
| | | polyclonal | ab10558 | | Rabbit HRP | 520 |
| 2 | SMA | Rabbit | Proteintech | 1:5000 | PowerVision | Opal |
| | | polyclonal | 80008-1-RR | | Rabbit HRP | 540 |
| 3 | cd8a | Rabbit | Cell signaling | 1:1500 | PowerVision | Opal |
| | | D4W2Z | 98941 | | Rabbit HRP | 570 |

SMA staining was done after stripping process in retrieval solution for 20 mins at 100° C. Before SMA staining, 3% H2O2 was used for endogenous peroxidase blocking. The process CD8a staining was repeated as SMA. Lastly, slides were stained with DAPI for 5 minutes, rinsed and coverslipped in Prolong Gold Antifade reagent (Invitrogen). Images were acquired on the Perkin Elmer Vectra 3.0 Automated Imaging System (Akoya Biosciences, Marlborough, MA) using the filters and exposure times in the table L below.

TABLE L

| Filter | Scan exposure time ²: | Field exposure time ²: |
|---|---|---|
| DAPI | 25 | 200 |
| FITC | 150 | 250 |
| CY3 | 30 | 70 |
| Texas Red | 25 | 40 |
| CY5 | 150 | 200 |

Briefly, the slides were first scanned using long pass filters at 10× magnification to capture the entire tissue section. These images were annotated for the Regions of Interests (ROIs) covering the entire tissue. Next, these ROIs were imaged using multispectral imaging settings for each biomarker. The resulting .im3 multispectral images were quantified for CD45, CD8a, SMA and DAPI. These ROIs were imported into the inForm software for further analyses. First, the images were annotated for biomarkers and fluorophores. The autofluorescence signal was isolated and the multiplexed fluorescence signals were unmixed. The images were normalized to the exposure time. The inForm software allows development of machine-learning based segmentation of tissues categories and segmentation of cells. A subset of ROIs was sampled to make training set for image processing, tissue segmentation, cell segmentation and phenotyping algorithms. These algorithms were applied to all ROIs of all images in the dataset for batch analyses. The resulting comprehensive data that was further analyzed using phenoptr package and R-programming for identifying and quantifying cells for each biomarker within each tissue compartment (defined as tumor and stroma) as well as in the entire tissue section.

### Region Definitions

The imaged cells were classified into stromal or tumor cell categories by a machine learning algorithm (inform software from Akoya). Next, we determined the largest cluster of tumor cells by using a flood-fill algorithm in the following way. The region was discretized into a square lattice with lattice constant 30 μm where a pixel is considered occupied if at least one tumor cell is present in it. The occupied pixels were connected to form clusters by joining face sharing nearest neighbors. We calculated the convex hull of the largest cluster of tumor cells to define the boundary between the tumor mass and the exterior stromal region. The center of mass of the tumor was calculated by taking the average position of all the tumor cells in the largest cluster of tumor cells. We then use the boundary between tumor mass and the stromal region and the center of mass of largest cluster of tumor cells to divide the tumor region into three regions (Intermediate I, Intermediate II, and Interior) (FIG. 30A) based on their proximity to the center of mass in the following way. If {xi(b), yi(b)} represent the positions of the tumor cells in the boundary where the center of mass is the origin, then the boundary of a region in the tumor mass is given by {αxi(b), αyi(b)}, where α<1. The α values corresponding to the boundaries are shown in FIG. 30A. The spatial distributions of CD8+ T cells and other cells were analyzed in these regions to evaluate the changes in the organization of these cells based on the proximity of the cells to the center of the tumor region.

### Density

Density (G) of a particular cell type, e.g., CD8+ T cell, in a region is calculated by the ratio of the total number (NTot) of the cells and the area (A) of that region, i.e.,

$$σ = \frac{N {}_{Tot}}{A} .$$

The area of a region is calculated numerically by partitioning the region (e.g., Intermediate II) into a square lattice with lattice constant a=30 μm and then calculating the area of the filled portion of the lattice.

### Two-Point Correlation

We compute spatial two point correlation for CD8+ T cells in a region (e.g., Intermediate II) in the following way (see page 34 for more on the two point correlation). For any CD8+ T cell (indexed by i) in the region, we draw an annular region of radius r and thickness δ (=3 μm) with the CD8+ T cell positioned at the center and compute the density of other CD8+ T cells in that annular region (FIG. 30B). Defining n_{i,(r-δ/2,r+δ/2)} as the number of CD8+ T cells in the annulus and Aannulus as the area of the annular region, the density σi(r) of the CD8+ T cells in the annular region surrounding the ith CD8+ T cell is given by,

$${σ}_{i} ( r ) = \frac{{n}_{i , ( r - \frac{δ}{2} , r + \frac{δ}{2} )}}{{A}_{Annulus}} ,$$

where Aₐₙₙᵤₗᵤₛ=πrδ. The total number (N_{CD8+}) of CD8⁺ T cells and density of the CD8⁺ T cells (σ_{CD8+}=N_{CD8+}/(area of the region)) in the region is also computed. The pair correlation function is then given by,

$$C ( r ) = \frac{1}{{N}_{CD {8}^{+}}} {\sum }_{i} {o}_{i} ( r ) - {σ}_{CD {8}^{+}}$$

This calculation is done for multiple radii r and the resulting function is plotted as a function of r.

### Bulk RNA-Seq Analysis

### Public Data Access and Analysis.

The bulk RNA-seq data of bladder cancer were downloaded from, in support of survival analysis and LRRC32 gene expression analysis. The 167 bladder tumor samples were selected based on the “Best Confirmed Overall Response” annotation, including 15 CR (complete response), PR (partial response), SD (stable disease), and PD (progressive disease). LRRC32-TGFB related signature includes: LRRC32, ITGB6, ITGB8, ITGAV, ITGA2B, SELP, F2, TGFB1 genes. The DESeq 2 (v.1.30) normalization method was applied before the survival analysis and GARP gene expression. The survival analysis was performed based on the package survival (v 3.1).

### Samples and Library Preparation

1×10⁵ MB-49 cells were injected s.c. on the right flank of hLRRC32KI male mice. PIIO-1 (200 μg/mouse, i.p.) were delivered on day 6 and 9 for 2 doses. Tumors were collected on day 10. Single cell suspension and RNA isolation were prepared. Total RNA was isolated by using RNeasy Kits (Qiagen) and then subjected to bulk RNA sequencing. RNA quality was verified with an Agilent Bioanalyser. Libraries were prepared using NEBNext Ultra™ RNA Library Prep Kit for Illumina (NEB, USA), following manufacturer's recommendations.

### Alignment and Quantification

Sequencing was outsourced to Macrogen and performed on an Illumina Hiseq6000 with the following requirement: 150 pb of read length, paired-end reads, and 300 M reads/sample. The reads were removed if they contained adapters, N was greater than 10% (N represents a base that could not be determined), or they were identified as low-quality reads in which the Q score (Quality value) was less than 5. Filtered reads were then aligned to the GRCm38 mouse genome using the Hisat2 (v.2.0.5) followed default settings, and read counts were determined with the featureCounts (v1.5.0-p3) software. Raw read counts were normalized using the DESeq2 package with default settings.

### Pathway Enrichment Analysis and Deconvolution Analysis

The DEGs were selected if the p-value were less than 0.001 and the absolute value of log-fold change was higher than 0.5. Based on the identified DEGs, the enrichment analyses of GO terms (Biological Process, Cell Component, and Molecular Function) were performed via the R package clusterProfiler (v.3.18.0). GSEA (v.4.0.3) was also implemented for enrichment analysis and visualization 7. The deconvolution was performed using TIMER 2.0 following its tutorial 8.

### Immunohistochemistry (IHC)

Mouse tumor slides were processed, and antigen retrieved. For mouse IHC, tissues were collected and place into 4% paraformaldehyde overnight for fixation, then fixed tissue was incubated in 70% ethanol overnight prior to paraffin embedding, and then cut for hematoxylin and eosin (H&E) staining. For pSMAD2/3 or α-SMA on paraffin tumor sections, 4 μm sections were incubated with 3% H2O2. To minimize nonspecific staining, sections were incubated with the appropriate animal serum for 20 min at RT, followed by incubation with primary anti-pSMAD2/3 antibody (Abcam) or α-SMA (Abcam) overnight at 4° C. Staining with secondary antibodies (Vectastain ABC Kit) was then performed before development using DAB substrate (Vector Labs SK-4100) The staining intensity of pSMAD2/3 or α-SMA was graded as follows with the sample identity blinded (0: negative; 1: faint; 2: moderate; 3: strong but less intense than 4; and 4: intense).

### Soluble TGFβ1 ELISA

Mouse blood was collected in Eppendorf tubes. Sera were collected after coagulation for 1 hour at RT and centrifugation at 5,000 rpm for 15 minutes. Capture ELISA for TGFβ1 was performed according to manufacturer instructions (BioLegend). Active TGFβ1 was measured with no additional manipulation. Total TGFβ1 was measured following acidic activation using 1 M HCl for 10 min at RT, and neutralization with 1.2N NaOH. Active TGFβ1 and total TGFβ1 levels were measured using TGFβ1 ELISA kits according to the manufacturer's protocols.

### Binding Assay

1×10⁵ Jurkat-hGARP cells were collected and washed with PBS twice. Cells were stained with live dead blue (1:1000, Cat. L23105, Invitrogen) at 4° C. for 15 min. Cells were washed with FACS buffer twice and incubated with isotype control or PIIO-1 at indicated concentration (20, 10, 5, 2.5, 1.25, 0.625, 0.3125, 0 μg/ml) for 30 min at 4° C. in FACS buffer. Then, washed with FACS buffer twice and further stained with anti-mouse Ig-PE or anti-human Fc-PE 30 min at 4° C. in FACS buffer. Surface GARP staining will be performed for flow cytometry.

### Groups

1. Murine IgG1 isotype control (BioXcell)
2. Murine PIIO-1 (Hybridoma, BioXcell)
3. mouse antibody
4. PBS control for humanized antibody
5. Humanized PIIO-1 (IgG4, Thermofisher)
6. Humanized PIIO-1 (IgG1, Ab studio)
7. Murine anti-GARP antibody (Plato-1, Enzo)
   Readout: Genomic mean fluorescence intensity of GARP in different antibody concentration.

### Competition Assay

1×10⁵ Jurkat-hGARP cells were incubated with 400 ng human recombinant LTGFβ1 (R&D) and
isotype control or PIIO-1 at indicated concentration (20, 10, 5, 2.5, 1.25, 0.625, 0.3125, 0 μg/ml) for 30 min at 37° C. Cells were washed with PBS twice and further performed flow cytometry to determine LAP (eBioscience) expression on cell surface.

### Groups:

1. Murine IgG1 isotype control (BioXcell)
2. Murine PIIO-1 (Hybridoma, BioXcell)
3. mouse antibody
4. PBS control for humanized antibody
5. Humanized PIIO-1 (IgG4, Thermofisher)
6. Humanized PIIO-1 (IgG1, Ab studio)
   Readout: Genomic mean fluorescence intensity of LAP in different antibody concentration.

### Example 19 Binding Data Regarding the Superiorly of PIIO-1 to 4D3

We generated multiple antibodies including 4D3, humanized PIIO-1 IgG1 and humanized PIIO-1 IgG4 against human GARP. The recombinant humanized PIIO-1 IgG4 was made by Thermo Fisher in CHO cells, and humanized PIIO-1 IgG1 was generated by Ab Studio. We also used Plato-1, a commercially available anti-GARP antibody (Enzo) for some of the experiments. We performed experiments to examine their ability to bind to GARP as well as their properties to inhibit the interaction between GARP and the extracellular latent TGFβ.

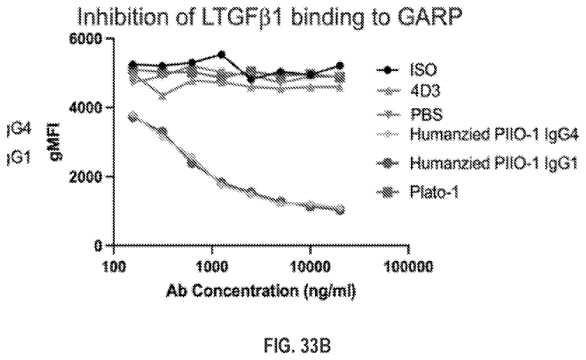

To determine if they were able to recognize GARP on cell surface, we utilized human GARP overexpressing Jurkat cells. In brief, 1×10⁵ Jurkat-hGARP cells (GARP overexpressing Jurkat cell) were incubated with anti-GARP antibodies at indicated concentration (312.5, 156.25, 78, 39, 20, 9.7, 0 ng/ml) for 30 min at 4° C. This was then followed by incubating with anti-mouse Ig-PE or anti-human Fc-PE secondary antibody. The GARP expression level was assessed by flow cytometry, with results quantified by the geometric mean of fluorescence intensity (gMFI). We found that all anti-GARP antibodies recognize GARP in a dose-dependent manner except isotype control antibody (ISO). However, 4D3 does not bind to GARP as efficiently as PIIO-1. (FIG. 33A). To determine if these antibodies are able to block the blinding between GARP and latent TGFβ1 (LTGFβ1), 1×10⁵ Jurkat-hGARP cells were incubated with 400 ng human recombinant LTGFβ1 (R&D), in the presence of isotype control or anti-GARP antibodies at indicated concentration (20, 10, 5, 2.5, 1.25, 0.625, 0.3125, 0 μg/ml) for 30 min at 37° C. Cells were then thoroughly washed with PBS twice to remove free unbound LTGFβ1. The cell surface LTGFβ1 was then detected by anti-LTGFβ1 antibody (eBioscience), followed by flow cytometry analysis and quantification. Using this competition binding assay, we found that PIIO-1 blocked all LTGFβ1 binding to GARP, however, 4D3 or Plato-1 failed to block the binding between GARP and LTGFβ1. Importantly, we found that the competition of PIIO-1 over LTGFβ1 for binding to GARP is dose-dependent (FIG. 33B).

In summary, these experiments demonstrated that original PIIO-1, humanized PIIO-1 IgG1 and humanized PIIO-1 IgG1 were able to effectively interact with GARP, resulting in robust blocking of the binding between GARP and LTGFβ1. 4D3 has the ability to recognize GARP but does not inhibit the interaction between GARP and LTGFβ1 as efficiently as PIIO-1.

### REFERENCES

Ahmadzadeh M, Rosenberg S A. TGF-beta 1 attenuates the acquisition and expression of effector function by tumor antigen-specific human memory CD8 T cells. J Immunol 2005; 174(9):5215-23. doi: 10.4049/jimmunol. 174.9.5215
Ahn, Y. O., Lee, J. C., Sung, M. W., and Heo, D. S. (2009). Presence of membrane-bound TGF-beta1 and its regulation by IL-2-activated immune cell-derived IFN-gamma in head and neck squamous cell carcinoma cell lines. Journal of Immunology 182, 6114-6120.
Akhurst R J. Targeting TGF-beta Signaling for Therapeutic Gain. Cold Spring Harb Perspect Biol 2017; 9(10) doi: 10.1101/cshperspect.a022301
Akhurst, R. J., and Hata, A. (2012). Targeting the TGFbeta signalling pathway in disease. Nature Reviews Drug Discovery 11, 790-811.
Alberti C, Manzenreither R A, Sowemimo I, et al. Cell-type specific sequencing of microRNAs from complex animal tissues. Nat Methods 2018; 15(4):283-89. doi: 10.1038/nmeth.4610
Alfei F, Kanev K, Hofmann M, Wu M, Ghoneim H E, Roelli P, Utzschneider D T, von Hoesslin M, Cullen J G, Fan Y, Eisenberg V, Wohlleber D, Steiger K, Merkler D, Delorenzi M, Knolle P A, Cohen C J, Thimme R, Youngblood B, Zehn D. TOX reinforces the phenotype and longevity of exhausted T cells in chronic viral infection. Nature. 2019; 571(7764):265-9.
Bagchi S, Yuan R, Engleman E G. Immune Checkpoint Inhibitors for the Treatment of Cancer: Clinical Impact and Mechanisms of Response and Resistance. Annu Rev Pathol 2021; 16:223-49. doi: 10.1146/annurev-pathol-042020-042741
Baker, K., Raut, P., and Jass, J. R. (2008). Colorectal cancer cells express functional cell surface-bound TGFbeta. International Journal of Cancer 122, 1695-1700.
Barber D L, Wherry E J, Masopust D, et al. Restoring function in exhausted CD8 T cells during chronic viral infection. Nature 2006; 439(7077):682-7. doi: 10.1038/nature04444
Batlle E, Massague J. Transforming Growth Factor-beta Signaling in Immunity and Cancer. Immunity. 2019; 50(4):924-40.
Bellomo C, Caja L, Moustakas A. Transforming growth factor beta as regulator of cancer stemness and metastasis. Br J Cancer. 2016; 115(7):761-9.
Beltra J C, Manne S, Abdel-Hakeem M S, et al. Developmental Relationships of Four Exhausted CD8(+) T Cell Subsets Reveals Underlying Transcriptional and Epigenetic Landscape Control Mechanisms. Immunity 2020; 52(5):825-41 e8. doi: 10.1016/j.immuni.2020.04.014
Beltra J C, Manne S, Abdel-Hakeem M S, Kurachi M, Giles J R, Chen Z, Casella V, Ngiow S F, Khan O, Huang Y J, Yan P, Nzingha K, Xu W, Amaravadi R K, Xu X, Karakousis G C, Mitchell T C, Schuchter L M, Huang A C, Wherry E J. Developmental Relationships of Four Exhausted CD8(+) T Cell Subsets Reveals Underlying Transcriptional and Epigenetic Landscape Control Mechanisms. Immunity. 2020; 52(5):825-41 e8.
Beyer, M., and Schultze, J. L. (2006). Regulatory T cells in Cancer. Blood 108, 804-811.
Bhowmick, N. A., Ghiassi, M., Bakin, A., Aakre, M., Lundquist, C. A., Engel, M. E., Arteaga, C. L., and Moses, H. L. (2001). Transforming growth factor-beta1 mediates epithelial to mesenchymal transdifferentiation through a RhoA-dependent mechanism. Molecular Biology of the Cell 12, 27-36.
Blank C U, Haining W N, Held W, Hogan P G, Kallies A, Lugli E, Lynn R C, Philip M, Rao A, Restifo N P, Schietinger A, Schumacher T N, Schwartzberg P L, Sharpe A H, Speiser D E, Wherry E J, Youngblood B A, Zehn D. Defining ‘T cell exhaustion’. Nat Rev Immunol. 2019; 19(11):665-74.
Boutet M, Gauthier L, Leclerc M, et al. TGFbeta Signaling Intersects with CD103 Integrin Signaling to Promote T-Lymphocyte Accumulation and Antitumor Activity in the Lung Tumor Microenvironment. Cancer Res 2016; 76(7):1757-69. doi: 10.1158/0008-5472.CAN-15-1545
Camp, R. L., Dolled-Filhart, M., and Rimm, D. L. (2004). X-tile: a new bio-informatics tool for biomarker assessment and outcome-based cut-point optimization. Clinical Cancer Research 10, 7252-7259.
Campbell M G, Cormier A, Ito S, Seed R I, Bondesson A J, Lou J, Marks J D, Baron J L, Cheng Y, Nishimura S L. Cryo-EM Reveals Integrin-Mediated TGF-beta Activation without Release from Latent TGF-beta. Cell. 2020; 180(3):490-501 e16.
Carrillo-Galvez A B, Cobo M, Cuevas-Ocana S, et al. Mesenchymal stromal cells express GARP/LRRC32 on their Surface: Effects on their biology and immunomodulatory capacity. Stem cells 2014 doi: 10.1002/stem.1821
Carrillo-Galvez, A. B., Cobo, M., Cuevas-Ocana, S., Gutierrez-Guerrero, A., Sanchez-Gilabert, A., Bongarzone, P., Garcia-Perez, A., Munoz, P., Benabdellah, K., Toscano, M. G., et al. (2015). Mesenchymal stromal cells express GARP/LRRC32 on their surface: effects on their biology and immunomodulatory capacity. Stem Cells 33, 183-195.
Chaikin P M, Lubensky T C Principles of condensed matter physics. Cambridge; New York, NY, USA: Cambridge University Press 1995.
Chen L, Han X. Anti-PD-1/PD-L1 therapy of human cancer: past, present, and future. J Clin Invest. 2015; 125(9):3384-91.
Chen S, Fan J, Zhang M, Qin L, Dominguez D, Long A, Wang G, Ma R, Li H, Zhang Y, Fang D. Sosman J, Zhang B. CD73 expression on effector T cells sustained by TGF-beta facilitates tumor resistance to anti-4-1BB/CD137 therapy. Nat Commun. 2019; 10(1):150.
Chen Z, Ji Z, Ngiow S F, Manne S, Cai Z, Huang A C, Johnson J, Staupe R P, Bengsch B, Xu C, Yu S, Kurachi M, Herati R S, Vella L A, Baxter A E, Wu J E, Khan O, Beltra J C, Giles J R, Stelekati E, McLane L M, Lau C W, Yang X, Berger S L, Vahedi G, Ji H, Wherry E J. TCF-1-Centered Transcriptional Network Drives an Effector versus Exhausted CD8 T Cell-Fate Decision. Immunity. 2019; 51(5):840-55 e5.
Chen, X., Yang, Y., Zhou, Q., Weiss, J. M., Howard, O. Z., McPherson, J. M., Wakefield, L. M., and Oppenheim, J. J. (2014). Effective chemoimmunotherapy with anti-TGFbeta antibody and cyclophosphamide in a mouse model of breast cancer. PloS One 9, e85398.
Chow M T, Ozga A J, Servis R L, et al. Intratumoral Activity of the CXCR3 Chemokine System Is Required for the Efficacy of Anti-PD-1 Therapy. Immunity 2019; 50(6):1498-512 e5. doi: 10.1016/j.immuni.2019.04.010
Conley J M, Gallagher M P, Berg L J. T Cells and Gene Regulation: The Switching On and Turning Up of Genes after T Cell Receptor Stimulation in CD8 T Cells. Front Immunol. 2016; 7:76. Epub 2016 Mar. 15. doi: 10.3389/fimmu.2016.00076. PubMed PMID: 26973653; PMCID: PMC4770016.
Cuende J, Lienart S, Dedobbeleer O, van der Woning B, De Boeck G, Stockis J, Huygens C, Colau D, Somja J, Delvenne P, Hannon M, Baron F, Dumoutier L, Renauld J C, De Haard H, Saunders M, Coulie P G, Lucas S. Monoclonal antibodies against GARP/TGF-beta1 complexes inhibit the immunosuppressive activity of human regulatory T cells in vivo. Sci Transl Med. 2015; 7(284):284ra56.
Cuende, J., Lienart, S., Dedobbeleer, O., van der Woning, B., De Boeck, G., Stockis, J., Huygens, C., Colau, D., Somja, J., Delvenne, P., et al. (2015). Monoclonal antibodies against GARP/TGF-beta1 complexes inhibit the immunosuppressive activity of human regulatory T cells in vivo. Science Translational Medicine 7, 284ra256.
de Streel G, Bertrand C, Chalon N, et al. Selective inhibition of TGF-beta1 produced by GARP-expressing Tregs overcomes resistance to PD-1/PD-L1 blockade in cancer. Nat Commun 2020; 11(1):4545. doi: 10.1038/s41467-020-17811-3
Derynck R. Turley S J, Akhurst R J. TGFbeta biology in cancer progression and immunotherapy. Nat Rev Clin Oncol. 2021; 18(1):9-34.
Derynek R, Turley S J, Akhurst R J. TGFbeta biology in cancer progression and immunotherapy. Nat Rev Clin Oncol 2021; 18(1):9-34. doi: 10.1038/s41571-020-0403-1
Derynck, R., Akhurst, R. J., and Balmain, A. (2001). TGF-beta signaling in tumor suppression and cancer progression. Nature Genetics 29, 117-129.
Edwards, J. P., Fujii, H., Zhou, A. X., Creemers, J., Unutmaz, D., and Shevach, E. M. (2013). Regulation of the expression of GARP/latent TGF-beta1 complexes on mouse T cells and their role in regulatory T cell and Th17 differentiation. Journal of Immunology 190, 5506-5515.
Fehrenbacher L, Spira A, Ballinger M, Kowanetz M, Vansteenkiste J, Mazieres J, Park K, Smith D, Artal-Cortes A, Lewanski C, Braiteh F, Waterkamp D, He P. Zou W, Chen D S, Yi J, Sandler A, Rittmeyer A, Group P S. Atezolizumab versus docetaxel for patients with previously treated non-small-cell lung cancer (POPLAR): a multicentre, open-label, phase 2 randomised controlled trial. Lancet. 2016; 387(10030):1837-46.
Flavell, R. A., Sanjabi, S., Wrzesinski, S. H., and Licona-Limon, P. (2010). The polarization of immune cells in the tumour environment by TGFbeta. Nature Reviews Immunology 10, 554-567.
Francisco L M, Sage P T, Sharpe A H. The PD-1 pathway in tolerance and autoimmunity. Immunol Rev 2010; 236:219-42. doi: 10.1111/j.1600-065X.2010.00923.x
Freeman G J, Long A J, Iwai Y, et al. Engagement of the PD-1 immunoinhibitory receptor by a novel B7 family member leads to negative regulation of lymphocyte activation. J Exp Med 2000; 192 (7):1027-34. doi: 10.1084/jem.192.7.1027
Fu C, Jiang A. Dendritic Cells and CD8 T Cell Immunity in Tumor Microenvironment. Front Immunol. 2018; 9:3059.
Gabriel S S, Tsui C, Chisanga D, et al. Transforming growth factor-beta-regulated mTOR activity preserves cellular metabolism to maintain long-term T cell responses in chronic infection. Immunity 2021; 54(8):1698-714 e5. doi: 10.1016/j.immuni. 2021.06.007
Gabriel S S, Tsui C, Chisanga D, Weber F, Llano-Leon M, Gubser P M, Bartholin L, Souza-Fonseca-Guimaraes F, Huntington N D, Shi W, Utzschneider D T, Kallies A. Transforming growth factor-beta-regulated mTOR activity preserves cellular metabolism to maintain long-term T cell responses in chronic infection. Immunity. 2021; 54(8):1698-714 e5.
Gao H, Cai H, Liu J, Wang X, Zheng P, Devenport M, Xu T, Dou F, Liu Y, Zhou A. Structure of CTLA-4 complexed with a pH-sensitive cancer immunotherapeutic antibody. Cell Discov. 2020; 6(1):79.
Gauthy, E., Cuende, J., Stockis, J., Huygens, C., Lethe, B., Collet, J. F., Bommer, G., Coulie, P. G., and Lucas, S. (2013). GARP is regulated by miRNAs and controls latent TGF-beta1 production by human regulatory T cells. PloS One 8, e76186.
Gomez G, Ramirez C D, Rivera J, et al. TGF-beta 1 inhibits mast cell Fe epsilon RI expression. J Immunol 2005; 174(10):5987-93. doi: 10.4049/jimmunol. 174.10.5987
Gonzalez-Junca A, Driscoll K E, Pellicciotta I, Du S, Lo C H, Roy R, Parry R, Tenvooren I, Marquez D M, Spitzer M H, Barcellos-Hoff M H. Autocrine TGFbeta Is a Survival Factor for Monocytes and Drives Immunosuppressive Lineage Commitment. Cancer Immunol Res. 2019; 7(2):306-20.
Griffith J W, Sokol C L, Luster A D. Chemokines and chemokine receptors: positioning cells for host defense and immunity. Annu Rev Immunol. 2014; 32:659-702.
Gunderson A J, Yamazaki T, McCarty K, et al. TGFbeta suppresses CD8(+) T cell expression of CXCR3 and tumor trafficking. Nat Commun 2020; 11(1):1749. doi: 10.1038/s41467-020-15404-8
Gunderson A J, Yamazaki T, McCarty K, Fox N, Phillips M, Alice A, Blair T, Whiteford M, O'Brien D, Ahmad R, Kiely M X, Hayman A, Crocenzi T, Gough M J, Crittenden M R, Young K H. TGFbeta suppresses CD8(+) Tcell expression of CXCR3 and tumor trafficking. Nat Commun. 2020; 11(1):1749.
Hahn, S. A., Stahl, H. F., Becker, C., Correll, A., Schneider, F. J., Tuettenberg, A., and Jonuleit, H. (2013). Soluble GARP has potent antiinflammatory and immunomodulatory impact on human CD4(+) T cells. Blood 122, 1182-1191.
Hammerl D, Martens J W M, Timmermans M, et al. Spatial immunophenotypes predict response to anti-PD1 treatment and capture distinct paths of T cell evasion in triple negative breast cancer. Nat Commun 2021; 12(1):5668. doi: 10.1038/s41467-021-25962-0
Hashimoto M, Kamphorst A O, Im S J, et al. CD8 T Cell Exhaustion in Chronic Infection and Cancer: Opportunities for Interventions. Annu Rev Med 2018; 69:301-18. doi: 10.1146/annurev-med-012017-043208
Hashimoto M, Kamphorst A O, Im S J, Kissick H T, Pillai R N, Ramalingam S S, Araki K, Ahmed R. CD8 T CellExhaustion in Chronic Infection and Cancer: Opportunities for Interventions. Annu Rev Med. 2018; 69:301-18.
Hogquist K A, Jameson S C, Heath W R, Howard J L, Bevan M J, Carbone F R. T cell receptor antagonist peptides induce positive selection. Cell. 1994; 76(1):17-27.
Hong, F., Liu, B., Chiosis, G., Gewirth, D. T., and Li, Z. (2013). alpha7 Helix Region of alphaI Domain Is Crucial for Integrin Binding to Endoplasmic Reticulum Chaperone gp96: A POTENTIAL THERAPEUTIC TARGET FOR CANCER METASTASIS. The Journal of Biological Chemistry 288, 18243-18248.
Hori S, Nomura T, Sakaguchi S. Control of regulatory T cell development by the transcription factor Foxp3.Science. 2003; 299(5609):1057-61.
Hori S, Nomura T, Sakaguchi S. Control of regulatory T cell development by the transcription factor Foxp3. Science 2003; 299(5609):1057-61. doi: 10.1126/science. 1079490
Howley, B. V., Hussey, G. S., Link, L. A., and Howe, P. H. (2015). Translational regulation of inhibin betaA by TGFbeta via the RNA-binding protein hnRNP E1 enhances the invasiveness of epithelial-to-mesenchymal transitioned cells. Oncogene.
Huang Y, Wange R L. T cell receptor signaling: beyond complex complexes. J Biol Chem. 2004; 279(28):28827-30.
Hussey, G. S., Chaudhury, A., Dawson, A. E., Lindner, D. J., Knudsen, C. R., Wilce, M. C., Merrick, W. C., and Howe, P. H. (2011). Identification of an mRNP complex regulating tumorigenesis at the translational elongation step. Molecular Cell 41, 419-431.
Huygens, C., Lienart, S., Dedobbeleer, O., Stockis, J., Gauthy, E., Coulie, P. G., and Lucas, S. (2015). Lysosomal-associated Transmembrane Protein 4B (LAPTM4B) Decreases Transforming Growth Factor beta1 (TGF-beta1) Production in Human Regulatory T Cells. Journal of Biological Chemistry 290, 20105-20116.
Jackson E L, Willis N, Mercer K, Bronson R T, Crowley D, Montoya R, Jacks T, Tuveson D A. Analysis of lung tumor initiation and progression using conditional expression of oncogenic K-ras. Genes Dev. 2001; 15(24):3243-8.
Jie H B, Schuler P J, Lee S C, et al. CTLA-4(+) Regulatory T Cells Increased in Cetuximab-Treated Head and Neck Cancer Patients Suppress NK Cell Cytotoxicity and Correlate with Poor Prognosis. Cancer Res 2015, 75(11):2200-10. doi: 10.1158/0008-5472 CAN-14-2788
Johnson C B, Win S Y. Combination therapy with PD-1/PD-L 1 blockade: An overview of ongoing clinical trials. Oncoimmunology. 2018; 7(4): e1408744.
Johnson L, Mercer K, Greenbaum D, Bronson R T, Crowley D, Tuveson D A, Jacks T. Somatic activation of the K-ras oncogene causes early onset lung cancer in mice. Nature. 2001; 410(6832):1111-6.
Kakarla S, Song X T, Gottschalk S. Cancer-associated fibroblasts as targets for immunotherapy. Immunotherapy 2012; 4(11):1129-38. doi: 10.2217/imt.12.112
Kalbasi A, Ribas A. Tumour-intrinsic resistance to immune checkpoint blockade. Nat Rev Immunol 2020; 20(1):25-39. doi: 10.1038/s41577-019-0218-4
Kehrl, J. H., Wakefield, L. M., Roberts, A. B., Jakowlew, S., Alvarez-Mon, M., Derynck, R., Sporn, M. B., and Fauci, A. S. (1986). Production of transforming growth factor beta by human T lymphocytes and its potential role in the regulation of T cell growth. The Journal of Experimental Medicine 163, 1037-1050.
Khan M, Arooj S, Wang H. NK Cell-Based Immune Checkpoint Inhibition. Front Immunol 2020; 11:167. doi: 10.3389/fimmu.2020.00167
Khan O, Giles J R, McDonald S, Manne S, Ngiow S F, Patel K P, Werner M T, Huang A C, Alexander K A, Wu J E, Attanasio J, Yan P. George S M, Bengsch B, Staupe R P, Donahue G, Xu W, Amaravadi R K, Xu X, Karakousis G C, Mitchell T C, Schuchter L M, Kaye J, Berger S L, Wherry E J. TOX transcriptionally and epigenetically programs CD8(+) T cell exhaustion. Nature. 2019; 571(7764):211-8.
Kopp, H. G., Placke, T., and Salih, H. R. (2009). Platelet-derived transforming growth factor-beta down-regulates NKG2D thereby inhibiting natural killer cell antitumor reactivity. Cancer Research 69, 7775-7783.
Kurtulus S, Madi A, Escobar G, Klapholz M, Nyman J, Christian E, Pawlak M, Dionne D, Xia J, Rozenblatt-Rosen O, Kuchroo V K, Regev A, Anderson AC. Checkpoint Blockade Immunotherapy Induces Dynamic Changes in PD-1(−) CD8(+) Tumor-Infiltrating T Cells. Immunity. 2019; 50(1):181-94 e6.
Kwon H, Chung D, Keneko S, Li A, Zhou L, Riesenberg B, Song N-J, Sundi D, Li X, Li Z. Distinct CD8+ T cell programming in the tumor 1 microenvironment contributes to sex bias in bladder cancer outcome. BioRxiv. 2020; 2020.04.13.039735:
Kwon H, Schafer J M, Song N J, et al. Androgen conspires with the CD8 (+) T cell exhaustion program and contributes to sex bias in cancer. Sci Immumol 2022; 7(73): eabq2630. doi: 10.1126/sciimmunol.abq2630
Lan Y, Zhang D, Xu C, et al. Enhanced preclinical antitumor activity of M7824, a bifunctional fusion protein simultaneously targeting PD-L1 and TGF-beta. Sei Transl Med 2018; 10(424) doi: 10.1126/scitranslmed.aan5488
Li H Y, McSharry M, Bullock B, Nguyen T T, Kwak J, Poczobutt J M, Sippel T R, Heasley L E, Weiser-Evans M C, Clambey E T, Nemenoff R A. The Tumor Microenvironment Regulates Sensitivity of Murine Lung Tumorsto PD-1/PD-L1 Antibody Blockade. Cancer Immunol Res. 2017; 5(9):767-77.
Li M O, Flavell R A. TGF-beta: a master of all T cell trades. Cell 2008; 134(3):392-404. doi: S0092-8674 (08) 00945-8 [pii]
Li T, Fu J, Zeng Z, et al. TIMER2.0 for analysis of tumor-infiltrating immune cells. Nucleic Acids Res 2020; 48(W1): W509-W14. doi: 10.1093/nar/gkaa407
Li, M. O., and Flavell, R. A. (2008). TGF-beta: a master of all T cell trades. Cell 134, 392-404.
Li, Y., Kim, B. G., Qian, S., Letterio, J. J., Fung, J. J., Lu, L., and Lin, F. (2015). Hepatic Stellate Cells Inhibit T Cells through Active TGF-beta1 from a Cell Surface-Bound Latent TGF-beta 1/GARP Complex. Journal of Immunology 195, 2648-2656.
Lienart S, Merceron R, Vanderaa C, et al. Structural basis of latent TGF-beta1 presentation and activation by GARP on human regulatory T cells. Science 2018; 362(6417):952-56. doi: 10.1126/science.aau2909
Lienart S, Merceron R, Vanderaa C, Lambert F, Colau D, Stockis J, van der Woning B, De Haard H, Saunders M, Coulie P G, Savvides S N, Lucas S. Structural basis of latent TGF-beta1 presentation and activation by GARP on human regulatory T cells. Science. 2018; 362(6417):952-6.
Lin J T, Martin S L, Xia L, Gorham J D. TGF-beta 1 uses distinct mechanisms to inhibit IFN-gamma expression in CD4+ T cells at priming and at recall: differential involvement of Stat4 and T-bet. J Immunol. 2005; 174 (10):5950-8.
Lind H, Gameiro S R, Jochems C, et al. Dual targeting of TGF-beta and PD-L1 via a bifunctional anti-PD-L1/TGF-betaRII agent: status of preclinical and clinical advances. J Immunother Cancer 2020; 8(1) doi: 10.1136/jite-2019-000433
Lines J L, Pantazi E, Mak J, et al. VISTA is an immune checkpoint molecule for human T cells. Cancer Res 2014; 74(7):1924-32. doi: 10.1158/0008-5472.CAN-13-1504
Liu S, Ren J, Ten Dijke P. Targeting TGFbeta signal transduction for cancer therapy. Signal Transduct Target Ther. 2021; 6(1):8.
Liu Y, Zheng P. Preserving the CTLA-4 Checkpoint for Safer and More Effective Cancer Immunotherapy. Trends Pharmacol Sci. 2020; 41(1):4-12.
Lyons, R. M., Gentry, L. E., Purchio, A. F., and Moses, H. L. (1990). Mechanism of activation of latent recombinant transforming growth factor beta 1 by plasmin. J Cell Biol 110, 1361-1367.
Ma A, Sun M, McDermaid A, Liu B, Ma Q. MetaQUBIC: a computational pipeline for gene-level functional profiling of metagenome and metatranscriptome. Bioinformatics. 2019; 35(21):4474-7.
Ma A, Wang C, Chang Y, Brennan F H, McDermaid A, Liu B, Zhang C, Popovich P G, Ma Q. IRIS3: integrated cell-type-specific regulon inference server from single-cell RNA-Seq. Nucleic Acids Res. 2020.
Macaulay I C, Tijssen M R, Thijssen-Timmer D C, Gusnanto A, Steward M, Burns P, Langford C F, Ellis P D, Dudbridge F, Zwaginga J J, Watkins N A, van der Schoot C E, Ouwehand W H. Comparative gene expression profiling of in vitro differentiated megakaryocytes and erythroblasts identifies novel activatory and inhibitory platelet membrane proteins. Blood. 2007; 109(8):3260-9.
Mann T H, Kaech S M. Tick-TOX, it's time for T cell exhaustion. Nat Immunol. 2019; 20(9):1092-4.
Mariathasan S, et al. TGFbeta attenuates tumour response to PD-L1 blockade by contributing to exclusion of T cells. Nature. 2018; 554(7693):544-8.
Mariathasan S, Turley S J, Nickles D, et al. TGFbeta attenuates tumour response to PD-L1 blockade by contributing to exclusion of T cells. Nature 2018; 554(7693):544-48. doi: 10.1038/nature25501
Mariathasan S, Turley S J, Nickles D, et al. TGFbeta attenuates tumour response to PD-L1 blockade by contributing to exclusion of T cells. Nature 2018; 554(7693):544-48. doi: 10.1038/nature25501
Massague, J. (2008). TGFbeta in Cancer Cell 134, 215-230.
McLane L M, Abdel-Hakeem M S, Wherry E J. CD8 T Cell Exhaustion During Chronic Viral Infection and Cancer. Annu Rev Immunol. 2019; 37:457-95.
Metelli A, Salem M, Wallace C H, Wu B X, Li A, Li X, Li Z. Immunoregulatory functions and the therapeutic implications of GARP-TGF-beta in inflammation and cancer. J Hematol Oncol. 2018; 11 (1):24.
Metelli A, Wu B X, Fugle C W, et al. Surface Expression of TGFbeta Docking Receptor GARP Promotes Oncogenesis and Immune Tolerance in Breast Cancer. Cancer Res 2016; 76(24):7106-17. doi: 10.1158/0008-5472.CAN-16-1456
Metelli A, Wu B X, Fugle C W, et al. Surface Expression of TGFbeta Docking Receptor GARP Promotes Oncogenesis and Immune Tolerance in Breast Cancer. Cancer Res 2016; 76(24):7106-17. doi: 10.1158/0008-5472.CAN-16-1456
Metelli A, Wu B X, Fugle C W, Rachidi S, Sun S, Zhang Y, Wu J, Tomlinson S, Howe P H, Yang Y, Garrett-Mayer E, Liu B, Li Z. Surface Expression of TGFbeta Docking Receptor GARP Promotes Oncogenesis and Immune Tolerance in Breast Cancer. Cancer Res. 2016; 76(24):7106-17.
Metelli A, Wu B X, Riesenberg B, et al. Thrombin contributes to cancer immune evasion via proteolysis of platelet-bound GARP to activate LTGF-beta. Sci Transl Med 2020; 12(525) doi: 10.1126/scitranslmed.aay4860
Metelli A, Wu B X, Riesenberg B, Guglietta S, Huck J D, Mills C, Li A, Rachidi S, Krieg C, Rubinstein M P, Gewirth D T, Sun S, Lilly M B, Wahlquist A H, Carbone D P, Yang Y, Liu B, Li Z. Thrombin contributes to cancer immune evasion via proteolysis of platelet-bound GARP to activate LTGF-beta. Sci Transl Med. 2020; 12(525).
Millar D G, Garza K M, Odermatt B, Elford A R, Ono N, Li Z, Ohashi PS. Hsp70 promotes antigen-presenting cell function and converts T-cell tolerance to autoimmunity in vivo. Nat Med. 2003; 9(12):1469-76.
Miller B C, et al. Subsets of exhausted CD8(+) T cells differentially mediate tumor control and respond to checkpoint blockade. Nat Immunol. 2019; 20(3):326-36.
Mohammad H P, et al. A DNA Hypomethylation Signature Predicts Antitumor Activity of LSD1 Inhibitors in SCLC. Cancer Cell. 2015; 28(1):57-69
Mohammed J, Beura L K, Bobr A, Astry B, Chicoine B, Kashem S W, Welty N E, Igyarto B Z, Wijeyesinghe S, Thompson E A, Matte C, Bartholin L, Kaplan A, Sheppard D, Bridges A G, Shlomchik W D, Masopust D, Kaplan D H. Stromal cells control the epithelial residence of DCs and memory T cells by regulated activation of TGF-beta. Nat Immunol. 2016; 17(4):414-21.
Morad G, Helmink B A, Sharma P, et al. Hallmarks of response, resistance, and toxicity to immune checkpoint blockade. Cell 2021; 184(21):5309-37. doi: 10.1016/j.cell.2021.09.020 [published Online First: 2021 Oct. 9]
Moreau J M, Velegraki M, Bolyard C, et al. Transforming growth factor-betal in regulatory T cell biology Sci Immunol 2022; 7(69): eabi4613. doi: 10.1126/sciimmunol.abi4613
Muranski, P., Boni, A., Antony, P. A., Cassard, L., Irvine, K. R., Kaiser, A., Paulos, C. M., Palmer, D. C., Touloukian, C. E., Ptak, K., et al. (2008). Tumor-specific Th17-polarized cells eradicate large established melanoma. Blood 112, 362-373.
Nanda R, Chow L Q, Dees E C, Berger R, Gupta S, Geva R, Pusztai L, Pathiraja K, Aktan G, Cheng J D, Karantza V, Buisseret L. Pembrolizumab in Patients With Advanced Triple-Negative Breast Cancer: PhaseIb KEYNOTE-012 Study. J Clin Oncol. 2016; 34(21):2460-7.
Nasrallah R, Imianowski C J, Bossini-Castillo L, Grant F M, Dogan M, Placek L, Kozhaya L, Kuo P, Sadiyah F, Whiteside S K, Mumbach M R, Glinos D, Vardaka P, Whyte C E, Lozano T, Fujita T, Fujii H, Liston A, Andrews S, Cozzani A, Yang J, Mitra S, Lugli E, Chang H Y, Unutmaz D, Trynka G, Roychoudhuri R. A distal enhancer at risk locus 11q13.5 promotes suppression of colitis by Treg cells Nature. 2020.
Niu J, Yue W, Le-Le Z, et al. Mesenchymal stem cells inhibit T cell activation by releasing TGF-beta1 from TGF-beta1/GARP complex. Oncotarget 2017; 8(59):99784-800. doi: 10.18632/oncotarget.21549
Nousbeck J, Irvine A D. Atopic Dermatitis According to GARP: New Mechanistic Insights in Disease Pathogenesis. J Invest Dermatol. 2016; 136(12):2340-1.
Oft, M., Heider, K. H., and Beug, H. (1998). TGFbeta signaling is necessary for carcinoma cell invasiveness and metastasis. Curr Biol 8, 1243-1252.
Oh S A, Li M O. TGF-beta: guardian of T cell function. J Immunol 2013; 191(8):3973-9. doi: 10.4049/jimmunol. 1301843
Ollendorff V, Noguchi T, deLapeyriere O, Birnbaum D. The GARP gene encodes a new member of the family of leucine-rich repeat-containing proteins. Cell Growth Differ. 1994; 5(2):213-9.
Ollendorff, V., Noguchi, T., deLapeyriere, O., and Birnbaum, D. (1994). The GARP gene encodes a new member of the family of leucine-rich repeat-containing proteins. Cell growth & differentiation: the molecular biology journal of the American Association for Cancer Research 5, 213-219.
O'Sullivan J A, Zloza A, Kohlhapp F J, Moore T V, Lacek A T, Dulin N O, Guevara-Patino J A. Priming with very low-affinity peptide ligands gives rise to CD8(+) T-cell effectors with enhanced function but with greater susceptibility to transforming growth factor (TGF) beta-mediated suppression. Cancer Immunol Immunother. 2011; 60(11):1543-51.
Overwijk, W. W., Theoret, M. R., Finkelstein, S. E., Surman, D. R., de Jong, L. A., Vyth-Dreese, F. A., Dellemijn, T. A., Antony, P. A., Spiess, P. J., Palmer, D. C., et al. (2003). Tumor regression and autoimmunity after reversal of a functionally tolerant state of self-reactive CD8+ T cells. The Journal of Experimental Medicine 198, 569-580.
Padua, D., Zhang, X. H., Wang, Q., Nadal, C., Gerald, W. L., Gomis, R. R., and Massague, J. (2008). TGFbeta primes breast tumors for lung metastasis seeding through angiopoietin-like 4. Cell 133, 66-77.
Parichatikanond W, Luangmonkong T, Mangmool S, et al. Therapeutic Targets for the Treatment of Cardiac Fibrosis and Cancer: Focusing on TGF-beta Signaling. Front Cardiovasc Med 2020; 7:34. doi: 10.3389/fcvm.2020.00034
Pender A, Titmuss E, Pleasance E D, et al. Genome and Transcriptome Biomarkers of Response to Immune Checkpoint Inhibitors in Advanced Solid Tumors. Clin Cancer Res 2021; 27(1):202-12. doi: 10.1158/1078-0432.CCR-20-1163
Peng L S, Zhang J Y, Teng Y S, Zhao Y L, Wang T T, Mao F Y, Lv Y P, Cheng P, Li W H, Chen N, Duan M, Chen W, Guo G, Zou Q M, Zhuang Y. Tumor-Associated Monocytes/Macrophages Impair NK-Cell Function via TGFbeta1 in Human Gastric Cancer. Cancer Immunol Res. 2017; 5(3):248-56.
Pertovaara, L., Kaipainen, A., Mustonen, T., Orpana, A., Ferrara, N., Saksela, O, and Alitalo, K. (1994). Vascular endothelial growth factor is induced in response to transforming growth factor-beta in fibroblastic and epithelial cells. J Biol Chem 269, 6271-6274.
Petty A J, Dai R, Lapalombella R, Baiocchi R A, Benson D M, Li Z, Huang X, Yang Y. Hedgehog-induced PD-L1 on tumor-associated macrophages is critical for suppression of tumor-infiltrating CD8⁺ T cell function. JCI Insight. 2021; 6 (6).
Petty A J, Li A, Wang X, Dai R, Heyman B. Hsu D, Huang X, Yang Y. Hedgehog signaling promotes tumor-associated macrophage polarization to suppress intratumoral CD8⁺ T cell recruitment. J Clin Invest. 2019; 129 (12):5151-62.

Pulaski, B. A., and Ostrand-Rosenberg, S. (2001). Mouse 4T1 breast tumor model. Current Protocols in Immunology, ed. John E Coligan et al. Chapter 20, Unit 20 22.
Rachidi S, Metelli A, Riesenberg B, et al. Platelets subvert T cell immunity against cancer via GARP-TGFbeta axis. Sci Immunol 2017; 2(11) doi: 10.1126/sciimmunol.aai7911
Rachidi S, Metelli A, Riesenberg B, Wu B X, Nelson M H, Wallace C, Paulos C M, Rubinstein M P, Garrett-Mayer E, Hennig M, Bearden D W, Yang Y, Liu B, Li Z. Platelets subvert T cell immunity against cancer via GARP-TGFbeta axis. Sci Immunol. 2017; 2(11).
Randow, F., and Seed, B. (2001). Endoplasmic reticulum chaperone gp96 is required for innate immunity but not cell viability. Nature Cell Biology 3, 891-896.
Ravi R, Noonan K A, Pham V, et al. Bifunctional immune checkpoint-targeted antibody-ligand traps that simultaneously disable TGFbeta enhance the efficacy of cancer immunotherapy. Nat Commun 2018; 9(1):741. doi: 10.1038/s41467-017-02696-6
Richter M, Yumul R, Saydaminova K, Wang H, Gough M, Baldessari A, Cattaneo R, Lee F, Wang C H, Jang H, Astier A, Gopal A, Carter D, Lieber A. Preclinical safety, pharmacokinetics, pharmacodynamics, and biodistribution studies with Ad35K++ protein: a novel rituximab cotherapeutic. Mol Ther Methods Clin Dev. 2016; 5:16013.
Riesenberg B P, Ansa-Addo E A, Gutierrez J, Timmers C D, Liu B, Li Z. Cutting Edge: Targeting Thrombocytes to Rewire Anticancer Immunity in the Tumor Microenvironment and Potentiate Efficacy of PD-1 Blockade. J Immunol. 2019; 203(5):1105-10.
Roberts, A. B., Sporn, M. B., Assoian, R. K., Smith, J. M., Roche, N. S., Wakefield, L. M., Heine, U. I., Liotta, L. A., Falanga, V., Kebrl, J. H., et al. (1986). Transforming growth factor type beta: rapid induction of fibrosis and angiogenesis in vivo and stimulation of collagen formation in vitro. PNAS USA 83, 4167-4171.
Rubinstein, M. P., Su, E. W., Suriano, S., Cloud, C. A., Andrijauskaite, K., Kesarwani, P., Schwartz, K. M., Williams, K. M., Johnson, C. B., Li, M., et al. (2015). Interleukin-12 enhances the function and anti-tumor activity in murine and human CD8(+) T cells. Cancer Immunology, Immunotherapy: CII 64, 539-549.
Saeidi A, Zandi K, Cheok Y Y, Saeidi H, Wong W F, Lee C Y Q, Cheong H C, Yong Y K, Larsson M, Shankar E M. T-Cell Exhaustion in Chronic Infections: Reversing the State of Exhaustion and Reinvigorating Optimal Protective Immune Responses. Front Immunol. 2018; 9:2569.
Salem M, Wallace C, Velegraki M, et al. GARP Dampens Cancer Immunity by Sustaining Function and Accumulation of Regulatory T Cells in the Colon. Cancer Res 2019; 79(6):1178-90. doi: 10.1158/0008-5472.CAN-18-2623
Salem M, Wallace C, Velegraki M, Li A, Ansa-Addo E, Metelli A, Kwon H, Riesenberg B, Wu B, Zhang Y, Guglietta S, Sun S, Liu B, Li Z. GARP Dampens Cancer Immunity by Sustaining Function and Accumulation of Regulatory T Cells in the Colon. Cancer Res. 2019; 79(6):1178-90.
Sanmamed M F, Chen L. A Paradigm Shift in Cancer Immunotherapy: From Enhancement to Normalization. Cell. 2018; 175(2):313-26.
Sanmamed M F, Chen L. A Paradigm Shift in Cancer Immunotherapy: From Enhancement to Normalization. Cell 2019; 176(3):677. doi: 10.1016/j.cell.2019.01.008
Sato, Y., and Rifkin, D. B. (1989). Inhibition of endothelial cell movement by pericytes and smooth muscle cells: activation of a latent transforming growth factor-beta 1-like molecule by plasmin during co-culture. J Cell Biol 109, 309-315.
Scott A C, Dundar F, Zumbo P, et al. TOX is a critical regulator of tumour-specific T cell differentiation. Nature 2019; 571(7764):270-74. doi: 10.1038/s41586-019-1324-y
Sekine T, Perez-Potti A, Nguyen S, Gorin J B, Wu V H, Gostick E, Llewellyn-Lacey S, Hammer Q, Falck-Jones S, Vangeti S, Yu M, Smed-Sorensen A, Gaballa A, Uhlin M, Sandberg J K, Brander C, Nowak P, Goepfert P A, Price D A, Betts M R, Buggert M. TOX is expressed by exhausted and polyfunctional human effector memory CD8(+) T cells. Sci Immunol. 2020; 5(49).
Seo H, Chen J, Gonzalez-Avalos E, Samaniego-Castruita D, Das A, Wang Y H, Lopez-Moyado I F, Georges R O, Zhang W, Onodera A, Wu C J, Lu L F, Hogan P G, Bhandoola A, Rao A. TOX and TOX2 transcription factors cooperate with NR4A transcription factors to impose CD8(+) T cell exhaustion. Proc Natl Acad Sci USA. 2019; 116(25):12410-5.
Shafer-Weaver K A, Anderson M J, Stagliano K, Malyguine A, Greenberg N M, Hurwitz A A. Cutting Edge: Tumor-specific CD8⁺ T cells infiltrating prostatic tumors are induced to become suppressor cells. J Immunol. 2009; 183(8):4848-52.
Sharma P, Allison J P. Immune checkpoint targeting in cancer therapy: toward combination strategies with curative potential. Cell. 2015; 161(2):205-14.
Sharma P, Allison J P. The future of immune checkpoint therapy. Science. 2015; 348(6230):56-61
Sharpe A H, Pauken K E. The diverse functions of the PD1 inhibitory pathway. Nat Rev Immmol 2018; 18(3):153-67. doi: 10.1038/nri.2017.108 [published Online First: 2017 Oct. 11]
Sheng W, Liu Y, Chakraborty D, Debo B, Shi Y. Simultaneous Inhibition of LSD1 and TGFbeta Enables Eradication of Poorly Immunogenic Tumors with Anti-PD-1 Treatment. Cancer Discov. 2021; 11(8):1970-81.Epub 2021 Mar. 11. doi: 10.1158/2159-8290.CD-20-0017. PubMed PMID: 33687985.
Siegel, P. M., Shu, W., Cardiff, R. D., Muller, W. J., and Massague, J. (2003). Transforming growth factor beta signaling impairs Neu-induced mammary tumorigenesis while promoting pulmonary metastasis. PNAS USA 100, 8430-8435.
Singer M, Wang C, Cong L, Marjanovic N D, Kowalczyk M S, Zhang H, Nyman J, Sakuishi K, Kurtulus S, Gennert D, Xia J, Kwon J Y H, Nevin J, Herbst R H, Yanai I, Rozenblatt-Rosen O, Kuchroo V K, Regev A, Anderson A C. A Distinct Gene Module for Dysfunction Uncoupled from Activation in Tumor-Infiltrating T Cells. Cell. 2016; 166(6):1500-11 e9.
Song N J, Allen C, Vilgelm A E, et al. Treatment with soluble CD24 attenuates COVID-19-associated systemic immunopathology. J Hematol Oncol 2022; 15(1):5. doi: 10.1186/s13045-021-01222-y
Stockis, J., Colau, D., Coulie, P. G., and Lucas, S. (2009). Membrane protein GARP is a receptor for latent TGF-beta on the surface of activated human Treg. European Journal of Immunology 39, 3315-3322.
Strauss L, Mahmoud M A A, Weaver J D, et al. Targeted deletion of PD-1 in myeloid cells induces antitumor immunity. Sci Immunol 2020; 5(43) doi: 10.1126/sciimmunol.aay 1863
Subramanian A, Tamayo P, Mootha V K, et al. Gene set enrichment analysis: a knowledge-based approach for interpreting genome-wide expression profiles. Proc Natl Acad Sci USA 2005; 102(43):15545-50. doi: 10.1073/pnas.0506580102
Sunderkotter, C., Goebeler, M., Schulze-Osthoff, K., Bhardwaj, R., and Sorg, C. (1991). Macrophage-derived angiogenesis factors. Pharmacol Ther 51, 195-216.
Szepetowski P, Ollendorff V, Grosgeorge J, et al. DNA amplification at 11q13.5-q14 in human breast cancer. Oncogene 1992; 7(12):2513-7
Szepetowski, P., Ollendorff, V., Grosgeorge, J., Courseaux, A., Bimbaum, D., Theillet, C., and Gaudray, P. (1992). DNA amplification at 11q13.5-q14 in human breast cancer. Oncogene 7, 2513-2517.
Tanaka A, Sakaguchi S. Regulatory T cells in cancer immunotherapy. Cell Res. 2017; 27(1):109-18.
Tauriello D V F, Palomo-Ponce S, Stork D, et al. TGFbeta drives immune evasion in genetically reconstituted colon cancer metastasis. Nature 2018; 554(7693):538-43. doi: 10.1038/nature25492
Terabe M, Robertson F C, Clark K, De Ravin E, Bloom A, Venzon D J, Kato S, Mirza A, Berzofsky J A. Blockade of only TGF-beta 1 and 2 is sufficient to enhance the efficacy of vaccine and PD-1 checkpoint blockade immunotherapy. Oncoimmunology. 2017; 6(5):e1308616.
Tesseur, I., Zou, K., Berber, E., Zhang, H., and Wyss-Coray, T. (2006). Highly sensitive and specific bioassay for measuring bioactive TGF-beta. BMC Cell Biology 7, 15.
Thomas D A, Massague J. TGF-beta directly targets cytotoxic T cell functions during tumor evasion of immune surveillance. Cancer Cell. 2005; 8(5):369-80.
Thorsson V, Gibbs D L, Brown S D, et al. The Immune Landscape of Cancer. Immunity 2019; 51(2):411-12. doi: 10.1016/j.immuni.2019.08.004
Tone Y, Furuuchi K, Kojima Y, Tykocinski M L, Greene M I, Tone M. Smad3 and NFAT cooperate to induce Foxp3 expression through its enhancer. Nat Immunol. 2008; 9(2):194-202.
Tran, D. Q. (2012). TGF-beta: the sword, the wand, and the shield of FOXP3(+) regulatory T cells. J Mol Cell Biol 4, 29-37.
Tran, D. Q., Andersson, J., Wang, R., Ramsey, H., Unutmaz, D., and Shevach, E. M. (2009). GARP (LRRC32) is essential for the surface expression of latent TGF-beta on platelets and activated FOXP3+ regulatory T cells. PNAS USA 106, 13445-13450.
Tu E, Chia C P Z, Chen W, et al. T Cell Receptor-Regulated TGF-beta Type I Receptor Expression Determines T Cell Quiescence and Activation. Immunity 2018; 48(4):745-59 e6. doi: 10.1016/j.immuni.2018.03.025
Wallace C H, Wu B X, Salem M, Ansa-Addo E A, Metelli A, Sun S, Gilkeson G, Shlomchik M J, Liu B, Li Z. B lymphocytes confer immune tolerance via cell surface GARP-TGF-beta complex. JCI Insight. 2018; 3(7).
Wallace C H, Wu B X, Salem M, et al. B lymphocytes confer immune tolerance via cell surface GARP-TGF-beta complex. JCI Insight 2018; 3(7) doi: 10.1172/jci.insight.99863
Wang P, Huang B, Gao Y, Yang J, Liang Z, Zhang N, Fu X, Li L. CD103(+)CD8(+) T lymphocytes in non-small cell lung cancer are phenotypically and functionally primed to respond to PD-1 blockade. Cell Immunol. 2018; 325:48-55.
Wang X, He Q, Shen H, Xia A, Tian W, Yu W, Sun B. TOX promotes the exhaustion of antitumor CD8(+) Tcells by preventing PDI degradation in hepatocellular carcinoma. J Hepatol. 2019; 71(4):731-41.
Wang, R., Kozhaya, L., Mercer, F., Khaitan, A., Fujii, H., and Unutmaz, D. (2009). Expression of GARP selectively identifies activated human FOXP3+ regulatory T cells. PNAS USA 106, 13439-13444.
Wang, R., Wan, Q., Kozhaya, L., Fujii, H., and Unutmaz, D. (2008). Identification of a regulatory T cell specific cell surface molecule that mediates suppressive signals and induces Foxp3 expression. PloS One 3, e2705.
Wang, R., Zhu, J., Dong, X., Shi, M., Lu, C., and Springer, T. A. (2012). GARP regulates the bioavailability and activation of TGFbeta. Molecular Biology of the Cell 23, 1129-1139.
Wherry E J, Kurachi M. Molecular and cellular insights into T cell exhaustion. Nat Rev Immunol. 2015; 15(8):486-99. Epub 2015 Jul. 25. doi: 10.1038/nri3862
Woicka-Kolejwa K, Jerzynska J, Majak P, Koniarek A, Stelmach I. Glycoprotein A (GARP) in children who outgrow food allergy. Allergol Immunopathol (Madr). 2020; 48(1):67-72.
Wu B X, Li A, Lei L, Kaneko S, Wallace C, Li X, Li Z. Glycoprotein A repetitions predominant (GARP) positively regulates transforming growth factor (TGF) beta3 and is essential for mouse palatogenesis. J Biol Chem. 2017; 292(44):18091-7.
Wu M, Chen X, Lou J, Zhang S, Zhang X, Huang L, Sun R, Huang P, Wang F, Pan S. TGF-beta1 contributes to CD8⁺ Treg induction through p38 MAPK signaling in ovarian cancer microenvironment. Oncotarget. 2016; 7(28):44534-44.
Wu T, Ji Y, Moseman E A, Xu H C, Manglani M, Kirby M, Anderson S M, Handon R, Kenyon E, Elkahloun A, Wu W, Lang P A, Gattinoni L, McGavern D B, Schwartzberg P L. The TCF1-Bcl6 axis counteracts type I interferon to repress exhaustion and maintain T cell stemness. Sci Immunol. 2016; 1 (6).
Wu, S., Hong, F., Gewirth, D., Guo, B., Liu, B., and Li, Z. (2012). The molecular chaperone gp96/GRP94 interacts with Toll-like receptors and integrins via its C-terminal hydrophobic domain. Journal of Biological Chemistry 287, 6735-6742.
Xie, L., Law, B. K., Aakre, M. E., Edgerton, M., Shyr, Y., Bhowmick, N. A., and Moses, H. L. (2003). Transforming growth factor beta-regulated gene expression in a mouse mammary gland epithelial cell line. Breast Cancer Research: BCR 5, R187-198.
Xu, J., Lamouille, S., and Derynck, R. (2009). TGF-beta-induced epithelial to mesenchymal transition. Cell Research 19, 156-172.
Yang B H, Wang K, Wan S, Liang Y, Yuan X, Dong Y, Cho S, Xu W, Jepsen K, Feng G S, Lu L F, Xue H H, Fu W. TCF1 and LEF1 Control Treg Competitive Survival and Tfr Development to Prevent Autoimmune Diseases. Cell Rep. 2019; 27 (12):3629-45 e6.
Yao C, Sun H W, Lacey N E, Ji Y, Moseman E A, Shih H Y, Heuston E F, Kirby M, Anderson S, Cheng J, Khan O, Handon R, Reilley J, Fioravanti J, Hu J, Gossa S, Wherry E J, Gattinoni L, McGavern D B, O'Shea J J, Schwartzberg P L, Wu T. Single-cell RNA-seq reveals TOX as a key regulator of CD8(+) T cell persistence in chronic infection. Nat Immunol. 2019; 20(7):890-901.
Yu G, Wang L G, Han Y, et al. cluster Profiler: an R package for comparing biological themes among gene clusters. OMICS 2012; 16(5):284-7. doi: 10.1089/omi.2011.0118
Yu, Q., and Stamenkovic, I. (2000). Cell surface-localized matrix metalloproteinase-9 proteolytically activates TGF-beta and promotes tumor invasion and angiogenesis. Genes & Development 14, 163-176.
Zhang F, Wang H, Wang X, Jiang G, Liu H, Zhang G, Wang H, Fang R, Bu X, Cai S, Du J TGF-beta induces M2-like macrophage polarization via SNAIL-mediated suppression of a pro-inflammatory phenotype. Oncotarget. 2016; 7(32):52294-306.
Zhang P, Xiong X, Rolfo C, Du X, Zhang Y, Yang H, Russo A, Devenport M, Zhou P, Liu Y, Zheng P. Mechanism- and Immune Landscape-Based Ranking of Therapeutic Responsiveness of 22 Major Human Cancers to Next Generation Anti-CTLA-4 Antibodies. Cancers (Basel). 2020; 12(2).
Zhang X, Huang H, Yuan J, Sun D, Hou W S, Gordon J, Xiang J. CD4-8-dendritic cells prime CD4+ T regulatory 1 cells to suppress antitumor immunity. J Immunol. 2005; 175(5):2931-7.
Zhang Y, Du X, Liu M, Tang F, Zhang P, Ai C, Fields J K, Sundberg E J, Latinovic O S, Devenport M, Zheng P, Liu Y. Hijacking antibody-induced CTLA-4 lysosomal degradation for safer and more effective cancer immunotherapy. Cell Res. 2019; 29(8):609-27.
Zhang, Y., Wu, B. X., Metelli, A., Thaxton, J. E., Hong, F., Rachidi, S., Ansa-Addo, E., Sun, S., Vasu, C., Yang, Y., et al. (2015). GP96 is a GARP chaperone and controls regulatory T cell functions. Journal of Clinical Investigation 125, 859-869.
Zheng H, Li Z. Cutting edge: cross-presentation of cell-associated antigens to MHC class I molecule is regulated by a major transcription factor for heat shock proteins. J Immunol. 2004; 173(10):5929-33.

Zou W, Wolchok J D, Chen L. PD-L1 (B7-H1) and PD-1 pathway blockade for cancer therapy: Mechanisms, response biomarkers, and combinations. Sci Transl Med 2016; 8(328):328rv4. doi: 10.1126/scitranslmed.aad7118

What is claimed is:

1. An isolated anti-glycoprotein A repetitions predominant (GARP) monoclonal antibody, wherein the antibody specifically binds to GARP and comprises i) a variable heavy chain (VH) complementarity determining region 1 (CDR1), CDR2, and CDR3 as set forth in SEQ ID NO: 1, SEQ ID NO: 2, and SEQ ID NO: 3, respectively and ii) a variable light chain (VL) complementarity determining region 1 (CDR1), CDR2, and CDR3 as set forth in SEQ ID NO: 5, SEQ ID NO: 6, and SEQ ID NO: 7, respectively; or the antibody comprises i) a variable heavy chain (VH) complementarity determining region 1 (CDR1), CDR2, and CDR3 as set forth in SEQ ID NO: 9, SEQ ID NO: 10, and SEQ ID NO: 11, respectively and ii) a variable light chain (VL) complementarity determining region 1 (CDR1), CDR2, and CDR3 as set forth in SEQ ID NO: 13, SEQ ID NO: 14, and SEQ ID NO: 15, respectively.

2. The antibody of claim 1, wherein the anti-GARP antibody comprises i) a variable heavy chain (VH) complementarity determining region 1 (CDR1), CDR2, and CDR3 as set forth in SEQ ID NO: 1, SEQ ID NO: 2, and SEQ ID NO: 3, respectively and ii) a variable light chain (VL) complementarity determining region 1 (CDR1), CDR2, and CDR3 as set forth in SEQ ID NO: 5, SEQ ID NO: 6, and SEQ ID NO: 7, respectively

3. The antibody of claim 2, wherein the antibody comprises a V_{H} domain at least about 80%, 90%, 95%, 98% or 99% identical to the V_{H} domain of the humanized PIIO-1 (huPIIO-1) antibodies as set forth in SEQ ID NO: 18, 19, 20 or 21 and/or a V_{L} domain at least about 80% 90%, 95%, 98% or 99% identical to the V_{L} domain of the huPIIO-1 antibodies as set forth in SEQ ID NO: 22, 23, or 24.

4. The antibody of claim 3, wherein the antibody comprises a V_{H} domain as set forth in SEQ ID NO: 18, 19, 20, or 21 and/or a V_{L} domain as set forth in SEQ ID NO: 22, 23 or 24.

5. The antibody of claim 3 or 4 wherein the antibody comprises a V_{H} domain as set forth in SEQ ID NO: 20 and V_{L} domain as set forth in SEQ ID NO: 23 (VH1VL1), a V_{H} domain as set forth in SEQ ID NO: 20 and V_{L} domain as set forth in SEQ ID NO: 24 (VH1VL2), a V_{H} domain as set forth in SEQ ID NO: 21 and V_{L} domain as set forth in SEQ ID NO: 23 (VH1VL1), SEQ ID NO: 20 and V_{L} domain as set forth in SEQ ID NO: 22 (VH1VL3), a V_{H} domain as set forth in SEQ ID NO: 21 and V_{L} domain as set forth in SEQ ID NO: 24 (VH2VL2), a V_{H} domain as set forth in SEQ ID NO: 21 and V_{L} domain as set forth in SEQ ID NO: 22 (VH2VL3), a V_{H} domain as set forth in SEQ ID NO: 19 and V_{L} domain as set forth in SEQ ID NO: 23 (VH3VL1), a V_{H} domain as set forth in SEQ ID NO: 19 and V_{L} domain as set forth in SEQ ID NO: 24 (VH3VL2), a V_{H} domain as set forth in SEQ ID NO: 19 and V_{L} domain as set forth in SEQ ID NO: 22 (VH3VL3), a V_{H} domain as set forth in SEQ ID NO: 18 and V_{L} domain as set forth in SEQ ID NO: 23 (VH4VL1), a V_{H} domain as set forth in SEQ ID NO: 18 and V_{L} domain as set forth in SEQ ID NO: 24 (VH4VL2), or a V_{H} domain as set forth in SEQ ID NO: 18 and V_{L} domain as set forth in SEQ ID NO: 22 (VH4VL3).

6. The isolated antibody of claim 1, wherein the antibody comprises) a variable heavy chain (VH) complementarity determining region 1 (CDR1), CDR2, and CDR3 as set forth in SEQ ID NO: 9, SEQ ID NO: 10, and SEQ ID NO: 11, respectively and ii) a variable light chain (VL) complementarity determining region 1 (CDR1), CDR2, and CDR3 as set forth in SEQ ID NO: 13, SEQ ID NO: 14, and SEQ ID NO: 15, respectively.

7. The antibody of claim 5, wherein the antibody comprises a V_{H} domain at least about 80% 90%, 95%, 98% or 99% identical to the V_{H} domain of 5c5 (SEQ ID NO: 12) and a V_{L} domain at least about 80% 90%, 95%, 98% or 99% identical to the V_{L} domain of 5c5 (SEQ ID NO: 16).

8. The antibody of claim 7, wherein the antibody comprises a V_{H} domain identical to the V_{H} domain of 5c5 (SEQ ID NO: 12) and a V_{L} domain identical to the V_{L} domain 5c5 (SEQ ID NO: 16).

9. The antibody of any one of claims 1-8, wherein the antibody is recombinant.

10. The antibody of any one of claims 1-9, wherein the antibody is an IgG, IgM, IgA or an antigen binding fragment thereof.

11. The antibody of any one of claims 1-10, wherein the antibody is a Fab′, a F(ab′)2, a F(ab′)3, a monovalent scFv, a bivalent scFv, nanobody, or a single domain antibody.

12. The antibody of any one of claims 1-11, wherein the antibody is a human, humanized antibody or de-immunized antibody.

13. The antibody of any one of claims 1-12, wherein the antibody is fused or conjugated to a platelet binding agent.

14. The antibody of claim 13, wherein the anti-platelet agent is selected from the group consisting of a cyclooxygenase inhibitor, adenosine diphosphate (ADP) inhibitor, phosphodiesterase inhibitor, protease-activated receptor-1 (PAR-1) antagonist, glycoprotein IIB/IIIA inhibitor, adenosine reuptake inhibitor, and thromboxane inhibitor.

15. The antibody of claim 14, wherein the ADP inhibitor is clopidogrel, prasugrel, or ticlopidine.

16. The antibody of any one of claims 1-12, wherein the antibody is conjugated to an imaging agent, a chemotherapeutic agent, a toxin or a radionuclide.

17. An isolated polynucleotide molecule comprising a nucleic acid sequence encoding an antibody of any one of claims 1-16.

18. A composition comprising an antibody of any one of claims 1-16 in a pharmaceutically acceptable carrier.

19. The composition of claim 18, further comprising an anti-cancer agent.

20. The composition of claim 19, wherein the anti-cancer agent comprises an immune checkpoint inhibitor.

21. The composition of claim 20, wherein the immune checkpoint inhibitor comprises an inhibitor of cytotoxic T-lymphocyte-associated protein 4 (CTLA-4), program cell death protein 1 (PD1), programmed death ligand 1 (PD-L1), programmed death ligand 2 (PD-L2), lymphocyte activation gene 3 (LAG-3), B- and T-lymphocyte attenuator (BTLA), B7 homolog 3 (B7H3), B7 homolog 4 (B7H4), T-cell immunoglobulin and mucin domain 3 (Tim-3), killer immunoglobulin-like receptor (KIR), V-domain Ig suppressor of T cell activation (VISTA), and T cell immunoreceptor with Ig and ITIM domains (TIGIT).

22. The composition of claim 21, wherein the immune checkpoint inhibitor is a PD1 inhibitor.

23. The composition of claim 22, wherein the PD1 inhibitor is selected from the group consisting of nivolumab, pembrolizumab, CT-011, BMS 936559, MPDL3280A or AMP-224.

24. A recombinant polypeptide comprising an antibody V_{H} domain comprising CDRs 1, 2, and 3 of the V_{H} domain of the huPIIO-1 antibodies as set forth in SEQ ID NOs: 1, 2, and 3, respectively or CDRs 1, 2, and 3 of the V_{H} domain of 5c5 as set forth in SEQ ID NOs: 9, 10, and 11, respectively.

25. A recombinant polypeptide comprising an antibody V_{L} domain comprising CDRs 1, 2, and 3 of the V_{L} domain of the huPIIO-1 antibodies as set forth in SEQ ID NOs: 5, 6, and 7, respectively or CDRs 1, 2, and 3 of the V_{L} domain of 5c5 as set forth in SEQ ID NOs: 13, 14, and 15, respectively.

26. An isolated polynucleotide molecule comprising a nucleic acid sequence encoding the antibody of any of claims 1-16 or the polypeptide of any of claims 18-23.

27. The isolated polynucleotide molecule of claim 22, wherein the nucleic acid comprises SEQ ID NO: 25, SEQ ID NO: 26, SEQ ID NO: 27, SEQ ID NO: 28, SEQ ID NO: 29, SEQ ID NO: 30, and/or SEQ ID NO: 31.

28. A host cell comprising one or more polynucleotide molecule(s) encoding an antibody of any one of claims 1-16. the recombinant polypeptide of any of claims 18-23, or the isolated nucleic acid of any of claim 17, 26, or 27.

29. The host cell of claim 28, wherein the host cell is a mammalian cell, a yeast cell, a bacterial cell, a ciliate cell or an insect cell.

30. A method for treating a cancer in a subject comprising administering to the subject an effective amount of an antibody of any one of claims 1-16 or the composition of any one of claims 18-23 to the subject.

31. The method of claim 26, wherein the cancer is a breast cancer, lung cancer, head & neck cancer, prostate cancer, esophageal cancer, tracheal cancer, skin cancer, brain cancer, liver cancer, bladder cancer, stomach cancer, pancreatic cancer, ovarian cancer, uterine cancer, cervical cancer, testicular cancer, colon cancer, rectal cancer, a hematological cancer, clear cell kidney cancer, head/neck squamous cell carcinoma, lung squamous cell carcinoma, melanoma, non-small-cell lung cancer (NSCLC), renal cell cancer, small-cell lung cancer (SCLC), triple negative breast cancer, acute lymphoblastic leukemia (ALL), acute myeloid leukemia (AML), chronic lymphocytic leukemia (CLL), chronic myeloid leukemia (CML), diffuse large B-cell lymphoma (DLBCL), follicular lymphoma, Hodgkin's lymphoma (HL), mantle cell lymphoma (MCL), multiple myeloma (MM), myeloid cell leukemia-1 protein (Mcl-1), myelodysplastic syndrome (MDS), non-Hodgkin's lymphoma (NHL), or small lymphocytic lymphoma (SLL).

32. The method of claim 30 or 31, wherein the cancer is a GARP positive cancer.

33. The method of any of claims 30-32, wherein the antibody is administered systemically.

34. The method of any of claims 30-33, wherein the antibody is administered intravenously, intradermally, intratumorally, intramuscularly, intraperitoneally, subcutaneously, or locally.

35. The method of any of claims 30-34, further comprising administering to the subject at an anticancer therapy and/or an anticancer agent to the subject.

36. The method of claim 35, wherein the anticancer agent comprises a TGFβ inhibitor.

37. The method of claim 36, wherein the TGFβ inhibitor is LY2157299, trabedersen, fresolimumab, LY2382770, lucanix, or PF-03446962.

38. The method of any of claims 30-37, further comprising administering to the subject a anti-platelet agent.

39. The method of claim 36, wherein the anti-platelet agent is selected from the group consisting of a cyclooxygenase inhibitor, adenosine diphosphate (ADP) inhibitor, phosphodiesterase inhibitor, protease-activated receptor-1 (PAR-1) antagonist, glycoprotein IIB/IIIA inhibitor, adenosine reuptake inhibitor, and thromboxane inhibitor.

40. The method of claim 39, wherein the ADP inhibitor is clopidogrel, prasugrel, or ticlopidine.

41. The method of claim 35, wherein the anticancer agent comprises an immune checkpoint inhibitor.

42. The method of claim 41, wherein the immune checkpoint inhibitor comprises an inhibitor of cytotoxic T-lymphocyte-associated protein 4 (CTLA-4), program cell death protein 1 (PD1), programmed death ligand 1 (PD-L1), programmed death ligand 2 (PD-L2), lymphocyte activation gene 3 (LAG-3), B- and T-lymphocyte attenuator (BTLA), B7 homolog 3 (B7H3), B7 homolog 4 (B7H4), T-cell immunoglobulin and mucin domain 3 (Tim-3), killer immunoglobulin-like receptor (KIR), V-domain Ig suppressor of T cell activation (VISTA), and T cell immunoreceptor with Ig and ITIM domains (TIGIT).

43. The method of claim 42, wherein the immune checkpoint inhibitor is a PD1 inhibitor.

44. The method of claim 43, wherein the PD-1 binding antagonist is nivolumab, pembrolizumab, CT-011, BMS 936559, MPDL3280A or AMP-224.

45. The method of claim 35, wherein the anticancer therapy is a surgical therapy, chemotherapy, radiation therapy, cryotherapy, hormonal therapy, immunotherapy or cytokine therapy.

46. The method of claim 45, wherein the immunotherapy comprises an adoptive cell transfer therapy.

47. The method of claim 46, wherein the adoptive cell transfer therapy comprises the transfer of T cells, chimeric antigen receptor (CAR) T cells, B cells, Natural Killer (NK) cells, CAR NK cells, CAR macrophage (CARMA), and/or NK T cells.

48. The method of claim 47, wherein the adoptive cell transfer therapy comprises a T cell transfer and wherein the T cells comprise tumor infiltrating lymphocytes (TILs), chimeric antigen receptor (CAR) T cells, CD8⁺ T cells and/or CD4⁺ T cells.

49. The method of any of claim 48, wherein the T cell therapy comprises administration of tumor-specific T cells.

50. The method of any of claim 49, wherein the tumor-specific T cells are engineered to express a T cell receptor (TCR) or chimeric antigen receptor (CAR) receptor having antigenic specificity for a tumor antigen.

51. The method of claim 50, wherein the tumor-antigen is selected from the group consisting of tEGFR, Her2, CD19, CD20, CD22, mesothelin, CEA, CD23, CD24, CD30, CD33, CD38, CD44, EGFR, EGP-2, EGP-4, EPHa2, ErbB2, FBP, MAGE-A1, MUC1, NY-ESO-1, and MART-1.

52. The method of any of claims 47-51, wherein the CAR comprises co-stimulatory molecule endodomains selected from the group consisting of CD28, CD27, 4-IBB, OX40 ICOS, and a combination thereof.

53. The method of any of claims 46-52, wherein the adoptively transferred cells are autologous.

54. The method of any of claims 45-53, wherein the immunotherapy is administered before the anti-platelet agent, simultaneous with the anti-platelet agent, or after the anti-platelet agent.

55. The method of claim 45-49, wherein the immunotherapy and anti-platelet agent are administered simultaneously.

56. The method of any of claims 30-55, further comprising lymphodepletion of the subject prior to administration of the T cell therapy.

57. The method of claim 56, wherein lymphodepletion comprises administration of cyclophosphamide and/or fludarabine.

58. A method for detecting a cancer in a subject comprising obtaining a potentially cancerous tissue sample form a subject and testing the tissue sample for the presence of increased levels of GARP relative to a noncancerous control.

59. The method of claim 58, wherein the GARP is soluble GARP.

60. The method of claim 58 or 59, further comprising testing for the presence of an increased level GARP expressing cells in the sample.

61. The method of any of claims 58-60, wherein the testing comprises contacting the sample with an antibody that binds to GARP.

62. The method of any of claims 58-61, wherein the antibody that binds to GARP is an antibody according to any one of claims 1-16.

63. The method of claim 62, further defined as an in vitro or ex vivo method.

64. A method of stimulating T cells and/or B cells in a subject with a cancer comprising administering to the subject an effective amount of the anti-GARP antibody of any of claims 1-16.

65. The method of claim 64, wherein the T cells are present in a tumor microenvironment

66. The method of claim 64 or 65, wherein the T cells are endogenous tumor infiltrating lymphocytes (TILs)

67. The method of claim 64 or 65, wherein the T cells are TILs or chimeric antigen receptor (CAR) T cells administered to the subject as a component of an immunotherapy.

68. The method of any of claims 64-67, wherein the T cells are CD8 T cells.

69. The method of claim 68, wherein the CD8 T cells are CD25+, CD45RA−+, CD45RO−, and CD127− effector CD8 T cells or CD25−, CD45RA−, CD45RO+, and CD127+ effector memory CD8 T cells.

70. The method of any of claims 64-67, wherein the T cells are CD4 T cells.

71. The method of claim 70, wherein the CD4 T cells are Th1 or Th2 CD4 T cells

72. A method of stimulating adoptively transferred donor T cells in a tumor microenvironment of a subject comprising administering the T cells and an anti-GARP antibody of any of claims 1-16.

73. The method of claim 72, wherein the T cells and the anti-GARP antibody are administered concurrently.

74. The method of claim 72, wherein the anti-GARP antibody is administered to the subject prior to the transfer of donor T cells.

75. The method of claim 72, wherein the anti-GARP antibody is administered to the subject after the transfer of donor T cells.

76. The method of claim 72, wherein the T cells are TILs or chimeric antigen receptor (CAR) T cells administered to the subject as a component of an immunotherapy.

77. A method of inducing T cell or B cell proliferation in a subject with a cancer comprising administering to the subject an effective amount of the anti-GARP antibody of any of claims 1-16.

78. A method of blocking T cell exhaustion of a CD8+ T cell comprising contacting the CD8+ T cell with an effective amount of the anti-GARP antibody of any of claims 1-16.

79. The method of claim 78, wherein the CD8+ T cell is contacted with the anti-GARP antibody ex vivo.

80. The method of claim 78, wherein the CD8+ T cell are located in the tumor microenvironment.

81. A method of inhibiting Tregs in a tumor microenvironment in a subject comprising administering to the subject a therapeutically effective amount of the anti-GARP antibody of any of claims 1-16.

82. A method of blocking GARP-LTGFβ1 complex formation in a cancer comprising contact the cancer with a therapeutically effective amount of the anti-GARP antibody of any of claims 1-16.

83. A method of increasing the efficacy of a immune checkpoint blockade (ICB) therapy in a subject comprising administering to a subject receiving ICB therapy a therapeutically effective amount of the anti-GARP antibody of any of claims 1-16.

84. A method of activating T cells or B cells comprising in a subject with a cancer comprising administering to the subject an effective amount of the anti-GARP antibody of any of claims 1-16.

85. The method of claim 83 or 84 wherein the T cells are CD8 T cells.

86. The method of claim 83 or 84 wherein the T cells are CD4 T cells.

87. The method of any of claims 83-86, wherein the T cells are located in a tumor microenvironment.

88. A method of assessing the sensitivity of a cancer to an immune checkpoint blockade (ICB) therapy comprising obtaining a cancerous tissue sample and assaying the sample for GARP expression; wherein elevated expression of GARP relative to a noncancerous control indicates the cancer is resistant to ICB therapy and low expression of GARP or equivalent expression of GARP relative to a noncancerous control indicates the cancer is sensitive to ICB therapy.

89. A method of making a cancer cell sensitive to immune checkpoint blockade (ICB) therapy comprising contacting an ICB therapy resistant cancer cell with the anti-GARP of any of claims 1-16.
